(19) United States
(12) Patent Application Publication
(10) Pub. No.: US 2026/0308161 A1
(43) Pub. Date: Oct. 8, 2026

(54) CHIMERIC COMPOUNDS USEFUL IN TREATING DISEASES

(71) Applicant: Padarn Therapeutics, Inc., Dover, DE (US)

(72) Inventors: Ruben Abagyan, La Jolla, CA (US); Vladislav Zenonovich Parchinsky, Moscow (RU); Alexander Khvat, San Diego, CA (US); Alexandre Vasilievich Ivachtchenko, Hallandale Beach, FL (US); Nikolay Savchuk, San Diego, CA (US)

(21) Appl. No.: 19/700,272

(22) Filed: Jun. 6, 2026

Related U.S. Application Data
(62) Division of application No. 18/701,216, filed on Oct. 7, 2022.
(60) Provisional application No. 63/255,742, filed on Oct. 14, 2021.

(51) Int. Cl. C07D 487/04; A61K 31/519; A61K 31/55; A61P 35/00; C07D 519/00

(52) U.S. Cl. CPC C07D 487/04; A61K 31/519; A61K 31/55; A61P 35/00; C07D 519/00

(57) ABSTRACT

The present invention is generally directed to dual inhibitors of poly(ADP-ribose) polymerase (PARP) and Wee1 useful in the treatment of diseases and disorders modulated by said enzymes and having the Formula (I):

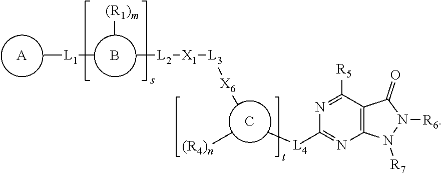

### CROSS-REFERENCE TO RELATED APPLICATIONS

This application is a divisional of U.S. patent application Ser. No. 18/701,216 filed Apr. 12, 2024, entitled “Chimeric Compounds Useful in Treating Diseases”, which is national stage application of International Patent Application No. PCT/US2022/046015 filed Oct. 7, 2022, entitled “Chimeric Compounds Useful in Treating Diseases”, which claims priority to and the benefit of U.S. Provisional Patent Application No. 63/255,742 filed Oct. 14, 2021, and entitled “Chimeric Compounds Useful in Treating Diseases”. The entire disclosure of each of the foregoing applications is incorporated herein by reference for all purposes.

### FIELD OF INVENTION

The present invention is directed to dual inhibitors of poly(ADP-ribose) polymerase (PARP) and Wee1. The inhibitors described herein can be useful in the treatment of diseases or

The present invention is directed to dual inhibitors of poly(ADP-ribose) polymerase (PARP) and Wee1. The inhibitors described herein can be useful in the treatment of diseases or disorders associated with PARP and/or Wee1 enzymes, such as a cancer. In particular, the invention is concerned with compounds and pharmaceutical compositions inhibiting PARP and/or Wee1, methods of treating diseases or disorders associated with PARP and/or Wee1, and methods of synthesizing these compounds.

### BACKGROUND OF THE INVENTION

In the treatment of cancer, single-agent/single-target therapeutics are often ineffective due to the complexity of cancer pathways and the emergence of drug resistance. Currently, 90% of failures in the chemotherapy are during the invasion and metastasis of cancers related to drug resistance. In the chemotherapy, by following the administration of a certain drug, a large number of patient tumor cells become resistant to the drug (Behzad Mansoori, et al., Adv Pharm Bull, 2017, 7 (3), 339-348). So, the drug resistance appears as a serious problem in the field of cancer. While combination therapies, targeting two or more pathways, constitute the mainstays of modern cancer treatment, they have their own drawbacks. Particularly, combination therapies may be hampered by complicated pharmacokinetics, intricate toxicity profiles, undesirable drug-drug interactions, and poor patient compliance. Additionally, the development of combination therapies is both expensive and time-consuming. Accordingly, single-agent, dual inhibitor compounds may be a desirable alternative to single agent and combination cancer therapies.

Poly(ADP-ribose) polymerases (PARP) are a family of enzymes that catalyze the transfer of ADP-ribose from nicotinamide adenine dinucleotide (NAD+) to acceptor proteins. PARP-1, the most abundant member of this family, plays a role in the repair of DNA single-strand breaks (SSBs), which has made it a highly pursued therapeutic target for cancer treatment. Additionally, the discovery of the synthetic lethality between PARP inhibition and BRCA1/2 mutations has suggested a novel strategy for treating patients with BRCA mutant tumors. While PARP inhibitors were initially developed as chemo- or radio-sensitizing agents by inhibiting DNA damage repair and promoting apoptosis of tumor cells, they are also promising monotherapy agents for patients with BRCA1/2 mutations.

Wee1 is a serine/threonine protein kinase that is a critical component of the ataxia-telangiectasia-mutated-and -Rad3-related (ATR)-mediated G2 cell cycle checkpoint control, that prevents entry into mitosis in response to cellular DNA damage. ATR phosphorylates and activates CHK1, which in turn activates Wee1, leading to the selective phosphorylation of cyclin-dependent kinase 1(CDK1) at Tyr5, thereby stabilizing the CDK1-cyclin B complex and halting cell-cycle progression. This process confers a survival advantage by allowing tumor cells time to repair damaged DNA prior to entering mitosis. Inhibition of Wee1 abrogates the G2 checkpoint, forcing cancer cells with DNA damage to enter into unscheduled mitosis and undergo cell death via mitotic catastrophe.

Accordingly, a dual poly(ADP-ribose) polymerase (PARP) and Wee1 inhibitor may produce pronounced synergistic anticancer effects and prevent to developing of cancer by changing pathway (with different mechanism). There is thus a need for therapeutic agents that can inhibit poly(ADP-ribose) polymerase (PARP) and/or Wee1. This invention is intended to fill this unmet need associated with current poly(ADP-ribose) polymerase (PARP) and Wee1 inhibition therapies.

### SUMMARY OF THE INVENTION

In general, this invention relates to compounds comprising three fragments: (1) inhibitor of poly(ADP-ribose) polymerase (PARP); (2) linker that connected two active parts of the molecule and could be indeed the part of one of these active molecules; and (3) inhibitor of Wee1.

A first aspect of the invention relates to compounds of general Formula (A):

$$P - L - W ( A ) ,$$

or pharmaceutically acceptable salts, solvates, prodrugs, or tautomers thereof,
wherein:
P is selected from inhibitors of poly(ADP-ribose) polymerase (PARP) or their derivatives, particularly selected from the group comprising: rucaparib, niraparib, senaparib, veliparib, talazoparib, stenoparib, pamiparib, fluzoparib, and simmiparib;
L is a linker bonding two active parts P and W of the molecule, and structure of linker may comprise structural parts of one or both of active parts P and W, and wherein L is of Formula (L):

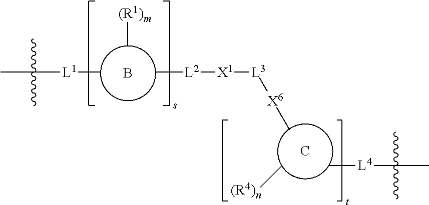

wherein,
Ring B and Ring C are each independently selected from C₃-C₁₀ cycloalkyl, aryl, 3- to 10-membered heterocyclyl, and heteroaryl;
each L¹, L², and L⁴ is independently selected from bond, C₁-C₆ alkylenyl, C₂-C₆ alkenylenyl, C₂-C₆ alkynylenyl, C(O)—, C(O)NR^{L}—, —C(O)O—, —NR^{L}—, —NR^{L}C(O), —NR^{L}C(O)NR^{L}—, —NR^{L}C(O)O—, —O—, —OC(O)—, —OC(O)NR^{L}—, —OC(O)O—, and —S(O)ₒ—, wherein the alkylenyl, alkenylenyl, or alkynylenyl is optionally substituted with one or more substituents independently selected from halogen, —OH, —CN, —NO₂, C₁-C₆ alkyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
L³ is selected from C₁-C₆ alkylenyl, C₂-C₆ alkenylenyl, and C₂-C₆ alkynylenyl, wherein the alkylenyl, alkenylenyl, or alkynylenyl is optionally substituted with one or more substituents independently selected from halogen, —OH, —CN, —NO₂, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
provided that when s is 0 then L¹ and L² form a single bond;
each R^{L} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
X¹ is selected from NR², CR², and C(R²)₂;
X⁶ is selected from NR³, CR³, and C(R³)₂;
R¹ and R⁴ are each independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
R² and R³ are each independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² and R³, together with the atoms to which they are attached and any intervening atoms form 4- to 10-membered heterocyclyl, aryl, or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² may come together with any one part of L² to form 3- to 10-membered heterocyclyl or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² may come together with any one part of L³ to form 3- to 10-membered heterocyclyl or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R³ may come together with any one part of L³ to form 3- to 10-membered heterocyclyl, aryl, or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
m is an integer selected from 0, 1, 2, 3, 4, 5, and 6;
n is an integer selected from 0, 1, 2, 3, 4, 5, and 6;
o is an integer selected from 0, 1, and 2;
s is an integer selected from 0 and 1; and
t is an integer selected from 0 and 1;
wherein:
aryl is cyclic, aromatic hydrocarbon groups that have 1 to 3 aromatic rings where the aromatic rings may be joined at a single point, or fused;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is monovalent monocyclic or polycyclic aromatic radical of 5 to 24 ring atoms, containing one or more ring heteroatoms selected from N, O, S, P, Se, or B, the remaining ring atoms being C;
W is selected from inhibitors of Wee1 kinase or their derivatives, in particular selected from the group comprising: adavosertib, ZN-c3, Debio-0123, milciclib, IMP-7068.

In various examples, compounds of the present disclosure have a structure of Formula (I):

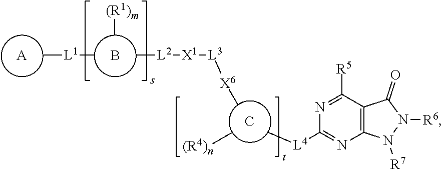

or a pharmaceutically acceptable salt, stereoisomer, solvate, prodrug, or tautomer thereof, wherein
Ring A is selected from

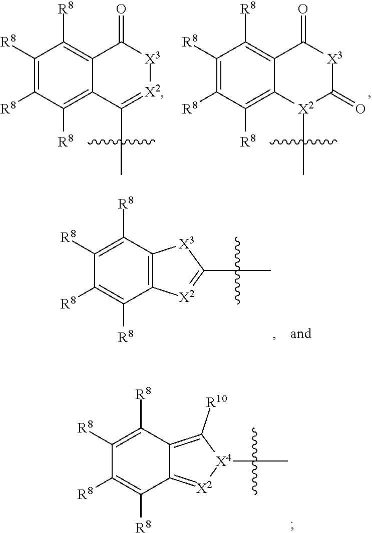

Ring B and Ring C are each independently selected from C₃-C₁₀ cycloalkyl, aryl, 3- to 10-membered heterocyclyl, and heteroaryl;
L¹, L², and L⁴ are each independently selected from bond, C₁-C₆ alkylenyl, C₂-C₆ alkenylenyl, C₂-C₆ alkynylenyl, C(O)—, C(O)NR^{L}—, —C(O)O—, —NR^{L}, —NR^{L}C(O)—, —NR^{L}C(O)NR^{L}—, —NR^{L}C(O)O—, —O—, —OC(O)—, —OC(O)NR^{L}—, —OC(O)O—, and —S(O)ₒ—, wherein the alkylenyl, alkenylenyl, or alkynylenyl is optionally substituted with one or more substituents independently selected from halogen, —OH, —CN, —NO₂, C₁-C₆ alkyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
L³ is selected from C₁-C₆ alkylenyl, C₂-C₆ alkenylenyl, and C₂-C₆ alkynylenyl, wherein the alkylenyl, alkenylenyl, or alkynylenyl is optionally substituted with one or more substituents independently selected from halogen, OH, CN, —NO₂, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
provided that when s is 0 then L¹ and L² form a single bond;
each R^{L} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
X¹ is selected from NR², CR², and C(R²)₂;
X² is selected from N and CR⁹;
X³ is selected from NR¹⁰, CR⁹, and C(R⁹)₂;
X⁴ is selected from N and CR⁹;
X⁶ is selected from NR³, CR³, and C(R³)₂;
R¹ and R⁴ are each independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
R² and R³ are each independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² and R³, together with the atoms to which they are attached, may come together to form 4- to 10-membered heterocyclyl, aryl, or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² may come together with any one part of L² to form 3- to 10-membered heterocyclyl or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² may come together with any one part of L³ to form 3- to 10-membered heterocyclyl or heteroaryl, optionally substituted with one more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R³ may come together with any one part of L³ to form 3- to 10-membered heterocyclyl, aryl, or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
R⁵ is selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
each from R⁶, R⁷, and R¹⁰ is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more R¹¹;
each from R⁸ and R⁹ is independently selected from hydrogen, halogen, —OH, —CN, —NO₂, —C(O)R^{x}, —C(O)N(R^{x})₂, —C(O)OR^{x}, —N(R^{X})₂, —OR^{x}, —S(O)ₒR^{x}, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or any one R⁸ and R⁹, together with the atoms to which they are attached, may come together to form 4- to 10-membered heterocyclyl or heteroaryl, optionally substituted with one or more substituents independently selected from halogen, —OH, oxo, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
each R¹¹ is independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
each R^{x} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
m is an integer selected from 0, 1, 2, 3, 4, 5, and 6;
n is an integer selected from 0, 1, 2, 3, 4, 5, and 6;
o is an integer selected from 0, 1, and 2;
s is an integer selected from 0 and 1; and
t is an integer selected from 0 and 1
wherein:
aryl is cyclic, aromatic hydrocarbon groups that have 1 to 3 aromatic rings where the aromatic rings may be joined at a single point, or fused;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is monovalent monocyclic or polycyclic aromatic radical of 5 to 24 ring atoms, containing one or more ring heteroatoms selected from N, O, S, P, Se, or B, the remaining ring atoms being C.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with modulation of poly ADP-ribose polymerase (PARP) enzyme and Wee1. The method comprises administering to a patient in need of a treatment for diseases or disorders associated with modulation of PARP and Wee1 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting poly ADP-ribose polymerase (PARP) enzyme and Wee1 kinase. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention relates to a method of treating a disease or disorder associated with modulation of poly ADP-ribose polymerase (PARP) enzyme. The method comprises administering to a patient in need of a treatment for diseases or disorders associated with modulation of PARP an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting poly ADP-ribose polymerase (PARP) enzyme. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention relates to a method of treating a disease or disorder associated with modulation of Wee1 kinase. The method comprises administering to a patient in need of a treatment for diseases or disorders associated with modulation of Wee1 kinase an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting Wee1. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting poly ADP-ribose polymerase (PARP) enzyme and Wee1 kinase.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting poly ADP-ribose polymerase (PARP) enzyme.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting Wee1.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting poly ADP-ribose polymerase (PARP) enzyme and Wee1 kinase.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting poly ADP-ribose polymerase (PARP) enzyme.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting

### Wee1.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with modulation of poly ADP-ribose polymerase (PARP) enzyme and Wee1, including cancer, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with modulation of poly ADP-ribose polymerase (PARP) enzyme, including cancer, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention further provides methods of treating a disease or disorder associated with modulation of Wee1 kinase, including cancer, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of poly ADP-ribose polymerase (PARP) enzyme and Wee1 that are therapeutic agents in the treatment of diseases and disorders, such as cancer.

The present invention provides inhibitors of poly ADP-ribose polymerase (PARP) enzyme that are therapeutic agents in the treatment of diseases and disorders, such as cancer.

The present invention provides inhibitors of Wee1 kinase that are therapeutic agents in the treatment of diseases and disorders, such as cancer.

The present invention further provides compounds and compositions with an improved efficacy and safety profile relative to known poly ADP-ribose polymerase (PARP) enzyme and Wee1 inhibitors. The present disclosure also provides agents with novel mechanisms of action toward protein tyrosine phosphatase enzymes in the treatment of various types of diseases, including cancer.

The present invention further provides compounds and compositions with an improved efficacy and safety profile relative to known poly ADP-ribose polymerase (PARP) enzyme inhibitors. The present disclosure also provides agents with novel mechanisms of action toward protein tyrosine phosphatase enzymes in the treatment of various types of diseases, including cancer.

The present invention further provides compounds and compositions with an improved efficacy and safety profile relative to known Wee1 kinase inhibitors. The present disclosure also provides agents with novel mechanisms of action toward protein tyrosine phosphatase enzymes in the treatment of various types of diseases, including cancer.

In some aspects, the present disclosure provides a compound obtainable by, or obtained by, a method for preparing compounds described herein (e.g., a method comprising one or more steps described in General Procedures A-B).

In some aspects, the present disclosure provides an intermediate as described herein, being suitable for use in a method for preparing a compound as described herein (e.g., the intermediate is selected from the intermediates described in Experimental part).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION OF THE INVENTION

The present disclosure relates to compounds and compositions that are capable of inhibiting the activity poly ADP-ribose polymerase (PARP) enzyme and/or Wee1 kinase. The disclosure features methods of treating, preventing or ameliorating a disease or disorder in which PARP enzyme and/or Wee1 kinase plays a role by administering to a patient in need thereof a therapeutically effective amount of a compound of Formula (I), or a pharmaceutically acceptable salt, hydrate, solvate, prodrug, stereoisomer, or tautomer thereof. The methods of the present invention can be used in the treatment of a variety of PARP- and/or Wee1-mediated diseases and disorders by inhibiting the activity of said enzymes. Inhibition of PARP and/or Wee1 can be an effective approach to the treatment, prevention, or amelioration of diseases including, but not limited to, cancer.

In general, this invention related to compound with molecules comprising three fragments: (1) inhibitor of poly(ADP-ribose) polymerase (PARP); (2) linker bounding two active parts of the molecule and could be indeed the part of one or two of these active molecules; (3) inhibitor of Wee1.

A first aspect of the invention relates to compounds of general Formula (A):

$$P - L - W ( A )$$

and pharmaceutically acceptable salts, solvates, prodrugs, and tautomers thereof, wherein
P is selected from inhibitors of poly(ADP-ribose) polymerase (PARP) or their derivatives, selected from the group comprising: rucaparib, niraparib, senaparib, veliparib; talazoparib, stenoparib, pamiparib, fluzoparib, simmiparib;
L is selected from linker bonding two active parts of the molecule and structural parts of this linker can be indeed the parts of one or two of these active molecules and presented by the structure:

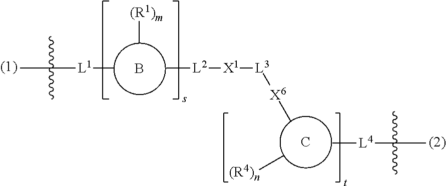

wherein the bond (1) connected to the P-part of the molecule and the bond (2) connected to the W-part of the molecule;
W is selected from inhibitors of Wee1 kinase or their derivatives, selected from the group comprising: adavosertib, ZN-c3, Debio-0123, milciclib, IMP-7068.

In more detail aspect the invention relates to the compounds of Formula (I):

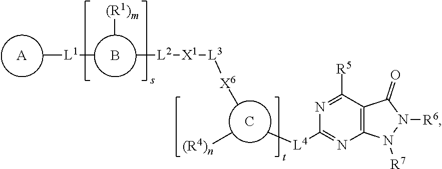

and pharmaceutically acceptable salts, hydrates, solvates, prodrugs, stereoisomers, and tautomers thereof, wherein Ring A, Ring B, Ring C, L¹, L², L³, L⁴, R¹, R⁴, R⁵, R⁶, R⁷, X¹, X⁶, m, n, s, and t are described herein.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆) haloalkyl, (C₁-C₆) haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O (C₁-C₆) alkyl, —NH₂, —NH((C₁-C₆)alkyl), —N((C₁-C₆)alkyl)₂, —NHC(O)(C₁-C₆)alkyl, —C(O)NH(C₁-C₆) alkyl, —S(O)₂(C₁-C₆)alkyl, —S(O)NH(C₁-C₆)alkyl, and S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆)alkynyl, (C₂-C₆) alkenyl, (C₂-C₆)alkynyl, —OH, —OP(O)(OH)₂, —OC(O)(C₁-C₆)alkyl, —C(O)(C₁-C₆)alkyl, —OC(O)O (C₁-C₆)alkyl, —NH₂, NH((C₁-C₆)alkyl), N((C₁-C₆)alkyl)₂, —S(O)₂—(C₁-C₆)alkyl, —S (O)NH(C₁-C₆)alkyl, and —S(O)N((C₁-C₆)alkyl)₂. The substituents can themselves be optionally substituted. Furthermore, when containing two fused rings, the aryl groups herein defined may have a saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-122-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7 (4H)-only, 3,4-dihydropyrazino[1,2-a]indol-1(2H)-onyl, or benzo[c][1,2]oxaborol-1(3H)-olyl.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆)alkyl group include, but are not limited to, methyl, ethyl, propyl, butyl, pentyl, hexyl, iso-propyl, iso-butyl, sec-butyl, tert-butyl, iso-pentyl, neo-pentyl, and iso-hexyl.

“Alkoxy” refers to a straight or branched chain saturated hydrocarbon containing 1-12 carbon atoms containing a terminal “O” in the chain, i.e., —O (alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, adamantyl, and derivatives thereof. In the case of polycyclic cycloalkyl, only one of the rings in the cycloalkyl needs to be non-aromatic.

“Heterocyclyl”, “heterocycle” or “heterocycloalkyl” refers to a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms (such as O, N, S, P, Se, or B), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted one or more halogen. Examples of haloalkoxy groups include, but are not limited to, trifluoromethoxy, difluoromethoxy, pentafluoroethoxy, trichloromethoxy, etc.

The term “amine” as used herein refers to primary (R—NH₂, R≠H), secondary (R²—NH, R²+H) and tertiary (R³—N, R≠H) amines. A substituted amine is intended to mean an amine where at least one of the hydrogen atoms has been replaced by the substituent.

The term “amino” as used herein means a substituent containing at least one nitrogen atom. Specifically, —NH₂, —NH(alkyl) or alkylamino, —N(alkyl)₂ or dialkylamino, amide-, carbamide-, urea, and sulfamide substituents are included in the term “amino”.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present invention also contemplates isotopically labelled compounds of Formula I (e.g., those labeled with ²H and ¹⁴C). Deuterated (i.e., ²H or D) and carbon-14 (i.e., 14° C.) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a disease or disorder in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The present invention relates to compounds or pharmaceutically acceptable salts, hydrates, solvates, prodrugs, stereoisomers, or tautomers thereof, capable of inhibiting poly ADP-ribose polymerase (PARP) enzyme and/or Wee1, which are useful for the treatment of diseases and disorders associated with modulation of said enzymes. The invention further relates to compounds, or pharmaceutically acceptable salts, hydrates, solvates, prodrugs, stereoisomers, or tautomers thereof, which can be useful for inhibiting poly ADP-ribose polymerase (PARP) enzyme and/or Wee1.

In some embodiments, the compound of Formula I is a compound of Formula (I-A):

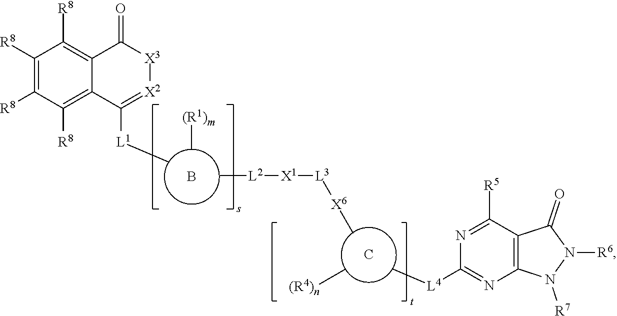

or a pharmaceutically acceptable salt, stereoisomer, solvate, prodrug, or tautomer thereof.

In some embodiments, the compound of Formula I is a compound of Formula (I-A-1):

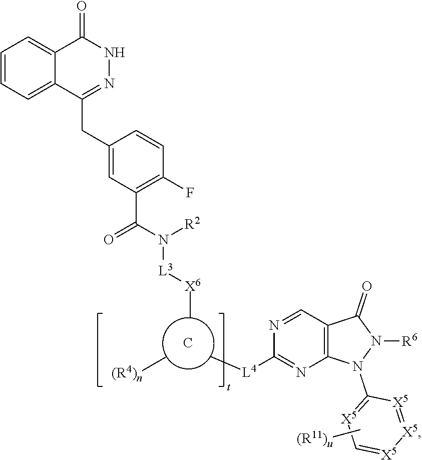

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound of Formula I is a compound of Formula (I-A-2):

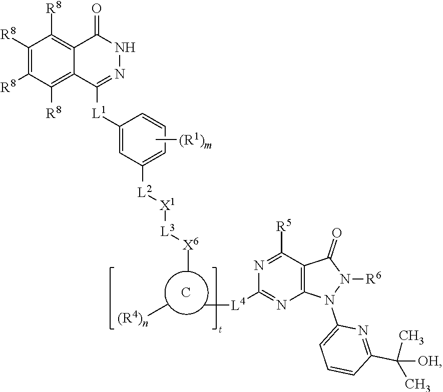

or a pharmaceutically acceptable salt, stereoisomer, solvate, prodrug, or tautomer thereof.

In some embodiments, the compound of Formula I is a compound of Formula (I-A-3):

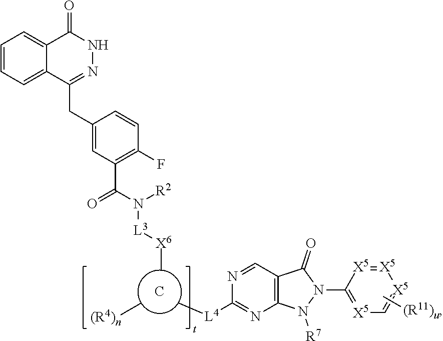

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound of Formula I is a compound of Formula (I-A-4):

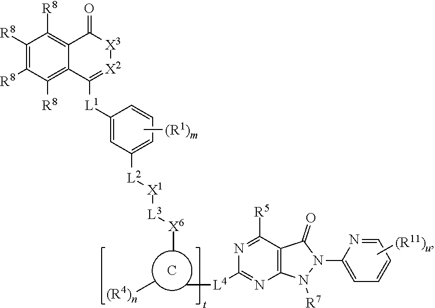

or a pharmaceutically acceptable salt, stereoisomer, solvate, prodrug, or tautomer thereof.

In some embodiments, the compound is of Formula (I-B):

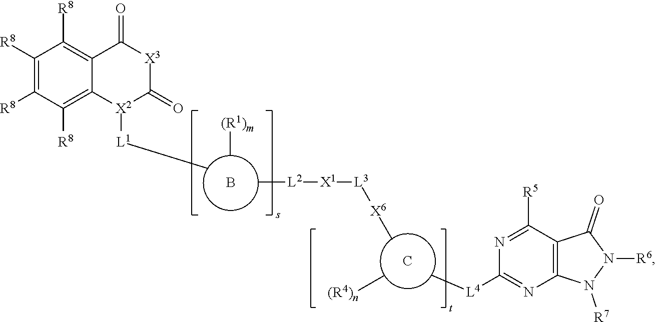

or a pharmaceutically acceptable salt, stereoisomer, solvate, prodrug, or tautomer thereof.

In some embodiments, the compound is of Formula (I-B-1):

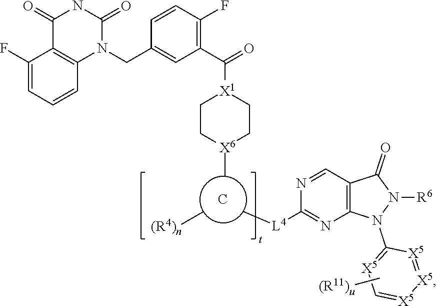

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound is of Formula (I-B-2):

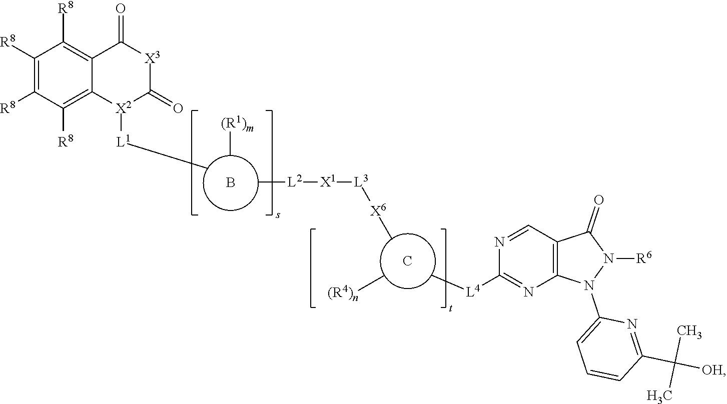

or a pharmaceutically acceptable salt, stereoisomer, solvate, prodrug, or tautomer thereof.

In some embodiments, the compound is of Formula (I-B-3):

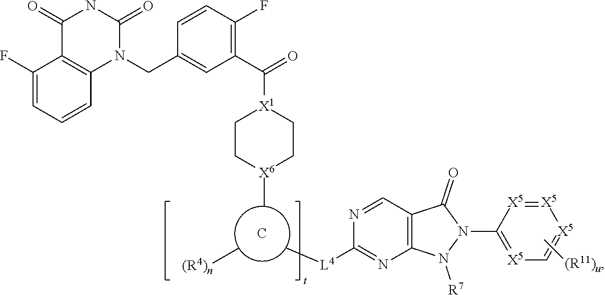

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound is of Formula (I-B-4):

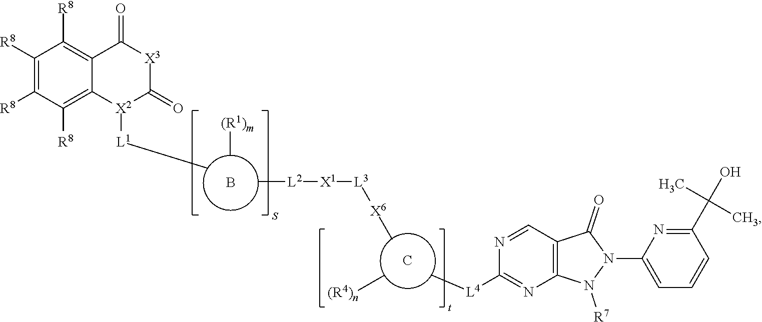

or a pharmaceutically acceptable salt, stereoisomer, solvate, prodrug, or tautomer thereof.

In some embodiments, the compound is of Formula (I-C):

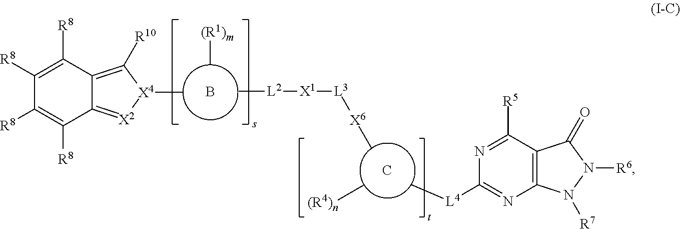

or a pharmaceutically acceptable salt, stereoisomer, solvate, prodrug, or tautomer thereof.

In some embodiments, the compound is of Formula (I-C-1):

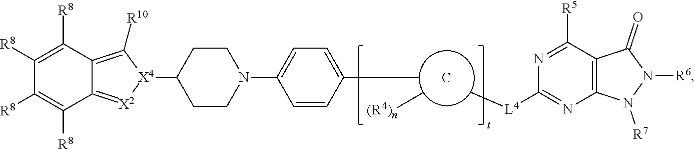

or a pharmaceutically acceptable salt, stereoisomer, solvate, prodrug, or tautomer thereof.

In some embodiments, the compound is of Formula (I-C-2):

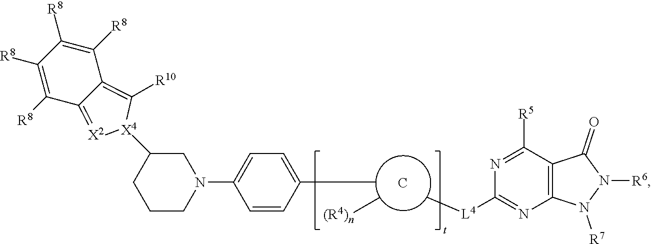

or a pharmaceutically acceptable salt, stereoisomer, solvate, prodrug, or tautomer thereof.

In some embodiments, the compound is of Formula (I-C-3):

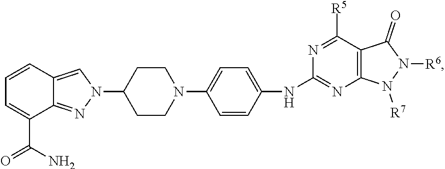

or a pharmaceutically acceptable salt, stereoisomer, solvate, prodrug, or tautomer thereof.

In some embodiments, the compound is of Formula (I-C-4):

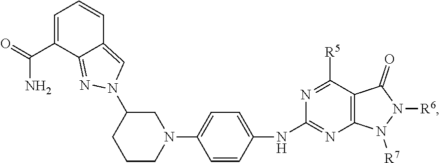

or a pharmaceutically acceptable salt, stereoisomer, solvate, prodrug, or tautomer thereof.

In some embodiments, the compound is of Formula (I-C-5) or Formula (I-C-6):

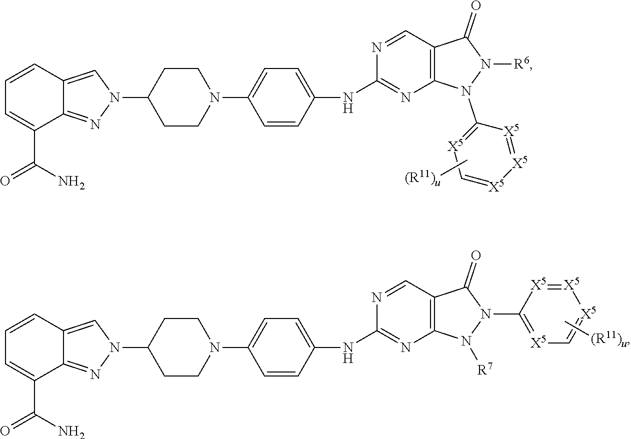

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound is of Formula (I-C-7) or Formula (I-C-8):

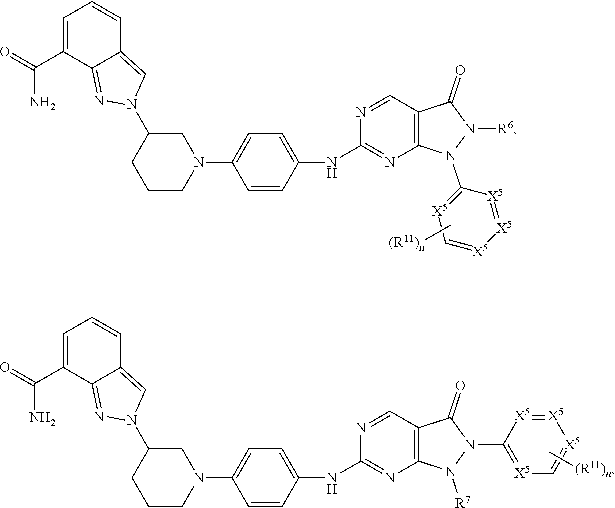

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound is of Formula (I-D):

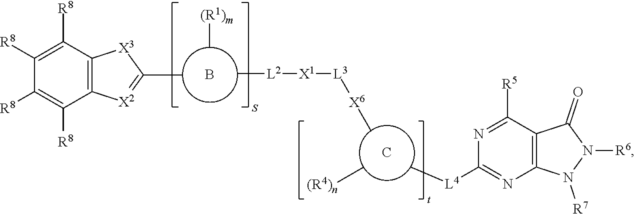

or a pharmaceutically acceptable salt, stereoisomer, solvate, prodrug, or tautomer thereof.

In some embodiments, the compound is of Formula (I-D-1):

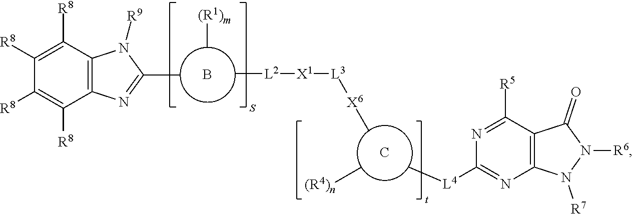

or a pharmaceutically acceptable salt, stereoisomer, solvate, prodrug, or tautomer thereof.

In some embodiments, the compound is of Formula (I-D-2):

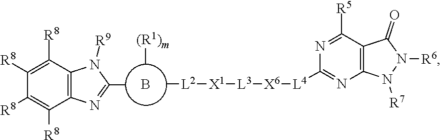

or a pharmaceutically acceptable salt, stereoisomer, solvate, prodrug, or tautomer thereof.

In some embodiments, the compound is of Formula (I-D-3), (I-D-4), or (I-D-5):

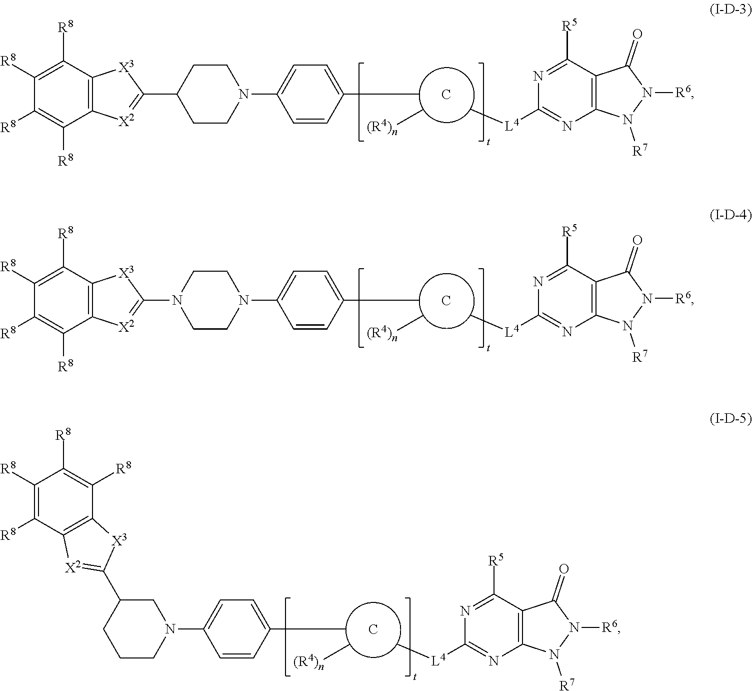

or a pharmaceutically acceptable salt, stereoisomer, solvate, prodrug, or tautomer thereof.

In some embodiments, the compound is of Formula (I-D-6), (I-D-7), or (I-D-8):

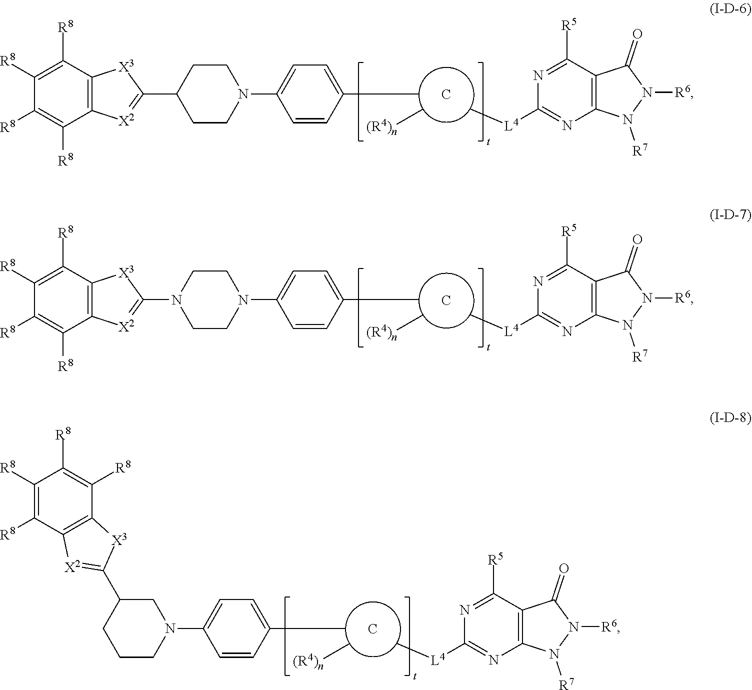

or a pharmaceutically acceptable salt, stereoisomer, solvate, prodrug, or tautomer thereof.

In some embodiments, the compound is of Formula (I-D-9):

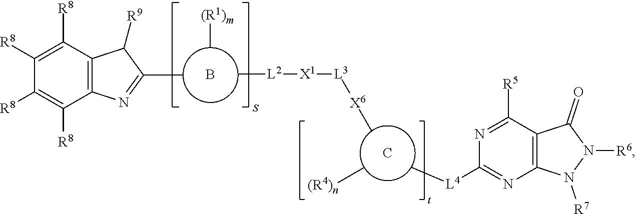

or a pharmaceutically acceptable salt, stereoisomer, solvate, prodrug, or tautomer thereof.

In some embodiments, the compound is of Formula (I-D-10):

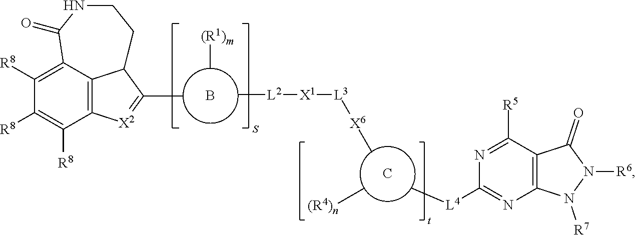

or a pharmaceutically acceptable salt, stereoisomer, solvate, prodrug, or tautomer thereof.

In some embodiments, the compound is of Formula (I-D-11):

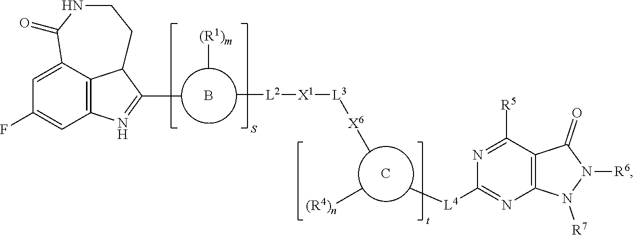

or a pharmaceutically acceptable salt, stereoisomer, solvate, prodrug, or tautomer thereof. In some embodiments, the compound is of Formula (I-D-12):

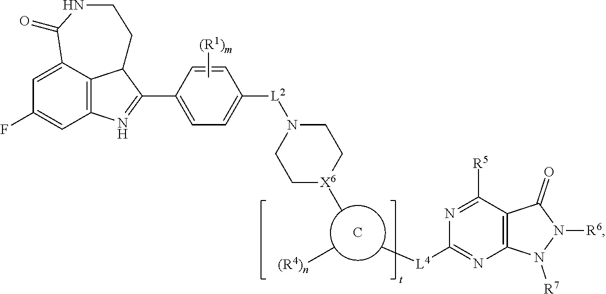

or a pharmaceutically acceptable salt, stereoisomer, solvate, prodrug, or tautomer thereof.

In some embodiments, the compound is of Formula (I-E):

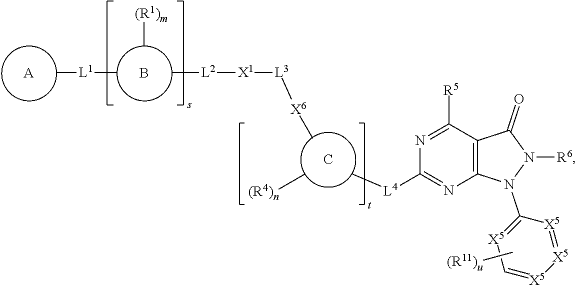

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound is of Formula (I-E-1):

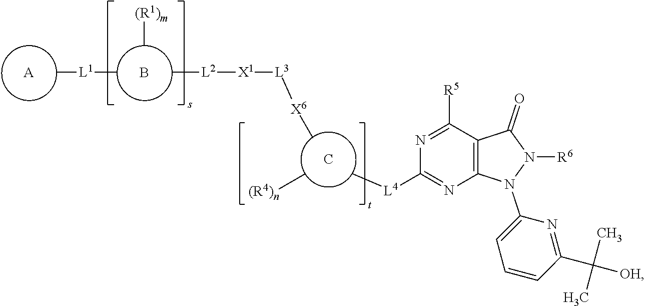

or a pharmaceutically acceptable salt, stereoisomer, solvate, prodrug, or tautomer thereof.

In some embodiments, the compound is of Formula (I-F):

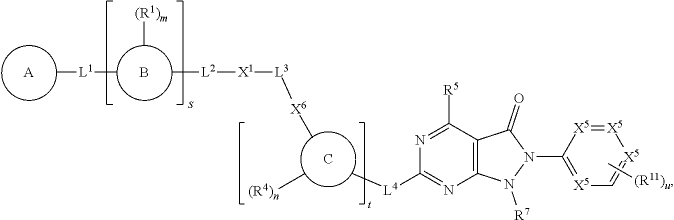

or a pharmaceutically acceptable salt, stereoisomer, solvate, prodrug, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

In some embodiments, the compound is of Formula (I-F-1):

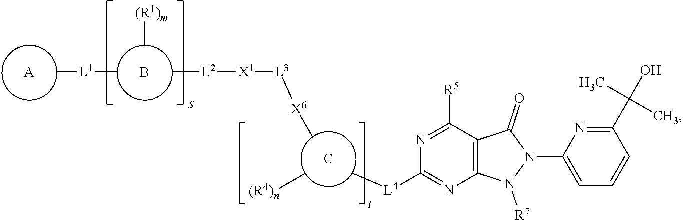

or a pharmaceutically acceptable salt, stereoisomer, solvate, prodrug, or tautomer thereof.

In some embodiments, the compound is of Formula (I-G):

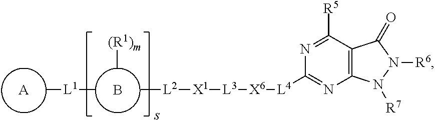

or a pharmaceutically acceptable salt, isomer, solvate, prodrug, or tautomer thereof.

In some embodiments, the compound is of Formula (I-G-1):

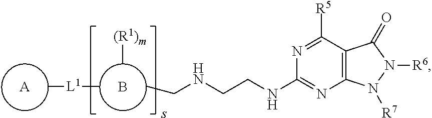

or a pharmaceutically acceptable salt, isomer, solvate, prodrug, or tautomer thereof.

In some embodiments, the compound is of Formula (I-G-2):

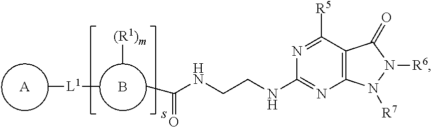

or a pharmaceutically acceptable salt, isomer, solvate, prodrug, or tautomer thereof.

In some embodiments, ring A is

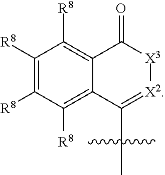

In some embodiments, ring A is

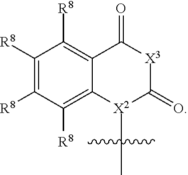

In some embodiments, ring A is

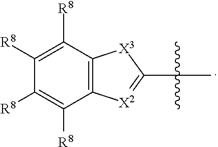

In some embodiments, ring A is

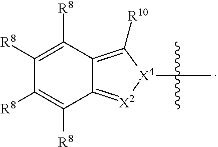

In some embodiments, ring A is

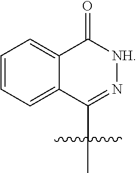 [SMILES: *c1n[nH]c(=O)c2ccccc12]

In some embodiments, ring A is

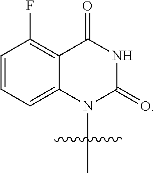 [SMILES: *n1c(=O)[nH]c(=O)c2c(F)cccc21]

In some embodiments, ring A is

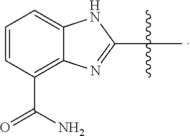 [SMILES: *c1nc2c(C(N)=O)cccc2[nH]1]

In some embodiments, ring A is

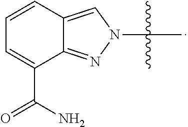 [SMILES: *n1cc2cccc(C(N)=O)c2n1]

In some embodiments, ring A is

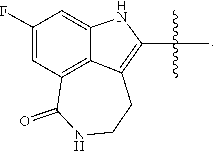 [SMILES: *c1[nH]c2cc(F)cc3c2c1CCNC3=O]

In some embodiments, L¹ is C₁-C₆ alkylenyl. In some embodiments, L¹ is methylenyl. In some embodiments L¹ is bond. In some embodiments, L¹ is C₂-C₆ alkenylenyl. In some embodiments, L¹ is C₂-C₆ alkynylenyl. In some embodiments, L¹ is —C(O)NR^{L}—. In some embodiments, L¹ is —C(O)O—. In some embodiments, L¹ is —NR^{L}—. In some embodiments, L¹ is —NR^{L}C(O)—. In some embodiments, L¹ is —NR^{L}C(O)NR^{L}—. In some embodiments, L¹ is —NR^{L}C(O)O—. In some embodiments, L¹ is —O—. In some embodiments, L¹ is —OC(O)—. In some embodiments, L¹ is —OC(O)NR^{L}—. In some embodiments, L¹ is —OC(O)O—. In some embodiments, L¹ is —S(O) . . . . In some embodiments, L¹ is selected from C₁-C₆ alkylenyl, C₂-C₆ alkenylenyl, C₂-C₆ alkynylenyl, wherein the alkylenyl, alkenylenyl, and alkynylenyl is optionally substituted with one or more substituents independently selected from halogen, —OH, —CN, —NO₂, C₁-C₆ alkyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, L¹ is —CH₂—or bond. In some embodiments L¹ is methylenyl. In some embodiments L¹ is bond.

In some embodiments, s is 1.

In some embodiments, ring B is selected from C₃-C₁₀ cycloalkyl, aryl, 3- to 10-membered heterocyclyl, and heteroaryl.

In some embodiments, ring B is selected from aryl, 3- to 10-membered heterocyclyl.

In some embodiments, ring B is selected from benzene,

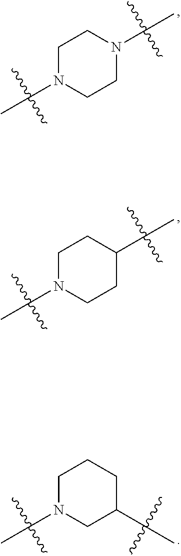 [SMILES: *N1CCN(*)CC1 ; *C1CCN(*)CC1 ; *C1CCCN(*)C1]

In some embodiments, m is an integer selected from 0, 1, 2, 3, 4, 5, and 6.

In some embodiments, m is 0.

In some embodiments, m is 1.

In some embodiments, m is 2.

In some embodiments, m is 3.

In some embodiments, m is selected from 4, 5, and 6.

In some embodiments, R¹ is selected from halogen, —OH, CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, and heteroaryl are optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R¹ is selected from halogen, C₁-C₆ alkyl.

In some embodiments, R¹ is selected from F, Cl, Br, I, CH₃, C₂H₅, C₃H₇, C₄H₉, C₅H₁₁, C₆H₁₃.

In some embodiments, R¹ is selected from F, C₁, CH₃, C₂H₅.

In some embodiments, s is 0.

In some embodiments L² is bond. In some embodiments, L² is C₁-C₆ alkylenyl. In some embodiments, L² is methylenyl. In some embodiments, L² is C₂-C₆ alkenylenyl. In some embodiments, L² is C₂-C₆ alkynylenyl. In some embodiments, L² is —C(O)NR^{L}—. In some embodiments, L² is —C(O)O—. In some embodiments, L² is —NR^{L}—. In some embodiments, L² is —NR^{L}C(O)—. In some embodiments, L² is —NR^{L}C(O)NR^{L}—. In some embodiments, L² is —NR^{L}C(O)O—. In some embodiments, L² is —O—. In some embodiments, L² is —OC(O)—. In some embodiments, L² is —OC(O)NR^{L}—. In some embodiments, L² is —OC(O)O—. In some embodiments, L² is —S(O)ₒ—. In some embodiments, L² is selected from C₁-C₆ alkylenyl, C₂-C₆ alkenylenyl, C₂-C₆ alkynylenyl, wherein the alkylenyl, alkenylenyl, and alkynylenyl is optionally substituted with one or more substituents independently selected from halogen, —OH, —CN, —NO₂, C₁-C₆ alkyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, L² is bond, —CH₂—, —CH(Me)-, —C(O)—, —C(O)NH—.

In some embodiments, L¹ and L² form a one single bond in case s is 0.

In some embodiments, X¹ is selected from NR², CR² and C(R²)₂.

In some embodiments, X¹ is selected from NH.

In some embodiments, L³ is selected from C₁-C₆ alkylenyl, C₂-C₆ alkenylenyl, and C₂-C₆ alkynylenyl, wherein the alkylenyl, alkenylenyl, or alkynylenyl is optionally substituted with one or more substituents independently selected from halogen, —OH, —CN, —NO₂, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, L³ is selected from—CH₂CH₂—.

In some embodiments, X⁶ is selected from NR³, CR³, and C(R³)₂.

In some embodiments, X⁶ is CR³.

In some embodiments, X⁶ is NR³.

In some embodiments, X⁶ is NH.

In some embodiments the fragment X¹—L³—X⁶ is selected from:

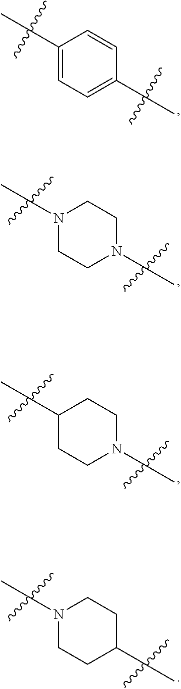 [SMILES: *c1ccc(*)cc1 ; *N1CCN(*)CC1 ; *C1CCN(*)CC1 ; *C1CCN(*)CC1]

In some embodiments, t is 1.

In some embodiments, ring C selected from:

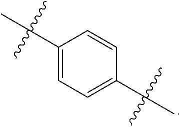 [SMILES: *c1ccc(*)cc1]

In some embodiments, n is an integer selected from 0, 1, 2, 3, 4, 5, and 6.

In some embodiments, n is 1.

In some embodiments, n is 0.

In some embodiments, t is 0.

In some embodiments, L⁴ is selected from bond, C₁-C₆ alkylenyl, C₂-C₆ alkenylenyl, C₂-C₆ alkynylenyl, —C(O)—, C(O)NR^{L}—, —C(O)O—, —NR^{L}—, —NR^{L}C(O)—, —NR^{L}C(O)NR^{L}, —NR^{o}C. (0)O—, —O—, —OC(O), OC(O)NR^{L}—, —OC(O)O—, and —S(O)ₒ—, wherein the alkylenyl, alkenylenyl, and alkynylenyl is optionally substituted with one or more halogen, —OH, —CN, —NO₂, C₁-C₆ alkyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, L⁴ is selected from bond, or NH.

In some embodiments, R^{L} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R^{L} is hydrogen.

In some embodiments R⁵ is selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R⁵ is hydrogen.

In some embodiments, R⁶ is selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more R¹¹.

In some embodiments, R¹¹ is independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R⁶ is

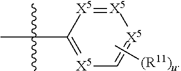

In some embodiments, R⁶ is

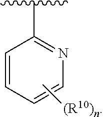

In some embodiments, R⁶ is

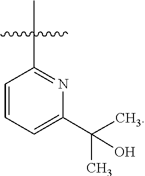 [SMILES: *c1cccc(C(C)(C)O)n1]

In some embodiments, R⁶ is selected from

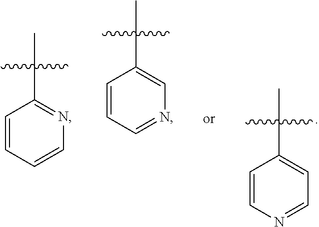

In some embodiments, R⁶ is selected from—CH₃,

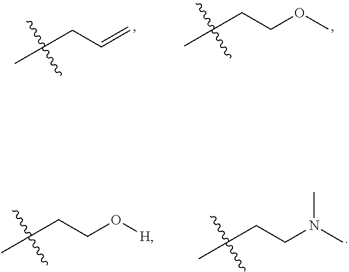

In some embodiments, R⁷ is

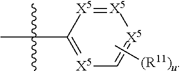

In some embodiments, R⁷ is

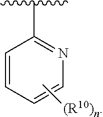

In some embodiments, R⁷ is

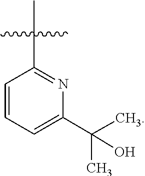 [SMILES: *c1cccc(C(C)(C)O)n1]

In some embodiments, R⁷ is selected from

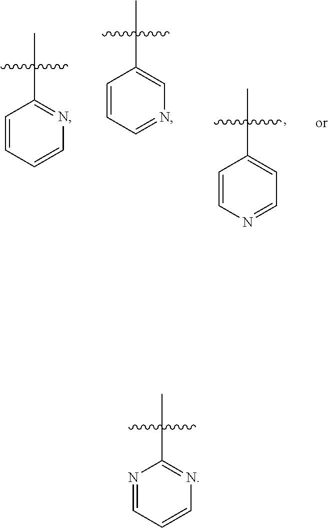

In some embodiments, R⁷ is selected from H, —CH₃,

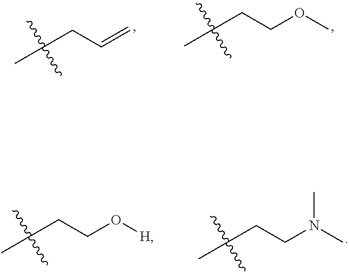

In some embodiments, u is 0.

In some embodiments, u is 1.

In some embodiments, R⁸ is selected from hydrogen, halogen, OH, CN, —NO₂, —C(O)R^{x}, —C(O)N(R^{x})₂, —C(O)OR^{x}, —N(R^{X})₂, —OR^{x}, —S(O)ₒR^{x}, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R⁸ is selected from hydrogen, halogen, —C(O)N(R^{x})₂.

In some embodiments, R⁸ is selected from H, F, —C(O)NH₂.

In some embodiments, R⁹ is selected from hydrogen, halogen, —OH, —CN, —NO₂, —C(O)R^{x}, —C(O)N(R)₂, —C(O)OR^{x}, —N(R^{x})₂, —OR^{x}, —S(O)ₒR^{x}, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R⁹ is H.

In some embodiments, one R⁸ and R⁹, together with the atoms to which they are attached, may come together to form 4- to 10-membered heterocyclyl or heteroaryl, optionally substituted with one or more optionally substituted with one or more halogen, —OH, oxo, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, one R⁸ and R⁹, together with the atoms to which they are attached, form the ring D:

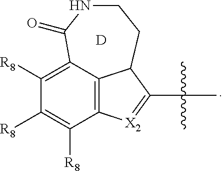

In some embodiments, one R⁸ and R⁹, together with the atoms to which they are attached, form the ring D′:

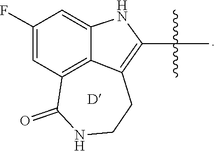 [SMILES: *c1[nH]c2cc(F)cc3c2c1CCNC3=O]

In some embodiments, R¹⁰ is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more R¹¹.

In some embodiments, R¹⁰ is hydrogen.

In some embodiments, R¹¹ is selected from halogen, —OH, CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, at least one R¹¹ is

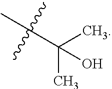 [SMILES: *C(C)(C)O]

In some embodiments, R^{X} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl.

In some embodiments, R^{x} is hydrogen or—CH₃.

In some embodiments, o is 0, 1, or 2.

In some embodiments, o is 0.

In some embodiments, o is 1.

In some embodiments, o is 2.

In some embodiments, Ring A is

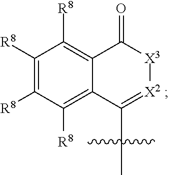

L¹ is C₁-C₆ alkylenyl; s is 1; Ring B is aryl; m is 1; R¹ is halogen; L² is —C(O)—; X¹—L³—X⁶ come together to form 4- to 10-membered heterocyclyl; t is 1; Ring C is aryl; n is 0; L⁴ is —NH—; R⁵ is hydrogen; R⁶ is —CH₃; R⁷ is

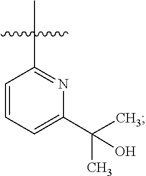 [SMILES: *c1cccc(C(C)(C)O)n1]

R⁸ is independently selected from H, and halogen; X² is N; X³ is NR¹⁰; R¹⁰ is H.

In some embodiments, Ring A is

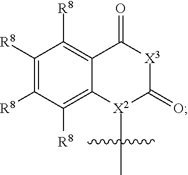

L¹ is C₁-C₆ alkylenyl; s is 1; Ring B is aryl; m is 1; R¹ is halogen; L² is C₁-C₆ alkylenyl; X¹—L³—X⁶ come together to form 4- to 10-membered heterocyclyl; t is 1; Ring C is aryl; n is 0; L⁴ is —NH—; R⁵ is hydrogen; R⁶ is

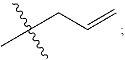 [SMILES: *CC=C]

### R⁷ is

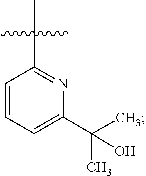 [SMILES: *c1cccc(C(C)(C)O)n1]

R⁸ is independently selected from H, and halogen; X² is N; X³ is NR¹⁰; R¹⁰ is H.

In some embodiments, Ring A is

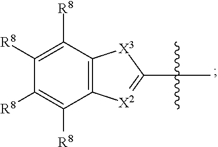

L¹ is bond; s is 0; R² may come together with any part of L² to form 3- to 10-membered heterocyclyl; R³ come together with any part of L³ to form aryl; t is 0; L⁴ is —NH—; R⁵ is hydrogen; R⁶ is C₁-C₆ alkyl; R⁷ is

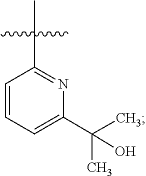 [SMILES: *c1cccc(C(C)(C)O)n1]

R⁸ is independently selected from H, and —C(O)N(R^{X})₂; R^{x} is hydrogen; X² is N; X³ is NR¹⁰; R¹⁰ is H.

In some embodiments, Ring A is

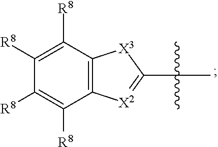

X² is N; X³ is CR⁹; one R⁸ and R⁹, together with the atoms to which they are attached, come together to form 4- to 10-membered heterocyclyl; others R⁸ is independently selected from H, and halogen; L¹ is bond; s is 1; Ring B is aryl; m is 0; L² is C₁-C₆ alkylenyl; X¹—L³—X⁶ come together to form 4- to 10-membered heterocyclyl; t is 1; Ring C is aryl; L⁴ is —NH—; R⁵ is hydrogen; R⁶ is C₂-C₆ alkenyl; R⁷ is

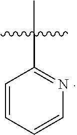 [SMILES: *c1ccccn1]

In some embodiments, Ring A is

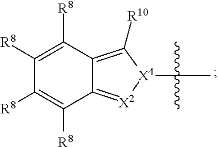

R¹⁰ is H; R⁸ is independently selected from H, halogen and —C(O)N(R^{X})₂; R^{x} is H; s is 0, L¹ and L² together form one single bond; X¹—L³_X⁶ come together to form 4- to 10-membered heterocyclyl; t is 1; Ring C is aryl; n is 0; L⁴ is —NH—; R⁵ is hydrogen; R⁶ is C₁-C₆ alkyl; R⁷ is

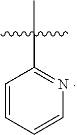 [SMILES: *c1ccccn1]

Non-limiting illustrative examples of compound of Formula (I) include:
4-[[3-[4-[4-[[2-allyl-1-[3-(1-hydroxy-1-methyl-ethyl)phenyl]-3-oxo-pyrazolo[3,4-d]pyrimidin-6-yl]amino]phenyl]piperazine-1-carbonyl]-4-fluoro-phenyl]methyl]-2H-phthalazin-1-one;
4-[[3-[4-[2-allyl-1-[6-(1-hydroxy-1-methyl-ethyl)-2-pyridyl]-3-oxo-pyrazolo[3,4-d]pyrimidin-6-yl]piperazine-1-carbonyl]-4-fluoro-phenyl]methyl]-2H-phthalazin-1-one;
4-[[4-fluoro-3-[4-[4-[[2-methyl-3-oxo-1-(2-pyridyl)pyrazolo[3,4-d]pyrimidin-6-yl]amino]phenyl]piperazine-1-carbonyl]phenyl]methyl]-2H-phthalazin-1-one;
4-[[3-[4-[[2-allyl-1-[6-(1-hydroxy-1-methyl-ethyl)-2-pyridyl]-3-oxo-pyrazolo[3,4-d]pyrimidin-6-yl]amino]piperidine-1-carbonyl]-4-fluoro-phenyl]methyl]-2H-phthalazin-1-one;
4-[[4-fluoro-3-[4-[2-methyl-3-oxo-1-(2-pyridyl)pyrazolo[3,4-d]pyrimidin-6-yl]piperazine-1-carbonyl]phenyl]methyl]-2H-phthalazin-1-one;
N-[2-[[2-allyl-1-[6-(1-hydroxy-1-methyl-ethyl)-2-pyridyl]-3-oxo-pyrazolo[3,4-d]pyrimidin-6-yl]amino]ethyl]-2-fluoro-5-[(4-oxo-3H-phthalazin-1-yl)methyl]benzamide;
1-[[3-[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazine-1-carbonyl]-4-fluoro-phenyl]methyl]-5-fluoro-quinazoline-2,4-dione;
5-fluoro-1-[[4-fluoro-3-[4-[4-[[1-methyl-3-oxo-2-(2-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[2-methyl-3-oxo-1-(2-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[1-methyl-3-oxo-2-(3-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[1-methyl-3-oxo-2-(4-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[2-methyl-3-oxo-1-(3-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[2-methyl-3-oxo-1-(4-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[2-(2-methoxyethyl)-3-oxo-1-(2-pyridyl)pyrazolo[3,4-d]pyrimidin-6-yl]amino]phenyl]piperazine-1-carbonyl]phenyl]methyl]quinazoline-2,4-dione;
1-[[3-[4-[4-[[2-[[2-(dimethylamino)ethyl]-3-oxo-1-(2-pyridyl)pyrazolo[3,4-d]pyrimidin-6-yl]amino]phenyl]piperazine-1-carbonyl]-4-fluoro-phenyl]methyl]-5-fluoro-quinazoline-2,4-dione;
5-fluoro-1-[[4-fluoro-3-[4-[4-[[2-(2-hydroxyethyl)-3-oxo-1-(2-pyridyl)pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[2-methyl-3-oxo-1H-pyrazolo[3,4-d]pyrimidin-6-yl)amino]phenyl]piperazine-1-carbonyl]phenyl]methyl]quinazoline-2,4-dione;
5-fluoro-1-[[4-fluoro-3-[4-[4-[[3-oxo-2-(2-pyridyl)-1H-pyrazolo[3,4-d]pyrimidin-6-yl]amino]phenyl]piperazine-1-carbonyl]phenyl]methyl]quinazoline-2,4-dione;
1-[[3-[4-[4-[[2-allyl-3-oxo-1-(2-pyridyl)pyrazolo[3,4-d]pyrimidin-6-yl]amino]phenyl]piperazine-1-carbonyl]-4-fluoro-phenyl]methyl]-5-fluoro-quinazoline-2,4-dione;
5-fluoro-1-[[4-fluoro-3-[4-[4-[(2-methyl-3-oxo-1-pyrimidin-2-yl-pyrazolo[3,4-d]pyrimidin-6-yl)amino]phenyl]piperazine-1-carbonyl]phenyl]methyl]quinazoline-2,4-dione;
5-fluoro-1-[[4-fluoro-3-[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazine-1-carbonyl]phenyl]methyl]quinazoline-2,4-dione;
5-fluoro-1-[[4-fluoro-3-[4-[4-[[2-(2-hydroxyethyl)-1-[6-(1-hydroxy-1-methyl-ethyl)-2-pyridyl]-3-oxo-pyrazolo[3,4-d]pyrimidin-6-yl]amino]phenyl]piperazine-1-carbonyl]phenyl]methyl]quinazoline-2,4-dione;
2-fluoro-5-[(5-fluoro-2,4-dioxo-quinazolin-1-yl)methyl]-N-[4-[[2-methyl-3-oxo-1-(2-pyridyl)pyrazolo[3,4-d]pyrimidin-6-yl]amino]phenyl]benzamide;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]phenyl]-6-fluoro-3,10-diazatricyclo[6.4.1.04,13]trideca-1,4,6,8 (13)-tetraen-9-one;
6-fluoro-2-[[4-[4-[4-[[2-methyl-3-oxo-1-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[1-methyl-3-oxo-2-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[2-(2-methoxyethyl)-3-oxo-1-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
2-[4-[[4-[4-[[2-allyl-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4,6,8 (13)-tetraen-9-one;
6-fluoro-2-[4-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[2-(2-hydroxyethyl)-1-[6-(1-hydroxy-1-methyl-ethyl)-2-pyridyl]-3-oxo-pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[2-(2-hydroxyethyl)-3-oxo-1-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
2-[4-[[4-[2-allyl-1-[6-(1-hydroxy-1-methyl-ethyl)-2-pyridyl]-3-oxo-pyrazolo[3,4-d]pyrimidin-6-yl]piperazin-1-yl]methyl]phenyl]-6-fluoro-3,10-diazatricyclo[6.4.1.04,13]trideca-1,4,6,8 (13)-tetraen-9-one;
2-[4-[[2-[[2-allyl-1-[6-(1-hydroxy-1-methyl-ethyl)-2-pyridyl]-3-oxo-pyrazolo[3,4-d]pyrimidin-6-yl]amino]ethylamino]methyl]phenyl]-6-fluoro-3,10-diazatricyclo[6.4.1.04,13]trideca-1,4,6,8 (13)-tetraen-9-one;
2-[4-[[[1-[2-allyl-1-[6-(1-hydroxy-1-methyl-ethyl)-2-pyridyl]-3-oxo-pyrazolo[3,4-d]pyrimidin-6-yl]-4-piperidyl]amino]methyl]phenyl]-6-fluoro-3,10-diazatricyclo[6.4.1.04,13]trideca-1,4,6,8 (13)-tetraen-9-one;
2-[4-[[4-[[2-allyl-1-[6-(1-hydroxy-1-methyl-ethyl)-2-pyridyl]-3-oxo-pyrazolo[3,4-d]pyrimidin-6-yl]amino]-1-piperidyl]methyl]phenyl]-6-fluoro-3,10-diazatricyclo[6.4.1.04,13]trideca-1,4,6,8 (13)-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-3-fluoro-phenyl]-6-fluoro-3,10-diazatricyclo[6.4.1.04,13]trideca-1,4 (13),5,7-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-2-methyl-phenyl]-6-fluoro-3,10-diazatricyclo[6.4.1.04,13]trideca-1,4,6,8 (13)-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-3-methyl-phenyl]-6-fluoro-3,10-diazatricyclo[6.4.1.04,13]trideca-1,4,6,8 (13)-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-3-chloro-5-fluoro-phenyl]-6-fluoro-3,10-diazatricyclo[6.4.1.04,13]trideca-1,4 (13),5,7-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-3-chloro-phenyl]-6-fluoro-3,10-diazatricyclo[6.4.1.04,13]trideca-1,4 (13),5,7-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-3,5-difluoro-phenyl]-6-fluoro-3,10-diazatricyclo[6.4.1.04,13]trideca-1,4 (13),5,7-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-2,6-difluoro-phenyl]-6-fluoro-3,10-diazatricyclo[6.4.1.04,13]trideca-1,4 (13),5,7-tetraen-9-one;
6-fluoro-2-[3-fluoro-4-[[4-[4-[[2-methyl-3-oxo-1-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4 (13),5,7-tetraen-9-one;
6-fluoro-2-[3-fluoro-4-[[4-[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazin-1-yl]methyl]phenyl]-3,10-diazatricyclo[6.4.1.04,13]trideca-1,4 (13),5,7-tetraen-9-one;
6-fluoro-2-[3-fluoro-4-[[4-[4-[[1-methyl-3-oxo-2-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4 (13),5,7-tetraen-9-one;
6-fluoro-2-[3-fluoro-4-[[4-[4-[[2-(2-hydroxyethyl)-3-oxo-1-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4 (13),5,7-tetraen-9-one;
6-fluoro-2-[3-fluoro-4-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]phenyl]-3,10-diazatricyclo[6.4.1.04,13]trideca-1,4 (13),5,7-tetraen-9-one;
6-fluoro-2-[3-methyl-4-[[4-[4-[[2-methyl-3-oxo-1-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazin-1-yl]methyl]-3-methyl-phenyl]-3,10-diazatricyclo[6.4.1.04,13]trideca-1,4,6,8 (13)-tetraen-9-one;
6-fluoro-2-[3-methyl-4-[[4-[4-[[1-methyl-3-oxo-2-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one;
6-fluoro-2-[4-[[4-[4-[[2-(2-hydroxyethyl)-3-oxo-1-(2-pyridyl)pyrazolo[3,4-d]pyrimidin-6-yl]amino]phenyl]piperazin-1-yl]methyl]-3-methyl-phenyl]-3,10-diazatricyclo[6.4.1.04,13]trideca-1,4,6,8 (13)-tetraen-9-one;
6-fluoro-2-[4-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]-3-methyl-phenyl]-3,10-diazatricyclo[6.4.1.04,13]trideca-1,4,6,8 (13)-tetraen-9-one;
2-[3,5-difluoro-4-[[4-[4-[[2-methyl-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[3,5-difluoro-4-[4-[[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[3,5-difluoro-4-[[4-[4-[[1-methyl-3-oxo-2-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[3,5-difluoro-4-[[4-[4-[[2-(2-hydroxyethyl)-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[3,5-difluoro-4-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-2-chloro-phenyl]-6-fluoro-3,10-diazatricyclo[6.4.1.04,13]trideca-1,4 (13),5,7-tetraen-9-one;
2-[2-chloro-4-[[4-[4-[[2-methyl-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[2-chloro-4-[4-[[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[2-chloro-4-[[4-[4-[[1-methyl-3-oxo-2-(2-pyridyl)pyrazolo[3,4-d]pyrimidin-6-ylamino]phenyl]piperazin-1-yl]methyl]phenyl]-6-fluoro-3,10-diazatricyclo[6.4.1.04,13]trideca-1,4 (13),5,7-tetraen-9-one;
2-[2-chloro-4-[[4-[4-[[2-(2-hydroxyethyl)-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[2-chloro-4-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]phenyl]-6-fluoro-3,10-diazatricyclo[6.4.1.04,13]trideca-1,4 (13),5,7-tetraen-9-one;
2-[4-[1-[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]ethyl]phenyl]-6-fluoro-3,10-diazatricyclo[6.4.1.04,13]trideca-1,4,6,8 (13)-tetraen-9-one;
6-fluoro-2-[4-[1-[4-[4-[[2-methyl-3-oxo-1-(2-pyridyl)pyrazolo[3,4-d]pyrimidin-6-yl]amino]phenyl]piperazin-1-yl]ethyl]phenyl]-3,10-diazatricyclo[6.4.1.04,13]trideca-1,4,6,8 (13)-tetraen-9-one;
6-fluoro-2-[4-[1-[4-[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazin-1-yl]ethyl]phenyl]-3,10-diazatricyclo[6.4.1.04,13]trideca-1,4,6,8 (13)-tetraen-9-one;
6-fluoro-2-[4-[1-[4-[4-[[1-methyl-3-oxo-2-(2-pyridyl)pyrazolo[3,4-d]pyrimidin-6-yl]amino]phenyl]piperazin-1-yl]ethyl]phenyl]-3,10-diazatricyclo[6.4.1.04,13]trideca-1,4,6,8 (13)-tetraen-9-one;
6-fluoro-2-[4-[1-[4-[4-[[2-(2-hydroxyethyl)-3-oxo-1-(2-pyridyl)pyrazolo[3,4-d]pyrimidin-6-yl]amino]phenyl]piperazin-1-yl]ethyl]phenyl]-3,10-diazatricyclo[6.4.1.04,13]trideca-1,4,6,8 (13)-tetraen-9-one;
6-fluoro-2-[4-[1-[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]ethyl]phenyl]-3,10-diazatricyclo[6.4.1.04,13]trideca-1,4,6,8 (13)-tetraen-9-one;
2-[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]-3H-benzimidazole-4-carboxamide;
2-[4-[4-[[2-methyl-3-oxo-1-(2-pyridyl)pyrazolo[3,4-d]pyrimidin-6-yl]amino]phenyl]piperazin-1-yl]-3H-benzimidazole-4-carboxamide;
2-[1-[4-[[2-allyl-1-[6-(1-hydroxy-1-methyl-ethyl)-2-pyridyl]-3-oxo-pyrazolo[3,4-d]pyrimidin-6-yl]amino]phenyl]-4-piperidyl]-3H-benzimidazole-4-carboxamide;
2-[1-[4-[[2-methyl-3-oxo-1-(2-pyridyl)pyrazolo[3,4-d]pyrimidin-6-yl]amino]phenyl]-4-piperidyl]-3H-benzimidazole-4-carboxamide;
2-[1-[4-[[2-allyl-1-[6-(1-hydroxy-1-methyl-ethyl)-2-pyridyl]-3-oxo-pyrazolo[3,4-d]pyrimidin-6-yl]amino]phenyl]-4-piperidyl]indazole-7-carboxamide;
2-[1-[4-[[2-methyl-3-oxo-1-(2-pyridyl)pyrazolo[3,4-d]pyrimidin-6-yl]amino]phenyl]-4-piperidyl]indazole-7-carboxamide;
2-[1-[4-[[2-allyl-1-[6-(1-hydroxy-1-methyl-ethyl)-2-pyridyl]-3-oxo-pyrazolo[3,4-d]pyrimidin-6-yl]amino]phenyl]-3-piperidyl]indazole-7-carboxamide;
2-[1-[4-[[2-methyl-3-oxo-1-(2-pyridyl)pyrazolo[3,4-d]pyrimidin-6-yl]amino]phenyl]-3-piperidyl]indazole-7-carboxamide;
2-[1-[4-[[1-methyl-3-oxo-2-(2-pyridyl)pyrazolo[3,4-d]pyrimidin-6-yl]amino]phenyl]-4-piperidyl]-3H-benzimidazole-4-carboxamide;
2-[1-[4-[[2-allyl-3-oxo-1-(2-pyridyl)pyrazolo[3,4-d]pyrimidin-6-yl]amino]phenyl]-4-piperidyl]-3H-benzimidazole-4-carboxamide;
2-[1-[4-[[1-methyl-3-oxo-2-(2-pyridyl)pyrazolo[3,4-d]pyrimidin-6-yl]amino]phenyl]-4-piperidyl]indazole-7-carboxamide;
2-[1-[4-[[2-allyl-3-oxo-1-(2-pyridyl)pyrazolo[3,4-d]pyrimidin-6-yl]amino]phenyl]-4-piperidyl]indazole-7-carboxamide;
2-[4-[4-[[1-methyl-3-oxo-2-(2-pyridyl)pyrazolo[3,4-d]pyrimidin-6-yl]amino]phenyl]piperazin-1-yl]-3H-benzimidazole-4-carboxamide;
2-[4-[4-[[2-allyl-3-oxo-1-(2-pyridyl)pyrazolo[3,4-d]pyrimidin-6-yl]amino]phenyl]piperazin-1-yl]-3H-benzimidazole-4-carboxamide;
4-[[4-fluoro-3-[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazine-1-carbonyl]phenyl]methyl]-2H-phthalazin-1-one;
2-[1-[4-[[2-methyl-3-oxo-1-(2-pyridyl)pyrazolo[3,4-d]pyrimidin-6-yl]amino]phenyl]-3-piperidyl]-3H-benzimidazole-4-carboxamide;
1-[[3-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-4-fluoro-phenyl]methyl]-5-fluoro-quinazoline-2,4-dione;
4-[[4-fluoro-3-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]phenyl]methyl]-2H-phthalazin-1-one;
4-[[3-[4-[4-[[2-allyl-3-oxo-1-(2-pyridyl)pyrazolo[3,4-d]pyrimidin-6-yl]amino]phenyl]piperazine-1-carbonyl]-4-fluoro-phenyl]methyl]-2H-phthalazin-1-one;
4-[[4-fluoro-3-[4-[4-[(2-methyl-3-oxo-1-pyrimidin-2-yl-pyrazolo[3,4-d]pyrimidin-6-yl)amino]phenyl]piperazine-1-carbonyl]phenyl]methyl]-2H-phthalazin-1-one;
4-[[4-fluoro-3-[4-[4-[[1-methyl-3-oxo-2-(2-pyridyl)pyrazolo[3,4-d]pyrimidin-6-yl]amino]phenyl]piperazine-1-carbonyl]phenyl]methyl]-2H-phthalazin-1-one;
4-[[4-fluoro-3-[4-[4-[[1-[6-(1-hydroxy-1-methyl-ethyl)-2-pyridyl]-2-(2-methoxyethyl)-3-oxo-pyrazolo[3,4-d]pyrimidin-6-yl]amino]phenyl]piperazine-1-carbonyl]phenyl]methyl]-2H-phthalazin-1-one;
4-[[3-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-4-fluoro-phenyl]methyl]-2H-phthalazin-1-one;
4-[[4-fluoro-3-[4-[4-[[2-(2-methoxyethyl)-3-oxo-1-(2-pyridyl)pyrazolo[3,4-d]pyrimidin-6-yl]amino]phenyl]piperazine-1-carbonyl]phenyl]methyl]-2H-phthalazin-1-one;
4-[[4-fluoro-3-[4-[4-[[2-(2-hydroxyethyl)-3-oxo-1-(2-pyridyl)pyrazolo[3,4-d]pyrimidin-6-yl]amino]phenyl]piperazine-1-carbonyl]phenyl]methyl]-2H-phthalazin-1-one;
2-[1-[4-[[1-methyl-3-oxo-2-(2-pyridyl)pyrazolo[3,4-d]pyrimidin-6-yl]amino]phenyl]-3-piperidyl]-3H-benzimidazole-4-carboxamide;
4-[[4-fluoro-3-[4-[4-[[2-(2-hydroxyethyl)-1-[6-(1-hydroxy-1-methyl-ethyl)-2-pyridyl]-3-oxo-pyrazolo[3,4-d]pyrimidin-6-yl]amino]phenyl]piperazine-1-carbonyl]phenyl]methyl]-2H-phthalazin-1-one;
5-fluoro-1-[[4-fluoro-3-[[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazin-1-yl]methyl]phenyl]methyl]quinazoline-2,4-dione;
5-fluoro-1-[[4-fluoro-3-[[4-[4-[[2-(2-methoxyethyl)-3-oxo-1-(2-pyridyl)pyrazolo[3,4-d]pyrimidin-6-yl]amino]phenyl]piperazin-1-yl]methyl]phenyl]methyl]quinazoline-2,4-dione;
1-[[3-[[4-[4-[[2-allyl-3-oxo-1-(2-pyridyl)pyrazolo[3,4-d]pyrimidin-6-yl]amino]phenyl]piperazin-1-yl]methyl]-4-fluoro-phenyl]methyl]-5-fluoro-quinazoline-2,4-dione;
5-fluoro-1-[[4-fluoro-3-[[4-[4-[[2-methyl-3-oxo-1-(2-pyridyl)pyrazolo[3,4-d]pyrimidin-6-yl]amino]phenyl]piperazin-1-yl]methyl]phenyl]methyl]quinazoline-2,4-dione;
5-fluoro-1-[[4-fluoro-3-[[4-[4-[[1-methyl-3-oxo-2-(2-pyridyl)pyrazolo[3,4-d]pyrimidin-6-yl]amino]phenyl]piperazin-1-yl]methyl]phenyl]methyl]quinazoline-2,4-dione;
4-[[4-fluoro-3-[[4-[4-[[2-(2-methoxyethyl)-3-oxo-1-(2-pyridyl)pyrazolo[3,4-d]pyrimidin-6-yl]amino]phenyl]piperazin-1-yl]methyl]phenyl]methyl]-2H-phthalazin-1-one;
4-[[3-[[4-[4-[[2-allyl-3-oxo-1-(2-pyridyl)pyrazolo[3,4-d]pyrimidin-6-yl]amino]phenyl]piperazin-1-yl]methyl]-4-fluoro-phenyl]methyl]-2H-phthalazin-1-one;
4-[[4-fluoro-3-[[4-[4-[[2-methyl-3-oxo-1-(2-pyridyl)pyrazolo[3,4-d]pyrimidin-6-yl]amino]phenyl]piperazin-1-yl]methyl]phenyl]methyl]-2H-phthalazin-1-one;
4-[[4-fluoro-3-[[4-[4-[[1-methyl-3-oxo-2-(2-pyridyl)pyrazolo[3,4-d]pyrimidin-6-yl]amino]phenyl]piperazin-1-yl]methyl]phenyl]methyl]-2H-phthalazin-1-one;
2-[1-[4-[[2-allyl-3-oxo-1-(2-pyridyl)pyrazolo[3,4-d]pyrimidin-6-yl]amino]phenyl]-3-piperidyl]-3H-benzimidazole-4-carboxamide;
4-[[4-fluoro-3-[4-[4-[[3-oxo-2-(2-pyridyl)-1H-pyrazolo[3,4-d]pyrimidin-6-yl]amino]phenyl]piperazine-1-carbonyl]phenyl]methyl]-2H-phthalazin-1-one;
4-[[4-fluoro-3-[4-[4-[(2-methyl-3-oxo-1H-pyrazolo[3,4-d]pyrimidin-6-yl)amino]phenyl]piperazine-1-carbonyl]phenyl]methyl]-2H-phthalazin-1-one;
4-[[3-[4-[4-[[2-[2-(dimethylamino)ethyl]-3-oxo-1-(2-pyridyl)pyrazolo[3,4-d]pyrimidin-6-yl]amino]phenyl]piperazine-1-carbonyl]-4-fluoro-phenyl]methyl]-2H-phthalazin-1-one;
4-[[4-fluoro-3-[4-[4-[[2-methyl-3-oxo-1-(4-pyridyl)pyrazolo[3,4-d]pyrimidin-6-yl]amino]phenyl]piperazine-1-carbonyl]phenyl]methyl]-2H-phthalazin-1-one;
4-[[4-fluoro-3-[4-[4-[[2-methyl-3-oxo-1-(3-pyridyl)pyrazolo[3,4-d]pyrimidin-6-yl]amino]phenyl]piperazine-1-carbonyl]phenyl]methyl]-2H-phthalazin-1-one;
4-[[4-fluoro-3-[4-[4-[[1-methyl-3-oxo-2-(4-pyridyl)pyrazolo[3,4-d]pyrimidin-6-yl]amino]phenyl]piperazine-1-carbonyl]phenyl]methyl]-2H-phthalazin-1-one;
4-[[4-fluoro-3-[4-[4-[[1-methyl-3-oxo-2-(3-pyridyl)pyrazolo[3,4-d]pyrimidin-6-yl]amino]phenyl]piperazine-1-carbonyl]phenyl]methyl]-2H-phthalazin-1-one;
2-[1-[4-[[2-allyl-1-[6-(1-hydroxy-1-methyl-ethyl)-2-pyridyl]-3-oxo-pyrazolo[3,4-d]pyrimidin-6-yl]amino]phenyl]-3-piperidyl]-3H-benzimidazole-4-carboxamide
2-fluoro-N-[4-[[2-methyl-3-oxo-1-(2-pyridyl)pyrazolo[3,4-d]pyrimidin-6-yl]amino]phenyl]-5-[(4-oxo-3H-phthalazin-1-yl)methyl]benzamide;
N-[4-[[2-allyl-1-[6-(1-hydroxy-1-methyl-ethyl)-2-pyridyl]-3-oxo-pyrazolo[3,4-d]pyrimidin-6-yl]amino]phenyl]-2-fluoro-5-[(4-oxo-3H-phthalazin-1-yl)methyl]benzamide;
N-[4-[[2-allyl-1-[6-(1-hydroxy-1-methyl-ethyl)-2-pyridyl]-3-oxo-pyrazolo[3,4-d]pyrimidin-6-yl]amino]phenyl]-2-fluoro-5-[(5-fluoro-2,4-dioxo-quinazolin-1-yl)methyl]benzamide;
1-[[3-[4-[[2-allyl-1-[6-(1-hydroxy-1-methyl-ethyl)-2-pyridyl]-3-oxo-pyrazolo[3,4-d]pyrimidin-6-yl]amino]piperidine-1-carbonyl]-4-fluoro-phenyl]methyl]-5-fluoro-quinazoline-2,4-dione;
N-[1-[2-allyl-1-[6-(1-hydroxy-1-methyl-ethyl)-2-pyridyl]-3-oxo-pyrazolo[3,4-d]pyrimidin-6-yl]-4-piperidyl]-2-fluoro-5-[(5-fluoro-2,4-dioxo-quinazolin-1-yl)methyl]benzamide;
N-[2-[[2-allyl-1-[6-(1-hydroxy-1-methyl-ethyl)-2-pyridyl]-3-oxo-pyrazolo[3,4-d]pyrimidin-6-yl]amino]ethyl]-2-fluoro-5-[(5-fluoro-2,4-dioxo-quinazolin-1-yl)methyl]benzamide;
1-[[3-[4-[2-allyl-1-[6-(1-hydroxy-1-methyl-ethyl)-2-pyridyl]-3-oxo-pyrazolo[3,4-d]pyrimidin-6-yl]piperazine-1-carbonyl]-4-fluoro-phenyl]methyl]-5-fluoro-quinazoline-2,4-dione;
2-fluoro-N-[2-[[2-methyl-3-oxo-1-(2-pyridyl)pyrazolo[3,4-d]pyrimidin-6-yl]amino]ethyl]-5-[(4-oxo-3H-phthalazin-1-yl)methyl]benzamide;
4-[[4-fluoro-3-[4-[[2-methyl-3-oxo-1-(2-pyridyl)pyrazolo[3,4-d]pyrimidin-6-yl]amino]piperidine-1-carbonyl]phenyl]methyl]-2H-phthalazin-1-one;
2-fluoro-N-[1-[2-methyl-3-oxo-1-(2-pyridyl)pyrazolo[3,4-d]pyrimidin-6-yl]-4-piperidyl]-5-[(4-oxo-3H-phthalazin-1-yl)methyl]benzamide;
N-[1-[2-allyl-1-[6-(1-hydroxy-1-methyl-ethyl)-2-pyridyl]-3-oxo-pyrazolo[3,4-d]pyrimidin-6-yl]-4-piperidyl]-2-fluoro-5-[(4-oxo-3H-phthalazin-1-yl)methyl]benzamide or a pharmaceutically acceptable salt, stereoisomer, solvate, prodrug, or tautomer thereof.

In some embodiments, the compound is 4-[[3-[4-[[4-[2-allyl-1-[3-(1-hydroxy-1-methyl-ethyl)phenyl]-3-oxo-pyrazolo[3,4-d]pyrimidin-6-yl]amino]phenyl]piperazine-1-carbonyl]-4-fluoro-phenyl]methyl]-2H-phthalazin-1-one or a pharmaceutically acceptable salt, stereoisomer, solvate, prodrug, or tautomer thereof.

In some embodiments, the compound is 1-[[3-[4-[[4-[2-allyl-1-[6-(1-hydroxy-1-methyl-ethyl)-2-pyridyl]-3-oxo-pyrazolo[3,4-d]pyrimidin-6-yl]amino]phenyl]piperazine-1-carbonyl]-4-fluoro-phenyl]methyl]-5-fluoro-quinazoline-2,4-dione or a pharmaceutically acceptable salt, stereoisomer, solvate, prodrug, or tautomer thereof.

In some embodiments, the compound is 2-[4-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]phenyl]-6-fluoro-3,10-diazatricyclo[6.4.1.04,13]trideca-1,4,6,8 (13)-tetraen-9-one or a pharmaceutically acceptable salt, stereoisomer, solvate, prodrug, or tautomer thereof.

In some embodiments, the compound is 2-[1-[4-[[2-methyl-3-oxo-1-(2-pyridyl)pyrazolo[3,4-d]pyrimidin-6-yl]amino]phenyl]-3-piperidyl]-3H-benzimidazole-4-carboxamide or a pharmaceutically acceptable salt, stereoisomer, solvate, prodrug, or tautomer thereof.

In some embodiments, the compound is 4-[[3-[4-[4-[[2-allyl-3-oxo-1-(2-pyridyl)pyrazolo[3,4-d]pyrimidin-6-yl]amino]phenyl]piperazine-1-carbonyl]-4-fluoro-phenyl]methyl]-2H-phthalazin-1-one or a pharmaceutically acceptable salt, stereoisomer, solvate, prodrug, or tautomer thereof.

In some embodiments, the compound is 1-[[3-[4-[4-[[2-allyl-3-oxo-1-(2-pyridyl)pyrazolo[3,4-d]pyrimidin-6-yl]amino]phenyl]piperazine-1-carbonyl]-4-fluoro-phenyl]methyl]-5-fluoro-quinazoline-2,4-dione or a pharmaceutically acceptable salt, stereoisomer, solvate, prodrug, or tautomer thereof.

In some embodiments, the compound is 4-[[4-fluoro-3-[4-[4-[[2-methyl-3-oxo-1-pyrimidin-2-yl-pyrazolo[3,4-d]pyrimidin-6-yl)amino]phenyl]piperazine-1-carbonyl]phenyl]methyl]-2H-phthalazin-1-one or a pharmaceutically acceptable salt, stereoisomer, solvate, prodrug, or tautomer thereof.

In some embodiments, the compound is 4-[[4-fluoro-3-[4-[4-[[1-[6-(1-hydroxy-1-methyl-ethyl)-2-pyridyl]-2-methyl-3-oxo-pyrazolo[3,4-d]pyrimidin-6-yl]amino]phenyl]piperazine-1-carbonyl]phenyl]methyl]-2H-phthalazin-1-one or a pharmaceutically acceptable salt, stereoisomer, solvate, prodrug, or tautomer thereof.

In some embodiments, the compound is 2-[4-[[4-[4-[[2-allyl-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4,6,8 (13)-tetraen-9-one or a pharmaceutically acceptable salt, stereoisomer, solvate, prodrug, or tautomer thereof.

In some embodiments, the compound is 1-[[3-[[4-[4-[[2-allyl-1-[6-(1-hydroxy-1-methyl-ethyl)-2-pyridyl]-3-oxo-pyrazolo[3,4-d]pyrimidin-6-yl]amino]phenyl]piperazin-1-yl]methyl]-4-fluoro-phenyl]methyl]-5-fluoro-quinazoline-2,4-dione or a pharmaceutically acceptable salt, stereoisomer, solvate, prodrug, or tautomer thereof.

It should be understood that all isomeric forms are included within the present invention, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the invention, and pharmaceutically acceptable salts, hydrates, solvates, stereoisomers and prodrugs thereof may exist in their tautomeric form (for example, as an amide or imino ether). All such tautomeric forms are contemplated herein as part of the present invention.

The compounds of the invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention. each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the invention may be atropisomers (e.g., substituted biaryls) and are considered as part of this invention. Enantiomers can also be separated by use of a chiral HPLC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula (I) may form salts which are also within the scope of this invention. Reference to a compound of the Formula herein is understood to include reference to salts thereof, unless otherwise indicated.

The present invention relates to compounds which are modulators of poly ADP-ribose polymerase (PARP) enzyme and/or Wee1.

The present invention relates to compounds which are modulators of poly ADP-ribose polymerase (PARP) enzyme and Wee1.

The present invention relates to compounds which are modulators of poly ADP-ribose polymerase (PARP) enzyme.

The present invention relates to compounds which are modulators of Wee1.

In some embodiments, the compounds of the present invention are inhibitors of poly ADP-ribose polymerase (PARP) enzyme and/or Wee1.

In some embodiments, the compounds of the present invention are inhibitors of poly ADP-ribose polymerase (PARP) enzyme and Wee1.

In some embodiments, the compounds of the present invention are inhibitors of poly ADP-ribose polymerase (PARP) enzyme.

In some embodiments, the compounds of the present invention are inhibitors of Wee1.

The invention is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures A-B which comprise different sequences of assembling intermediates or compounds. Starting materials are either commercially available or made by known procedures in the reported literature or as illustrated below.

### General Procedure A

In general, the compound of the Formula (A) can be prepared using reaction of substitution of an appropriate leaving group (halogen, —SO₂Me, etc.) in the intermediate (P-L′-LG) with amino derivatives (H₂N—W):

$$P - {L}^{′} - {NH}_{2} + LG - W \to P - L - W$$

In more specific aspect, the compound of the Formula (I) can be obtained according to the scheme presented below:

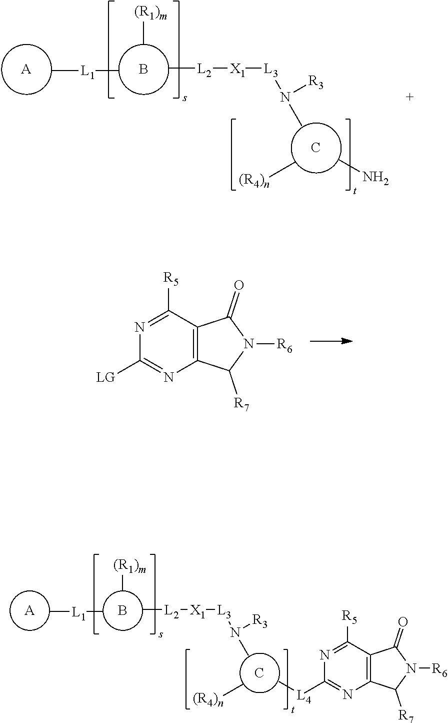

As a specific non-limiting example of this procedure can be presented the reaction of preparation of the compound 29:

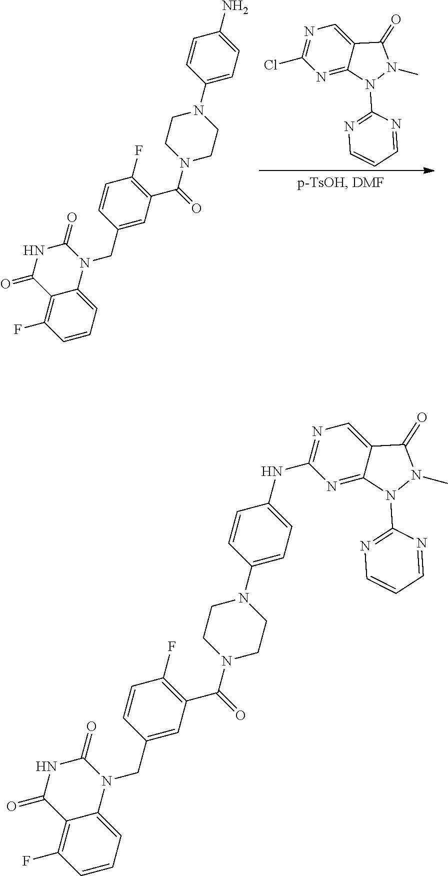

### General Procedure B

In general, the compound of the Formula (A) can be prepared using reaction of addition of an amino substituted intermediate (H₂N-L″-W) to carbonyl derivative of other intermediate (P-L′-C(O)H) with further reduction of imino-derivatives (P-L′—CH═N-L″-W):

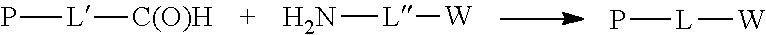

In more specific aspect, the compound of the Formula (I) can be obtained according to the scheme presented below:

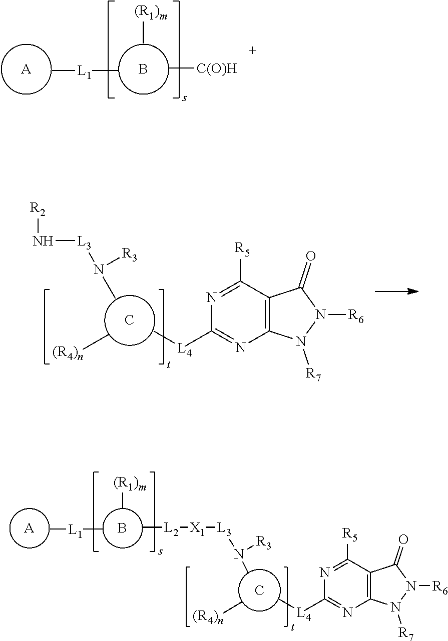

As a specific non-limiting example of this procedure can be presented the reaction of preparation of the compound 14:

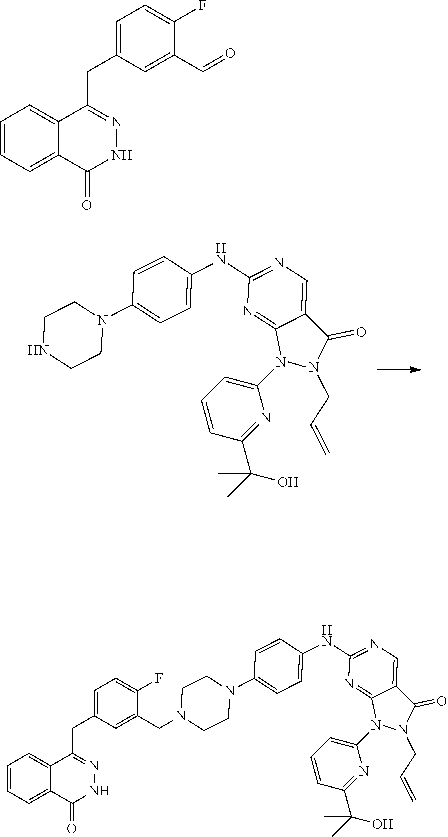 [SMILES: O=Cc1cc(Cc2n[nH]c(=O)c3ccccc23)ccc1F ; C=CCn1c(=O)c2cnc(Nc3ccc(N4CCNCC4)cc3)nc2n1-c1cccc(C(C)(C)O)n1 ; C=CCn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1]

### Methods of Using the Disclosed Compounds

Another aspect of the invention relates to a method of treating a disease or disorder associated with modulation of poly ADP-ribose polymerase (PARP) enzyme and/or Wee1. The method comprises administering to a patient in need of a treatment for diseases or disorders associated with modulation of poly ADP-ribose polymerase (PARP) enzyme and/or Wee1 an effective amount the compositions and compounds of Formula (I).

Another aspect of the invention relates to a method of treating a disease or disorder associated with modulation of poly ADP-ribose polymerase (PARP) enzyme and Wee1. The method comprises administering to a patient in need of a treatment for diseases or disorders associated with modulation of poly ADP-ribose polymerase (PARP) enzyme and Wee1 an effective amount the compositions and compounds of Formula (I).

Another aspect of the invention relates to a method of treating a disease or disorder associated with modulation of poly ADP-ribose polymerase (PARP) enzyme. The method comprises administering to a patient in need of a treatment for diseases or disorders associated with modulation of poly ADP-ribose polymerase (PARP) enzyme an effective amount the compositions and compounds of Formula (I).

Another aspect of the invention relates to a method of treating a disease or disorder associated with modulation of Wee1. The method comprises administering to a patient in need of a treatment for diseases or disorders associated with modulation of Wee1 an effective amount the compositions and compounds of Formula (I).

In another aspect, the present invention is directed to a method of inhibiting poly ADP-ribose polymerase (PARP) enzyme and/or Wee1. The method involves administering to a patient in need thereof an effective amount of a compound of Formula (I).

In another aspect, the present invention is directed to a method of inhibiting poly ADP-ribose polymerase (PARP) enzyme and Wee1. The method involves administering to a patient in need thereof an effective amount of a compound of Formula (I).

In another aspect, the present invention is directed to a method of inhibiting poly ADP-ribose polymerase (PARP) enzyme. The method involves administering to a patient in need thereof an effective amount of a compound of Formula (I).

In another aspect, the present invention is directed to a method of inhibiting Wee1. The method involves administering to a patient in need thereof an effective amount of a compound of Formula (I).

Another aspect of the present invention relates to a method of treating, preventing, inhibiting or eliminating a disease or disorder in a patient associated with the inhibition of poly ADP-ribose polymerase (PARP) enzyme and/or Wee1, the method comprising administering to a patient in need thereof an effective amount of a compound of Formula (I). In some embodiments, the disease may be, but not limited to, cancer.

Another aspect of the present invention relates to a method of treating, preventing, inhibiting or eliminating a disease or disorder in a patient associated with the inhibition of poly ADP-ribose polymerase (PARP) enzyme and Wee1, the method comprising administering to a patient in need thereof an effective amount of a compound of Formula (I). In some embodiments, the disease may be, but not limited to, cancer.

Another aspect of the present invention relates to a method of treating, preventing, inhibiting or eliminating a disease or disorder in a patient associated with the inhibition of poly ADP-ribose polymerase (PARP) enzyme, the method comprising administering to a patient in need thereof an effective amount of a compound of Formula (I). In some embodiments, the disease may be, but not limited to, cancer.

Another aspect of the present invention relates to a method of treating, preventing, inhibiting or eliminating a disease or disorder in a patient associated with the inhibition of Wee1, the method comprising administering to a patient in need thereof an effective amount of a compound of Formula (I). In some embodiments, the disease may be, but not limited to, cancer.

The present invention also relates to the use of an inhibitor of poly ADP-ribose polymerase (PARP) enzyme and/or Wee1 for the preparation of a medicament used in the treatment, prevention, inhibition or elimination of a disease or condition mediated by said enzymes, wherein the medicament comprises a compound of Formula (I).

The present invention also relates to the use of an inhibitor of poly ADP-ribose polymerase (PARP) enzyme and Wee1 for the preparation of a medicament used in the treatment, prevention, inhibition or elimination of a disease or condition mediated by said enzymes, wherein the medicament comprises a compound of Formula (I).

The present invention also relates to the use of an inhibitor of poly ADP-ribose polymerase (PARP) enzyme for the preparation of a medicament used in the treatment, prevention, inhibition or elimination of a disease or condition mediated by said enzyme, wherein the medicament comprises a compound of Formula (I).

The present invention also relates to the use of an inhibitor of Wee1 for the preparation of a medicament used in the treatment, prevention, inhibition or elimination of a disease or condition mediated by said enzyme, wherein the medicament comprises a compound of Formula (I).

In another aspect, the present invention relates to a method for the manufacture of a medicament for treating, preventing, inhibiting, or eliminating a disease or condition mediated by poly ADP-ribose polymerase (PARP) enzyme and/or Wee1, wherein the medicament comprises a compound of Formula (I).

In another aspect, the present invention relates to a method for the manufacture of a medicament for treating, preventing, inhibiting, or eliminating a disease or condition mediated by poly ADP-ribose polymerase (PARP) enzyme and Wee1, wherein the medicament comprises a compound of Formula (I).

In another aspect, the present invention relates to a method for the manufacture of a medicament for treating, preventing, inhibiting, or eliminating a disease or condition mediated by poly ADP-ribose polymerase (PARP) enzyme, wherein the medicament comprises a compound of Formula (I).

In another aspect, the present invention relates to a method for the manufacture of a medicament for treating, preventing, inhibiting, or eliminating a disease or condition mediated by Wee1, wherein the medicament comprises a compound of Formula (I).

Another aspect of the present invention relates to a compound of Formula (I) for use in the manufacture of a medicament for treating a disease associated with inhibiting poly ADP-ribose polymerase (PARP) enzyme and/or Wee1.

Another aspect of the present invention relates to a compound of Formula (I) for use in the manufacture of a medicament for treating a disease associated with inhibiting poly ADP-ribose polymerase (PARP) enzyme and Wee1.

Another aspect of the present invention relates to a compound of Formula (I) for use in the manufacture of a medicament for treating a disease associated with inhibiting poly ADP-ribose polymerase (PARP) enzyme.

Another aspect of the present invention relates to a compound of Formula (I) for use in the manufacture of a medicament for treating a disease associated with inhibiting Wee1.

In another aspect, the present invention relates to the use of a compound of Formula (I) in the treatment of a disease associated with inhibiting poly ADP-ribose polymerase (PARP) enzyme and/or Wee1.

In another aspect, the present invention relates to the use of a compound of Formula (I) in the treatment of a disease associated with inhibiting poly ADP-ribose polymerase (PARP) enzyme and Wee1.

In another aspect, the present invention relates to the use of a compound of Formula (I) in the treatment of a disease associated with inhibiting poly ADP-ribose polymerase (PARP) enzyme.

In another aspect, the present invention relates to the use of a compound of Formula (I) in the treatment of a disease associated with inhibiting Wee1.

Another aspect of the invention relates to a method of treating or preventing cancer. The method comprises administering to a patient in need thereof an effective amount of a compound of Formula (I).

Another aspect of the invention relates to a method of treating cancer. The method comprises administering to a patient in need thereof an effective amount of a compound of Formula (I).

In some embodiments, the present invention relates to the use of an inhibitor of poly ADP-ribose polymerase (PARP) enzyme and/or Wee1 for the preparation of a medicament used in treatment, prevention, inhibition or elimination of a disease or disorder associated with cancer.

In some embodiments, the present invention relates to the use of an inhibitor of poly ADP-ribose polymerase (PARP) enzyme and Wee1 for the preparation of a medicament used in treatment, prevention, inhibition or elimination of a disease or disorder associated with cancer.

In some embodiments, the present invention relates to the use of an inhibitor of poly ADP-ribose polymerase (PARP) enzyme for the preparation of a medicament used in treatment, prevention, inhibition or elimination of a disease or disorder associated with cancer.

In some embodiments, the present invention relates to the use of an inhibitor of Wee1 for the preparation of a medicament used in treatment, prevention, inhibition or elimination of a disease or disorder associated with cancer.

In some embodiments, the PARP enzyme is selected from PARP1, PARP 2, PARP3, PARP4, PARP-5A, PARP-5B, PARP6, PARP7, PARP8, PARP9, PARP10, PARP11, PARP12, PARTP14, PARP15, and PARP16.

In some embodiments, the PARP enzyme is PARP1.

In some embodiments, the PARP enzyme is PARP2.

In some embodiments, the present invention relates to a compound of Formula (I) or a pharmaceutical composition comprising a compound of the present invention and a pharmaceutically acceptable carrier used for the treatment of cancers including, but not limited to, bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, marginal zone lymphoma (MZL), follicular lymphoma (FL), diffuse large B-cell lymphoma (DLBCL), fallopian tube cancer, peritoneal cancer, and chronic lymphocytic leukemia/small lymphocytic lymphoma (CLL/SLL).

In some embodiments, the cancer is selected from ovarian cancer, breast cancer, prostate cancer, pancreatic cancer, fallopian tube cancer, and peritoneal cancer.

In some embodiments, the cancer is selected from ovarian cancer, pancreatic cancer, fallopian tube cancer, and peritoneal cancer.

In some embodiments, the cancer is ovarian cancer.

In some embodiments, the ovarian cancer is epithelial ovarian cancer.

In some embodiments, the ovarian cancer is somatic BRCA-mutated (sBRCAm) advanced ovarian cancer.

In some embodiments, the ovarian cancer is germline BRCA mutated (gBRCAm) advanced ovarian cancer.

In some embodiments, the cancer is breast cancer.

In some embodiments, the breast cancer is germline BRCA mutated (gBRCAm) HER2-negative metastatic breast cancer.

In some embodiments, the cancer is prostate cancer.

In some embodiments, the prostate cancer is metastatic castration-resistant prostate cancer.

In some embodiments, the cancer is pancreatic cancer.

In some embodiments, the cancer is fallopian tube cancer.

In some embodiments, the cancer is peritoneal cancer.

In some embodiments, the peritoneal cancer is primary peritoneal cancer.

In some embodiments, the cancer comprises a solid tumor.

In some embodiments, the solid tumor is a primary tumor or a metastatic tumor.

In some embodiments, the cancer is a recurrent cancer.

In some embodiments, the subject has a BRCA mutation.

In some embodiments, the BRCA mutation is a BRCA1 mutation.

In some embodiments, the BRCA mutation is a BRCA2 mutation.

In some embodiments, the BRCA mutation is a hereditary BRCA mutation.

In some embodiments, the subject has a human epidermal growth factor receptor (HER) mutation.

In some embodiments, the HER mutation is a HER2 mutation.

In some embodiments, the subject is in complete response to first-line platinum-based chemotherapy.

In some embodiments, the subject is in partial response to first-line platinum-based chemotherapy.

In some embodiments, the subject is a mammal.

In some embodiments, the subject is a human.

In some embodiments, the human is 18 years or older.

In some embodiments, the contacting is in vitro or in vivo.

Another aspect of the invention is directed to pharmaceutical compositions comprising a compound of Formula (I) and a pharmaceutically acceptable carrier. The pharmaceutical acceptable carrier may further include an excipient, diluent, or surfactant.

In some embodiments, are provided methods of treating a disease or disorder associated with modulation of poly ADP-ribose polymerase (PARP) enzyme and/or Wee1, including cancer, comprising administering to a patient suffering from at least one of said diseases or disorder a compound of Formula (I).

In some embodiments, are provided methods of treating a disease or disorder associated with modulation of poly ADP-ribose polymerase (PARP) enzyme and Wee1, including cancer, comprising administering to a patient suffering from at least one of said diseases or disorder a compound of Formula (I).

In some embodiments, are provided methods of treating a disease or disorder associated with modulation of poly ADP-ribose polymerase (PARP) enzyme, including cancer, comprising administering to a patient suffering from at least one of said diseases or disorder a compound of Formula (I).

In some embodiments, are provided methods of treating a disease or disorder associated with modulation of Wee1, including cancer, comprising administering to a patient suffering from at least one of said diseases or disorder a compound of Formula (I).

One therapeutic use of the compounds or compositions of the present invention which inhibit poly ADP-ribose polymerase (PARP) enzyme and/or Wee1 is to provide treatment to patients or subjects suffering from a cancer.

One therapeutic use of the compounds or compositions of the present invention which inhibit poly ADP-ribose polymerase (PARP) enzyme and Wee1 is to provide treatment to patients or subjects suffering from a cancer.

One therapeutic use of the compounds or compositions of the present invention which inhibit poly ADP-ribose polymerase (PARP) enzyme is to provide treatment to patients or subjects suffering from a cancer.

One therapeutic use of the compounds or compositions of the present invention which inhibit Wee1 is to provide treatment to patients or subjects suffering from a cancer.

The disclosed compounds of the invention can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In some embodiments, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention is directed to pharmaceutical compositions comprising a compound of Formula (I) and a pharmaceutically acceptable carrier. The pharmaceutical acceptable carrier may further include an excipient, diluent, or surfactant. In some embodiments, the pharmaceutical composition can further comprise an additional pharmaceutically active agent.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In some embodiments, the compositions are in the form of a tablet that can be scored.

### Examples

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Abbreviations used in the following examples and elsewhere herein are:

| | |
|---|---|
| AcOH | acetic acid |
| aq. | aqueous |
| br. | broad |
| BSA | bovine serum albumin |
| conc. | concentrated |
| d | duplet |
| DCM | dichloromethane |
| DIPEA | N,N-diisopropylethylamine |
| DMEM | Dulbecco's modified Eagle's medium |
| DMF | N,N-dimethyl formamide |
| DMSO | dimethyl sulfoxide |
| DTT | dithiothreitol |
| EDCI | 1-(3-Dimethylaminopropyl)-3-ethylcarbodiimide |
| FBS | fetal bovine serum |
| h | hour(s) |
| HMDS | hexamethyldisilazane |
| HOBt | 1-hydroxybenzotriazole |
| HPLC | high pressure (or performance) liquid chromatography |
| LCMS | liquid chromatography mass spectrometry |
| m | multiplet |
| M | molar |
| m-CPBA | 3-chloroperbenzoic acid |
| MHz | megahertz |
| min | minutes |
| NBS | N-bromosuccinimide |
| NMR | nuclear magnetic resonance |
| PEG | polyethylene glycol |
| p-TsOH | 4-methylbenzene-1-sulfonic acid |
| q | quadruplet |
| RP-HPLC | Reversed-phase high pressure (or performance) |
| | liquid chromatography |
| rt | room temperature |
| S | singlet |
| STAB | sodium triacetoxyborohydride |
| t | temperature, triplet |
| T3P | Propylphosphonic anhydride |
| TFA | trifluoroacetic acid |
| THF | tetrahydrofuran |
| TLC | thin layer chromatography |
| TRIS | 2-amino-2-(hydroximethyl)propane-1,3-diol |
| TsOH | toluenesulfonic acid (e.g. p-TsOH) |

Purity and identity of all synthesized compounds were confirmed by LC-MS analysis performed on Shimadzu Analytical 10Avp equipped with PE SCIEX API 165 mass-, Sedex 75 ELSD-, and Shimadzu UV-(254 and 215) detectors. Separation was achieved with C₁₈ column 100× 4.6 mm, 5.0 μm, pore size 100 Å, water-acetonitrile+0.1 TFA, gradient 5 to 87 for 10 min.

Preparative HPLC purification was carried out on Shimadzu instrument equipped with SPD-10Avp detector and FRC-10A fraction collector. Separation was achieved with a column YMC-Pack ODS-AQ 250×20 mml, S-10 μm, 12 nm, gradient solution A-solution B (A: 1000 mL H₂O-226 μL TFA; B: 1000 mL CH₃CN-226 μL TFA).

In the Table 1 presented examples of the compound synthesized in the frame of this invention, results of MS analysis and ID numbers for each compound for the further reference.

TABLE 1
Examples of the compounds and analytical data

| | | | [MH]⁺ | [MH]⁺ |
|---|---|---|---|---|
| # | Structure | IUPAC name | calc. | found |
| 1 | 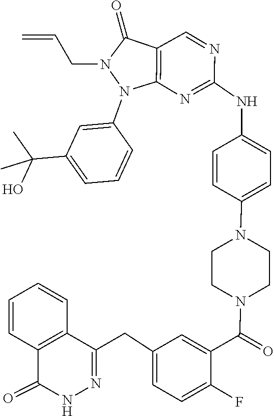 [SMILES: C=CCn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)c1] | 4-[[3-[4-[4-[[2-allyl-1-[3-(1-hydroxy- 1-methyl-ethyl)phenyl]-3-oxo- pyrazolo[3,4-d]pyrimidin-6- yl]amino]phenyl]piperazine-1- carbonyl]-4-fluoro-phenyl]methyl]- 2H-phthalazin-1-one | 766.3 | 766 |
| 2 | 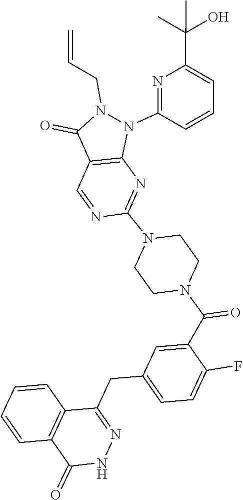 [SMILES: C=CCn1c(=O)c2cnc(N3CCN(C(=O)c4cc(Cc5n[nH]c(=O)c6ccccc56)ccc4F)CC3)nc2n1-c1cccc(C(C)(C)O)n1] | 4-[[3-[4-[2-allyl-1-[6-(1-hydroxy-1- methyl-ethyl)-2-pyridyl]-3-oxo- pyrazolo[3,4-d]pyrimidin-6- yl]piperazine-1-carbonyl]-4-fluoro- phenyl]methyl]-2H-phthalazin-1-one | 676.3 | 676 |
| 3 | 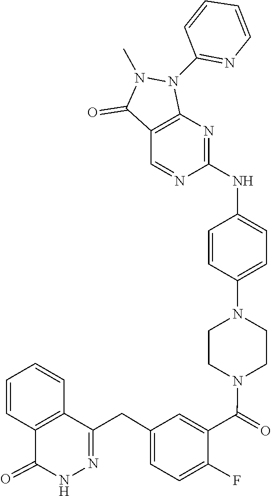 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1ccccn1] | 4-[[4-fluoro-3-[4-[4-[2-methyl-3- oxo-1-(2-pyridyl)pyrazolo[3,4- d]pyrimidin-6- yl]amino]phenyl]piperazine-1- carbonyl]phenyl]methyl]-2H- phthalazin-1-one | 683.3 | 683 |
| 4 | 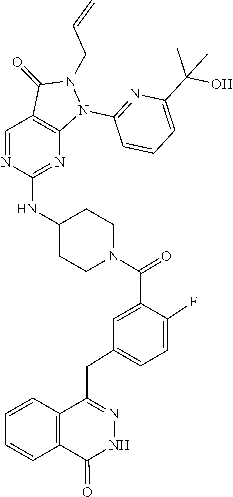 [SMILES: C=CCn1c(=O)c2cnc(NC3CCN(C(=O)c4cc(Cc5n[nH]c(=O)c6ccccc56)ccc4F)CC3)nc2n1-c1cccc(C(C)(C)O)n1] | 4-[[3-[4-[[2-allyl-1-[6-(1-hydroxy-1- methyl-ethyl)-2-pyridyl]-3-oxo- pyrazolo[3,4-d]pyrimidin-6- yl]amino]piperidine-1-carbonyl]-4- fluoro-phenyl]methyl]-2H-phthalazin- 1-one | 690.3 | 690 |
| 5 | 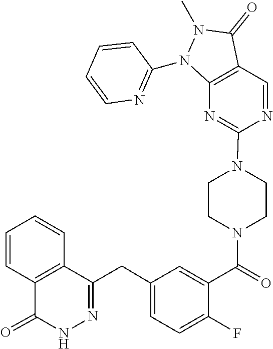 [SMILES: Cn1c(=O)c2cnc(N3CCN(C(=O)c4cc(Cc5n[nH]c(=O)c6ccccc56)ccc4F)CC3)nc2n1-c1ccccn1] | 4-[[4-fluoro-3-[4-[2-methyl-3-oxo-1- (2-pyridyl)pyrazolo[3,4-d]pyrimidin- 6-yl]piperazine-1- carbonyl]phenyl]methyl]-2H- phthalazin-1-one | 592.2 | 592 |
| 6 | 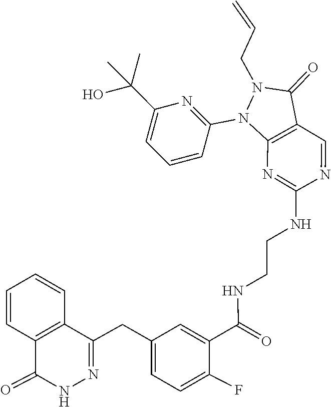 [SMILES: C=CCn1c(=O)c2cnc(NCCNC(=O)c3cc(Cc4n[nH]c(=O)c5ccccc45)ccc3F)nc2n1-c1cccc(C(C)(C)O)n1] | N-[2-[2-allyl-1-[6-(1-hydroxy-1- methyl-ethyl)-2-pyridyl]-3-oxo- pyrazolo[3,4-d]pyrimidin-6- yl]amino]ethyl]-2-fluoro-5-[(4-oxo- 3H-phthalazin-1- yl)methyl]benzamide | 650.3 | 650 |
| 7 | 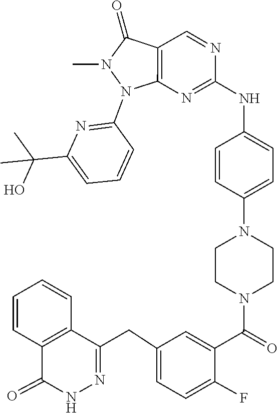 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)n1] | 4-[[4-fluoro-3-[4-[4-[1-[6-(1- hydroxy-1-methyl-ethyl)-2-pyridyl]- 2-methyl-3-oxo-pyrazolo[3,4- d]pyrimidin-6- yl]amino]phenyl]piperazine-1- carbonyl]phenyl]methyl]-2H- phthalazin-1-one | 741.3 | 741 |
| 8 | 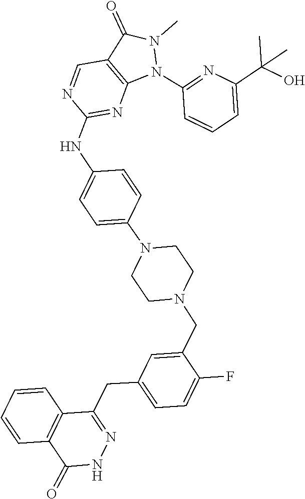 [SMILES: Cn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1] | 4-[[4-fluoro-3-[[4-[4-[[1-[6-(1- hydroxy-1-methyl-ethyl)-2-pyridyl]- 2-methyl-3-oxo-pyrazolo[3,4- d]pyrimidin-6- yl]amino]phenyl]piperazin-1- yl]methyl]phenyl]methyl]-2H- phthalazin-1-one | 727.3 | 727 |
| 9 | 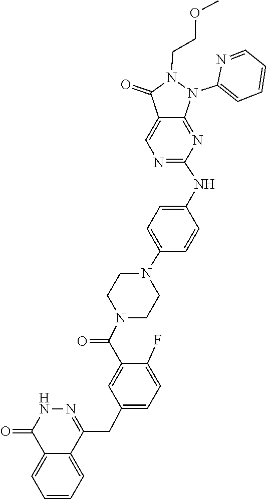 [SMILES: COCCn1c(=O)c2cnc(Nc3ccc(N4CCN(C(=O)c5cc(Cc6n[nH]c(=O)c7ccccc67)ccc5F)CC4)cc3)nc2n1-c1ccccn1] | 4-[[4-fluoro-3-[4-[4-[2-(2- methoxyethyl)-3-oxo-1-(2- pyridyl)pyrazolo[3,4-d]pyrimidin-6- yl]amino]phenyl]piperazine-1- carbonyl]phenyl]methyl]-2H- phthalazin-1-one | 727.3 | 727 |
| 10 | 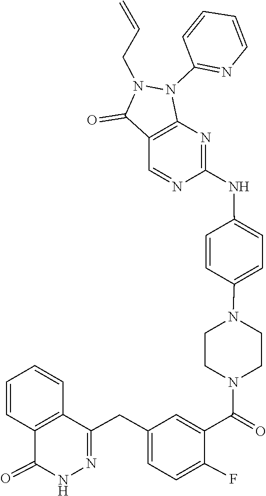 [SMILES: C=CCn1c(=O)c2cnc(Nc3ccc(N4CCN(C(=O)c5cc(Cc6n[nH]c(=O)c7ccccc67)ccc5F)CC4)cc3)nc2n1-c1ccccn1] | 4-[[3-[4-[4-[[2-allyl-3-oxo-1-(2- pyridyl)pyrazolo[3,4-d]pyrimidin-6- yl]amino]phenyl]piperazine-1- carbonyl]-4-fluoro-phenyl]methyl]- 2H-phthalazin-1-one | 709.3 | 709 |
| 11 | 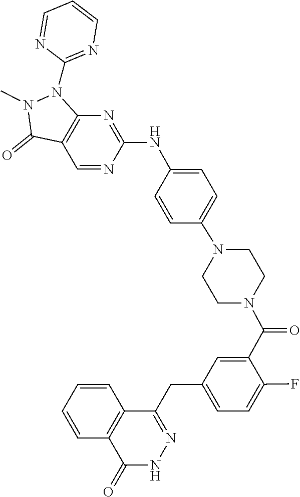 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1ncccn1] | 4-[[4-fluoro-3-[4-[4-[(2-methyl-3- oxo-1-pyrimidin-2-yl-pyrazolo[3,4- d]pyrimidin-6- yl)amino]phenyl]piperazine-1- carbonyl]phenyl]methyl]-2H- phthalazin-1-one | 684.3 | 684 |
| 12 | 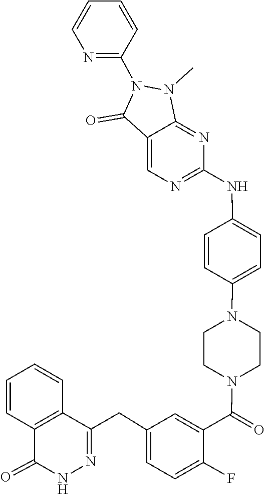 [SMILES: Cn1c2nc(Nc3ccc(N4CCN(C(=O)c5cc(Cc6n[nH]c(=O)c7ccccc67)ccc5F)CC4)cc3)ncc2c(=O)n1-c1ccccn1] | 4-[[4-fluoro-3-[4-[4-[[1-methyl-3- oxo-2-(2-pyridyl)pyrazolo[3,4- d]pyrimidin-6- yl]amino]phenyl]piperazine-1- carbonyl]phenyl]methyl]-2H- phthalazin-1-one | 683.3 | 683 |
| 13 | 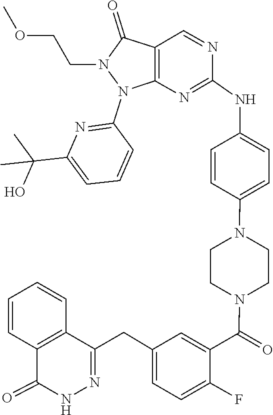 [SMILES: COCCn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)n1] | 4-[[4-fluoro-3-[4-[4-[[1-[6-(1- hydroxy-1-methyl-ethyl)-2-pyridyl]- 2-(2-methoxyethyl)-3-oxo- pyrazolo[3,4-d]pyrimidin-6- yl]amino]phenyl]piperazine-1- carbonyl]phenyl]methyl]-2H- phthalazin-1-one | 785.3 | 785 |
| 14 | 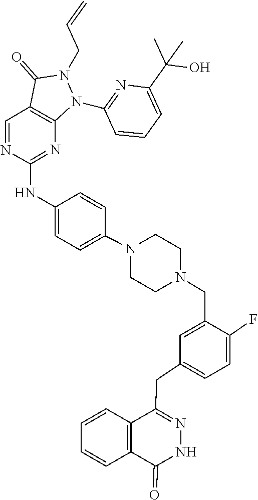 [SMILES: C=CCn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1] | 4-[[3-[[4-[4-[[2-allyl-1-[6-(1- hydroxy-1-methyl-ethyl)-2-pyridyl]- 3-oxo-pyrazolo[3,4-d]pyrimidin-6- yl]amino]phenyl]piperazin-1- yl]methyl]-4-fluoro-phenyl]methyl]- 2H-phthalazin-1-one | 753.3 | 753 |
| 15 | 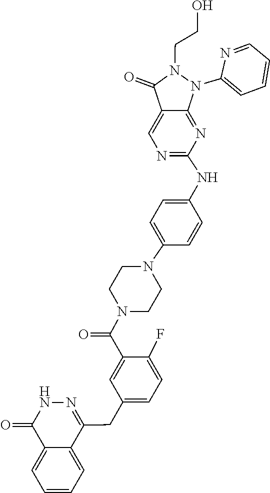 [SMILES: O=C(c1cc(Cc2n[nH]c(=O)c3ccccc23)ccc1F)N1CCN(c2ccc(Nc3ncc4c(=O)n(CCO)n(-c5ccccn5)c4n3)cc2)CC1] | 4-[[4-fluoro-3-[4-[4-[[2-(2- hydroxyethyl)-3-oxo-1-(2- pyridyl)pyrazolo[3,4-d]pyrimidin-6- yl]amino]phenyl]piperazine-1- carbonyl]phenyl]methyl]-2H- phthalazin-1-one | 713.3 | 713 |
| 16 | 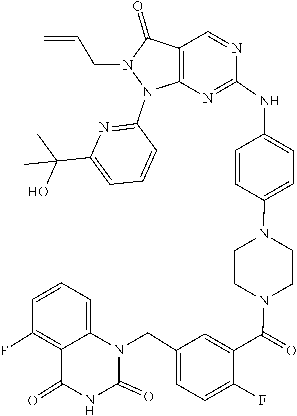 [SMILES: C=CCn1c(=O)c2cnc(Nc3ccc(N4CCN(C(=O)c5cc(Cn6c(=O)[nH]c(=O)c7c(F)cccc76)ccc5F)CC4)cc3)nc2n1-c1cccc(C(C)(C)O)n1] | 1-[[3-[4-[4-[[2-allyl-1-[6-(1-hydroxy- 1-methyl-ethyl)-2-pyridyl]-3-oxo- pyrazolo[3,4-d]pyrimidin-6- yl]amino]phenyl]piperazine-1- carbonyl]-4-fluoro-phenyl]methyl]-5- fluoro-quinazoline-2,4-dione | 801.3 | 801 |
| 17 | 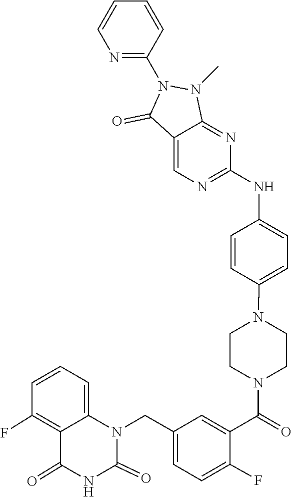 [SMILES: Cn1c2nc(Nc3ccc(N4CCN(C(=O)c5cc(Cn6c(=O)[nH]c(=O)c7c(F)cccc76)ccc5F)CC4)cc3)ncc2c(=O)n1-c1ccccn1] | 5-fluoro-1-[[4-fluoro-3-[4-[4-[[1- methyl-3-oxo-2-(2- pyridyl)pyrazolo[3,4-d]pyrimidin-6- yl]amino]phenyl]piperazine-1- carbonyl]phenyl]methyl]quinazoline- 2,4-dione | 717.2 | 717 |
| 18 | 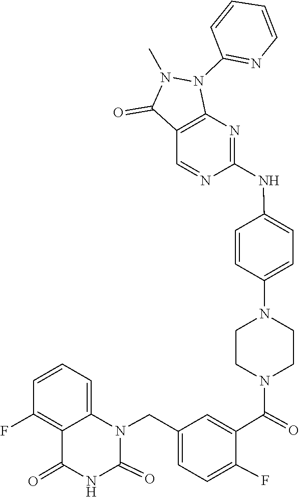 [SMILES: Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1] | 5-fluoro-1-[[4-fluoro-3-[4-[4-[[2- methyl-3-oxo-1-(2- pyridyl)pyrazolo[3,4-d]pyrimidin-6- yl]amino]phenyl]piperazine-1- carbonyl]phenyl]methyl]quinazoline- 2,4-dione | 717.2 | 717 |
| 19 | 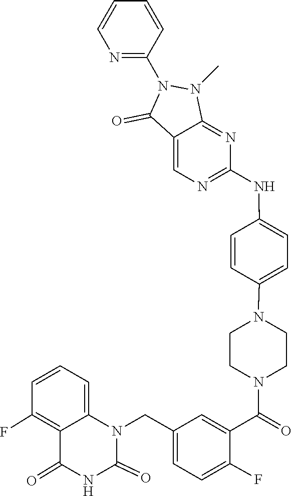 [SMILES: Cn1c2nc(Nc3ccc(N4CCN(C(=O)c5cc(Cn6c(=O)[nH]c(=O)c7c(F)cccc76)ccc5F)CC4)cc3)ncc2c(=O)n1-c1ccccn1] | 5-fluoro-1-[[4-fluoro-3-[4-[4-[1- methyl-3-oxo-2-(3- pyridyl)pyrazolo[3,4-d]pyrimidin-6- yl]amino]phenyl]piperazine-1- carbonyl]phenyl]methyl]quinazoline- 2,4-dione | 717.2 | 717 |
| 20 | 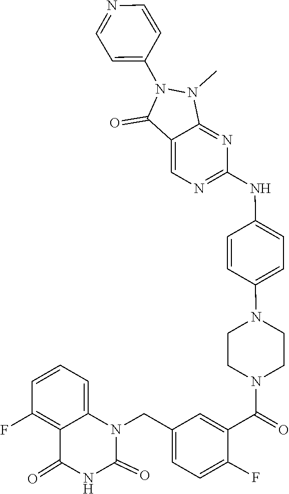 [SMILES: Cn1c2nc(Nc3ccc(N4CCN(C(=O)c5cc(Cn6c(=O)[nH]c(=O)c7c(F)cccc76)ccc5F)CC4)cc3)ncc2c(=O)n1-c1ccncc1] | 5-fluoro-1-[[4-fluoro-3-[4-[4-[[1- methyl-3-oxo-2-(4- pyridyl)pyrazolo[3,4-d]pyrimidin-6- yl]amino]phenyl]piperazine-1- carbonyl]phenyl]methyl]quinazoline- 2,4-dione | 717.2 | 717 |
| 21 | 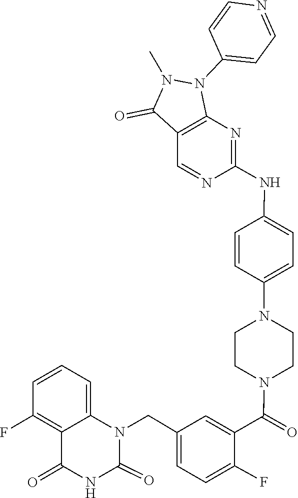 [SMILES: Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccncc1] | 5-fluoro-1-[[4-fluoro-3-[4-[4-[[2- methyl-3-oxo-1-(3- pyridyl)pyrazolo[3,4-d]pyrimidin-6- yl]amino]phenyl]piperazine-1- carbonyl]phenyl]methyl]quinazoline- 2,4-dione | 717.2 | 717 |
| 22 | 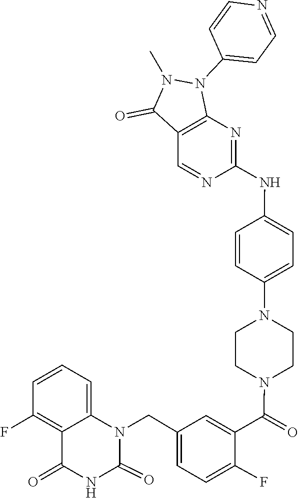 [SMILES: Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccncc1] | 5-fluoro-1-[[4-fluoro-3-[4-[4-[[2- methyl-3-oxo-1-(4- pyridyl)pyrazolo[3,4-d]pyrimidin-6- yl]amino]phenyl]piperazine-1- carbonyl]phenyl]methyl]quinazoline- 2,4-dione | 717.2 | 717 |
| 23 | 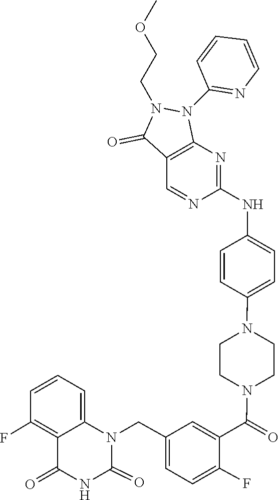 [SMILES: COCCn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1] | 5-fluoro-1-[[4-fluoro-3-[4-[4-[2-(2- methoxyethyl)-3-oxo-1-(2- pyridyl)pyrazolo[3,4-d]pyrimidin-6- yl]amino]phenyl]piperazine-1- carbonyl]phenyl]methyl]quinazoline- 2,4-dione | 761.3 | 761 |
| 24 | 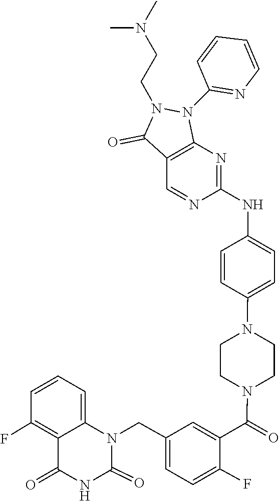 [SMILES: CN(C)CCn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1] | 1-[[3-[4-[4-[[2-[2- (dimethylamino)ethyl]-3-oxo-1-(2- pyridyl)pyrazolo[3,4-d]pyrimidin-6- yl]amino]phenyl]piperazine-1- carbonyl]-4-fluoro-phenyl]methyl]-5- fluoro-quinazoline-2,4-dione | 774.3 | 774 |
| 25 | 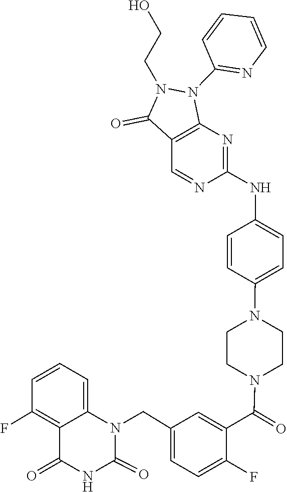 [SMILES: O=C(c1cc(Cn2c(=O)[nH]c(=O)c3c(F)cccc32)ccc1F)N1CCN(c2ccc(Nc3ncc4c(=O)n(CCO)n(-c5ccccn5)c4n3)cc2)CC1] | 5-fluoro-1-[[4-fluoro-3-[4-[4-[[2-(2- hydroxyethyl)-3-oxo-1-(2- pyridyl)pyrazolo[3,4-d]pyrimidin-6- yl]amino]phenyl]piperazine-1- carbonyl]phenyl]methyl]quinazoline- 2,4-dione | 747.3 | 747 |
| 26 | 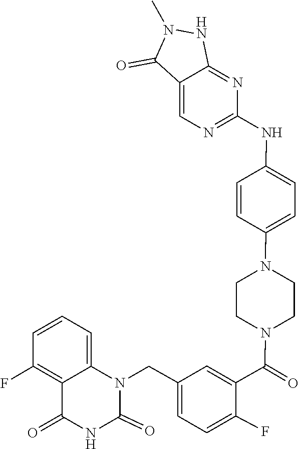 [SMILES: Cn1[nH]c2nc(Nc3ccc(N4CCN(C(=O)c5cc(Cn6c(=O)[nH]c(=O)c7c(F)cccc76)ccc5F)CC4)cc3)ncc2c1=O] | 5-fluoro-1-[[4-fluoro-3-[4-[4-[(2- methyl-3-oxo-1H-pyrazolo[3,4- d]pyrimidin-6- yl)amino]phenyl]piperazine-1- carbonyl]phenyl]methyl]quinazoline- 2,4-dione | 640.2 | 640 |
| 27 | 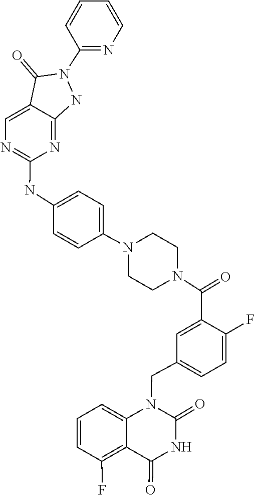 | 5-fluoro-1-[[4-fluoro-3-[4-[4-[[3-oxo- 2-(2-pyridyl)-1H-pyrazolo[3,4- d]pyrimidin-6- yl]amino]phenyl]piperazine-1- carbonyl]phenyl]methyl]quinazoline- 2,4-dione | 703.2 | 703 |
| 28 | 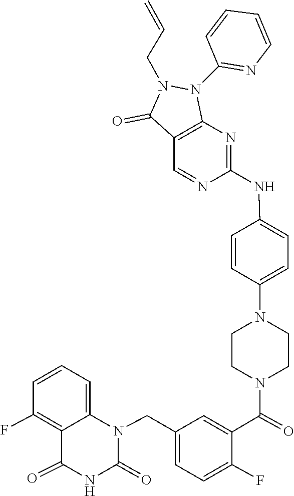 [SMILES: C=CCn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1] | 1-[[3-[4-[4-[[2-allyl-3-oxo-1-(2- pyridyl)pyrazolo[3,4-d]pyrimidin-6- yl]amino]phenyl]piperazine-1- carbonyl]-4-fluoro-phenyl]methyl]-5- fluoro-quinazoline-2,4-dione | 743.3 | 743 |
| 29 | 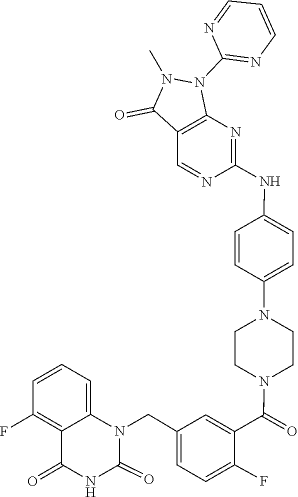 [SMILES: Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1ncccn1] | 5-fluoro-1-[[4-fluoro-3-[4-[4-[(2- methyl-3-oxo-1-pyrimidin-2-yl- pyrazolo[3,4-d]pyrimidin-6- yl)amino]phenyl]piperazine-1- carbonyl]phenyl]methyl]quinazoline- 2,4-dione | 718.2 | 718 |
| 30 | 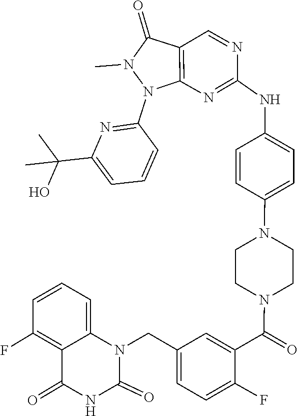 [SMILES: Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1cccc(C(C)(C)O)n1] | 5-fluoro-1-[[4-fluoro-3-[4-[4-[1-[6- (1-hydroxy-1-methyl-ethyl)-2- pyridyl]-2-methyl-3-oxo- pyrazolo[3,4-d]pyrimidin-6- yl]amino]phenyl]piperazine-1- carbonyl]phenyl]methyl]quinazoline- 2,4-dione | 775.3 | 775 |
| 31 | 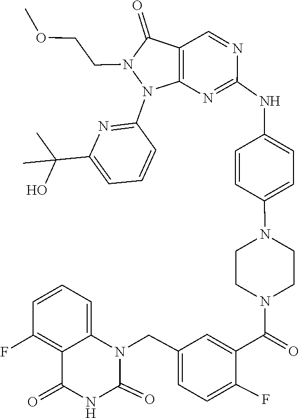 [SMILES: COCCn1c(=O)c2cnc(Nc3ccc(N4CCN(C(=O)c5cc(Cn6c(=O)[nH]c(=O)c7c(F)cccc76)ccc5F)CC4)cc3)nc2n1-c1cccc(C(C)(C)O)n1] | 5-fluoro-1-[[4-fluoro-3-[4-[4-[1-[6- (1-hydroxy-1-methyl-ethyl)-2- pyridyl]-2-(2-methoxyethyl)-3-oxo- pyrazolo[3,4-d]pyrimidin-6- yl]amino]phenyl]piperazine-1- carbonyl]phenyl]methyl]quinazoline- 2,4-dione | 819.3 | 819 |
| 32 | 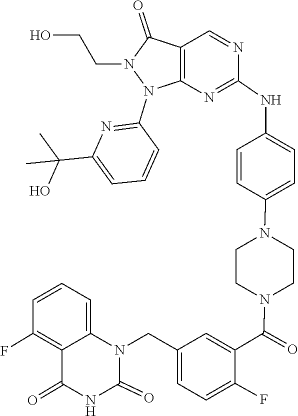 [SMILES: CC(C)(O)c1cccc(-n2c3nc(Nc4ccc(N5CCN(C(=O)c6cc(Cn7c(=O)[nH]c(=O)c8c(F)cccc87)ccc6F)CC5)cc4)ncc3c(=O)n2CCO)n1] | 5-fluoro-1-[[4-fluoro-3-[4-[4-[2-(2- hydroxyethyl)-1-[6-(1-hydroxy-1- methyl-ethyl)-2-pyridyl]-3-oxo- pyrazolo[3,4-d]pyrimidin-6- yl]amino]phenyl]piperazine-1- carbonyl]phenyl]methyl]quinazoline- 2,4-dione | 805.3 | 805 |
| 33 | 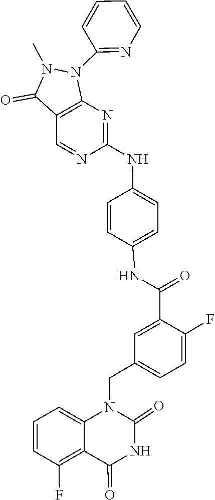 [SMILES: Cn1c(=O)c2cnc(Nc3ccc(NC(=O)c4cc(Cn5c(=O)[nH]c(=O)c6c(F)cccc65)ccc4F)cc3)nc2n1-c1ccccn1] | 2-fluoro-5-[(5-fluoro-2,4-dioxo- quinazolin-1-yl)methyl]-N-[4-[2- methyl-3-oxo-1-(2- pyridyl)pyrazolo[3,4-d]pyrimidin-6- yl]amino]phenyl]benzamide | 648.2 | 648 |
| 34 | 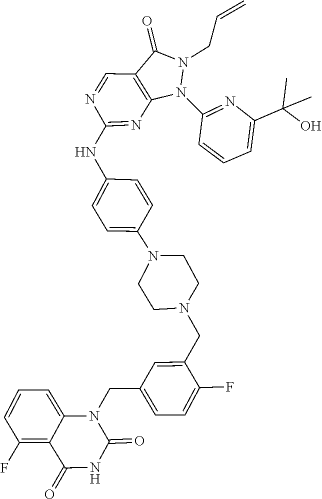 [SMILES: C=CCn1c(=O)c2cnc(Nc3ccc(N4CCN(Cc5cc(Cn6c(=O)[nH]c(=O)c7c(F)cccc76)ccc5F)CC4)cc3)nc2n1-c1cccc(C(C)(C)O)n1] | 1-[[3-[[4-[4-[2-allyl-1-[6-(1- hydroxy-1-methyl-ethyl)-2-pyridyl]- 3-oxo-pyrazolo[3,4-d]pyrimidin-6- yl]amino]phenyl]piperazin-1- yl]methyl]-4-fluoro-phenyl]methyl]- 5-fluoro-quinazoline-2,4-dione | 787.3 | 787 |
| 35 | 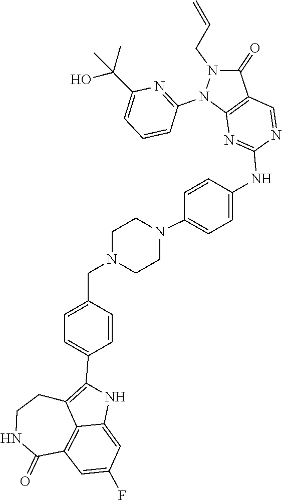 [SMILES: C=CCn1c(=O)c2cnc(Nc3ccc(N4CCN(Cc5ccc(-c6[nH]c7cc(F)cc8c7c6CCNC8=O)cc5)CC4)cc3)nc2n1-c1cccc(C(C)(C)O)n1] | 2-[4-[[4-[4-[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]phenyl]-6-fluoro-3,10- diazatricyclo[6.4.1.04,13]trideca- 1,4,6,8(13)-tetraen-9-one | 779.4 | 779 |
| 36 | 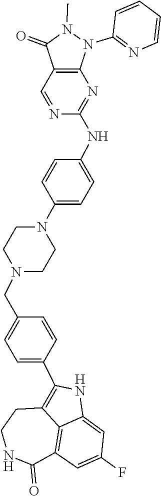 [SMILES: Cn1c(=O)c2cnc(Nc3ccc(N4CCN(Cc5ccc(-c6[nH]c7cc(F)cc8c7c6CCNC8=O)cc5)CC4)cc3)nc2n1-c1ccccn1] | 6-fluoro-2-[4-[[4-[4-[[2-methyl-3- oxo-1-(2-pyridyl)pyrazolo[3,4- d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 695.3 | 695 |
| 37 | 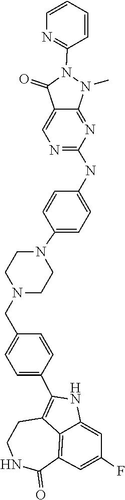 | 6-fluoro-2-[4-[[4-[4-[1-methyl-3- oxo-2-(2-pyridyl)pyrazolo[3,4- d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 695.3 | 695 |
| 38 | 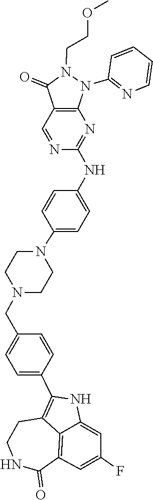 [SMILES: COCCn1c(=O)c2cnc(Nc3ccc(N4CCN(Cc5ccc(-c6[nH]c7cc(F)cc8c7c6CCNC8=O)cc5)CC4)cc3)nc2n1-c1ccccn1] | 6-fluoro-2-[4-[[4-[4-[[2-(2- methoxyethyl)-3-oxo-1-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 739.3 | 739 |
| 39 | 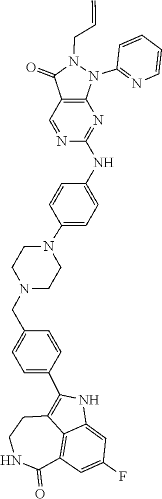 [SMILES: C=CCn1c(=O)c2cnc(Nc3ccc(N4CCN(Cc5ccc(-c6[nH]c7cc(F)cc8c7c6CCNC8=O)cc5)CC4)cc3)nc2n1-c1ccccn1] | 2-[4-[[4-[4-[[2-allyl-3-oxo-1-(2- pyridyl)pyrazolo[3,4-d]pyrimidin-6- yl]amino]phenyl]piperazin-1- yl]methyl]phenyl]-6-fluoro-3,10- diazatricyclo[6.4.1.04,13]trideca- 1,4,6,8(13)-tetraen-9-one | 721.3 | 721 |
| 40 | 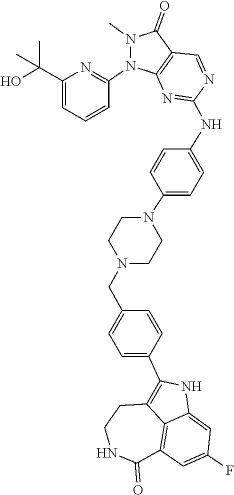 [SMILES: Cn1c(=O)c2cnc(Nc3ccc(N4CCN(Cc5ccc(-c6[nH]c7cc(F)cc8c7c6CCNC8=O)cc5)CC4)cc3)nc2n1-c1cccc(C(C)(C)O)n1] | 6-fluoro-2-[4-[[4-[4-[[1-[6-(1- hydroxy-1-methyl-ethyl)-2-pyridyl]- 2-methyl-3-oxo-pyrazolo[3,4- d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 753.3 | 753 |
| 41 | 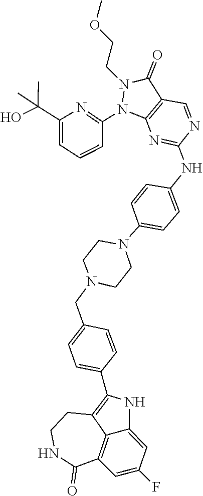 [SMILES: COCCn1c(=O)c2cnc(Nc3ccc(N4CCN(Cc5ccc(-c6[nH]c7cc(F)cc8c7c6CCNC8=O)cc5)CC4)cc3)nc2n1-c1cccc(C(C)(C)O)n1] | 6-fluoro-2-[4-[[4-[4-[[1-[6-(1- hydroxy-1-methyl-ethyl)-2-pyridyl]- 2-(2-methoxyethyl)-3-oxo- pyrazolo[3,4-d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 797.4 | 797 |
| 42 | 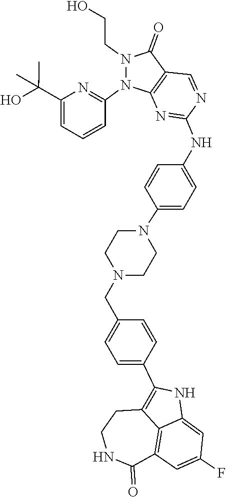 [SMILES: CC(C)(O)c1cccc(-n2c3nc(Nc4ccc(N5CCN(Cc6ccc(-c7[nH]c8cc(F)cc9c8c7CCNC9=O)cc6)CC5)cc4)ncc3c(=O)n2CCO)n1] | 6-fluoro-2-[4-[[4-[4-[[2-(2- hydroxyethyl)-1-[6-(1-hydroxy-1- methyl-ethyl)-2-pyridyl]-3-oxo- pyrazolo[3,4-d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 783.4 | 783 |
| 43 | 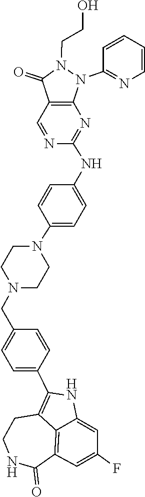 [SMILES: O=C1NCCc2c(-c3ccc(CN4CCN(c5ccc(Nc6ncc7c(=O)n(CCO)n(-c8ccccn8)c7n6)cc5)CC4)cc3)[nH]c3cc(F)cc1c23] | 6-fluoro-2-[4-[[4-[4-[2-(2- hydroxyethyl)-3-oxo-1-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 725.3 | 725 |
| 44 | 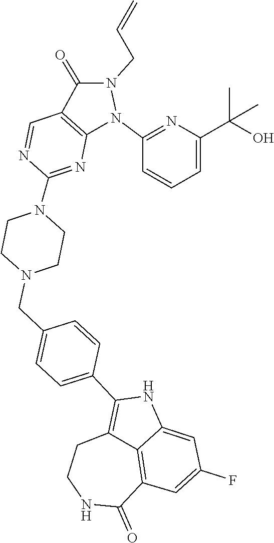 | 2-[4-[[4-[2-allyl-1-[6-(1-hydroxy-1- methyl-ethyl)-2-pyridyl]-3-oxo- pyrazolo[3,4-d]pyrimidin-6- yl]piperazin-1-yl]methyl]phenyl]-6- fluoro-3,10- diazatricyclo[6.4.1.04,13]trideca- 1,4,6,8(13)-tetraen-9-one | 688.3 | 688 |
| 45 | 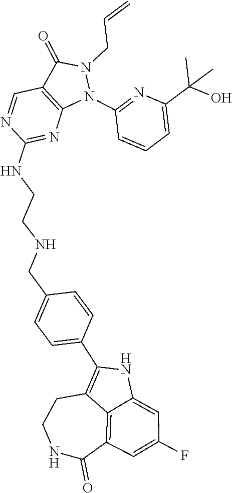 [SMILES: C=CCn1c(=O)c2cnc(NCCNCc3ccc(-c4[nH]c5cc(F)cc6c5c4CCNC6=O)cc3)nc2n1-c1cccc(C(C)(C)O)n1] | 2-[4-[2-[[2-allyl-1-[6-(1-hydroxy-1- methyl-ethyl)-2-pyridyl]-3-oxo- pyrazolo[3,4-d]pyrimidin-6- yl]amino]ethylamino]methyl]phenyl]- 6-fluoro-3,10- diazatricyclo[6.4.1.04,13]trideca- 1,4,6,8(13)-tetraen-9-one | 662.3 | 662 |
| 46 | 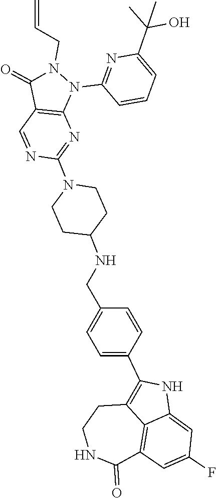 [SMILES: C=CCn1c(=O)c2cnc(N3CCC(NCc4ccc(-c5[nH]c6cc(F)cc7c6c5CCNC7=O)cc4)CC3)nc2n1-c1cccc(C(C)(C)O)n1] | 2-[4-[[[1-[2-allyl-1-[6-(1-hydroxy-1- methyl-ethyl)-2-pyridyl]-3-oxo- pyrazolo[3,4-d]pyrimidin-6-yl]-4- piperidyl]amino]methyl]phenyl]-6- fluoro-3,10- diazatricyclo[6.4.1.04,13]trideca- 1,4,6,8(13)-tetraen-9-one | 702.3 | 702 |
| 47 | 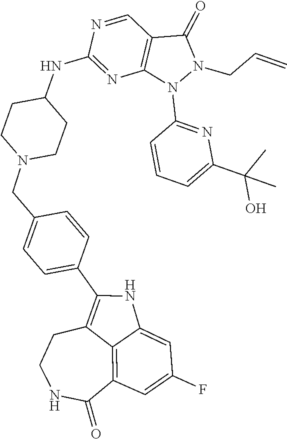 [SMILES: C=CCn1c(=O)c2cnc(NC3CCN(Cc4ccc(-c5[nH]c6cc(F)cc7c6c5CCNC7=O)cc4)CC3)nc2n1-c1cccc(C(C)(C)O)n1] | 2-[4-[[4-[[2-allyl-1-[6-(1-hydroxy-1- methyl-ethyl)-2-pyridyl]-3-oxo- pyrazolo[3,4-d]pyrimidin-6- yl]amino]-1- piperidyl]methyl]phenyl]-6-fluoro- 3,10- diazatricyclo[6.4.1.04,13]trideca- 1,4,6,8(13)-tetraen-9-one | 702.3 | 702 |
| 48 | 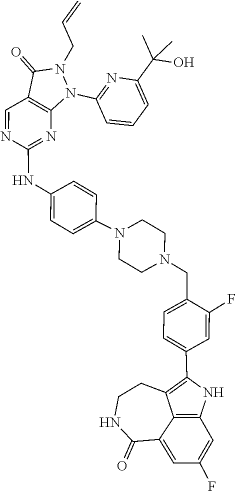 [SMILES: C=CCn1c(=O)c2cnc(Nc3ccc(N4CCN(Cc5ccc(-c6[nH]c7cc(F)cc8c7c6CCNC8=O)cc5F)CC4)cc3)nc2n1-c1cccc(C(C)(C)O)n1] | 2-[4-[4-[4-[[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-3-fluoro-phenyl]-6-fluoro- 3,10- diazatricyclo[6.4.1.04,13]trideca- 1,4(13),5,7-tetraen-9-one | 797.3 | 797 |
| 49 | 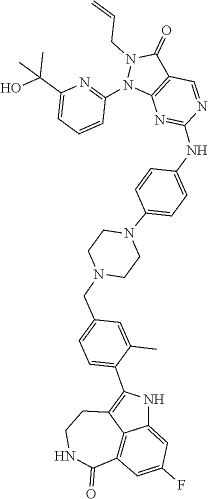 [SMILES: C=CCn1c(=O)c2cnc(Nc3ccc(N4CCN(Cc5ccc(-c6[nH]c7cc(F)cc8c7c6CCNC8=O)c(C)c5)CC4)cc3)nc2n1-c1cccc(C(C)(C)O)n1] | 2-[4-[[4-[4-[[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-2-methyl-phenyl]-6- fluoro-3,10- diazatricyclo[6.4.1.04,13]trideca- 1,4,6,8(13)-tetraen-9-one | 793.4 | 793 |
| 50 | 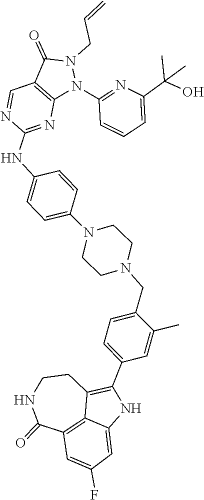 [SMILES: C=CCn1c(=O)c2cnc(Nc3ccc(N4CCN(Cc5ccc(-c6[nH]c7cc(F)cc8c7c6CCNC8=O)cc5C)CC4)cc3)nc2n1-c1cccc(C(C)(C)O)n1] | 2-[4-[[4-[4-[[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-3-methyl-phenyl]-6- fluoro-3,10- diazatricyclo[6.4.1.04,13]trideca- 1,4,6,8(13)-tetraen-9-one | 793.4 | 793 |
| 51 | 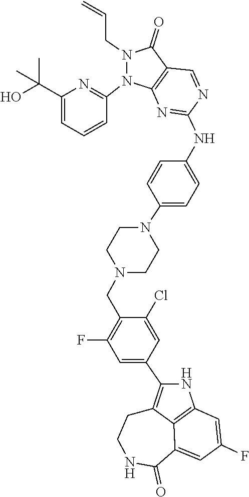 [SMILES: C=CCn1c(=O)c2cnc(Nc3ccc(N4CCN(Cc5c(F)cc(-c6[nH]c7cc(F)cc8c7c6CCNC8=O)cc5Cl)CC4)cc3)nc2n1-c1cccc(C(C)(C)O)n1] | 2-[4-[[4-[4-[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-3-chloro-5-fluoro-phenyl]- 6-fluoro-3,10- diazatricyclo[6.4.1.04,13]trideca- 1,4(13),5,7-tetraen-9-one | 831.3 | 831 |
| 52 | 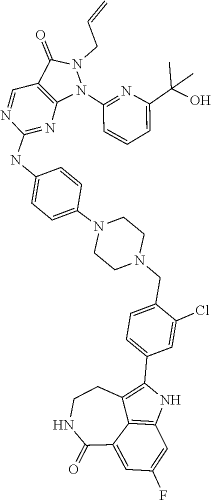 | 2-[4-[[4-[4-[[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-3-chloro-phenyl]-6-fluoro- 3,10- diazatricyclo[6.4.1.04,13]trideca- 1,4(13),5,7-tetraen-9-one | 813.3 | 813 |
| 53 | 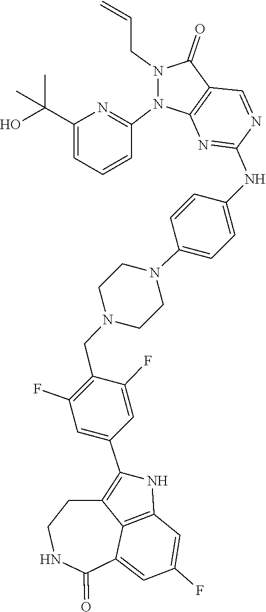 [SMILES: C=CCn1c(=O)c2cnc(Nc3ccc(N4CCN(Cc5c(F)cc(-c6[nH]c7cc(F)cc8c7c6CCNC8=O)cc5F)CC4)cc3)nc2n1-c1cccc(C(C)(C)O)n1] | 2-[4-[[4-[4-[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-3,5-difluoro-phenyl]-6- fluoro-3,10- diazatricyclo[6.4.1.04,13]trideca- 1,4(13),5,7-tetraen-9-one | 815.3 | 815 |
| 54 | 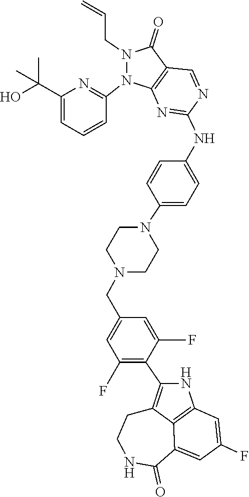 [SMILES: C=CCn1c(=O)c2cnc(Nc3ccc(N4CCN(Cc5cc(F)c(-c6[nH]c7cc(F)cc8c7c6CCNC8=O)c(F)c5)CC4)cc3)nc2n1-c1cccc(C(C)(C)O)n1] | 2-[4-[[4-[4-[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-2,6-difluoro-phenyl]-6- fluoro-3,10- diazatricyclo[6.4.1.04,13]trideca- 1,4(13),5,7-tetraen-9-one | 815.3 | 815 |
| 55 | 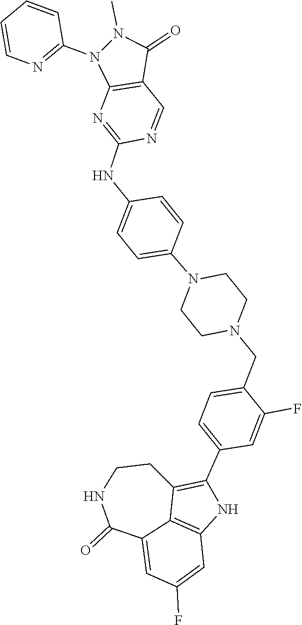 [SMILES: Cn1c(=O)c2cnc(Nc3ccc(N4CCN(Cc5ccc(-c6[nH]c7cc(F)cc8c7c6CCNC8=O)cc5F)CC4)cc3)nc2n1-c1ccccn1] | 6-fluoro-2-[3-fluoro-4-[[4-[4-[[2- methyl-3-oxo-1-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4(13),5,7-tetraen-9-one | 713.3 | 713 |
| 56 | 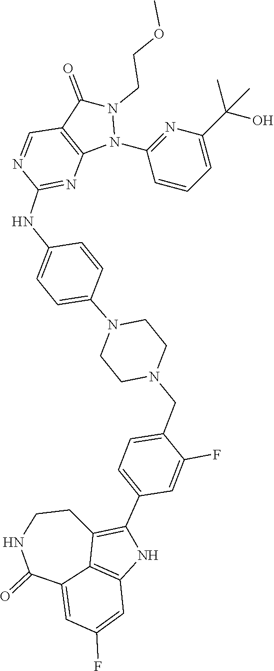 [SMILES: COCCn1c(=O)c2cnc(Nc3ccc(N4CCN(Cc5ccc(-c6[nH]c7cc(F)cc8c7c6CCNC8=O)cc5F)CC4)cc3)nc2n1-c1cccc(C(C)(C)O)n1] | 6-fluoro-2-[3-fluoro-4-[[4-[4-[1-[6- (1-hydroxy-1-methyl-ethyl)-2- pyridyl]-2-(2-methoxyethyl)-3-oxo- pyrazolo[3,4-d]pyrimidin-6- yl]amino]phenyl]piperazin-1- yl]methyl]phenyl]-3,10- diazatricyclo[6.4.1.04,13]trideca- 1,4(13),5,7-tetraen-9-one | 815.4 | 815 |
| 57 | 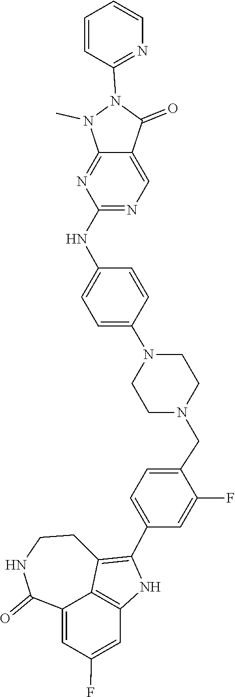 [SMILES: Cn1c2nc(Nc3ccc(N4CCN(Cc5ccc(-c6[nH]c7cc(F)cc8c7c6CCNC8=O)cc5F)CC4)cc3)ncc2c(=O)n1-c1ccccn1] | 6-fluoro-2-[3-fluoro-4-[[4-[4-[1- methyl-3-oxo-2-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4(13),5,7-tetraen-9-one | 713.3 | 713 |
| 58 | 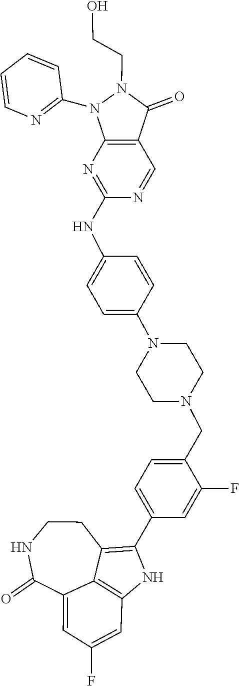 [SMILES: O=C1NCCc2c(-c3ccc(CN4CCN(c5ccc(Nc6ncc7c(=O)n(CCO)n(-c8ccccn8)c7n6)cc5)CC4)c(F)c3)[nH]c3cc(F)cc1c23] | 6-fluoro-2-[3-fluoro-4-[4-[4-[2-(2- hydroxyethyl)-3-oxo-1-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4(13),5,7-tetraen-9-one | 743.3 | 743 |
| 59 | 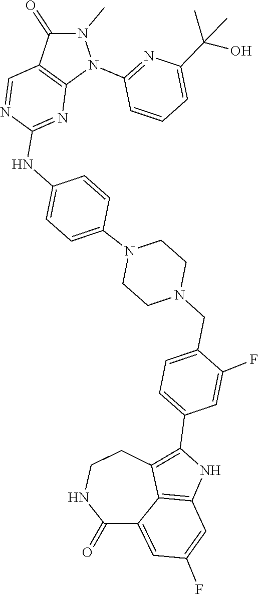 [SMILES: Cn1c(=O)c2cnc(Nc3ccc(N4CCN(Cc5ccc(-c6[nH]c7cc(F)cc8c7c6CCNC8=O)cc5F)CC4)cc3)nc2n1-c1cccc(C(C)(C)O)n1] | 6-fluoro-2-[3-fluoro-4-[[4-[4-[1-[6- (1-hydroxy-1-methyl-ethyl)-2- pyridyl]-2-methyl-3-oxo- pyrazolo[3,4-d]pyrimidin-6- yl]amino]phenyl]piperazin-1- yl]methyl]phenyl]-3,10- diazatricyclo[6.4.1.04,13]trideca- 1,4(13),5,7-tetraen-9-one | 771.3 | 771 |
| 60 | 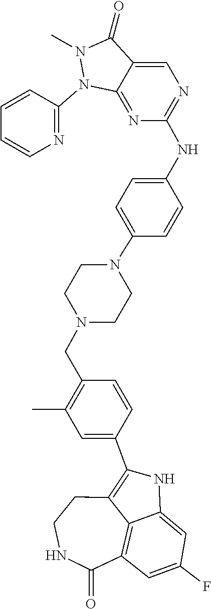 [SMILES: Cc1cc(-c2[nH]c3cc(F)cc4c3c2CCNC4=O)ccc1CN1CCN(c2ccc(Nc3ncc4c(=O)n(C)n(-c5ccccn5)c4n3)cc2)CC1] | 6-fluoro-2-[3-methyl-4-[[4-[4-[[2- methyl-3-oxo-1-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 709.3 | 709 |
| 61 | 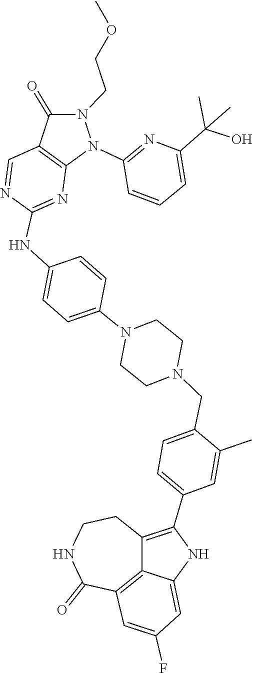 [SMILES: COCCn1c(=O)c2cnc(Nc3ccc(N4CCN(Cc5ccc(-c6[nH]c7cc(F)cc8c7c6CCNC8=O)cc5C)CC4)cc3)nc2n1-c1cccc(C(C)(C)O)n1] | 6-fluoro-2-[4-[[4-[4-[1-[6-(1- hydroxy-1-methyl-ethyl)-2-pyridyl]- 2-(2-methoxyethyl)-3-oxo- pyrazolo[3,4-d]pyrimidin-6- yl]amino]phenyl]piperazin-1- yl]methyl]-3-methyl-phenyl]-3,10- diazatricyclo[6.4.1.04,13]trideca- 1,4,6,8(13)-tetraen-9-one | 811.4 | 811 |
| 62 | 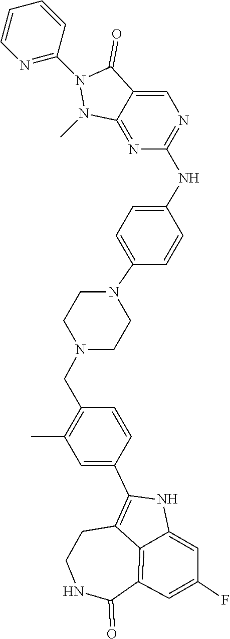 [SMILES: Cc1cc(-c2[nH]c3cc(F)cc4c3c2CCNC4=O)ccc1CN1CCN(c2ccc(Nc3ncc4c(=O)n(-c5ccccn5)n(C)c4n3)cc2)CC1] | 6-fluoro-2-[3-methyl-4-[[4-[4-[[1- methyl-3-oxo-2-(2- pyridyl)pyrazolo[3,4-d]pyrimidin-6- yl]amino]phenyl]piperazin-1- yl]methyl]phenyl]-3,10- diazatricyclo[6.4.1.04,13]trideca- 1,4,6,8(13)-tetraen-9-one | 709.3 | 709 |
| 63 | 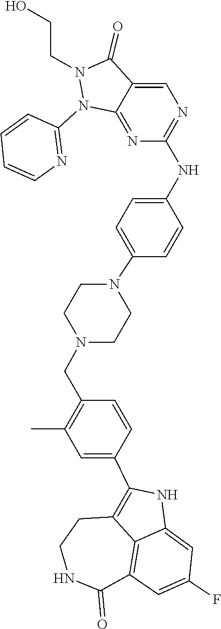 [SMILES: Cc1cc(-c2[nH]c3cc(F)cc4c3c2CCNC4=O)ccc1CN1CCN(c2ccc(Nc3ncc4c(=O)n(CCO)n(-c5ccccn5)c4n3)cc2)CC1] | 6-fluoro-2-[4-[[4-[4-[[2-(2- hydroxyethyl)-3-oxo-1-(2- pyridyl)pyrazolo[3,4-d]pyrimidin-6- yl]amino]phenyl]piperazin-1- yl]methyl]-3-methyl-phenyl]-3,10- diazatricyclo[6.4.1.04,13]trideca- 1,4,6,8(13)-tetraen-9-one | 739.3 | 739 |
| 64 | 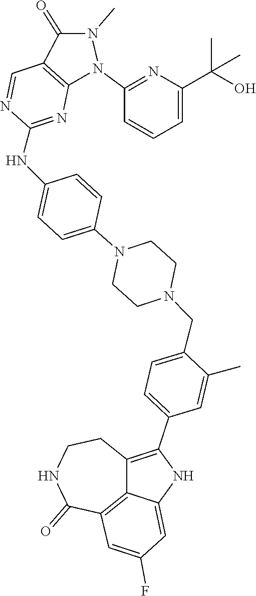 | 6-fluoro-2-[4-[[4-[4-[1-[6-(1- hydroxy-1-methyl-ethyl)-2-pyridyl]- 2-methyl-3-oxo-pyrazolo[3,4- d]pyrimidin-6- yl]amino]phenyl]piperazin-1- yl]methyl]-3-methyl-phenyl]-3,10- diazatricyclo[6.4.1.04,13]trideca- 1,4,6,8(13)-tetraen-9-one | 767.4 | 767 |
| 65 | 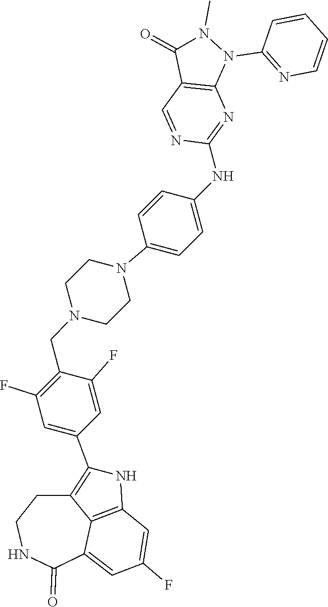 [SMILES: Cn1c(=O)c2cnc(Nc3ccc(N4CCN(Cc5c(F)cc(-c6[nH]c7cc(F)cc8c7c6CCNC8=O)cc5F)CC4)cc3)nc2n1-c1ccccn1] | 2-[3,5-difluoro-4-[4-[4-[[2-methyl-3- oxo-1-(2-pyridyl)pyrazolo[3,4- d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 731.3 | 731 |
| 66 | 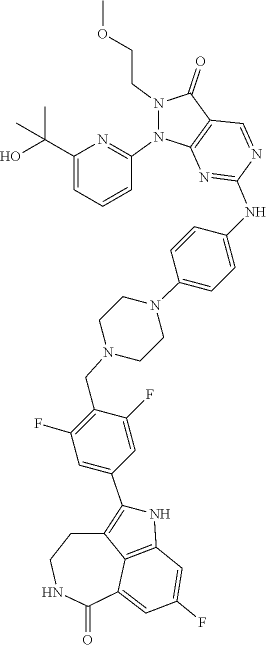 [SMILES: COCCn1c(=O)c2cnc(Nc3ccc(N4CCN(Cc5c(F)cc(-c6[nH]c7cc(F)cc8c7c6CCNC8=O)cc5F)CC4)cc3)nc2n1-c1cccc(C(C)(C)O)n1] | 2-[3,5-difluoro-4-[[4-[4-[[1-[6-(1- hydroxy-1-methyl-ethyl)-2-pyridyl]- 2-(2-methoxyethyl)-3-oxo- pyrazolo[3,4-d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 833.3 | 833 |
| 67 | 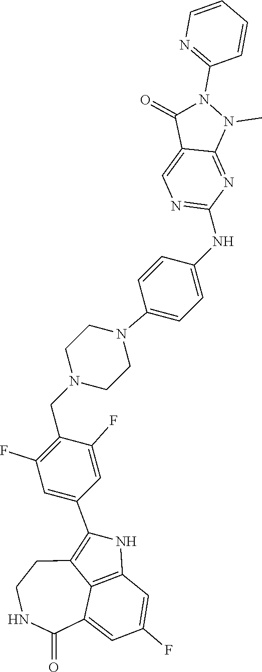 [SMILES: Cn1c2nc(Nc3ccc(N4CCN(Cc5c(F)cc(-c6[nH]c7cc(F)cc8c7c6CCNC8=O)cc5F)CC4)cc3)ncc2c(=O)n1-c1ccccn1] | 2-[3,5-difluoro-4-[[4-[4-[[1-methyl-3- oxo-2-(2-pyridyl)pyrazolo[3,4- d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 731.3 | 731 |
| 68 | 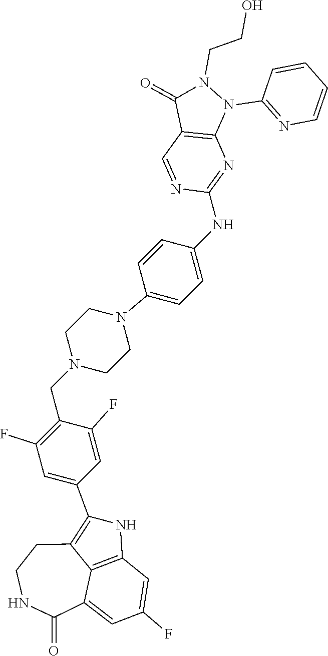 [SMILES: O=C1NCCc2c(-c3cc(F)c(CN4CCN(c5ccc(Nc6ncc7c(=O)n(CCO)n(-c8ccccn8)c7n6)cc5)CC4)c(F)c3)[nH]c3cc(F)cc1c23] | 2-[3,5-difluoro-4-[[4-[4-[[2-(2- hydroxyethyl)-3-oxo-1-(2- pyridyl)pyrazolo[3,4-d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 761.3 | 761 |
| 69 | 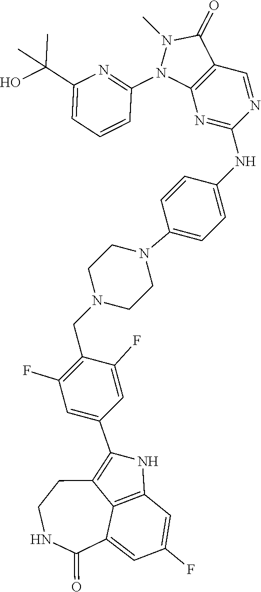 [SMILES: Cn1c(=O)c2cnc(Nc3ccc(N4CCN(Cc5c(F)cc(-c6[nH]c7cc(F)cc8c7c6CCNC8=O)cc5F)CC4)cc3)nc2n1-c1cccc(C(C)(C)O)n1] | 2-[3,5-difluoro-4-[[4-[4-[[1-[6-(1- hydroxy-1-methyl-ethyl)-2-pyridyl]- 2-methyl-3-oxo-pyrazolo[3,4- d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 789.3 | 789 |
| 70 | 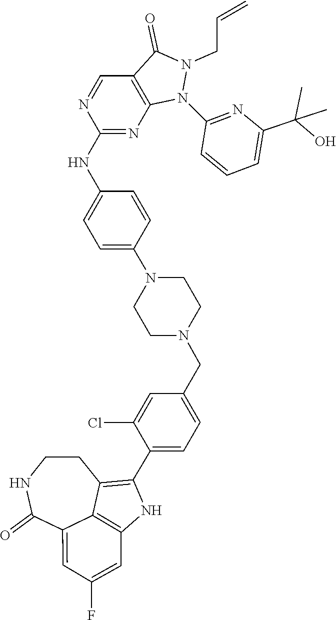 [SMILES: C=CCn1c(=O)c2cnc(Nc3ccc(N4CCN(Cc5ccc(-c6[nH]c7cc(F)cc8c7c6CCNC8=O)c(Cl)c5)CC4)cc3)nc2n1-c1cccc(C(C)(C)O)n1] | 2-[4-[[4-[4-[[2-allyl-1-[6-(1-hydroxy- 1-methyl-ethyl)-2-pyridyl]-3-oxo- pyrazolo[3,4-d]pyrimidin-6- yl]amino]phenyl]piperazin-1- yl]methyl]-2-chloro-phenyl]-6-fluoro- 3,10- diazatricyclo[6.4.1.04,13]trideca- 1,4(13),5,7-tetraen-9-one | 813.3 | 813 |
| 71 | 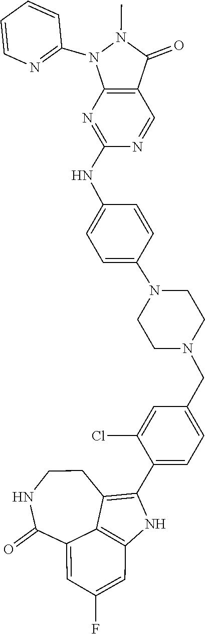 [SMILES: Cn1c(=O)c2cnc(Nc3ccc(N4CCN(Cc5ccc(-c6[nH]c7cc(F)cc8c7c6CCNC8=O)c(Cl)c5)CC4)cc3)nc2n1-c1ccccn1] | 2-[2-chloro-4-[[4-[4-[2-methyl-3- oxo-1-(2-pyridyl)pyrazolo[3,4- d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 729.3 | 729 |
| 72 | 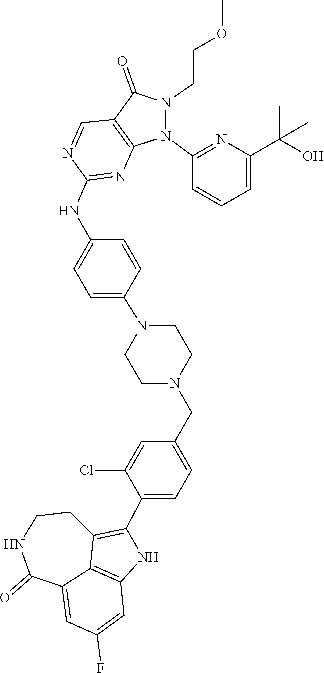 [SMILES: COCCn1c(=O)c2cnc(Nc3ccc(N4CCN(Cc5ccc(-c6[nH]c7cc(F)cc8c7c6CCNC8=O)c(Cl)c5)CC4)cc3)nc2n1-c1cccc(C(C)(C)O)n1] | 2-[2-chloro-4-[4-[4-[1-[6-(1- hydroxy-1-methyl-ethyl)-2-pyridyl]- 2-(2-methoxyethyl)-3-oxo- pyrazolo[3,4-d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 831.3 | 831 |
| 73 | 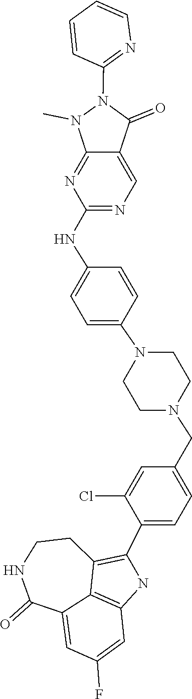 | 2-[2-chloro-4-[[4-[4-[1-methyl-3- oxo-2-(2-pyridyl)pyrazolo[3,4- d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 729.3 | 729 |
| 74 | 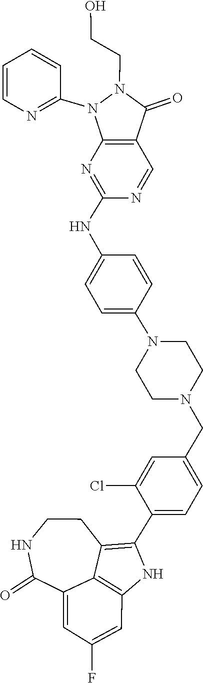 [SMILES: O=C1NCCc2c(-c3ccc(CN4CCN(c5ccc(Nc6ncc7c(=O)n(CCO)n(-c8ccccn8)c7n6)cc5)CC4)cc3Cl)[nH]c3cc(F)cc1c23] | 2-[2-chloro-4-[[4-[4-[2-(2- hydroxyethyl)-3-oxo-1-(2- pyridyl)pyrazolo[3,4-d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 759.3 | 759 |
| 75 | 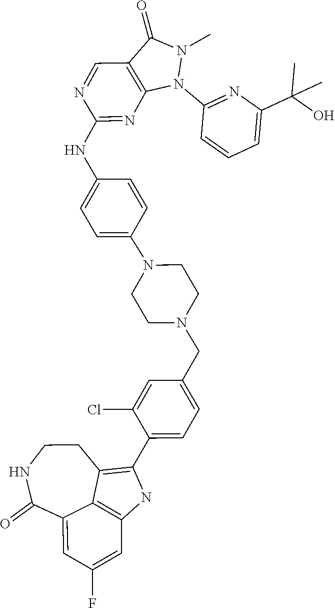 | 2-[2-chloro-4-[[4-[4-[1-[6-(1- hydroxy-1-methyl-ethyl)-2-pyridyl]- 2-methyl-3-oxo-pyrazolo[3,4- d]pyrimidin-6- yl]amino]phenyl]piperazin-1- yl]methyl]phenyl]-6-fluoro-3,10- diazatricyclo[6.4.1.04,13]trideca- 1,4(13),5,7-tetraen-9-one | 787.3 | 787 |
| 76 | 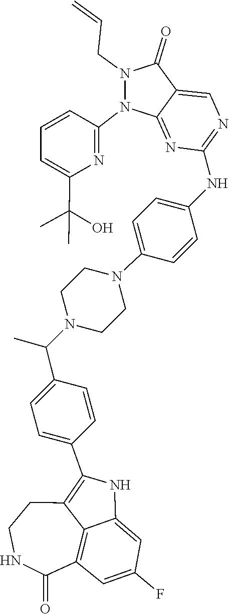 [SMILES: C=CCn1c(=O)c2cnc(Nc3ccc(N4CCN(C(C)c5ccc(-c6[nH]c7cc(F)cc8c7c6CCNC8=O)cc5)CC4)cc3)nc2n1-c1cccc(C(C)(C)O)n1] | 2-[4-[1-[4-[4-[[2-allyl-1-[6-(1- hydroxy-1-methyl-ethyl)-2-pyridyl]- 3-oxo-pyrazolo[3,4-d]pyrimidin-6- yl]amino]phenyl]piperazin-1- yl]ethyl]phenyl]-6-fluoro-3,10- diazatricyclo[6.4.1.04,13]trideca- 1,4,6,8(13)-tetraen-9-one | 793.4 | 793 |
| 77 | 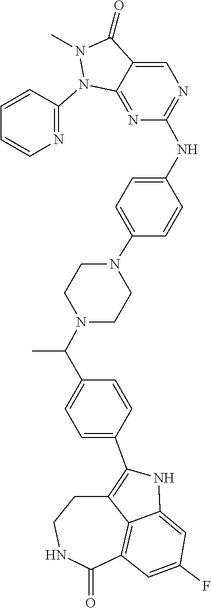 [SMILES: CC(c1ccc(-c2[nH]c3cc(F)cc4c3c2CCNC4=O)cc1)N1CCN(c2ccc(Nc3ncc4c(=O)n(C)n(-c5ccccn5)c4n3)cc2)CC1] | 6-fluoro-2-[4-[1-[4-[4-[2-methyl-3- oxo-1-(2-pyridyl)pyrazolo[3,4- d]pyrimidin-6- yl]amino]phenyl]piperazin-1- yl]ethyl]phenyl]-3,10- diazatricyclo[6.4.1.04,13]trideca- 1,4,6,8(13)-tetraen-9-one | 709.3 | 709 |
| 78 | 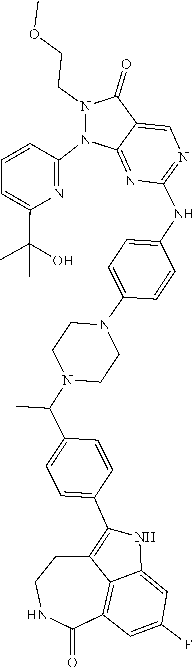 [SMILES: COCCn1c(=O)c2cnc(Nc3ccc(N4CCN(C(C)c5ccc(-c6[nH]c7cc(F)cc8c7c6CCNC8=O)cc5)CC4)cc3)nc2n1-c1cccc(C(C)(C)O)n1] | 6-fluoro-2-[4-[1-[4-[4-[1-[6-(1- hydroxy-1-methyl-ethyl)-2-pyridyl]- 2-(2-methoxyethyl)-3-oxo- pyrazolo[3,4-d]pyrimidin-6- yl]amino]phenyl]piperazin-1- yl]ethyl]phenyl]-3,10- diazatricyclo[6.4.1.04,13]trideca- 1,4,6,8(13)-tetraen-9-one | 811.4 | 811 |
| 79 | 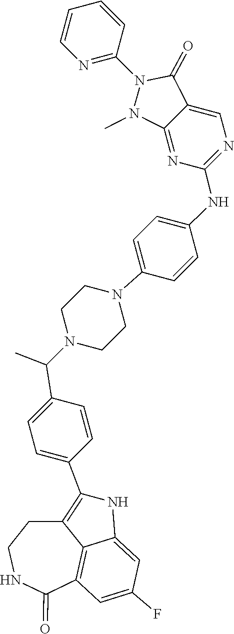 [SMILES: CC(c1ccc(-c2[nH]c3cc(F)cc4c3c2CCNC4=O)cc1)N1CCN(c2ccc(Nc3ncc4c(=O)n(-c5ccccn5)n(C)c4n3)cc2)CC1] | 6-fluoro-2-[4-[1-[4-[4-[1-methyl-3- oxo-2-(2-pyridyl)pyrazolo[3,4- d]pyrimidin-6- yl]amino]phenyl]piperazin-1- yl]ethyl]phenyl]-3,10- diazatricyclo[6.4.1.04,13]trideca- 1,4,6,8(13)-tetraen-9-one | 709.3 | 709 |
| 80 | 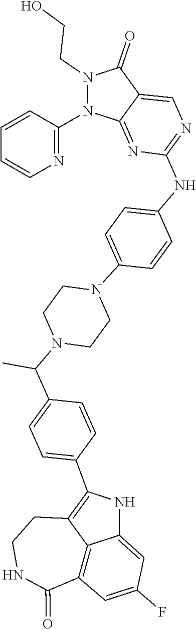 [SMILES: CC(c1ccc(-c2[nH]c3cc(F)cc4c3c2CCNC4=O)cc1)N1CCN(c2ccc(Nc3ncc4c(=O)n(CCO)n(-c5ccccn5)c4n3)cc2)CC1] | 6-fluoro-2-[4-[1-[4-[4-[2-(2- hydroxyethyl)-3-oxo-1-(2- pyridyl)pyrazolo[3,4-d]pyrimidin-6- yl]amino]phenyl]piperazin-1- yl]ethyl]phenyl]-3,10- diazatricyclo[6.4.1.04,13]trideca- 1,4,6,8(13)-tetraen-9-one | 739.3 | 739 |
| 81 | 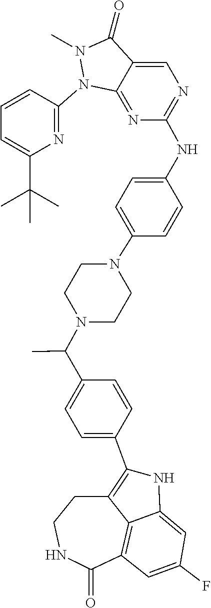 [SMILES: CC(c1ccc(-c2[nH]c3cc(F)cc4c3c2CCNC4=O)cc1)N1CCN(c2ccc(Nc3ncc4c(=O)n(C)n(-c5cccc(C(C)(C)C)n5)c4n3)cc2)CC1] | 6-fluoro-2-[4-[1-[4-[4-[1-[6-(1- hydroxy-1-methyl-ethyl)-2-pyridyl]- 2-methyl-3-oxo-pyrazolo[3,4- d]pyrimidin-6- yl]amino]phenyl]piperazin-1- yl]ethyl]phenyl]-3,10- diazatricyclo[6.4.1.04,13]trideca- 1,4,6,8(13)-tetraen-9-one | 767.4 | 767 |
| 82 | 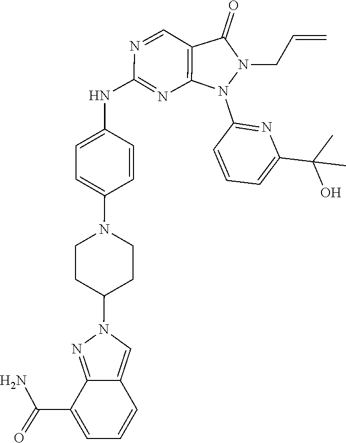 [SMILES: C=CCn1c(=O)c2cnc(Nc3ccc(N4CCC(n5cc6cccc(C(N)=O)c6n5)CC4)cc3)nc2n1-c1cccc(C(C)(C)O)n1] | 2-[1-[4-[2-allyl-1-[6-(1-hydroxy-1- methyl-ethyl)-2-pyridyl]-3-oxo- pyrazolo[3,4-d]pyrimidin-6- yl]amino]phenyl]-4- piperidyl]indazole-7-carboxamide | 645.3 | 645 |
| 83 | 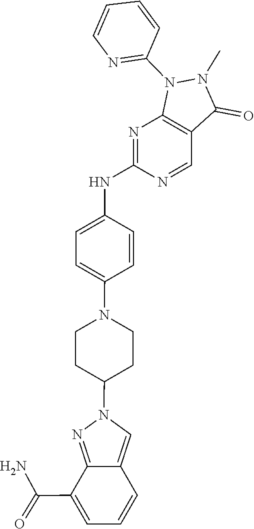 [SMILES: Cn1c(=O)c2cnc(Nc3ccc(N4CCC(n5cc6cccc(C(N)=O)c6n5)CC4)cc3)nc2n1-c1ccccn1] | 2-[1-[4-[2-methyl-3-oxo-1-(2- pyridyl)pyrazolo[3,4-d]pyrimidin-6- yl]amino]phenyl]-4- piperidyl]indazole-7-carboxamide | 560.2 | 560 |
| 84 | 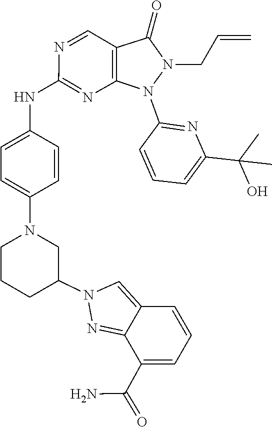 [SMILES: C=CCn1c(=O)c2cnc(Nc3ccc(N4CCCC(n5cc6cccc(C(N)=O)c6n5)C4)cc3)nc2n1-c1cccc(C(C)(C)O)n1] | 2-[1-[4-[2-allyl-1-[6-(1-hydroxy-1- methyl-ethyl)-2-pyridyl]-3-oxo- pyrazolo[3,4-d]pyrimidin-6- yl]amino]phenyl]-3- piperidyl]indazole-7-carboxamide | 645.3 | 645 |
| 85 | 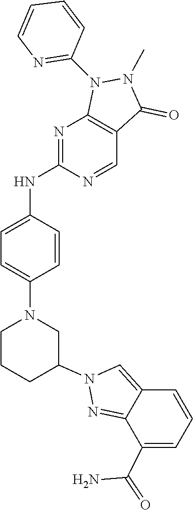 [SMILES: Cn1c(=O)c2cnc(Nc3ccc(N4CCCC(n5cc6cccc(C(N)=O)c6n5)C4)cc3)nc2n1-c1ccccn1] | 2-[1-[4-[2-methyl-3-oxo-1-(2- pyridyl)pyrazolo[3,4-d]pyrimidin-6- yl]amino]phenyl]-3- piperidyl]indazole-7-carboxamide | 561.2 | 561 |
| 86 | 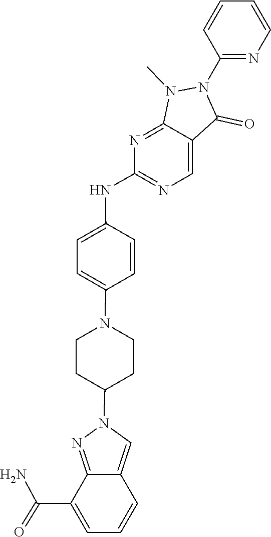 [SMILES: Cn1c2nc(Nc3ccc(N4CCC(n5cc6cccc(C(N)=O)c6n5)CC4)cc3)ncc2c(=O)n1-c1ccccn1] | 2-[1-[4-[1-methyl-3-oxo-2-(2- pyridyl)pyrazolo[3,4-d]pyrimidin-6- yl]amino]phenyl]-4- piperidyl]indazole-7-carboxamide | 561.2 | 561 |
| 87 | 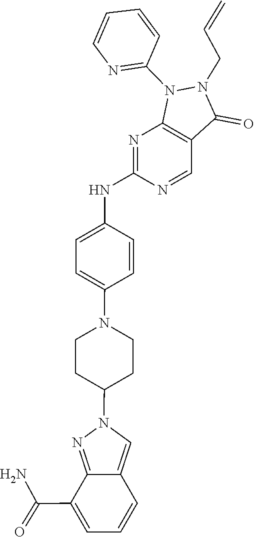 [SMILES: C=CCn1c(=O)c2cnc(Nc3ccc(N4CCC(n5cc6cccc(C(N)=O)c6n5)CC4)cc3)nc2n1-c1ccccn1] | 2-[1-[4-[[2-allyl-3-oxo-1-(2- pyridyl)pyrazolo[3,4-d]pyrimidin-6- yl]amino]phenyl]-4- piperidyl]indazole-7-carboxamide | 587.3 | 587 |
| 88 | 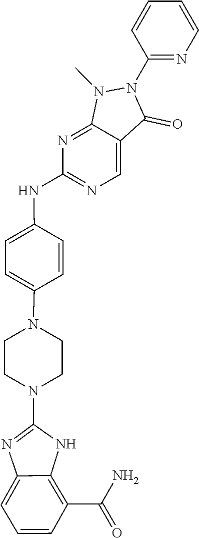 [SMILES: Cn1c2nc(Nc3ccc(N4CCN(c5nc6cccc(C(N)=O)c6[nH]5)CC4)cc3)ncc2c(=O)n1-c1ccccn1] | 2-[4-[4-[[1-methyl-3-oxo-2-(2- pyridyl)pyrazolo[3,4-d]pyrimidin-6- yl]amino]phenyl]piperazin-1-yl]-3H- benzimidazole-4-carboxamide | 562.2 | 562 |
| 89 | 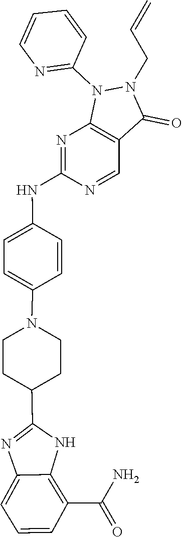 [SMILES: C=CCn1c(=O)c2cnc(Nc3ccc(N4CCC(c5nc6cccc(C(N)=O)c6[nH]5)CC4)cc3)nc2n1-c1ccccn1] | 2-[4-[4-[[2-allyl-3-oxo-1-(2- pyridyl)pyrazolo[3,4-d]pyrimidin-6- yl]amino]phenyl]piperazin-1-yl]-3H- benzimidazole-4-carboxamide | 588.3 | 588 |
| 90 | 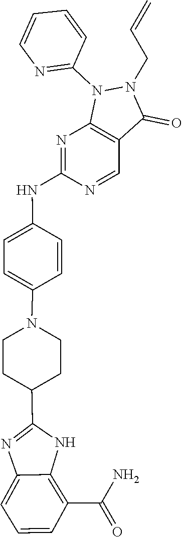 [SMILES: C=CCn1c(=O)c2cnc(Nc3ccc(N4CCC(c5nc6cccc(C(N)=O)c6[nH]5)CC4)cc3)nc2n1-c1ccccn1] | 2-[1-[4-[2-allyl-3-oxo-1-(2- pyridyl)pyrazolo[3,4-d]pyrimidin-6- yl]amino]phenyl]-4-piperidyl]-3H- benzimidazole-4-carboxamide | 587.3 | 587 |
| 91 | 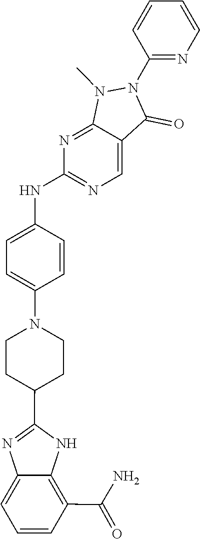 [SMILES: Cn1c2nc(Nc3ccc(N4CCC(c5nc6cccc(C(N)=O)c6[nH]5)CC4)cc3)ncc2c(=O)n1-c1ccccn1] | 2-[1-[4-[1-methyl-3-oxo-2-(2- pyridyl)pyrazolo[3,4-d]pyrimidin-6- yl]amino]phenyl]-4-piperidyl]-3H- benzimidazole-4-carboxamide | 561.2 | 561 |
| 92 | 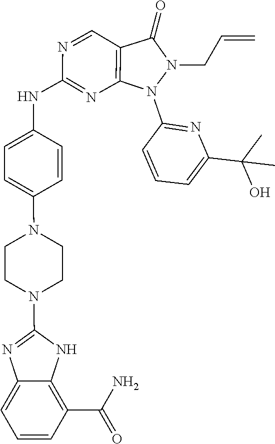 [SMILES: C=CCn1c(=O)c2cnc(Nc3ccc(N4CCN(c5nc6cccc(C(N)=O)c6[nH]5)CC4)cc3)nc2n1-c1cccc(C(C)(C)O)n1] | 2-[4-[4-[2-allyl-1-[6-(1-hydroxy-1- methyl-ethyl)-2-pyridyl]-3-oxo- pyrazolo[3,4-d]pyrimidin-6- yl]amino]phenyl]piperazin-1-yl]-3H- benzimidazole-4-carboxamide | 646.3 | 646 |
| 93 | 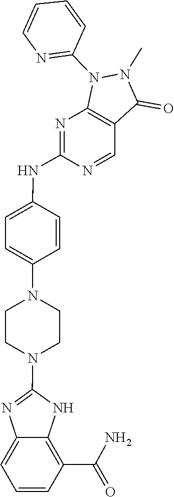 [SMILES: Cn1c(=O)c2cnc(Nc3ccc(N4CCN(c5nc6cccc(C(N)=O)c6[nH]5)CC4)cc3)nc2n1-c1ccccn1] | 2-[4-[4-[2-methyl-3-oxo-1-(2- pyridyl)pyrazolo[3,4-d]pyrimidin-6- yl]amino]phenyl]piperazin-1-yl]-3H- benzimidazole-4-carboxamide | 562.2 | 562 |
| 94 | 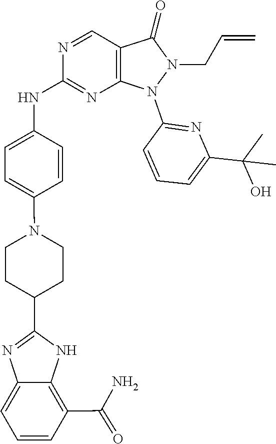 [SMILES: C=CCn1c(=O)c2cnc(Nc3ccc(N4CCC(c5nc6cccc(C(N)=O)c6[nH]5)CC4)cc3)nc2n1-c1cccc(C(C)(C)O)n1] | 2-[1-[4-[2-allyl-1-[6-(1-hydroxy-1- methyl-ethyl)-2-pyridyl]-3-oxo- pyrazolo[3,4-d]pyrimidin-6- yl]amino]phenyl]-4-piperidyl]-3H- benzimidazole-4-carboxamide | 645.3 | 645 |
| 95 | 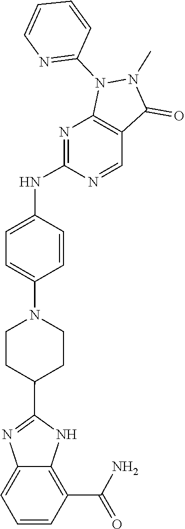 [SMILES: Cn1c(=O)c2cnc(Nc3ccc(N4CCC(c5nc6cccc(C(N)=O)c6[nH]5)CC4)cc3)nc2n1-c1ccccn1] | 2-[1-[4-[[2-methyl-3-oxo-1-(2- pyridyl)pyrazolo[3,4-d]pyrimidin-6- yl]amino]phenyl]-4-piperidyl]-3H- benzimidazole-4-carboxamide | 561.2 | 561 |
| 96 | 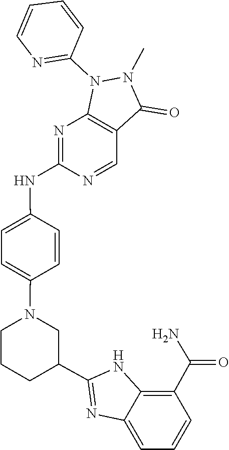 [SMILES: Cn1c(=O)c2cnc(Nc3ccc(N4CCCC(c5nc6cccc(C(N)=O)c6[nH]5)C4)cc3)nc2n1-c1ccccn1] | 2-[1-[4-[[2-methyl-3-oxo-1-(2- pyridyl)pyrazolo[3,4-d]pyrimidin-6- yl]amino]phenyl]-3-piperidyl]-3H- benzimidazole-4-carboxamide | 561.2 | 561 |
| 97 | 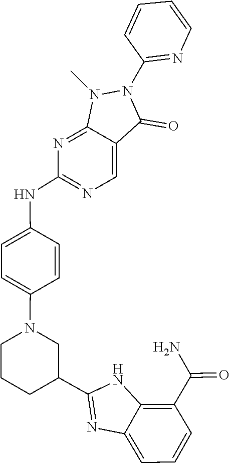 [SMILES: Cn1c2nc(Nc3ccc(N4CCCC(c5nc6cccc(C(N)=O)c6[nH]5)C4)cc3)ncc2c(=O)n1-c1ccccn1] | 2-[1-[4-[1-methyl-3-oxo-2-(2- pyridyl)pyrazolo[3,4-d]pyrimidin-6- yl]amino]phenyl]-3-piperidyl]-3H- benzimidazole-4-carboxamide | 561.2 | 561 |
| 98 | 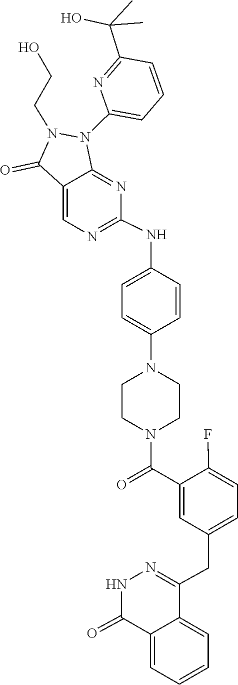 [SMILES: CC(C)(O)c1cccc(-n2c3nc(Nc4ccc(N5CCN(C(=O)c6cc(Cc7n[nH]c(=O)c8ccccc78)ccc6F)CC5)cc4)ncc3c(=O)n2CCO)n1] | 4-[[4-fluoro-3-[4-[4-[[2-(2- hydroxyethyl)-1-[6-(1-hydroxy-1- methyl-ethyl)-2-pyridyl]-3-oxo- pyrazolo[3,4-d]pyrimidin-6- yl]amino]phenyl]piperazine-1- carbonyl]phenyl]methyl]-2H- phthalazin-1-one | 771.3 | 771 |
| 99 | 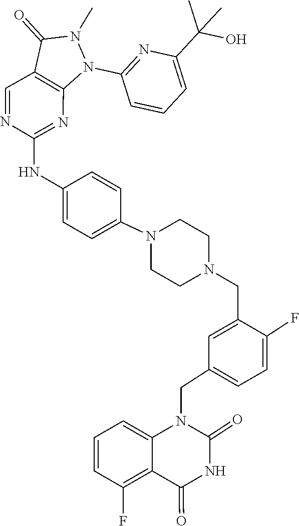 [SMILES: Cn1c(=O)c2cnc(Nc3ccc(N4CCN(Cc5cc(Cn6c(=O)[nH]c(=O)c7c(F)cccc76)ccc5F)CC4)cc3)nc2n1-c1cccc(C(C)(C)O)n1] | 5-fluoro-1-[[4-fluoro-3-[[4-[4-[[1-[6- (1-hydroxy-1-methyl-ethyl)-2- pyridyl]-2-methyl-3-oxo- pyrazolo[3,4-d]pyrimidin-6- yl]amino]phenyl]piperazin-1- yl]methyl]phenyl]methyl]quinazoline- 2,4-dione | 761.3 | 761 |
| 100 | 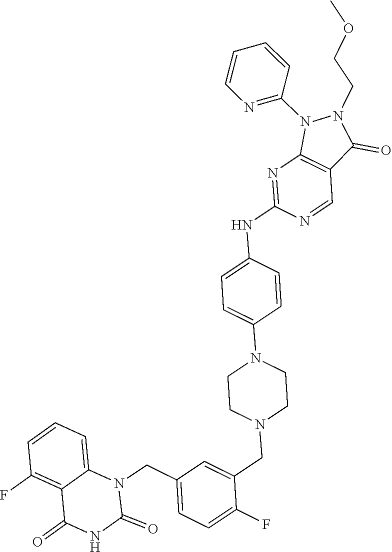 [SMILES: COCCn1c(=O)c2cnc(Nc3ccc(N4CCN(Cc5cc(Cn6c(=O)[nH]c(=O)c7c(F)cccc76)ccc5F)CC4)cc3)nc2n1-c1ccccn1] | 5-fluoro-1-[4-fluoro-3-[4-[4-[2-(2- methoxyethyl)-3-oxo-1-(2- pyridyl)pyrazolo[3,4-d]pyrimidin-6- yl]amino]phenyl]piperazin-1- yl]methyl]phenyl]methyl]quinazoline- 2,4-dione | 747.3 | 747 |
| 101 | 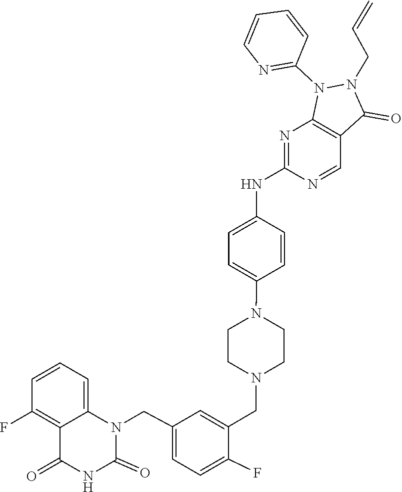 [SMILES: C=CCn1c(=O)c2cnc(Nc3ccc(N4CCN(Cc5cc(Cn6c(=O)[nH]c(=O)c7c(F)cccc76)ccc5F)CC4)cc3)nc2n1-c1ccccn1] | 1-[[3-[[4-[4-[2-allyl-3-oxo-1-(2- pyridyl)pyrazolo[3,4-d]pyrimidin-6- yl]amino]phenyl]piperazin-1- yl]methyl]-4-fluoro-phenyl]methyl]- 5-fluoro-quinazoline-2,4-dione | 729.3 | 729 |
| 102 | 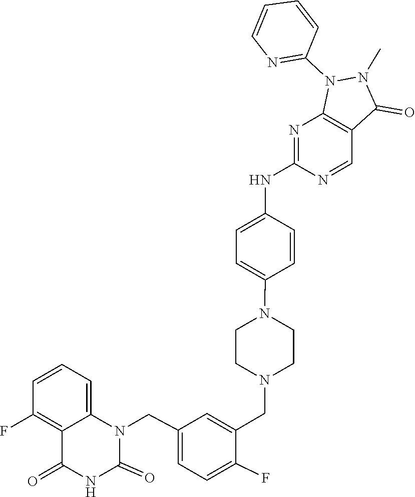 [SMILES: Cn1c(=O)c2cnc(Nc3ccc(N4CCN(Cc5cc(Cn6c(=O)[nH]c(=O)c7c(F)cccc76)ccc5F)CC4)cc3)nc2n1-c1ccccn1] | 5-fluoro-1-[[4-fluoro-3-[[4-[4-[[2- methyl-3-oxo-1-(2- pyridyl)pyrazolo[3,4-d]pyrimidin-6- yl]amino]phenyl]piperazin-1- yl]methyl]phenyl]methyl]quinazoline- 2,4-dione | 703.3 | 703 |
| 103 | 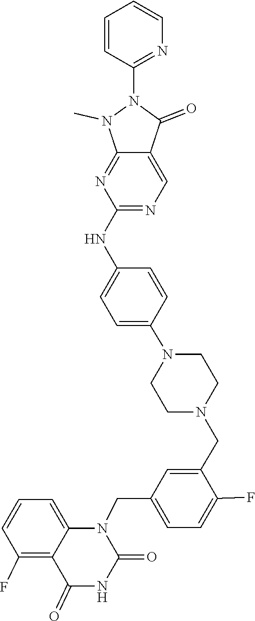 [SMILES: Cn1c2nc(Nc3ccc(N4CCN(Cc5cc(Cn6c(=O)[nH]c(=O)c7c(F)cccc76)ccc5F)CC4)cc3)ncc2c(=O)n1-c1ccccn1] | 5-fluoro-1-[[4-fluoro-3-[[4-[4-[[1- methyl-3-oxo-2-(2- pyridyl)pyrazolo[3,4-d]pyrimidin-6- yl]amino]phenyl]piperazin-1- yl]methyl]phenyl]methyl]quinazoline- 2,4-dione | 703.3 | 703 |
| 104 | 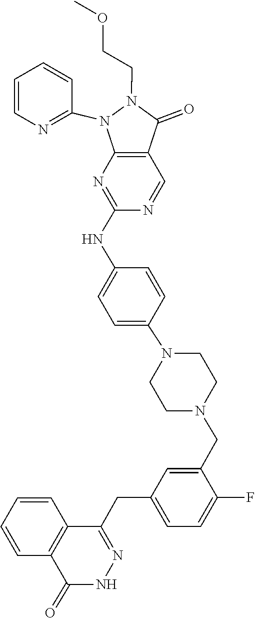 [SMILES: COCCn1c(=O)c2cnc(Nc3ccc(N4CCN(Cc5cc(Cc6n[nH]c(=O)c7ccccc67)ccc5F)CC4)cc3)nc2n1-c1ccccn1] | 4-[4-fluoro-3-[[4-[4-[[2-(2- methoxyethyl)-3-oxo-1-(2- pyridyl)pyrazolo[3,4-d]pyrimidin-6- yl]amino]phenyl]piperazin-1- yl]methyl]phenyl]methyl]-2H- phthalazin-1-one | 713.3 | 713 |
| 105 | 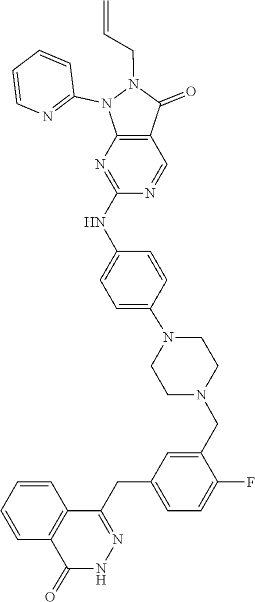 [SMILES: C=CCn1c(=O)c2cnc(Nc3ccc(N4CCN(Cc5cc(Cc6n[nH]c(=O)c7ccccc67)ccc5F)CC4)cc3)nc2n1-c1ccccn1] | 4-[[3-[[4-[4-[2-allyl-3-oxo-1-(2- pyridyl)pyrazolo[3,4-d]pyrimidin-6- yl]amino]phenyl]piperazin-1- yl]methyl]-4-fluoro-phenyl]methyl]- 2H-phthalazin-1-one | 695.3 | 695 |
| 106 | 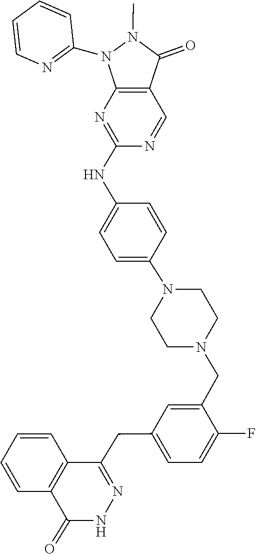 [SMILES: Cn1c(=O)c2cnc(Nc3ccc(N4CCN(Cc5cc(Cc6n[nH]c(=O)c7ccccc67)ccc5F)CC4)cc3)nc2n1-c1ccccn1] | 4-[[4-fluoro-3-[[4-[4-[[2-methyl-3- oxo-1-(2-pyridyl)pyrazolo[3,4- d]pyrimidin-6- yl]amino]phenyl]piperazin-1- yl]methyl]phenyl]methyl]-2H- phthalazin-1-one | 669.3 | 669 |
| 107 | 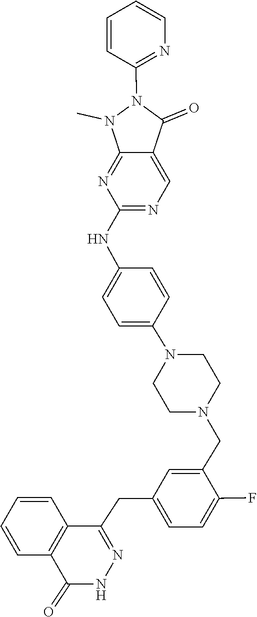 [SMILES: Cn1c2nc(Nc3ccc(N4CCN(Cc5cc(Cc6n[nH]c(=O)c7ccccc67)ccc5F)CC4)cc3)ncc2c(=O)n1-c1ccccn1] | 4-[[4-fluoro-3-[[4-[4-[[1-methyl-3- oxo-2-(2-pyridyl)pyrazolo[3,4- d]pyrimidin-6- yl]amino]phenyl]piperazin-1- yl]methyl]phenyl]methyl]-2H- phthalazin-1-one | 669.3 | 669 |
| 108 | 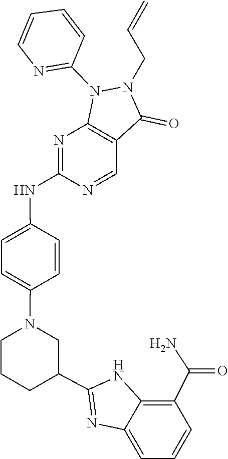 [SMILES: C=CCn1c(=O)c2cnc(Nc3ccc(N4CCCC(c5nc6cccc(C(N)=O)c6[nH]5)C4)cc3)nc2n1-c1ccccn1] | 2-[1-[4-[[2-allyl-3-oxo-1-(2- pyridyl)pyrazolo[3,4-d]pyrimidin-6- yl]amino]phenyl]-3-piperidyl]-3H- benzimidazole-4-carboxamide | 587.3 | 587 |
| 109 | 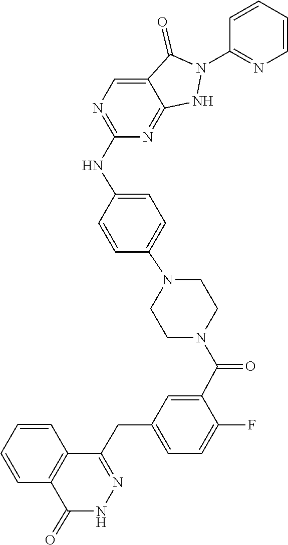 [SMILES: O=C(c1cc(Cc2n[nH]c(=O)c3ccccc23)ccc1F)N1CCN(c2ccc(Nc3ncc4c(=O)n(-c5ccccn5)[nH]c4n3)cc2)CC1] | 4-[[4-fluoro-3-[4-[4-[[3-oxo-2-(2- pyridyl)-1H-pyrazolo[3,4- d]pyrimidin-6- yl]amino]phenyl]piperazine-1- carbonyl]phenyl]methyl]-2H- phthalazin-1-one | 669.2 | 669 |
| 110 | 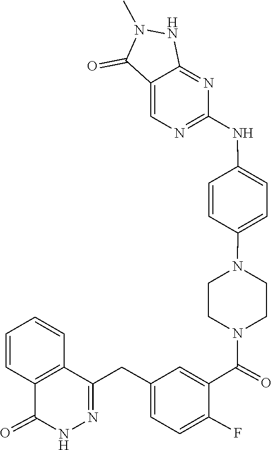 [SMILES: Cn1[nH]c2nc(Nc3ccc(N4CCN(C(=O)c5cc(Cc6n[nH]c(=O)c7ccccc67)ccc5F)CC4)cc3)ncc2c1=O] | 4-[[4-fluoro-3-[4-[4-[(2-methyl-3- oxo-1H-pyrazolo[3,4-d]pyrimidin-6- yl)amino]phenyl]piperazine-1- carbonyl]phenyl]methyl]-2H- phthalazin-1-one | 606.2 | 606 |
| 111 | 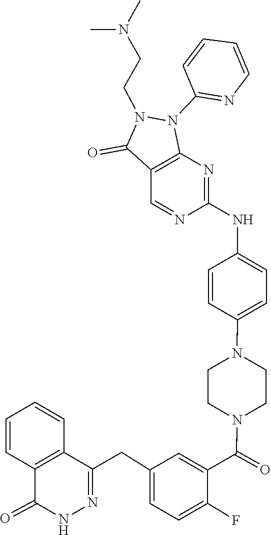 [SMILES: CN(C)CCn1c(=O)c2cnc(Nc3ccc(N4CCN(C(=O)c5cc(Cc6n[nH]c(=O)c7ccccc67)ccc5F)CC4)cc3)nc2n1-c1ccccn1] | 4-[[3-[4-[4-[[2-[2- (dimethylamino)ethyl]-3-oxo-1-(2- pyridyl)pyrazolo[3,4-d]pyrimidin-6- yl]amino]phenyl]piperazine-1- carbonyl]-4-fluoro-phenyl]methyl]- 2H-phthalazin-1-one | 740.3 | 740 |
| 112 | 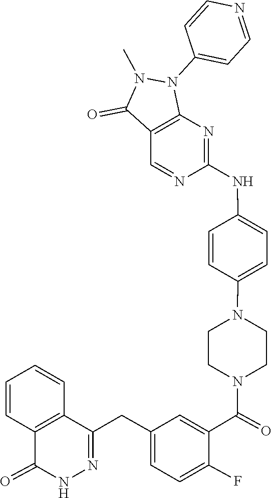 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1ccncc1] | 4-[[4-fluoro-3-[4-[4-[[2-methyl-3- oxo-1-(4-pyridyl)pyrazolo[3,4- d]pyrimidin-6- yl]amino]phenyl]piperazine-1- carbonyl]phenyl]methyl]-2H- phthalazin-1-one | 683.3 | 683 |
| 113 | 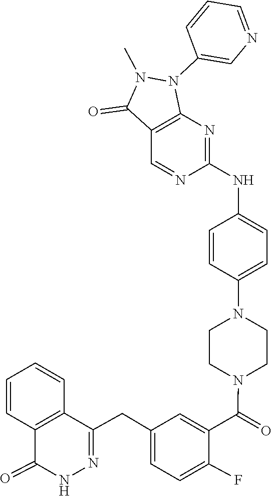 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1cccnc1] | 4-[[4-fluoro-3-[4-[4-[[2-methyl-3- oxo-1-(3-pyridyl)pyrazolo[3,4- d]pyrimidin-6- yl]amino]phenyl]piperazine-1- carbonyl]phenyl]methyl]-2H- phthalazin-1-one | 683.3 | 683 |
| 114 | 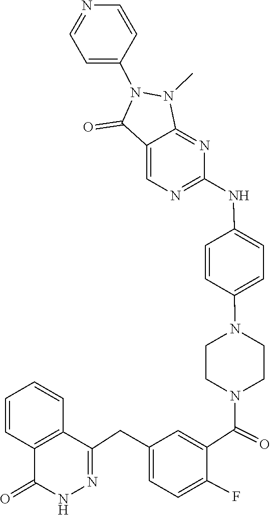 [SMILES: Cn1c2nc(Nc3ccc(N4CCN(C(=O)c5cc(Cc6n[nH]c(=O)c7ccccc67)ccc5F)CC4)cc3)ncc2c(=O)n1-c1ccncc1] | 4-[[4-fluoro-3-[4-[4-[[1-methyl-3- oxo-2-(4-pyridyl)pyrazolo[3,4- d]pyrimidin-6- yl]amino]phenyl]piperazine-1- carbonyl]phenyl]methyl]-2H- phthalazin-1-one | 683.3 | 683 |
| 115 | 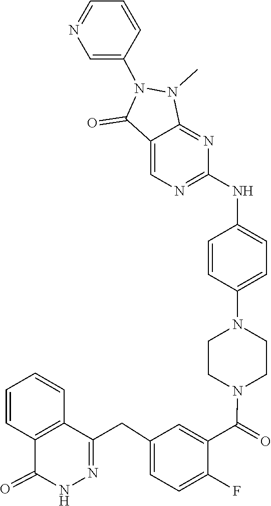 [SMILES: Cn1c2nc(Nc3ccc(N4CCN(C(=O)c5cc(Cc6n[nH]c(=O)c7ccccc67)ccc5F)CC4)cc3)ncc2c(=O)n1-c1cccnc1] | 4-[[4-fluoro-3-[4-[4-[[1-methyl-3- oxo-2-(3-pyridyl)pyrazolo[3,4- d]pyrimidin-6- yl]amino]phenyl]piperazine-1- carbonyl]phenyl]methyl]-2H- phthalazin-1-one | 683.3 | 683 |
| 116 | 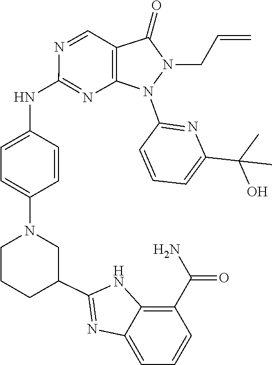 [SMILES: C=CCn1c(=O)c2cnc(Nc3ccc(N4CCCC(c5nc6cccc(C(N)=O)c6[nH]5)C4)cc3)nc2n1-c1cccc(C(C)(C)O)n1] | 2-[1-[4-[[2-allyl-1-[6-(1-hydroxy-1- methyl-ethyl)-2-pyridyl]-3-oxo- pyrazolo[3,4-d]pyrimidin-6- yl]amino]phenyl]-3-piperidyl]-3H- benzimidazole-4-carboxamide | 645.3 | 645 |
| 117 | 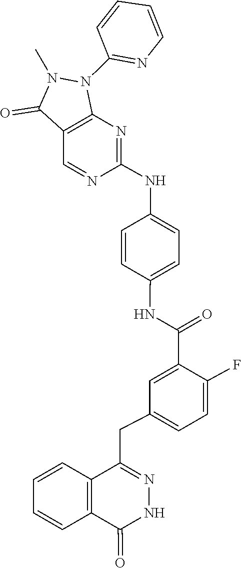 [SMILES: Cn1c(=O)c2cnc(Nc3ccc(NC(=O)c4cc(Cc5n[nH]c(=O)c6ccccc56)ccc4F)cc3)nc2n1-c1ccccn1] | 2-fluoro-N-[4-[2-methyl-3-oxo-1-(2- pyridyl)pyrazolo[3,4-d]pyrimidin-6- yl]amino]phenyl]-5-[(4-oxo-3H- phthalazin-1-yl)methyl]benzamide | 614.2 | 614 |
| 118 | 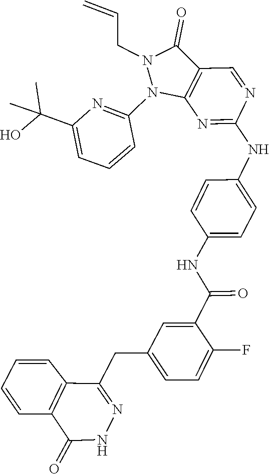 [SMILES: C=CCn1c(=O)c2cnc(Nc3ccc(NC(=O)c4cc(Cc5n[nH]c(=O)c6ccccc56)ccc4F)cc3)nc2n1-c1cccc(C(C)(C)O)n1] | N-[4-[[2-allyl-1-[6-(1-hydroxy-1- methyl-ethyl)-2-pyridyl]-3-oxo- pyrazolo[3,4-d]pyrimidin-6- yl]amino]phenyl]-2-fluoro-5-[(4-oxo- 3H-phthalazin-1- yl)methyl]benzamide | 698.3 | 698 |
| 119 | 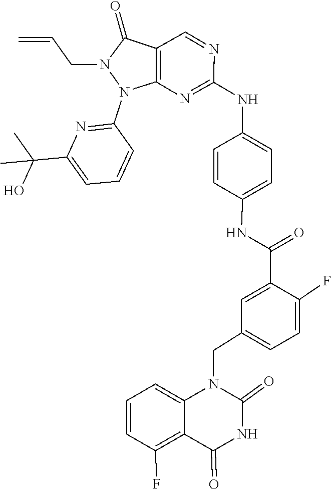 [SMILES: C=CCn1c(=O)c2cnc(Nc3ccc(NC(=O)c4cc(Cn5c(=O)[nH]c(=O)c6c(F)cccc65)ccc4F)cc3)nc2n1-c1cccc(C(C)(C)O)n1] | N-[4-[2-allyl-1-[6-(1-hydroxy-1- methyl-ethyl)-2-pyridyl]-3-oxo- pyrazolo[3,4-d]pyrimidin-6- yl]amino]phenyl]-2-fluoro-5-[(5- fluoro-2,4-dioxo-quinazolin-1- yl)methyl]benzamide | 732.2 | 732 |
| 120 | 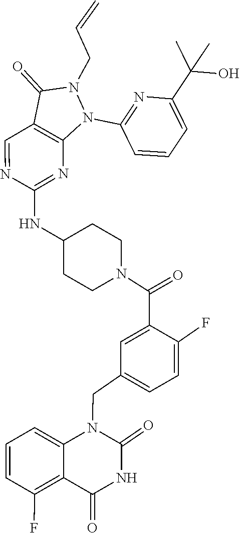 [SMILES: C=CCn1c(=O)c2cnc(NC3CCN(C(=O)c4cc(Cn5c(=O)[nH]c(=O)c6c(F)cccc65)ccc4F)CC3)nc2n1-c1cccc(C(C)(C)O)n1] | 1-[[3-[4-[2-allyl-1-[6-(1-hydroxy-1- methyl-ethyl)-2-pyridyl]-3-oxo- pyrazolo[3,4-d]pyrimidin-6- yl]amino]piperidine-1-carbonyl]-4- fluoro-phenyl]methyl]-5-fluoro- quinazoline-2,4-dione | 724.3 | 724 |
| 121 | 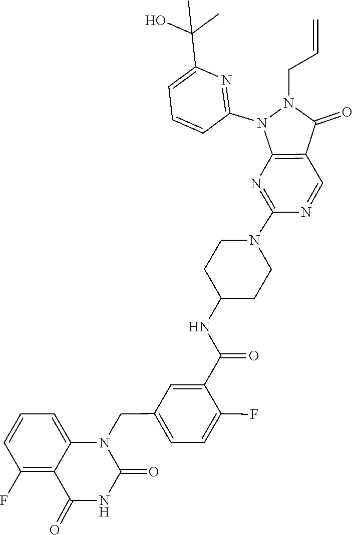 [SMILES: C=CCn1c(=O)c2cnc(N3CCC(NC(=O)c4cc(Cn5c(=O)[nH]c(=O)c6c(F)cccc65)ccc4F)CC3)nc2n1-c1cccc(C(C)(C)O)n1] | N-[1-[2-allyl-1-[6-(1-hydroxy-1- methyl-ethyl)-2-pyridyl]-3-oxo- pyrazolo[3,4-d]pyrimidin-6-yl]-4- piperidyl]-2-fluoro-5-[(5-fluoro-2,4- dioxo-quinazolin-1- yl)methyl]benzamide | 724.3 | 724 |
| 122 | 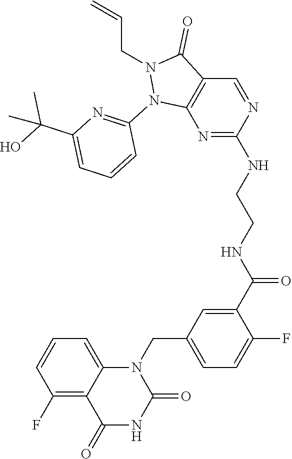 [SMILES: C=CCn1c(=O)c2cnc(NCCNC(=O)c3cc(Cn4c(=O)[nH]c(=O)c5c(F)cccc54)ccc3F)nc2n1-c1cccc(C(C)(C)O)n1] | N-[2-[2-allyl-1-[6-(1-hydroxy-1- methyl-ethyl)-2-pyridyl]-3-oxo- pyrazolo[3,4-d]pyrimidin-6- yl]amino]ethyl]-2-fluoro-5-[(5-fluoro- 2,4-dioxo-quinazolin-1- yl)methyl]benzamide | 684.2 | 684 |
| 123 | 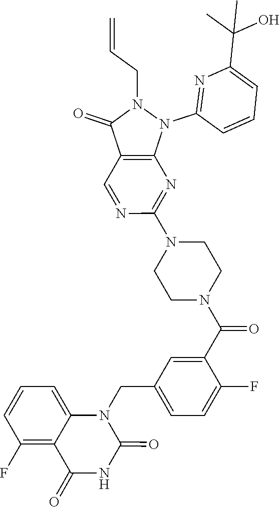 [SMILES: C=CCn1c(=O)c2cnc(N3CCN(C(=O)c4cc(Cn5c(=O)[nH]c(=O)c6c(F)cccc65)ccc4F)CC3)nc2n1-c1cccc(C(C)(C)O)n1] | 1-[[3-[4-[2-allyl-1-[6-(1-hydroxy-1- methyl-ethyl)-2-pyridyl]-3-oxo- pyrazolo[3,4-d]pyrimidin-6- yl]piperazine-1-carbonyl]-4-fluoro- phenyl]methyl]-5-fluoro-quinazoline- 2,4-dione | 710.3 | 710 |
| 124 | 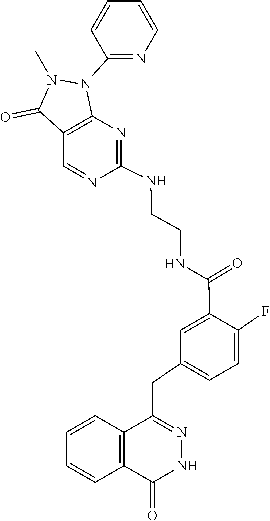 [SMILES: Cn1c(=O)c2cnc(NCCNC(=O)c3cc(Cc4n[nH]c(=O)c5ccccc45)ccc3F)nc2n1-c1ccccn1] | 2-fluoro-N-[2-[2-methyl-3-oxo-1-(2- pyridyl)pyrazolo[3,4-d]pyrimidin-6- yl]amino]ethyl]-5-[(4-oxo-3H- phthalazin-1-yl)methyl]benzamide | 566.2 | 566 |
| 125 | 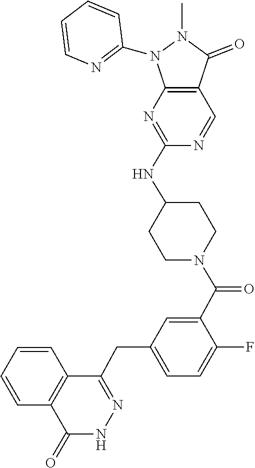 [SMILES: Cn1c(=O)c2cnc(NC3CCN(C(=O)c4cc(Cc5n[nH]c(=O)c6ccccc56)ccc4F)CC3)nc2n1-c1ccccn1] | 4-[[4-fluoro-3-[4-[[2-methyl-3-oxo-1- (2-pyridyl)pyrazolo[3,4-d]pyrimidin- 6-yl]amino]piperidine-1- carbonyl]phenyl]methyl]-2H- phthalazin-1-one | 606.2 | 606 |
| 126 | 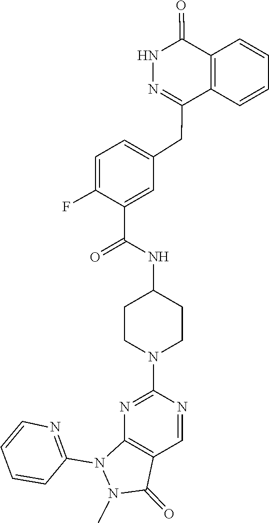 [SMILES: Cn1c(=O)c2cnc(N3CCC(NC(=O)c4cc(Cc5n[nH]c(=O)c6ccccc56)ccc4F)CC3)nc2n1-c1ccccn1] | 2-fluoro-N-[1-[2-methyl-3-oxo-1-(2- pyridyl)pyrazolo[3,4-d]pyrimidin-6- yl]-4-piperidyl]-5-[(4-oxo-3H- phthalazin-1-yl)methyl]benzamide | 606.2 | 606 |
| 127 | 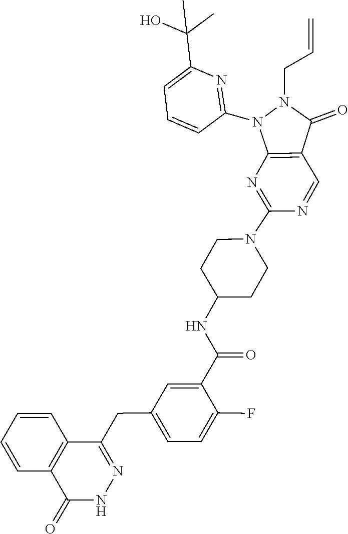 [SMILES: C=CCn1c(=O)c2cnc(N3CCC(NC(=O)c4cc(Cc5n[nH]c(=O)c6ccccc56)ccc4F)CC3)nc2n1-c1cccc(C(C)(C)O)n1] | N-[1-[2-allyl-1-[6-(1-hydroxy-1- methyl-ethyl)-2-pyridyl]-3-oxo- pyrazolo[3,4-d]pyrimidin-6-yl]-4- piperidyl]-2-fluoro-5-[(4-oxo-3H- phthalazin-1-yl)methyl]benzamide | 690.3 | 690 |

### General Synthetical Procedures and Examples of the Compound's Preparation. Synthesis of Building Blocks

### Preparation 1. 2-Methyl-6-methylsulfanyl-1H-pyrazolo[3,4-d]pyrimidin-3-one (P1)

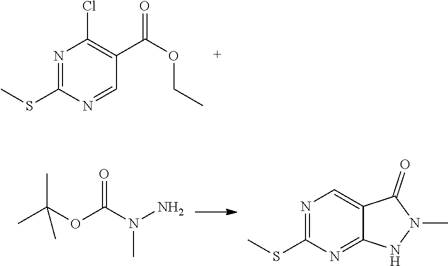

DIPEA (18.71 mL, 107.4 mmol) and tert-butyl N-amino-N-methylcarbamate (6.59 g, 45.11 mmol) were added to a solution of ethyl 4-chloro-2-methylthio-5-pyrimidinecarboxylate (10.0 g, 42.9 mmol) in THF (80 mL). The reaction mixture was heated at reflux for 72 h, before being concentrated in vacuo. Et₂O (43 mL) was added to the residue, and the resultant precipitate was collected by filtration. The filtrate was evaporated to dryness, and the residue was cooled in an ice bath, after which TFA (43 mL) was added. The resultant solution was stirred at rt for 1 h, followed by stirring at 70° C. for 1 h. The solvent was removed in vacuo and the residue was dissolved in EtOH (43 mL) and cooled in an ice bath, after which 6 M NaOH (86 mL) was added. The resultant solution was stirred at rt for 15 min, before being acidified (pH 3) by addition of conc. HCl. The solution was evaporated to dryness and the resultant residue was partitioned between chloroform (100 mL) and water (100 mL), and the organic phase was washed with brine, dried over Na₂SO₄, filtered, and concentrated in vacuo to afford the target compound (P1, 6.79 g, 80%) as yellow solid. ¹H NMR (400 MHz, DMSO-d₆) δ 12.65 (s, 1H), 8.67 (s, 1H), 3.35 (s, 3H), 2.51 (s, 3H).

### Preparation 2. 2-Methyl-6-methylsulfanyl-1-pyrimidin-2-yl-pyrazolo[3,4-d]pyrimidin-3-one (P2)

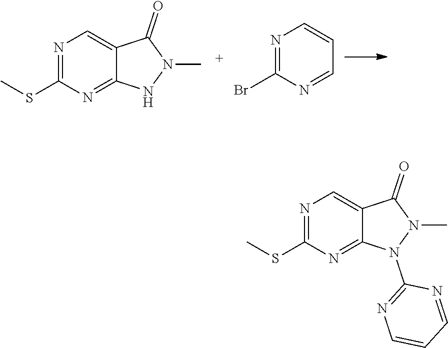

N,N′-Dimethylethylenediamine (449 mg, 5.0 mmol) was added to a solution of the 2-methyl-6-methylsulfanyl-1H-pyrazolo[3,4-d]pyrimidin-3-one (P1, 500 mg, 2.5 mmol), 2-bromopyrimidine (486 mg, 3.0 mmol), CuI (485 mg, 2.54 mmol) and K₂CO₃ (493 mg, 3.55 mmol) in 1,4-dioxane (5 mL) at 80° C. The resultant suspension was heated at 95° C. for 18 h, over which time a color changed from orange to dark green. The reaction mixture was cooled to ambient temperature and diluted with NH₄OH (25 mL) before being extracted with EtOAc (2×30 mL). The combined organic extracts were washed with brine, dried over Na₂SO₄, and evaporated to dryness before the crude material was purified via chromatography on silica with hexane-EtOAc (1:1) to yield 2-methyl-6-methylsulfanyl-1-pyrimidin-2-yl-pyrazolo[3,4-d]pyrimidin-3-one (P2, 70 mg, 10%) as a white solid. ¹H NMR (400 MHz, DMSO-d₆) δ 9.04 (d, J=1.3 Hz, 1H), 8.97 (dd, J=4.8, 1.3 Hz, 2H), 7.55 (td, J=4.8, 1.3 Hz, 1H), 3.45 (s, 3H), 2.56 (s, 3H).

### Preparation 3. 6-Chloro-2-methyl-1-pyrimidin-2-yl-1,2-dihydro-3H-pyrazolo[3,4-d]pyrimidin-3-one (P3)

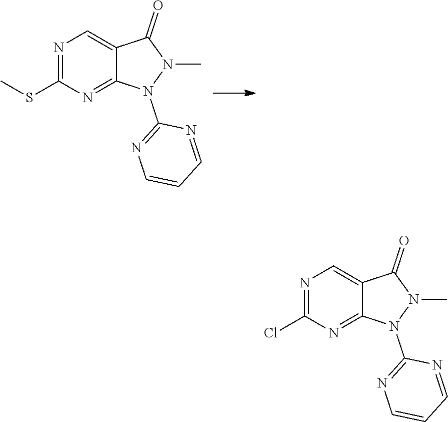 [SMILES: CSc1ncc2c(=O)n(C)n(-c3ncccn3)c2n1 ; Cn1c(=O)c2cnc(Cl)nc2n1-c1ncccn1]

To a solution of 2-methyl-6-methylsulfanyl-1-pyrimidin-2-yl-pyrazolo[3,4-d]pyrimidin-3-one (P2, 0.1 g, 0.36 mmol) in DCM-MeCN, 3:1 (10 mL), SO₂Cl₂(0.25 g, 1.8 mmol) was added portionwise at ambient temperature. The mixture was stirred at ambient temperature for 1 h and carefully poured into aq. NaHCO₃. The mixture was extracted with DCM, the combined organic extracts were dried with Na₂SO₄ and evaporated giving 6-chloro-2-methyl-1-pyrimidin-2-yl-1,2-dihydro-3H-pyrazolo[3,4-d]pyrimidin-3-one (P3, 0.094 g, 99%) as a white solid that was used for the next steps without additional purification. LCMS (m/z): 263.4 [MH]⁺.

### Preparation 4. 5-Fluoro-1H-quinazoline-2,4-dione (P4)

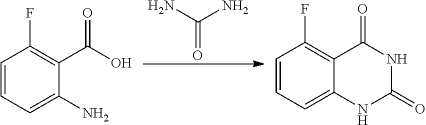

2-Amino-6-fluoro-benzoic acid (5 g, 32.2 mmol) was dissolved in water (23 mL) with a few drops of 2M NaOH, and the mixture was refluxed with urea (15.5 g, 258.3 mmol) for 72 h. The reaction was monitored by TLC (methanol:DCM, 10:90). The crude was then evaporated and 5-fluoro-1H-quinazoline-2,4-dione was precipitated in ethanol to yield 5-fluoro-1H-quinazoline-2,4-dione (P4, 3.98 g, 68.6%) as a white solid. ¹H NMR (400 MHz, DMSO-d₆) δ 11.25 (s, 2H), 7.60 (td, J=8.2, 5.5 Hz, 1H), 6.96 (d, J=8.3 Hz, 1H), 6.91 (dd, J=11.3, 8.2 Hz, 1H).

### Preparation 5. Methyl 2-fluoro-5-[(5-fluoro-2,4-dioxo-quinazolin-1-yl)methyl]benzoate (P5)

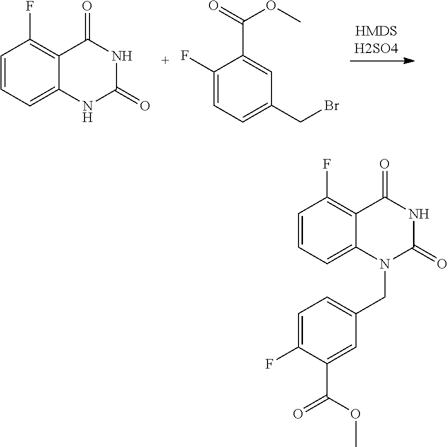 [SMILES: O=c1[nH]c(=O)c2c(F)cccc2[nH]1 ; COC(=O)c1cc(CBr)ccc1F ; COC(=O)c1cc(Cn2c(=O)[nH]c(=O)c3c(F)cccc32)ccc1F]

To a suspension of 5-fluoro-1H-quinazoline-2,4-dione (P4, 1.5 g, 8.3 mmol) in toluene (13 mL) and HMDS (2.69 g, 16.6 mmol), few drops of sulfuric acid were added with caution. The mixture was heated to reflux and stirred under reflux for 8 h until a clear solution was obtained. After the removal of toluene and excess HMDS under vacuum distillation, methyl 5-(bromomethyl)-2-fluorobenzoate (3.08 g, 12.5 mmol) was added to the residue. The reaction mixture was heated to 140° C. and was stirred at this temperature for 3 h, the reaction mixture was diluted with 1,4-dioxane (9.5 mL) at 100° C., and then methanol (7 mL) was added at 70° C. for 30 min. The suspension was cooled below 5° C. and precipitates were collected by filtration. After washing with methanol and water the crude product was dried under vacuum conditions to yield methyl 2-fluoro-5-[(5-fluoro-2,4-dioxo-quinazolin-1-yl)methyl]benzoate (P5, 1.83 g, 63%) as a white solid. ¹H NMR (400 MHz, DMSO-d₆) δ 11.70 (s, 1H), 7.86 (dd, J=6.8, 2.4 Hz, 1H), 7.61 (ddt, J=16.0, 7.4, 4.2 Hz, 2H), 7.30 (dd, J=10.8, 8.5 Hz, 1H), 7.07 (d, J=8.6 Hz, 1H), 7.02(dd, J=11.0, 8.3 Hz, 1H), 5.32 (s, 2H), 3.84 (s, 3H).

### Preparation 6. 2-Fluoro-5-[(5-fluoro-2,4-dioxo-3,4-dihydroquinazolin-1(2H)-yl)methyl]benzoic acid (P6)

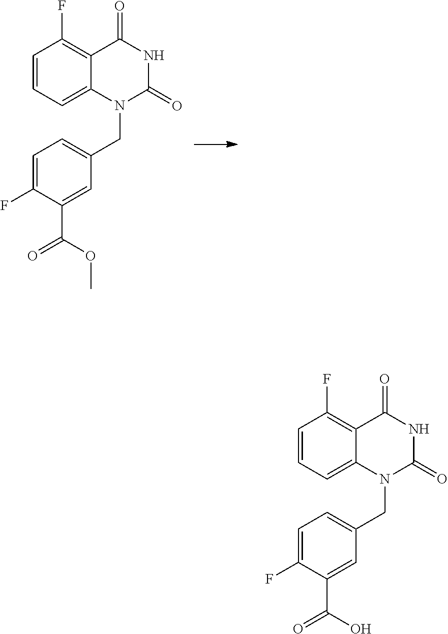 [SMILES: COC(=O)c1cc(Cn2c(=O)[nH]c(=O)c3c(F)cccc32)ccc1F ; O=C(O)c1cc(Cn2c(=O)[nH]c(=O)c3c(F)cccc32)ccc1F]

To a solution of methyl 2-fluoro-5-[(5-fluoro-2,4-dioxo-quinazolin-1-yl)methyl]benzoate (P5, 1.73 g, 5.0 mmol) in 20 mL H₂O, 10 mL 1N NaOH was added, and the mixture was stirred at ambient temperature for 15 h. MeOH was evaporated, the residue was acidified with 10% HCl to pH 2, formed white pellets were washed with H₂O and dried giving 2-fluoro-5-[(5-fluoro-2,4-dioxo-3,4-dihydroquinazolin-1(2H)-yl)methyl]benzoic acid (P6, 1.66 g, 100%). ¹H NMR (400 MHz, DMSO-d₆) δ 13.29 (s, 1H), 11.70 (s, 1H), 7.91-7.75 (m, 1H), 7.63 (dd, J=14.2, 8.3 Hz, 1H), 7.59-7.48 (m, 1H), 7.26 (dd, J=10.5, 8.7 Hz, 1H), 7.08 (d, J=8.6 Hz, 1H), 7.06-6.97 (m, 1H), 5.31 (s, 2H).

### Preparation 7. 5-Fluoro-1-(4-fluoro-3-{[4-(4-nitrophenyl) piperidin-1-yl]carbonyl}benzyl) quinazoline-2,4 (1H,3H)-dione (P7)

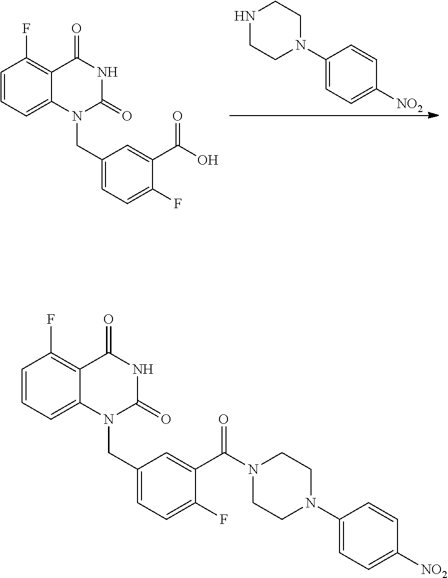 [SMILES: O=C(O)c1cc(Cn2c(=O)[nH]c(=O)c3c(F)cccc32)ccc1F ; O=[N+]([O-])c1ccc(N2CCNCC2)cc1 ; O=C(c1cc(Cn2c(=O)[nH]c(=O)c3c(F)cccc32)ccc1F)N1CCN(c2ccc([N+](=O)[O-])cc2)CC1]

To a solution of 2-fluoro-5-[(5-fluoro-2,4-dioxo-3,4-dihydroquinazolin-1(2H)-yl)methyl]benzoic acid) (P6, 0.20 g, 0.60 mmol) in 20 mL CH₂Cl₂, DIPEA was added (0.31 g, 2.4 mmol), following by addition of T3P (0.38 g, 1.2 mmol). The mixture was stirred for 15 h at ambient temperature. The mixture was diluted with Et₂O, the formed yellow pellet was filtered off, washed with H₂O, and dried giving 5-fluoro-1-(4-fluoro-3-{[4-(4-nitrophenyl) piperidin-1-yl]carbonyl}benzyl) quinazoline-2,4 (1H,3H)-dione (P7, 0.19 g, 66%). ¹H NMR (400 MHz, DMSO-d₆) δ 11.67 (s, 1H), 8.08 (d, J=9.3 Hz, 2H), 7.64 (dd, J=14.1, 8.4 Hz, 1H), 7.47 (s, 1H), 7.41 (d, J=6.1 Hz, 1H), 7.30 (t, J=9.0 Hz, 1H), 7.10-6.92(m, 4H), 5.31 (s, 2H), 3.75 (s, 2H), 3.58 (s, 2H), 3.33 (d, J=9.3 Hz, 4H).

### Preparation 8. 1-(3-{[4-(4-Aminophenyl) piperidin-1-yl]carbonyl}-4-fluorobenzyl)-5-fluoroquinazoline-2,4 (1H,3H)-dione (P8)

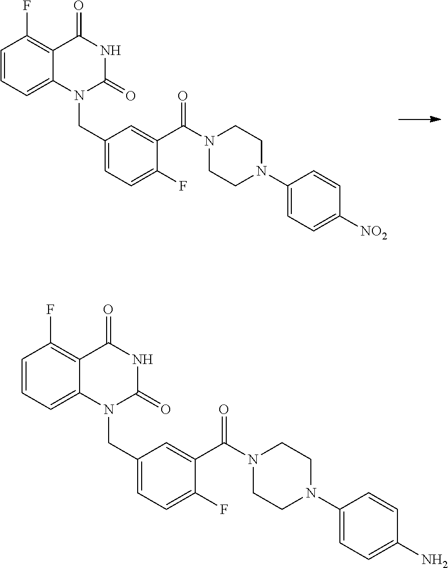 [SMILES: O=C(c1cc(Cn2c(=O)[nH]c(=O)c3c(F)cccc32)ccc1F)N1CCN(c2ccc([N+](=O)[O-])cc2)CC1 ; Nc1ccc(N2CCN(C(=O)c3cc(Cn4c(=O)[nH]c(=O)c5c(F)cccc54)ccc3F)CC2)cc1]

A mixture of 5-fluoro-1-(4-fluoro-3-{[4-(4-nitrophenyl) piperidin-1-yl]carbonyl}benzyl) quinazoline-2,4 (1H,3H)-dione (P7, 0.19 g, 0.38 mmol) in 10 mL DMF and 20 mg 10% Pd/C was stirred in the atmosphere of H₂ at ambient temperature and 4 MPa for 15 h. The catalyst was filtered off through Celite pad and filtrate was evaporated giving 1-(3-{[4-(4-aminophenyl) piperidin-1-yl]carbonyl}-4-fluorobenzyl)-5-fluoroquinazoline-2,4 (1H,3H)-dione (P8, 0.19 g, 95%) as a grey powder. ¹H NMR (400 MHz, DMSO-d₆) δ 11.50 (br. s, 1H), 7.63 (dd, J=14.3, 8.2 Hz, 1H), 7.44 (s, 1H), 7.38 (d, J=6.2 Hz, 1H), 7.34-7.18 (m, 2H), 7.04 (d, J=8.7 Hz, 1H), 6.74 (d, J=8.9 Hz, 2H), 6.49 (d, J=8.8 Hz, 2H), 5.31 (s, 2H), 3.73 (s, 2H), 3.25 (s, 4H), 2.94 (d, J=6.9 Hz, 2H), 2.76 (s, 2H).

### Preparation 9. tert-Butyl [4-({2-fluoro-5-[(5-fluoro-2,4-dioxo-3,4-dihydroquinazolin-1(2H)-yl)methyl]benzoyl}amino)phenyl]carbamate (P9)

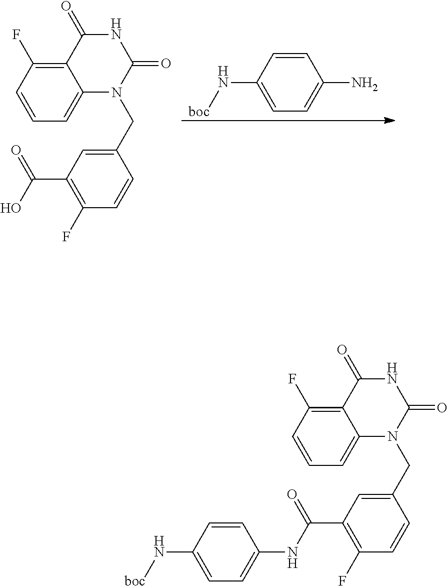

To a solution of 2-fluoro-5-[(5-fluoro-2,4-dioxo-3,4-dihydroquinazolin-1(2H)-yl)methyl]benzoic acid (P6, 0.21 g, 0.64 mmol) and DIPEA (0.66 g, 5.12 mmol) in 20 mL DCM, T3P was added (0.41 g, 1.28 mmol) and the mixture was stirred at ambient temperature for 5 h. The mixture was diluted with Et₂O, the formed yellow pellet was filtered off, washed with Et₂O and dried giving tert-butyl [4-({2-fluoro-5-[(5-fluoro-2,4-dioxo-3,4-dihydroquinazolin-1(2H)-yl)methyl]benzoyl}amino)phenyl]carbamate (P9, 0.33 g, 98%). ¹H NMR (400 MHz, DMSO-d₆) δ 11.63 (s, 1H), 10.20 (s, 1H), 9.21 (s, 1H), 7.63 (d, J=5.9 Hz, 2H), 7.56 (d, J=8.6 Hz, 2H), 7.47 (s, 1H), 7.40 (d, J=8.6 Hz, 2H), 7.28 (t, J=9.2 Hz, 1H), 7.10 (d, J=8.3 Hz, 1H), 7.06-6.95 (m, 1H), 5.33 (s, 2H), 1.48 (s, 9H).

### Preparation 10. N-(4-Aminophenyl)-2-fluoro-5-[(5-fluoro-2,4-dioxo-3,4-dihydroquinazolin-1(2H)-yl)methyl]benzamide (P10)

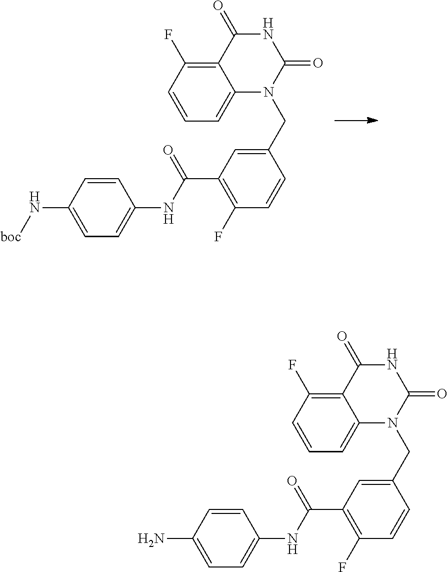

To a solution of tert-butyl [4-({2-fluoro-5-[(5-fluoro-2,4-dioxo-3,4-dihydroquinazolin-1(2H)-yl)methyl]benzoyl}amino)phenyl]carbamate (P9, 0.33 g, 0.63 mmol) in 5 mL dioxane, 5 mL 3M HCl in dioxane was added and the mixture was kept at ambient temperature for 48 h. The mixture was diluted with Et₂O, the formed pellet was filtered off, washed with H₂O, and dried giving N-(4-Aminophenyl)-2-fluoro-5-[(5-fluoro-2,4-dioxo-3,4-dihydroquinazolin-1(2H)-yl)methyl]benzamide (P10, 0.27 g, 100%). ¹H NMR (400 MHz, DMSO-d₆) δ 11.69 (s, 1H), 10.65 (s, 1H), 10.39 (s, 2H), 7.80 (d, J=8.6 Hz, 1H), 7.64 (dd, J=15.4, 7.4 Hz, 1H), 7.51 (s, 1H), 7.39 (d, J=8.7 Hz, 2H), 7.31 (t, J=9.2 Hz, 1H), 7.10 (d, J=8.6 Hz, 1H), 7.07-6.97 (m, 1H), 5.33 (s, 2H), 4.28 (s, 2H).

### Preparation 11. 2-(6-Bromo-2-pyridyl) propan-2-ol (P11)

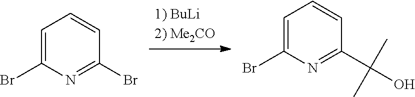

2.5 M Solution of n-butyllithium (10.1 mL, 25.3 mmol) in hexane was placed in a dry 100 mL round bottomed flask fitted with a stir bar, septum, and temperature probe. The flask was cooled in a dry ice/acetone bath to −78° C. THF (15 mL) was added to the solution, following by addition of a solution of 2,6-dibromopyridine (6 g, 25.3 mmol) in THF (30 mL) slowly via syringe maintaining the temperature under −60° C. The dark yellow-brown solution was stirred for 30 minutes in the dry ice bath, then acetone was added (2.98 mL, 40.48 mmol). The deep green solution was stirred in the dry-ice bath for 15 minutes then the reaction was allowed to warm to room temperature. After 1 h a 5% aqueous solution of ammonium chloride was added (50 mL) carefully. The mixture was extracted with dichloromethane, dried over anhydrous Na₂SO₄, and evaporated. The residue was purified by column chromatography on silica with hexane-DCM (1:2) to yield 2-(6-bromo-2-pyridyl) propan-2-ol (P11, 3.35 g, 61%) as oil. ¹H NMR (400 MHz, DMSO-d₆) δ 7.76-7.63 (m, 2H), 7.45 (dd, J=7.6, 1.1 Hz, 1H), 5.29 (s, 1H), 1.41 (s, 6H).

### Preparation 12. 2-Allyl-6-methylsulfanyl-1H-pyrazolo[3,4-d]pyrimidin-3-one (P12)

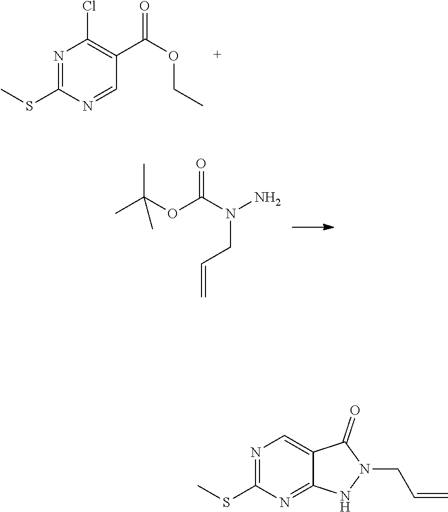

DIPEA (9.36 mL, 53.7 mmol) and tert-butyl N-allyl-N-amino-carbamate (3.88 g, 22.55 mmol) were added to a solution of ethyl 4-chloro-2-methylthio-5-pyrimidinecarboxylate (5.0 g, 21.48 mmol) in THF (65 mL). The reaction mixture was heated at reflux for 72 h, before being concentrated in vacuo. Et₂O (22 mL) was added to the residue, and the resultant precipitate was collected by filtration. The filtrate was evaporated to dryness, and the residue was cooled in an ice bath, after which TFA (22 mL) was added. The resultant solution was stirred at rt for 1 h, followed by stirring at 70° C. for 1 h. The solvent was removed in vacuo and the residue was dissolved in EtOH (22 mL) and cooled in an ice bath, after which 6M NaOH (44 mL) was added. The resultant solution was stirred at rt for 15 min, before being acidified (pH 3) by addition of conc. HCl. The solution was evaporated to dryness and the resultant residue was partitioned between chloroform (44 mL) and water (44 mL), and the organic phase was washed with brine, dried over Na₂SO₄, filtered, and concentrated in vacuo to afford the target compound (P12, 3.8 g, 79%) as yellow solid. ¹H NMR (400 MHz, DMSO-d₆) δ 8.65 (s, 1H), 5.94-5.79 (m, 1H), 5.21-5.04 (m, 3H), 4.38 (d, J=5.3 Hz, 2H).

### Preparation 13. 2-Allyl-1-[6-(1-hydroxy-1-methyl-ethyl)-2-pyridyl]-6-methylsulfanyl-pyrazolo[3,4-d]pyrimidin-3-one (P13)

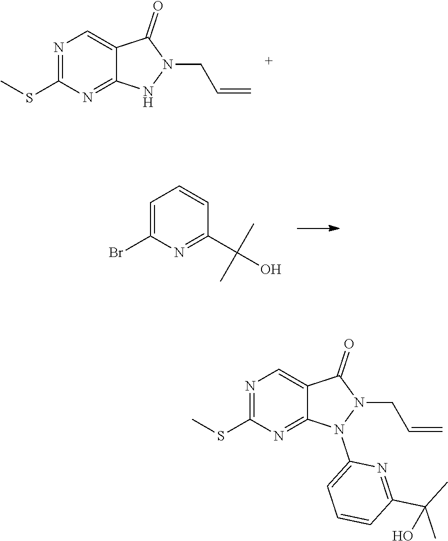 [SMILES: C=CCn1[nH]c2nc(SC)ncc2c1=O ; CC(C)(O)c1cccc(Br)n1 ; C=CCn1c(=O)c2cnc(SC)nc2n1-c1cccc(C(C)(C)O)n1]

N,N′-Dimethylethylenediamine (1.59 g, 17.98 mmol) was added to a solution of the 2-allyl-6-methylsulfanyl-1H-pyrazolo[3,4-d]pyrimidin-3-one (P12, 2.0 g, 8.99 mmol), 2-(6-bromo-2-pyridyl) propan-2-ol (P11, 2.52 g, 11.69 mmol), CuI (1.71 g, 8.99 mmol) and K₂CO₃ (1.74 g, 12.59 mmol) in 1,4-dioxane (18 mL) at 80° C. The resultant suspension was heated at 95° C. for 18 h, over which time a color change of orange to dark green occurred. The reaction mixture was cooled to ambient temperature and diluted with NH₄OH (90 mL) before being extracted with EtOAc (2×90 mL). The combined organic extracts were washed with brine, dried over Na₂SO₄, and evaporated to dryness before the crude material was purified via chromatography on silica with hexane-EtOAc (1:1) to yield 2-allyl-1-[6-(1-hydroxy-1-methyl-ethyl)-2-pyridyl]-6-methylsulfanyl-pyrazolo[3,4-d]pyrimidin-3-one (P13, 1.7 g, 53%) as a white solid. ¹H NMR (400 MHz, DMSO-d₆) δ 9.01 (s, 1H), 8.03 (t, J=7.9 Hz, 1H), 7.75 (dd, J=8.1, 0.8 Hz, 1H), 7.65 (dd, J=7.7, 0.9 Hz, 1H), 5.74-5.60 (m, 1H), 5.33 (s, 1H), 4.99 (dd, J=10.2, 1.4 Hz, 1H), 4.81 (dd, J=17.2, 1.6 Hz, 1H), 4.78-4.72(m, 2H), 2.56 (s, 3H), 1.46 (s, 6H).

### Preparation 14. 2-Allyl-6-methylsulfanyl-1-(2-pyridyl)pyrazolo[3,4-d]pyrimidin-3-one (P14)

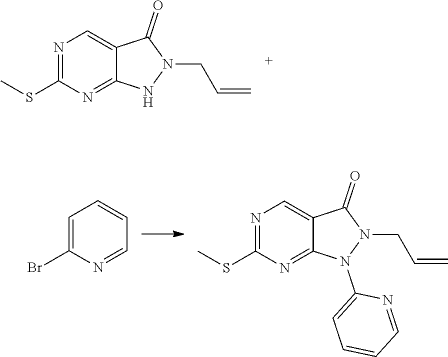 [SMILES: C=CCn1[nH]c2nc(SC)ncc2c1=O ; Brc1ccccn1 ; C=CCn1c(=O)c2cnc(SC)nc2n1-c1ccccn1]

N,N′-Dimethylethylenediamine (1.29 g, 14.7 mmol) was added to a solution of the 2-allyl-6-methylsulfanyl-1H-pyrazolo[3,4-d]pyrimidin-3-one (P12, 1.63 g, 7.35 mmol), 2-bromopyridine (1.51 g, 9.55 mmol), CuI (1.4 g, 7.35 mmol) and K₂CO₃ (1.42 g, 10.29 mmol) in 1,4-dioxane (15 mL) at 80° C. The resultant suspension was heated at 95° C. for 18 h, over which time a color changed from orange to dark green. The reaction mixture was cooled to ambient temperature and diluted with NH₄OH (75 mL) before being extracted with EtOAc (2×75 mL). The combined organic extracts were washed with brine, dried over Na₂SO₄, and evaporated to dryness before the crude material was purified via chromatography on silica with hexane-EtOAc (4:1 to 0:1) to yield 2-allyl-6-methylsulfanyl-1-(2-pyridyl)pyrazolo[3,4-d]pyrimidin-3-one (P14, 1.39 g, 63%) as a white solid. ¹H NMR (400 MHz, DMSO-d₆) δ 9.02(s, 1H), 8.55 (ddd, J=5.0, 2.0, 0.9 Hz, 1H), 8.08 (ddd, J=8.3, 7.4, 1.9 Hz, 1H), 7.91 (dt, J=8.3, 0.9 Hz, 1H), 7.43 (ddd, J=7.5, 4.9, 1.0 Hz, 1H), 5.69 (ddt, J=16.3, 10.3, 5.9 Hz, 1H), 5.00 (dt, J=10.2, 1.3 Hz, 1H), 4.85 (dq, J=17.0, 1.4 Hz, 1H), 4.69 (dt, J=6.0, 1.5 Hz, 2H), 2.55 (s, 3H).

### Preparation 15. 2-(2-Methoxyethyl)-6-methylsulfanyl-1H-pyrazolo[3,4-d]pyrimidin-3-one (P15)

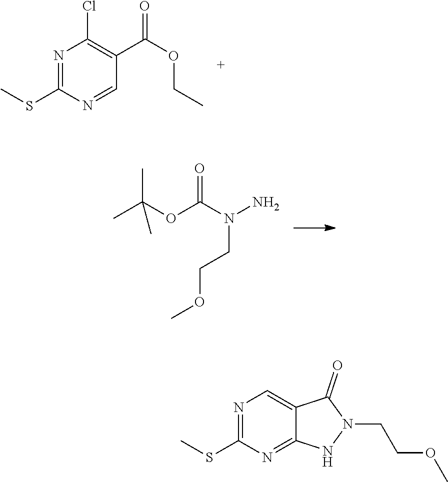

DIPEA (2.62 mL, 12 mmol) and tert-butyl N-amino-N-(2-methoxyethyl)carbamate (1.2 g, 6.3 mmol) were added to a solution of ethyl 4-chloro-2-methylthio-5-pyrimidinecarboxylate (1.4 g, 6.0 mmol) in THF (18 mL). The reaction mixture was heated at reflux for 72 h, before being concentrated in vacuo. Et₂O (6 mL) was added to the residue, and the resultant precipitate was collected by filtration. The filtrate was evaporated to dryness, and the residue was cooled in an ice bath, after which TFA (6 mL) was added. The resultant solution was stirred at rt for 1 h, followed by 70° C. for 1 h. The solvent was removed in vacuo and the residue was dissolved in EtOH (6 mL) and cooled in an ice bath, after which 6M NaOH (12 mL) was added. The resultant solution was stirred at rt for 15 min, before being acidified (pH 3) via the addition of conc. HCl. The solution was evaporated to dryness and the resultant residue was partitioned between chloroform (12 mL) and water (12 mL), and the organic phase was washed with brine, dried over Na₂SO₄, filtered, and concentrated in vacuo to afford the target compound (P15, 1.0 g, 69%) as a yellow solid. ¹H NMR (400 MHz, DMSO-d₆) δ 8.64 (s, 1H), 3.91 (t, J=5.6 Hz, 2H), 3.60 (t, J=5.6 Hz, 2H), 3.22(s, 3H), 2.53 (s, 3H).

### Preparation 16. 2-(2-Methoxyethyl)-6-methylsulfanyl-1-(2-pyridyl)pyrazolo[3,4-d]pyrimidin-3-one (P16)

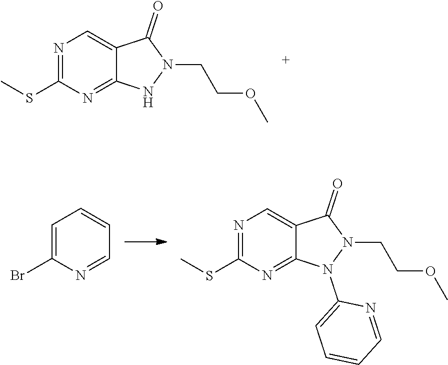

N,N′-Dimethylethylenediamine (0.403 g, 4.58 mmol) was added to a solution of the 2-(2-methoxyethyl)-6-methylsulfanyl-1H-pyrazolo[3,4-d]pyrimidin-3-one (P15, 0.55 g, 2.29 mmol), 2-bromopyridine (0.470 g, 2.97 mmol), CuI (0.436 g, 2.29 mmol) and K₂CO₃ (0.429 g, 3.2 mmol) in 1,4-dioxane (5 mL) at 80° C. The resultant suspension was heated at 95° C. for 18 h, over which time a color changed from orange to dark green. The reaction mixture was cooled to ambient temperature and diluted with NH₄OH (25 mL) before being extracted with EtOAc (2×25 mL). The combined organic extracts were washed with brine, dried over Na₂SO₄, and evaporated to dryness before the crude material was purified via chromatography on silica with hexane-EtOAc (1:1 to 0:1) yield 2-(2-methoxyethyl)-6-methylsulfanyl-1-(2-pyridyl)pyrazolo[3,4-d]pyrimidin-3-one (P16, 0.409 g, 56%) as a white solid. ¹H NMR (400 MHz, DMSO-d₆) δ 9.00 (s, 1H), 8.57-8.50 (m, 1H), 8.14-8.05 (m, 1H), 8.04-7.95 (m, 1H), 7.47-7.39 (m, 1H), 4.24 (t, J=5.3 Hz, 2H), 3.35 (t, J=5.2 Hz, 2H), 3.07 (s, 3H), 2.56 (s, 3H).

### Preparation 17. 1-[6-(1-Hydroxy-1-methyl-ethyl)-2-pyridyl]-2-(2-methoxyethyl)-6-methylsulfanyl-pyrazolo[3,4-d]pyrimidin-3-one (P17)

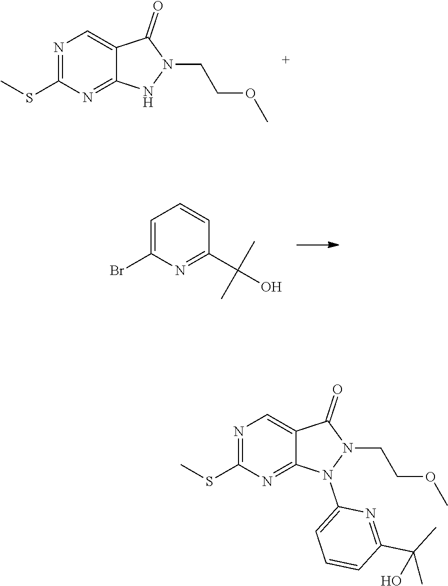

N,N′-Dimethylethylenediamine (0.330 g, 3.74 mmol) was added to a solution of the 2-(2-methoxyethyl)-6-methylsulfanyl-1H-pyrazolo[3,4-d]pyrimidin-3-one (P15, 0.45 g, 1.87 mmol), 2-(6-bromo-2-pyridyl) propan-2-ol (0.526 g, 2.43 mmol), CuI (0.357 g, 1.87 mmol) and K₂CO₃ (0.362 g, 2.62 mmol) in 1,4-dioxane (4 mL) at 80° C. The resultant suspension was heated at 95° C. for 18 h, over which time a color changed from orange to dark green. The reaction mixture was cooled to ambient temperature and diluted with NH₄OH (20 mL) before being extracted with EtOAc (2×20 mL). The combined organic extracts were washed with brine, dried over Na₂SO₄, and evaporated to dryness before the crude material was purified via chromatography on silica with hexane-EtOAc (1:2) yield 1-[6-(1-hydroxy-1-methyl-ethyl)-2-pyridyl]-2-(2-methoxyethyl)-6-methylsulfanyl-pyrazolo[3,4-d]pyrimidin-3-one (P17, 0.38 g, 54%) as a white solid. ¹H NMR (400 MHz, DMSO-d₆) δ 8.99 (s, 1H), 8.05 (t, J=7.9 Hz, 1H), 7.82(d, J=8.0 Hz, 1H), 7.65 (d, J=7.6 Hz, 1H), 5.32(s, 1H), 4.29 (t, J=5.3 Hz, 2H), 3.35 (t, J=5.4 Hz, 2H), 3.06 (s, 3H), 2.56 (s, 3H), 1.44 (s, 6H).

### Preparation 18. 2-(2-Hydroxyethyl)-6-methylsulfanyl-1H-pyrazolo[3,4-d]pyrimidin-3-one (P18)

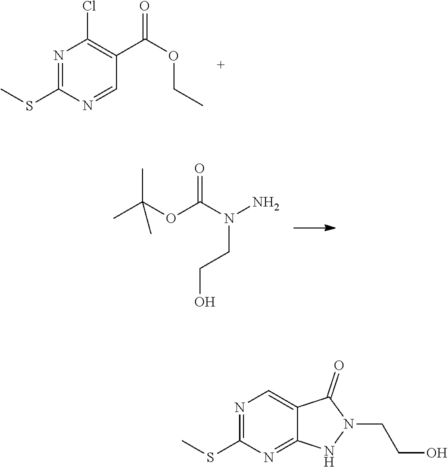

DIPEA (3.74 mL, 17.18 mmol) and tert-butyl N-amino-N-(2-hydroxyethyl)carbamate (1.59 g, 9.02 mmol) were added to a solution of ethyl 4-chloro-2-methylthio-5-pyrimidinecarboxylate (2 g, 8.59 mmol) in THF (25 mL). The reaction mixture was heated at reflux for 72 h, before being concentrated in vacuo. Et₂O (10 mL) was added to the residue, and the resultant precipitate was collected by filtration. The filtrate was evaporated to dryness, and the residue was cooled in an ice bath, after which TFA (10 mL) was added. The resultant solution was stirred at rt for 1 h, followed by 70° C. for 1 h. The solvent was removed in vacuo and the residue was dissolved in EtOH (10 mL) and cooled in an ice bath, after which 6M NaOH (18 mL) was added. The resultant solution was stirred at rt for 15 min, before being acidified (pH 3) via the addition of conc. HCl. The solution was evaporated to dryness and the resultant residue was partitioned between chloroform (20 mL) and water (20 mL), and the organic phase was washed with brine, dried over Na₂SO₄, filtered, and concentrated in vacuo to afford the target compound (P18, 0.65 g, 33%) as a yellow solid. ¹H NMR (400 MHz, DMSO-d₆) δ 12.56 (s, 1H), 8.67 (s, 1H), 3.81 (t, J=6.3 Hz, 2H), 3.68-3.59 (m, 2H), 2.53 (s, 3H).

### Preparation 19. 2-(2-Hydroxyethyl)-6-methylsulfanyl-1-(2-pyridyl)pyrazolo[3,4-d]pyrimidin-3-one (P19)

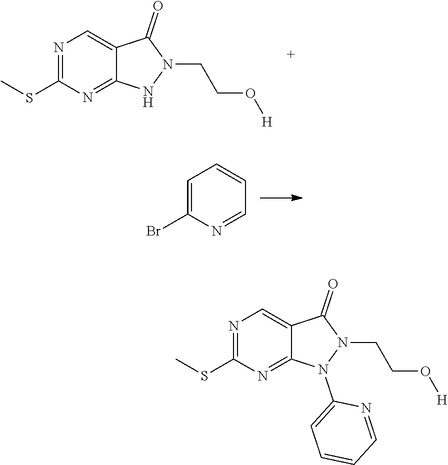 [SMILES: CSc1ncc2c(=O)n(CCO)[nH]c2n1 ; Brc1ccccn1 ; CSc1ncc2c(=O)n(CCO)n(-c3ccccn3)c2n1]

N,N′-Dimethylethylenediamine (0.272 g, 3.08 mmol) was added to a solution of the 2-(2-hydroxyethyl)-6-methylsulfanyl-1H-pyrazolo[3,4-d]pyrimidin-3-one (P15, 0.35 g, 1.54 mmol), 2-bromopyridine (0.317 g, 2 mmol), CuI (0.295 g, 1.54 mmol) and K₂CO₃ (0.299 g, 2.15 mmol) in 1,4-dioxane (3 mL) at 80° C. The resultant suspension was heated at 95° C. for 18 h, over which time a color changed from orange to dark green. The reaction mixture was cooled to ambient temperature and diluted with NH₄OH (15 mL) before being extracted with EtOAc (2×15 mL). The combined organic extracts were washed with brine, dried over Na₂SO₄, and evaporated to dryness before the crude material was purified via chromatography on silica with hexane-EtOAc (1:1 to 0:1) yield 2-(2-hydroxyethyl)-6-methylsulfanyl-1-(2-pyridyl)pyrazolo[3,4-d]pyrimidin-3-one (P19, 0.17 g, 36%) as a white solid. ¹H NMR (400 MHz, DMSO-d₆) δ 9.00 (s, 1H), 8.53 (d, J=4.1 Hz, 1H), 8.10 (td, J=7.7, 2.0 Hz, 1H), 8.00-7.93 (m, 1H), 7.42(ddd, J=7.4, 4.8, 1.2 Hz, 1H), 4.76-4.68 (m, 1H), 4.16-4.08 (m, 3H), 3.38 (t, J=5.8 Hz, 3H), 2.56 (s, 3H).

### Preparation 20. 2-(2-Hydroxyethyl)-1-[6-(1-hydroxy-1-methyl-ethyl)-2-pyridyl]-6-methylsulfanyl-pyrazolo[3,4-d]pyrimidin-3-one (P20)

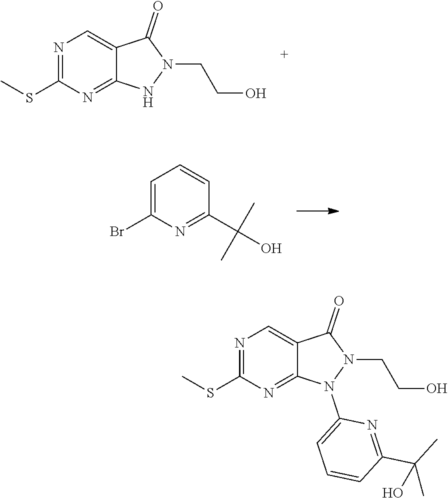 [SMILES: CSc1ncc2c(=O)n(CCO)[nH]c2n1 ; CC(C)(O)c1cccc(Br)n1 ; CSc1ncc2c(=O)n(CCO)n(-c3cccc(C(C)(C)O)n3)c2n1]

N,N′-Dimethylethylenediamine (P18, 0.389 g, 4.42 mmol) was added to a solution of the 2-(2-hydroxyethyl)-6-methylsulfanyl-1H-pyrazolo[3,4-d]pyrimidin-3-one (0.5 g, 2.21 mmol), 2-(6-bromo-2-pyridyl) propan-2-ol (0.62 g, 2.87 mmol), CuI (0.421 g, 2.21 mmol) and K₂CO₃ (0.427 g, 3.09 mmol) in 1,4-dioxane (5 mL) at 80° C. The resultant suspension was heated at 95° C. for 18 h, over which time a color changed from orange to dark green. The reaction mixture was cooled to ambient temperature and diluted with NH₄OH (20 mL) before being extracted with EtOAc (2×20 mL). The combined organic extracts were washed with brine, dried over Na₂SO₄, and evaporated to dryness before the crude material was purified via chromatography on silica with DCM-MeOH (20:1) to yield 2-(2-hydroxyethyl)-1-[6-(1-hydroxy-1-methyl-ethyl)-2-pyridyl]-6-methylsulfanyl-pyrazolo[3,4-d]pyrimidin-3-one (P20, 0.454 g, 57%) as a white solid. ¹H NMR (400 MHz, DMSO-d₆) δ 8.98 (s, 1H), 8.05 (t, J=7.9 Hz, 1H), 7.80 (d, J=8.0 Hz, 1H), 7.65 (d, J=7.7 Hz, 1H), 5.31 (s, 1H), 4.72(t, J=5.6 Hz, 1H), 4.18 (t, J=5.6 Hz, 2H), 3.38 (q, J=5.6 Hz, 2H), 2.56 (s, 3H), 1.44 (s, 6H).

### Preparation 21. 2-[2-(Dimethylamino)ethyl]-6-methylsulfanyl-1H-pyrazolo[3,4-d]pyrimidin-3-one (P21)

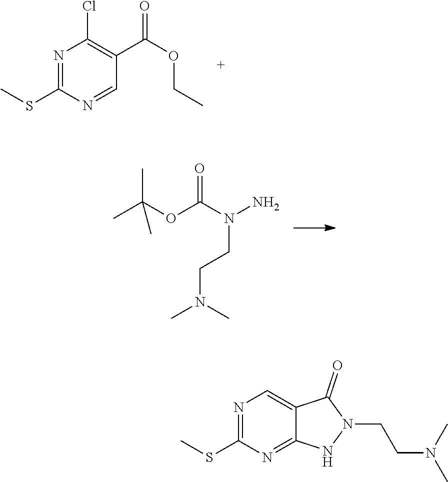

DIPEA (9.35 mL, 42.96 mmol) and tert-butyl N-amino-N-[2-(dimethylamino)ethyl]carbamate (4.8 g, 23.62 mmol) were added to a solution of ethyl 4-chloro-2-methylthio-5-pyrimidinecarboxylate (5.0 g, 21.48 mmol) in THF (60 mL). The reaction mixture was heated at reflux for 72 h, before being concentrated in vacuo. Et₂O (25 mL) was added to the residue, and the resultant precipitate was collected by filtration. The filtrate was evaporated to dryness, and the residue was cooled in an ice bath, after which TFA (20 mL) was added. The resultant solution was stirred at rt for 1h, followed by 70° C. for 1 h. The solvent was removed in vacuo and the residue was dissolved in EtOH (20 mL) and cooled in an ice bath, after which 6M NaOH (45 mL) was added. The resultant solution was stirred at ambient temperature for 15 min, before being acidified (pH 3) via the addition of conc. HCl. The solution was evaporated to dryness and the resultant residue was partitioned between chloroform (45 mL) and water (45 mL), and the organic phase was washed with brine, dried over Na₂SO₄, filtered, and concentrated in vacuo to afford the target compound (P21, 5.0 g, 91%) as a yellow solid. ¹H NMR (400 MHz, DMSO-d₆) δ 8.14 (s, 1H), 3.82-3.74 (m, 2H), 2.52-2.46 (m, 4H), 2.36 (s, 3H), 2.15 (s, 6H).

### Preparation 22. 2-[2-(Dimethylamino)ethyl]-6-methylsulfanyl-1-(2-pyridyl)pyrazolo[3,4-d]pyrimidin-3-one (P22)

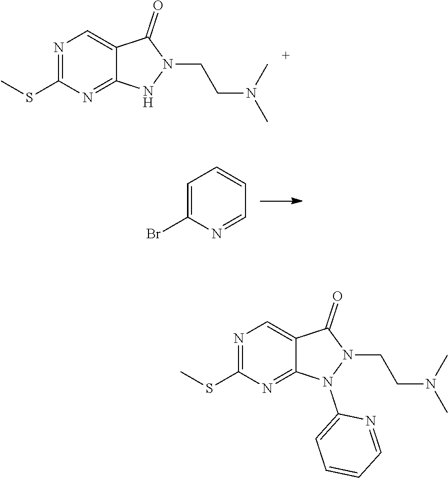

N,N′-Dimethylethylenediamine (0.696 g, 7.88 mmol) was added to a solution of the 2-[2-(dimethylamino)ethyl]-6-methylsulfanyl-1H-pyrazolo[3,4-d]pyrimidin-3-one (P21, 1.0 g, 3.94 mmol), 2-bromopyridine (0.81 g, 5.12 mmol), CuI (0.752 g, 3.94 mmol) and K₂CO₃ (0.763 g, 5.51 mmol) in 1,4-dioxane (10 mL) at 80° C. The resultant suspension was heated at 95° C. for 18 h, over which time a color changed from orange to dark green. The reaction mixture was cooled to ambient temperature and diluted with NH₄OH (40 mL) before being extracted with EtOAc (2×40 mL). The combined organic extracts were washed with brine, dried over Na₂SO₄, and evaporated to dryness before the crude material was purified via chromatography on silica with DCM-MeOH (95:5) to yield 2-[2-(dimethylamino)ethyl]-6-methylsulfanyl-1-(2-pyridyl)pyrazolo[3,4-d]pyrimidin-3-one (P22, 0.687 g, 52%) as a white solid. ¹H NMR (400 MHz, DMSO-d₆) δ 8.99 (s, 1H), 8.59-8.51 (m, 1H), 8.10 (td, J=7.8, 1.9 Hz, 1H), 7.98 (d, J=8.1 Hz, 1H), 7.44 (dd, J=7.3, 4.9 Hz, 1H), 4.14 (t, J=6.1 Hz, 2H), 2.55 (s, 3H), 2.24 (t, J=6.2 Hz, 2H), 2.00 (s, 6H).

### Preparation 23. 2-[2-(Dimethylamino)ethyl]-1-[6-(1-hydroxy-1-methyl-ethyl)-2-pyridyl]-6-methylsulfanyl-pyrazolo[3,4-d]pyrimidin-3-one (P23)

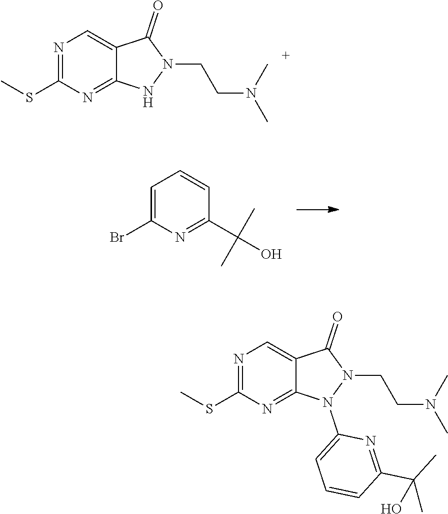

N,N′-Dimethylethylenediamine (0.835 g, 9.46 mmol) was added to a solution of the 2-[2-(dimethylamino)ethyl]-6-methylsulfanyl-1H-pyrazolo[3,4-d]pyrimidin-3-one (P21, 1.2 g, 4.73 mmol), 2-(6-bromo-2-pyridyl) propan-2-ol (1.33 g, 6.15 mmol), CuI (0.902 g, 4.73 mmol) and K₂CO₃ (0.9.16 g, 6.62 mmol) in 1,4-dioxane (10 mL) at 80° C. The resultant suspension was heated at 95° C. for 18 h, over which time a color changed from orange to dark green. The reaction mixture was cooled to ambient temperature and diluted with NH₄OH (50 mL) before being extracted with EtOAc (2×50 mL). The combined organic extracts were washed with brine, dried over Na₂SO₄, and evaporated to dryness before the crude material was purified via chromatography on silica with DCM-MeOH (20:1) to yield 2-[2-(dimethylamino)ethyl]-1-[6-(1-hydroxy-1-methyl-ethyl)-2-pyridyl]-6-methylsulfanyl-pyrazolo[3,4-d]pyrimidin-3-one (P23, 0.98 g, 53%) as a white solid. ¹H NMR (400 MHz, DMSO-d₆) δ 8.98 (s, 1H), 8.06 (t, J=7.9 Hz, 1H), 7.81 (d, J=8.0 Hz, 1H), 7.66 (d, J=7.8 Hz, 1H), 5.33 (s, 1H), 4.21 (t, J=6.1 Hz, 2H), 2.56 (s, 3H), 2.22(t, J=6.1 Hz, 2H), 1.98 (s, 6H), 1.44 (s, 6H).

### Preparation 24. 6-Chloro-2-methyl-1-pyridin-2-yl-1,2-dihydro-3H-pyrazolo[3,4-d]pyrimidin-3-one (P24)

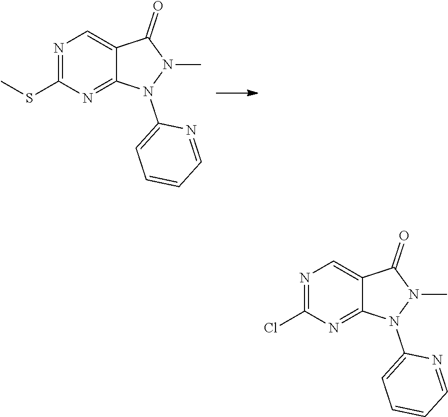 [SMILES: CSc1ncc2c(=O)n(C)n(-c3ccccn3)c2n1 ; Cn1c(=O)c2cnc(Cl)nc2n1-c1ccccn1]

To a solution of 2-methyl-6-(methylthio)-1-pyridin-2-yl-1,2-dihydro-3H-pyrazolo[3,4-d]pyrimidin-3-one (P63, 0.5 g, 1.8 mmol) in CH₂Cl₂-MeCN, 3:1 (20 mL), SO₂Cl₂(2.47 g, 18 mmol) was added portionwise at ambient temperature. The mixture was stirred at ambient temperature for 1 h and carefully poured into aq. NaHCO₃. The mixture was extracted with CH₂Cl₂, the combined organic extracts were dried with Na₂SO₄ and evaporated giving 6-chloro-2-methyl-1-pyridin-2-yl-1,2-dihydro-3H-pyrazolo[3,4-d]pyrimidin-3-one (P24, 0.37 g, 77%) as a white solid that was used for the next steps without additional purification. ¹H NMR (400 MHz, DMSO-d₆) δ 9.17 (s, 1H), 8.61 (d, J=4.5 Hz, 1H), 8.19 (m, 1H), 7.84 (d, J=8.2 Hz, 1H), 7.51 (dd, J=7.2, 5.0 Hz, 1H), 3.45 (s, 3H).

### Preparation 25. 4-(8-fluoro-6-oxo-3,4,5,6-tetrahydro-1H-azepino[5,4,3-cd]indol-2-yl)-3-methoxybenzaldehyde (P25)

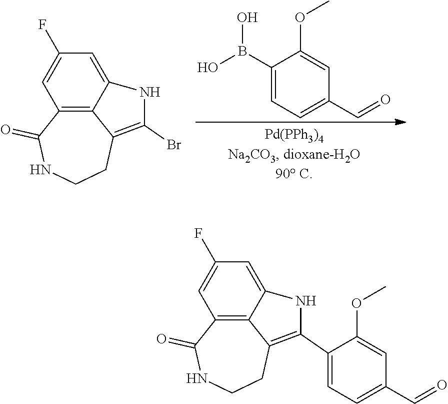 [SMILES: O=C1NCCc2c(Br)[nH]c3cc(F)cc1c23 ; COc1cc(C=O)ccc1B(O)O ; COc1cc(C=O)ccc1-c1[nH]c2cc(F)cc3c2c1CCNC3=O]

To a solution of (4-formyl-2-methoxyphenyl) boronic acid (0.35 g, 2.0 mmol) in 15 mL of 1,4-dioxane 5 mL of saturated aqueous sodium carbonate solution were added. Argon gas was purged for 10 min at room temperature. 2-Bromo-8-fluoro-1,3,4,5-tetrahydro-6H-azepino[5,4,3-cd]indol-6-one (0.5 g, 1.8 mmol) and tetrakis(triphenylphosphine) palladium (0) (0.10 g, 0.18 mmol) were added to the reaction mixture simultaneously and argon gas was bubbled in for another 5 min. The reaction mixture was heated to reflux for 12 h. After completion of the reaction as indicated by TLC the mixture was concentrated under reduced pressure. The residue was partitioned between dichloromethane and water. The organic layer was separated, washed with water and brine, dried over sodium sulfate and concentrated. The obtained crude residue was purified by column chromatography eluting with 15% ethyl acetate in dichloromethane to provide 4-(8-fluoro-6-oxo-3,4,5,6-tetrahydro-1H-azepino[5,4,3-cd]indol-2-yl)-3-methoxybenzaldehyde (P25, 0.38 g, 63%). ¹H NMR (400 MHz, DMSO-d₆) δ 11.52(s, 1H), 10.06 (s, 1H), 8.25 (s, 1H), 7.65 (d, J=4.9 Hz, 3H), 7.44 (dd, J=10.9, 2.4 Hz, 1H), 7.35 (dd, J=9.2, 2.4 Hz, 1H), 3.94 (s, 3H), 3.35 (s, 2H), 2.86 (s, 2H).

### Preparation 26. 8-fluoro-2-{2-methoxy-4-[(methylamino)methyl]phenyl}-1,3,4,5-tetrahydro-6H-azepino[5,4,3-cd]indol-6-one (P26)

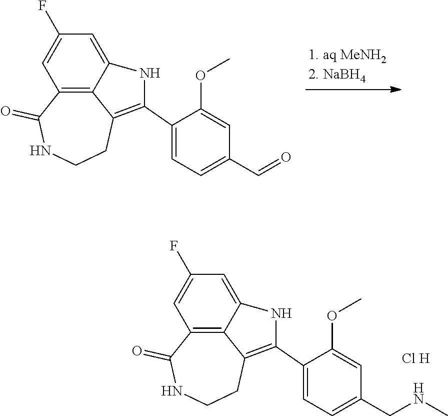

4-(8-fluoro-6-oxo-3,4,5,6-tetrahydro-1H-azepino[5,4,3-cd]indol-2-yl)-3-methoxybenzaldehyde (P25, 0.38 g, 1.1 mmol) was stirred in 33% aqueous solution of MeNH₂(1.0 mL) at ambient temperature for 15 h. After completion of the reaction the mixture was concentrated under reduced pressure. The residue was partitioned between dichloromethane and water. The organic layer was separated, washed with water and brine, dried over sodium sulfate and concentrated. The obtained crude residue was dissolved by adding methanol, and sodium borohydride (0.042 g, 1.1 mmol) was carefully added in portions under an ice bath. They were reacted for 1 h at room temperature. Methanol was removed, and it was diluted with dichloromethane, washed with saturated aqueous solution of sodium bicarbonate, extracted with dichloromethane for three times. The combined organic phase was washed with brine, dried over sodium sulfate, filtered, and concentrated in vacuo. The residue was suspended in H₂O, few drops of conc. HCl were added, and the solution was evaporated to dryness to give 8-fluoro-2-{2-methoxy-4-[(methylamino)methyl]phenyl}-1,3,4,5-tetrahydro-6H-azepino[5,4,3-cd]indol-6-one (P26, 0.17 g, 42%). ¹H NMR (400 MHz, DMSO-d₆) δ 11.43 (s, 1H), 9.36 (s, 2H), 8.22(t, J=5.6 Hz, 1H), 7.51-7.29 (m, 4H), 7.21 (t, J=7.5 Hz, 1H), 4.17 (t, J=5.7 Hz, 2H), 3.87 (s, 3H), 2.81 (s, 3H), 2.57 (t, J=5.3 Hz, 2H).

### Preparation 27. 3-chloro-4-(8-fluoro-6-oxo-3,4,5,6-tetrahydro-1H-azepino[5,4,3-cd]indol-2-yl)benzaldehyde (P27)

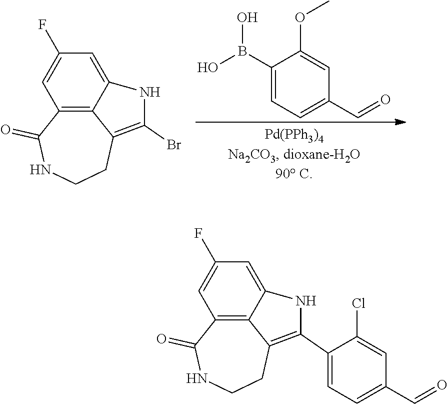 [SMILES: O=C1NCCc2c(Br)[nH]c3cc(F)cc1c23 ; COc1cc(C=O)ccc1B(O)O ; O=Cc1ccc(-c2[nH]c3cc(F)cc4c3c2CCNC4=O)c(Cl)c1]

To a solution of (2-chloro-4-formylphenyl) boronic acid (0.72 g, 4.0 mmol) in 30 mL of 1,4-dioxane 10 mL of saturated aqueous sodium carbonate solution were added. Argon gas was purged for 10 min at rt. 2-Bromo-8-fluoro-1,3,4,5-tetrahydro-6H-azepino[5,4,3-cd]indol-6-one (1.0 g, 3.6 mmol) and tetrakis(triphenylphosphine) palladium (0) (0.40 g, 0.035 mmol) were added to the reaction mixture simultaneously and argon gas was bubbled in for another 5 min. The reaction mixture was heated to reflux for 12 h. After completion of the reaction as indicated by TLC the mixture was concentrated under reduced pressure. The residue was partitioned between dichloromethane and water. The organic layer was separated, washed with water and brine, dried over sodium sulfate and concentrated. The obtained crude residue was purified by column chromatography eluting with 15% ethyl acetate in dichloromethane to provide 3-chloro-4-(8-fluoro-6-oxo-3,4,5,6-tetrahydro-1H-azepino[5,4,3-cd]indol-2-yl)benzaldehyde (P27, 0.38 g, 63%). ¹H NMR (400 MHz, DMSO-d₆) δ 11.77 (s, 1H), 10.07 (s, 1H), 8.25 (d, J=15.0 Hz, 2H), 8.14 (d, J=1.4 Hz, 1H), 7.98 (d, J=7.9 Hz, 1H), 7.79 (d, J=7.8 Hz, 1H), 7.28 (dd, J=9.1, 2.4 Hz, 1H), 3.47-3.34 (m, 2H), 2.80 (s, 2H).

### Preparation 28. 2-{2-chloro-4-[(methylamino)methyl]phenyl}-8-fluoro-1,3,4,5-tetrahydro-6H-azepino[5,4,3-cd]indol-6-one (P28)

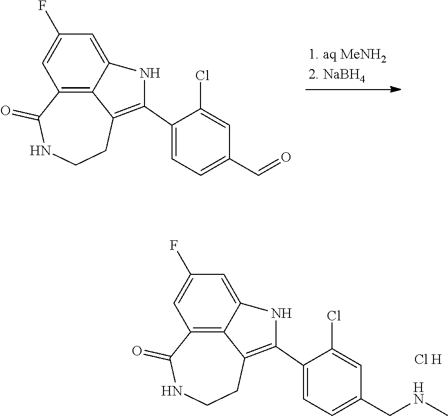

3-Chloro-4-(8-fluoro-6-oxo-3,4,5,6-tetrahydro-1H-azepino[5,4,3-cd]indol-2-yl)benzaldehyde (P27, 0.09 g, 0.26 mmol) was stirred in 33% aqueous solution of MeNH₂(1.0 mL) at ambient temperature for 15 h. After completion of the reaction the mixture was concentrated under reduced pressure. The residue was partitioned between dichloromethane and water. The organic layer was separated, washed with water and brine, dried over sodium sulfate and concentrated. The obtained crude residue was dissolved by adding methanol, and sodium borohydride (0.010 g, 0.26 mmol) was carefully added in portions under an ice bath. They were reacted for 1 h at room temperature. Methanol was removed, and it was diluted with dichloromethane, washed with saturated aqueous solution of sodium bicarbonate, extracted with dichloromethane for three times. The combined organic phase was washed with brine, dried over sodium sulfate, filtered, and concentrated in vacuo. The residue was suspended in H₂O, few drops of conc. HCl were added, and the solution was evaporated to dryness to give 2-{2-chloro-4-[(methylamino)methyl]phenyl}-8-fluoro-1,3,4,5-tetrahydro-6H-azepino[5,4,3-cd]indol-6-one (P28, 0.05 g, 53%). LCMS (m/z): 358.5 [MH]⁺.

### Preparation 29. 4-(8-fluoro-6-oxo-3,4,5,6-tetrahydro-1H-azepino[5,4,3-cd]indol-2-yl)-3-methylbenzaldehyde (P29)

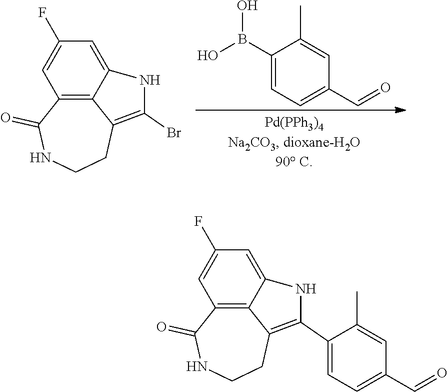 [SMILES: O=C1NCCc2c(Br)[nH]c3cc(F)cc1c23 ; Cc1cc(C=O)ccc1B(O)O ; Cc1cc(C=O)ccc1-c1[nH]c2cc(F)cc3c2c1CCNC3=O]

To a solution of (4-formyl-2-methylphenyl) boronic acid (0.64 g, 4.0 mmol) in 30 mL of 1,4-dioxane 10 mL of saturated aqueous sodium carbonate solution were added. Argon gas was purged for 10 min at room temperature. 2-Bromo-8-fluoro-1,3,4,5-tetrahydro-6H-azepino[5,4,3-cd]indol-6-one (1.0 g, 3.6 mmol) and tetrakis(triphenylphosphine) palladium (0) (0.40 g, 0.035 mmol) were added to the reaction mixture simultaneously and argon gas was bubbled in for another 5 min. The reaction mixture was heated to reflux for 12 h. After completion of the reaction as indicated by TLC the mixture was concentrated under reduced pressure. The residue was partitioned between dichloromethane and water. The organic layer was separated, washed with water and brine, dried over sodium sulfate and concentrated. The obtained crude residue was purified by column chromatography eluting with 15% ethyl acetate in dichloromethane to provide 4-(8-fluoro-6-oxo-3,4,5,6-tetrahydro-1H-azepino[5,4,3-cd]indol-2-yl)-3-methylbenzaldehyde (P29, 0.42 g, 39%). ¹H NMR (400 MHz, DMSO-d₆) δ 11.72(s, 1H), 10.05 (s, 1H), 8.23 (d, J=15.0 Hz, 2H), 8.15 (d, J=1.4 Hz, 1H), 7.98 (d, J=7.9 Hz, 1H), 7.79 (d, J=7.8 Hz, 1H), 7.25 (dd, J=9.1, 2.4 Hz, 1H), 3.45-3.35 (m, 2H), 2.81 (s, 2H), 2.31 (s, 3H).

### Preparation 30. 8-fluoro-2-{2-methyl-4-[(methylamino)methyl]phenyl}-1,3,4,5-tetrahydro-6H-azepino[5,4,3-cd]indol-6-one (P30)

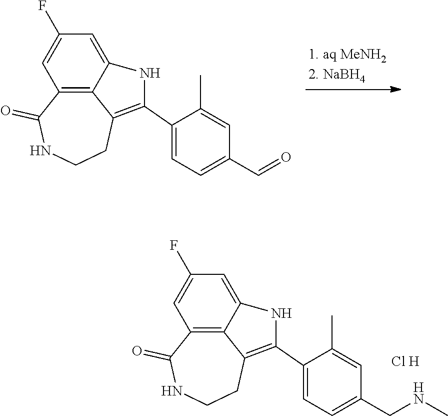

4-(8-fluoro-6-oxo-3,4,5,6-tetrahydro-1H-azepino[5,4,3-cd]indol-2-yl)-3-methylbenzaldehyde (P29, 0.42 g, 1.3 mmol) was stirred in 33% aqueous solution of MeNH₂(1.0 mL) at ambient temperature for 15 h. After completion of the reaction the mixture was concentrated under reduced pressure. The residue was partitioned between dichloromethane and water. The organic layer was separated, washed with water and brine, dried over sodium sulfate and concentrated. The obtained crude residue was dissolved by adding methanol, and sodium borohydride (0.042 g, 1.1 mmol) was carefully added in portions under an ice bath. They were reacted for 1 h at room temperature. Methanol was removed, and it was diluted with dichloromethane, washed with saturated aqueous solution of sodium bicarbonate, extracted with dichloromethane for three times. The combined organic phase was washed with brine, dried over sodium sulfate, filtered, and concentrated in vacuo. The residue was suspended in H₂O, few drops of conc. HCl were added, and the solution was evaporated to dryness to give 8-fluoro-2-{2-methyl-4-[(methylamino)methyl]phenyl}-1,3,4,5-tetrahydro-6H-azepino[5,4,3-cd]indol-6-one (P30, 0.15 g, 40%). ¹H NMR (400 MHz, DMSO-d₆) δ 11.61 (s, 1H), 9.36 (s, 2H), 8.24 (t, J=5.7 Hz, 1H), 7.64-7.28 (m, 5H), 5.00 (s, 2H), 4.13 (t, J=5.7 Hz, 2H), 3.38 (s, 2H), 2.71 (s, 3H), 2.28 (s, 3H).

### Preparation 31. 4-(8-Fluoro-6-oxo-3,4,5,6-tetrahydro-1H-azepino[5,4,3-cd]indol-2-yl)benzaldehyde (P31)

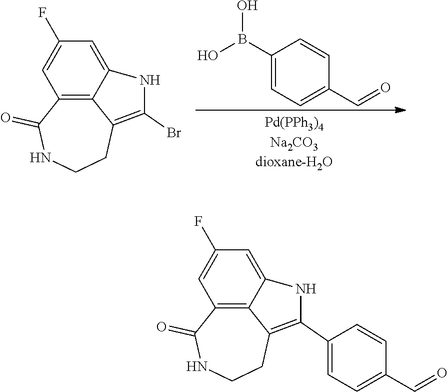 [SMILES: O=C1NCCc2c(Br)[nH]c3cc(F)cc1c23 ; O=Cc1ccc(B(O)O)cc1 ; O=Cc1ccc(-c2[nH]c3cc(F)cc4c3c2CCNC4=O)cc1]

To a solution of (4-formylphenyl) boronic acid (1.17 g, 7.7 mmol) in 20 mL of 1,4-dioxane 5 mL of saturated aq. sodium carbonate solution was added. Argon gas was purged for 10 min at room temperature. 2-Bromo-8-fluoro-1,3,4,5-tetrahydro-6H-azepino[5,4,3-cd]indol-6-one (2.0 g, 7.1 mmol) and tetrakis(triphenylphosphine) palladium (0) (0.41 g, 0.39 mmol) were added to the reaction mixture simultaneously and argon gas was bubbled in for another 5 min. The reaction mixture was heated to reflux for 12 h. After completion of the reaction as indicated by TLC the mixture was concentrated under reduced pressure. The residue was partitioned between dichloromethane and water. The organic layer was separated, washed with water and brine, dried over sodium sulfate and concentrated. The obtained crude residue was purified by column chromatography eluting with 15% ethyl acetate in dichloromethane to provide 4-(8-fluoro-6-oxo-3,4,5,6-tetrahydro-1H-azepino[5,4,3-cd]indol-2-yl)benzaldehyde (P31, 1.58 g, 72.5%). ¹H NMR (400 MHz, DMSO-d₆) δ 10.06 (s, 1H), 8.30 (s, 1H), 8.05 (d, J=8.1 Hz, 2H), 7.86 (d, J=8.0 Hz, 2H), 7.45 (d, J=10.9 Hz, 2H), 7.37 (d, J=9.0 Hz, 1H), 3.41 (s, 2H), 3.10 (s, 2H).

### Preparation 32. 8-Fluoro-2-{4-[(pyridin-2-ylamino)methyl]phenyl}-1,3,4,5-tetrahydro-6H-azepino[5,4,3-cd]indol-6-one (P32)

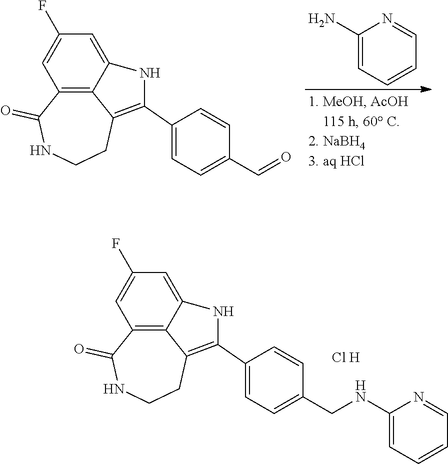

To a mixture of 4-(8-fluoro-6-oxo-3,4,5,6-tetrahydro-1H-azepino[5,4,3-cd]indol-2-yl)benzaldehyde (P31, 0.17 g, 0.055 mmol) and 2-aminopyridine (0.21 g, 0.22 mmol) in 10 mL MeOH, few drops of AcOH were added, and the mixture was stirred at 60° C. for 15 h. The mixture was cooled down to ambient temperature, and NaBH₄ was added (0.25 g, 0.66 mmol) and the mixture was stirred at ambient temperature for 15 h. MeOH was evaporated, the residue was diluted with H₂O, extracted with EtOAc, the combined organic extracts were dried with Na₂SO₄ and evaporated. The residue was purified by RP-HPLC eluting with a gradient MeCN-H₂O+0.1% TFA. The target fractions were collected. Few drops of conc. HCl were added, and the solution was evaporated to dryness giving 8-fluoro-2-{4-[(pyridin-2-ylamino)methyl]phenyl}-1,3,4,5-tetrahydro-6H-azepino[5,4,3-cd]indol-6-one (P32, 0.041 g, 19%). ¹H NMR (400 MHz, DMSO-d₆) δ 14.11 (s, 1H), 11.80 (s, 1H), 9.36 (s, 1H), 8.25 (d, J=5.6 Hz, 1H), 8.14-7.82(m, 2H), 7.66 (d, J=8.2 Hz, 2H), 7.57 (d, J=8.1 Hz, 2H), 7.42(dd, J=11.0, 2.3 Hz, 1H), 7.34 (dd, J=9.1, 2.3 Hz, 1H), 7.17 (d, J=8.9 Hz, 1H), 6.90 (t, J=6.6 Hz, 1H), 4.74 (d, J=5.1 Hz, 2H), 3.39 (s, 2H), 3.03 (s, 2H).

### Preparation 33. 8-Fluoro-2-{4-[(pyridin-3-ylamino)methyl]phenyl}-1,3,4,5-tetrahydro-6H-azepino[5,4,3-cd]indol-6-one (P33)

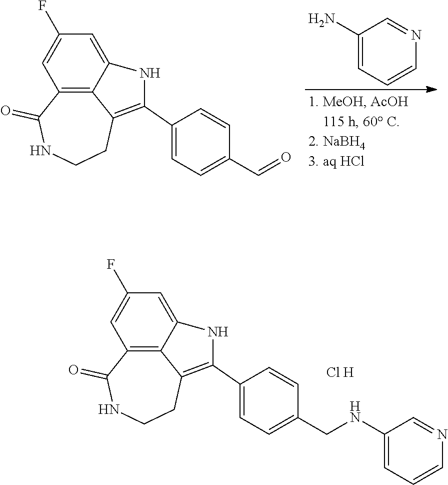

To a mixture of 4-(8-fluoro-6-oxo-3,4,5,6-tetrahydro-1H-azepino[5,4,3-cd]indol-2-yl)benzaldehyde (P31, 0.17 g, 0.055 mmol) and 3-aminopyridine (0.21 g, 0.22 mmol) in 10 mL MeOH, few drops of AcOH were added, and the mixture was stirred at 60° C. for 15 h. The mixture was cooled down to ambient temperature, and NaBH₄ was added (0.25 g, 0.66 mmol) and the mixture was stirred at ambient temperature for 15 h. MeOH was evaporated, the residue was diluted with H₂O, extracted with EtOAc, the combined organic extracts were dried with Na₂SO₄ and evaporated. The residue was purified by RP-HPLC eluting with a gradient MeCN-H₂O+0.1% TFA. The target fractions were collected. Few drops of conc. HCl were added, and the solution was evaporated to dryness giving 8-fluoro-2-{4-[(pyridin-3-ylamino)methyl]phenyl}-1,3,4,5-tetrahydro-6H-azepino[5,4,3-cd]indol-6-one (P33, 0.027 g, 13%). ¹H NMR (400 MHz, DMSO-d₆) δ 11.77 (s, 1H), 8.25 (s, 1H), 8.11 (s, 1H), 8.03 (s, 1H), 7.84 (s, 2H), 7.73 (s, 2H), 7.64 (d, J=8.1 Hz, 2H), 7.53 (d, J=8.1 Hz, 2H), 7.42(dd, J=11.0, 2.3 Hz, 1H), 7.33 (dd, J=9.1, 2.3 Hz, 1H), 5.65 (s, 2H), 4.51 (s, 2H), 3.38 (s, 2H), 3.03 (s, 2H).

### Preparation 34. tert-Butyl [2-(4-bromophenyl)ethyl]methylcarbamate (P34)

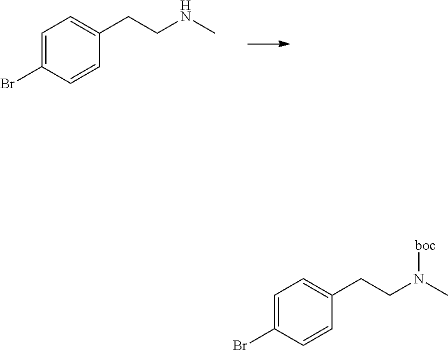

To a solution of [2-(4-bromophenyl)ethyl]methylamine (0.52 g, 2.6 mmol) in 20 mL CH₂Cl₂, boc₂O was added (0.62 g, 2.86 mmol) following by addition of Et₃N (0.79 g, 7.8 mmol) and the mixture was stirred for 15 h at ambient temperature. The mixture was washed with aq. citric acid, the organic layer was dried over Na₂SO₄ and evaporated giving tert-butyl [2-(4-bromophenyl)ethyl]methylcarbamate (P34, 0.82 g, 100%) that was used for the next step without additional purification. ¹H NMR (400 MHz, DMSO-d₆) δ 7.47 (d, J=8.1 Hz, 2H), 7.15 (d, J=7.4 Hz, 2H), 3.36 (t, J=7.0 Hz, 2H), 2.71 (t, J=7.0 Hz, 2H), 1.44-1.15 (m, 9H).

### Preparation 35. tert-Butyl methyl {2-[4-(4,4,5,5-tetramethyl-1,3,2-dioxaborolan-2-yl)phenyl]ethyl}carbamate (P35)

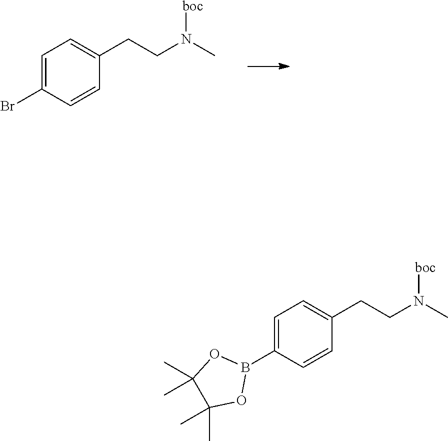

A mixture of tert-butyl [2-(4-bromophenyl)ethyl]methylcarbamate (P34, 0.82 g, 2.6 mmol), bis(pinacolato)diboron (0.80 g, 3.1 mmol, PdCl₂dppf (0.11 g, 0.13 mmol) and freshly calcined AcOK (0.77 g, 7.8 mmol) in 20 mL dioxane was purged with N₂ and stirred at 90° C. for 15 h. Dioxane was evaporated, the residue was diluted with H₂O, extracted with EtOAc, the combined organic extracts were dried with Na₂SO₄ and evaporated. The residue was purified on silica gel eluting with 1-20% EtOAc in hexane giving tert-butyl methyl {2-[4-(4,4,5,5-tetramethyl-1,3,2-dioxaborolan-2-yl)phenyl]ethyl}carbamate (P35, 0.87 g, 92%). ¹H NMR (400 MHz, DMSO-d₆) δ 7.59 (d, J=7.9 Hz, 2H), 7.21 (d, J=7.3 Hz, 2H), 3.39-3.34 (m, 2H), 2.75 (t, J=12.7 Hz, 2H), 1.28 (s, 12H), 1.17 (s, 9H).

### Preparation 36. tert-Butyl {2-[4-(8-fluoro-6-oxo-3,4,5,6-tetrahydro-1H-azepino[5,4,3-cd]indol-2-yl)phenyl]ethyl}methylcarbamate (P36)

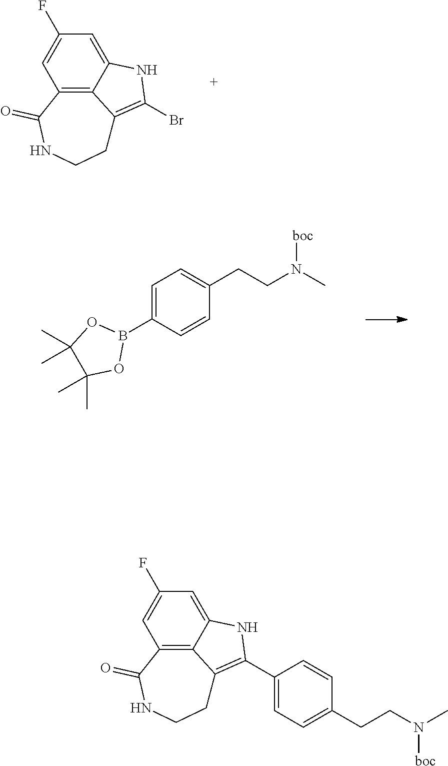

To a solution of tert-butyl methyl {2-[4-(4,4,5,5-tetramethyl-1,3,2-dioxaborolan-2-yl)phenyl]ethyl}carbamate (P35, 0.43 g, 1.2 mmol) in 10 mL of 1,4-dioxane 3 mL of saturated aqueous sodium carbonate solution were added. Argon gas was purged for 10 min at room temperature. 2-Bromo-8-fluoro-1,3,4,5-tetrahydro-6H-azepino[5,4,3-cd]indol-6-one (0.34 g, 1.2 mmol) and tetrakis(triphenylphosphine) palladium (0) (0.14 g, 0.12 mmol) were added to the reaction mixture simultaneously and argon gas was bubbled in for another 5 min. The reaction mixture was heated to reflux for 12 h. After completion of the reaction as indicated by TLC the mixture was concentrated under reduced pressure. The residue was partitioned between dichloromethane and water. The organic layer was separated, washed with water and brine, dried over sodium sulfate and concentrated. The obtained crude residue was purified by column chromatography eluting with 15% ethyl acetate in dichloromethane to provide tert-butyl {2-[4-(8-fluoro-6-oxo-3,4,5,6-tetrahydro-1H-azepino[5,4,3-cd]indol-2-yl)phenyl]ethyl}methylcarbamate (P36, 0.50 g, 96%). ¹H NMR (400 MHz, DMSO-d₆) δ 11.63 (s, 1H), 8.23 (d, J=5.9 Hz, 1H), 7.69-7.50 (m, 2H), 7.42(dd, J=11.0, 2.3 Hz, 2H), 7.38-7.28 (m, 1H), 7.25 (d, J=7.9 Hz, 1H), 3.49-3.35 (m, 4H), 3.03 (s, 2H), 2.87-2.79 (m, 2H), 2.79 (s, 3H), 1.29 (2s, 9H).

### Preparation 37. 8-Fluoro-2-{4-[2-(methylamino)ethyl]phenyl}-1,3,4,5-tetrahydro-6H-azepino[5,4,3-cd]indol-6-one (P37)

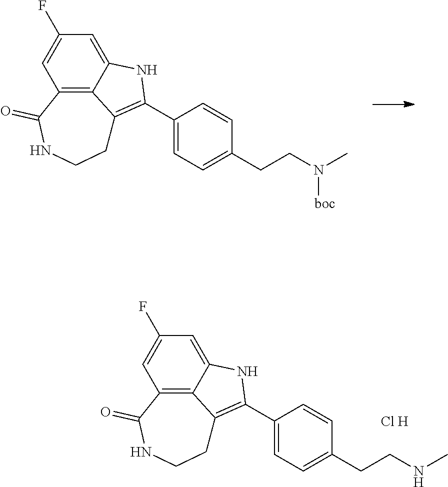

To a solution of tert-butyl {2-[4-(8-fluoro-6-oxo-3,4,5,6-tetrahydro-1H-azepino[5,4,3-cd]indol-2-yl)phenyl]ethyl}methylcarbamate (P36, 0.50 g, 1.14 mmol) in 5 mL dioxane, 5 mL 3M HCl in dioxane was added and the mixture was stirred at ambient temperature for 15 h. The mixture was diluted with Et₂O, the formed solid was filtered off, washed with Et₂O and dried. The solid residue was dissolved in H₂O and basified with aq. NaOH to pH 12. The formed mixture was extracted with CHCl₃, the combined organic extracts were dried with Na₂SO₄ and evaporated. The residue was purified by a reverse-phase HPLC eluting with a gradient MeCN-H₂O+0.1% TFA. The target fractions were collected and evaporated. The mixture was re-dissolved in H₂O, basified with aq. NaHCO₃ to pH 8, extracted with CHCl₃, the combined organic extracts were dried with Na₂SO₄ and evaporated giving 8-fluoro-2-{4-[2-(methylamino)ethyl]phenyl}-1,3,4,5-tetrahydro-6H-azepino[5,4,3-cd]indol-6-one (P37, 0.23 g, 59%). ¹H NMR (400 MHz, DMSO-d₆) δ 11.65 (d, J=28.1 Hz, 1H), 8.24 (s, 1H), 7.84-7.10 (m, 4H), 6.64 (s, 2H), 3.39 (m, 2H), 3.19-2.83 (m, 7H), 2.46 (s, 3H).

### Preparation 38. 8-Fluoro-2-(4-{[4-(4-nitrophenyl) piperazin-1-yl]methyl}phenyl)-1,3,4,5-tetrahydro-6H-azepino[5,4,3-cd]indol-6-one (P38)

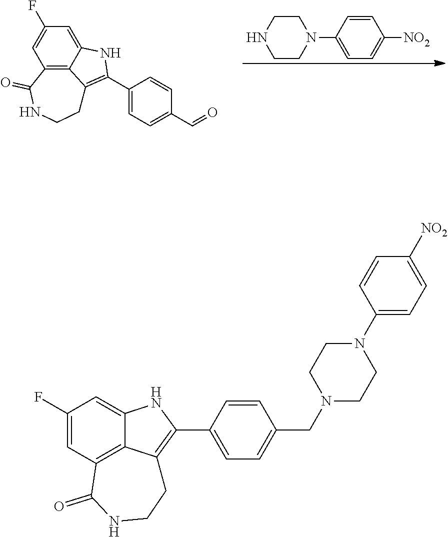 [SMILES: O=Cc1ccc(-c2[nH]c3cc(F)cc4c3c2CCNC4=O)cc1 ; O=[N+]([O-])c1ccc(N2CCNCC2)cc1 ; O=C1NCCc2c(-c3ccc(CN4CCN(c5ccc([N+](=O)[O-])cc5)CC4)cc3)[nH]c3cc(F)cc1c23]

1-(4-Nitrophenyl) piperazine (0.52 g, 2.50 mmol) and 4-(8-fluoro-6-oxo-3,4,5,6-tetrahydro-1H-azepino[5,4,3-cd]indol-2-yl)benzaldehyde (P31, 0.70 g, 2.27 mmol) in methanol (5 mL) was treated with acetic acid (1 drop), stirred for 15 minutes, cooled to 0° C., treated with sodium cyanoborohydride (0.29 g, 4.54 mmol), warmed to room temperature overnight, and partitioned between ethyl acetate and saturated sodium bicarbonate. The organic layer was dried (MgSO₄), filtered, and concentrated. The concentrate was purified by flash column chromatography on silica gel with 5% methanol/DCM to provide 8-fluoro-2-(4-{[4-(4-nitrophenyl) piperazin-1-yl]methyl}phenyl)-1,3,4,5-tetrahydro-6H-azepino[5,4,3-cd]indol-6-one (P38, 0.38 g, 33%). 1H NMR (400 MHz, DMSO-d₆) δ 11.75 (s, 1H), 8.21 (s, 1H), 8.05 (d, J=9.0 Hz, 2H), 7.62(d, J=7.8 Hz, 2H), 7.49 (d, J=7.8 Hz, 2H), 7.43 (d, J=11.2 Hz, 1H), 7.33 (d, J=8.9 Hz, 1H), 7.02(d, J=9.4 Hz, 2H), 3.60 (s, 2H), 3.48 (s, 4H), 3.40 (s, 2H), 3.06 (s, 2H), 2.54 (s, 4H).

### Preparation 39. 2-(4-{[4-(4-Aminophenyl) piperazin-1-yl]methyl}phenyl)-8-fluoro-1,3,4,5-tetrahydro-6H-azepino[5,4,3-cd]indol-6-one (P39)

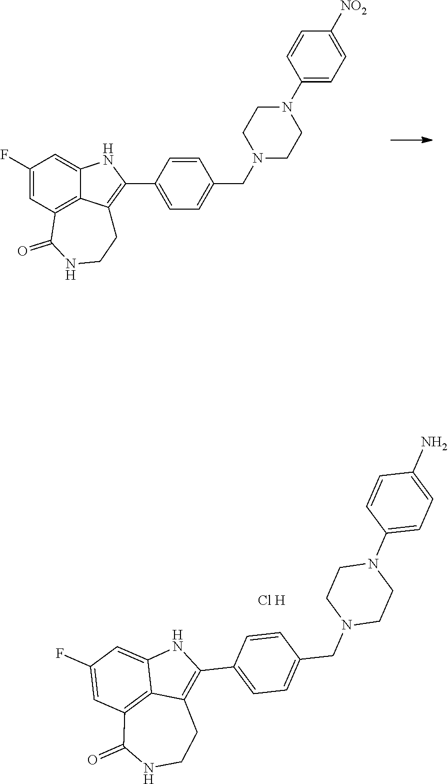

A solution of 8-fluoro-2-(4-{[4-(4-nitrophenyl) piperazin-1-yl]methyl}phenyl)-1,3,4,5-tetrahydro-6H-azepino[5,4,3-cd]indol-6-one P38 in EtOH (20 mL) was purged with nitrogen for 10 minutes. Palladium on carbon (10% Pd/C, 50% water, 20 mg) was added, hydrogen was bubbled into the reaction mixture, and the reaction was rapidly stirred under a hydrogen atmosphere for 2 h. The reaction was purged with nitrogen and was filtered through Celite. The concentrate was purified by flash column chromatography on silica gel with 5-20% methanol/DCM with 0.5% NH₄OH to provide the desired product. The fractions containing target compound were combined, triturated with HCl/dioxane, evaporated to dryness to give 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.19 g, 98%). ¹H NMR (400 MHz, DMSO-d₆) δ 11.86 (d, J=15.7 Hz, 1H), 10.18 (s, 2H), 8.27 (s, 1H), 7.82(d, J=8.2 Hz, 2H), 7.73 (d, J=8.2 Hz, 2H), 7.46-7.41 (m, 1H), 7.37 (dd, J=9.2, 2.4 Hz, 1H), 7.28 (d, J=8.8 Hz, 2H), 7.07 (d, J=8.9 Hz, 2H), 4.44 (s, 2H), 3.83 (s, 2H), 3.41 (s, 4H), 3.22(m, 3H), 3.08 (s, 4H).

### Preparation 40. tert-butyl 4-[4-(8-fluoro-6-oxo-3,4,5,6-tetrahydro-1H-azepino[5,4,3-cd]indol-2-yl)benzyl]piperazine-1-carboxylate (P40)

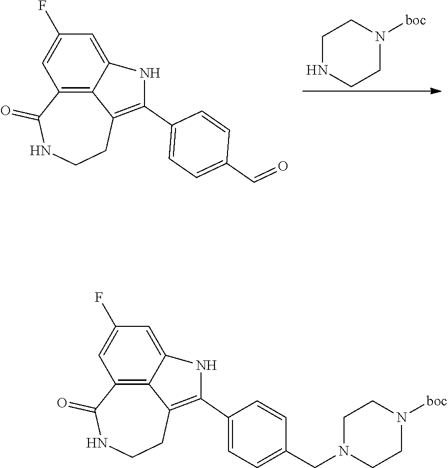

To a solution of 4-(8-fluoro-6-oxo-3,4,5,6-tetrahydro-1H-azepino[5,4,3-cd]indol-2-yl)benzaldehyde (P31, 0.3 g, 0.97 mmol) and tert-butyl piperazine-1-carboxylate (0.23 g, 1.26 mmol) in 20 mL DCM, few drops of AcOH were added following by addition of STAB (0.83 g, 3.88 mmol) and the reaction mixture was stirred at ambient temperature for 15 h. The mixture was quenched with aq. NaHCO₃, extracted with DCM, the combined organic extracts were dried with Na₂SO₄ and evaporated. The residue was purified on silica gel eluting with 50-100% EtOAc in hexane→5% MeOH in EtOAc giving tert-butyl 4-[4-(8-fluoro-6-oxo-3,4,5,6-tetrahydro-1H-azepino[5,4,3-cd]indol-2-yl)benzyl]piperazine-1-carboxylate (P40, 0.38 g, 80%). ¹H NMR (400 MHz, DMSO-d₆) δ 11.65 (s, 1H), 8.24 (s, 1H), 7.59 (d, J=8.0 Hz, 2H), 7.44 (t, J=10.3 Hz, 3H), 7.31 (d, J=7.0 Hz, 1H), 3.54 (s, 2H), 3.39 (s, 2H), 3.30-3.34 (m, 4H), 3.04 (s, 2H), 2.34 (s, 4H), 1.39 (s, 9H).

### Preparation 41. 8-fluoro-2-[4-(piperazin-1-ylmethyl)phenyl]-1,3,4,5-tetrahydro-6H-azepino[5,4,3-cd]indol-6-one (P41)

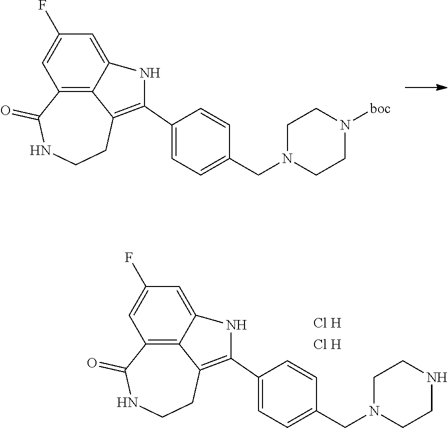

To a solution of tert-butyl 4-[4-(8-fluoro-6-oxo-3,4,5,6-tetrahydro-1H-azepino[5,4,3-cd]indol-2-yl)benzyl]piperazine-1-carboxylate (P40, 0.38 g, 0.79 mmol) in 5 mL dioxane, 5 mL of 3M HCl in dioxane was added and the mixture was stirred at ambient temperature for 15 h. Dioxane was evaporated, the solid residue was washed with Et₂O giving 8-fluoro-2-[4-(piperazin-1-ylmethyl)phenyl]-1,3,4,5-tetrahydro-6H-azepino[5,4,3-cd]indol-6-one (P41, 0.30 g, 84%). 1H NMR (400 MHz, DMSO-d₆) δ 11.92(s, 1H), 9.89 (s, 2H), 8.28 (s, 1H), 7.83 (d, J=8.1 Hz, 2H), 7.73 (d, J=8.2 Hz, 2H), 7.43 (dd, J=10.9, 2.3 Hz, 1H), 7.37 (dd, J=9.1, 2.2 Hz, 1H), 4.47 (s, 2H), 3.52(t, J=52.3 Hz, 11H), 3.07 (s, 2H).

### Preparation 42. tert-Butyl {1-[4-(8-fluoro-6-oxo-3,4,5,6-tetrahydro-1H-azepino[5,4,3-cd]indol-2-yl)benzyl]piperidin-4-yl}carbamate (P42)

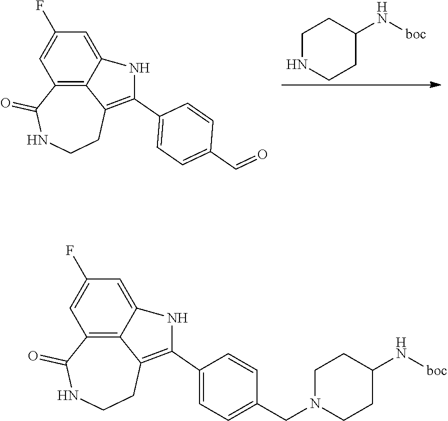

To a solution of 4-(8-fluoro-6-oxo-3,4,5,6-tetrahydro-1H-azepino[5,4,3-cd]indol-2-yl)benzaldehyde (P31, 0.3 g, 0.97 mmol) and tert-butyl piperidin-4-ylcarbamate (0.25 g, 1.26 mmol) in 20 mL DCM, few drops of AcOH were added following by addition of STAB (0.83 g, 3.88 mmol) and the reaction mixture was stirred at ambient temperature for 15 h. The mixture was quenched with aq. NaHCO₃, extracted with DCM, the combined organic extracts were dried with Na₂SO₄ and evaporated. The residue was purified on silica gel eluting with 50-100% EtOAc in hexane→5% MeOH in EtOAc giving tert-butyl {1-[4-(8-fluoro-6-oxo-3,4,5,6-tetrahydro-1H-azepino[5,4,3-cd]indol-2-yl)benzyl]piperidin-4-yl}carbamate (P42, 0.28 g, 59%). ¹H NMR (400 MHz, DMSO-d₆) δ 11.65 (s, 1H), 8.24 (t, J=5.6 Hz, 1H), 7.58 (d, J=8.0 Hz, 2H), 7.44 (t, J=12.3 Hz, 3H), 7.31 (dd, J=9.1, 2.3 Hz, 1H), 6.76 (d, J=7.9 Hz, 1H), 3.49 (s, 2H), 3.27 (d, J=41.5 Hz, 2H), 3.04 (s, 2H), 2.78 (d, J=11.4 Hz, 2H), 2.00 (d, J=9.2 Hz, 2H), 1.91 (s, 1H), 1.69 (d, J=10.4 Hz, 2H), 1.39 (d, J=8.0 Hz, 2H), 1.39 (d, J=11.9 Hz, 9H).

### Preparation 43. 2-{4-[(4-Aminopiperidin-1-yl)methyl]phenyl}-8-fluoro-1,3,4,5-tetrahydro-6H-azepino[5,4,3-cd]indol-6-one (P43)

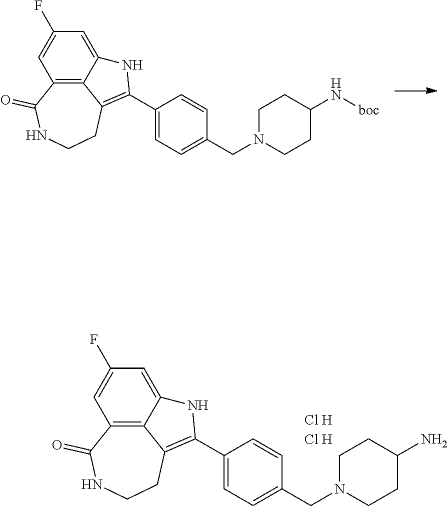

To a solution of tert-butyl {1-[4-(8-fluoro-6-oxo-3,4,5,6-tetrahydro-1H-azepino[5,4,3-cd]indol-2-yl)benzyl]piperidin-4-yl}carbamate (P42, 0.28 g, 0.57 mmol) in 5 mL dioxane, 5 mL of 3M HCl in dioxane was added and the mixture was stirred at ambient temperature for 15 h. Dioxane was evaporated, the solid residue was washed with Et₂O giving 2-{4-[(4-aminopiperidin-1-yl)methyl]phenyl}-8-fluoro-1,3,4,5-tetrahydro-6H-azepino[5,4,3-cd]indol-6-one (P43, 0.21 g, 80%). ¹H NMR (400 MHz, DMSO-d₆) δ 11.94 (s, 1H), 8.45 (s, 2H), 8.28 (s, 1H), 7.78 (d, J=8.2 Hz, 2H), 7.72(d, J=8.2 Hz, 2H), 7.43 (dd, J=10.9, 2.4 Hz, 1H), 7.37 (dd, J=9.1, 2.4 Hz, 1H), 4.95-5.05 (m, 2H), 4.31 (d, J=3.9 Hz, 2H), 3.40 (s, 4H), 3.26 (s, 1H), 3.07 (s, 4H), 2.07 (dt, J=21.7, 11.2 Hz, 4H).

### Preparation 44. 6-Methylsulfanyl-2-(2-pyridyl)-1H-pyrazolo[3,4-d]pyrimidin-3-one (P44)

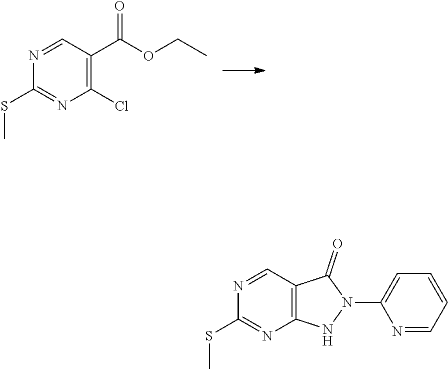 [SMILES: CCOC(=O)c1cnc(SC)nc1Cl ; CSc1ncc2c(=O)n(-c3ccccn3)[nH]c2n1]

DIPEA (1.96 mL, 11.28 mmol) and 2-pyridylhydrazine (0.49 g, 4.5 mmol) were added to a solution of ethyl 4-chloro-2-methylthio-5-pyrimidinecarboxylate (1.05 g, 4.5 mmol) in THF (15 mL). The reaction mixture was heated at reflux for 72 h, before being concentrated in vacuo. Et₂O (10 mL) was added to the residue, and the resultant precipitate was collected by filtration. The filtrate was evaporated to dryness, and the residue was cooled in an ice bath, after which TFA (5 mL) was added. The resultant solution was stirred at ambient temperature for 1 h, followed by 70° C. for 1 h. The solvent was removed in vacuo and the residue was dissolved in EtOH (5 mL) and cooled in an ice bath, after which 6M NaOH (10 mL) was added. The resultant solution was stirred at ambient temperature for 15 min, before being acidified (pH 6) via the addition of conc. HCl, resulting precipitate was filtering and washed with water to provide 6-methylsulfanyl-2-(2-pyridyl)-1H-pyrazolo[3,4-d]pyrimidin-3-one (P44, 1.1 g, 94%). ¹H NMR (400 MHz, DMSO-d₆) δ 8.48 (s, 1H), 8.43 (ddd, J=4.9, 2.0, 0.9 Hz, 1H), 8.31 (d, J=8.4 Hz, 1H), 7.89-7.80 (m, 1H), 7.15 (ddd, J=7.3, 4.9, 1.1 Hz, 1H), 2.47 (s, 3H).

### Preparation 45. 1-Methyl-6-methylsulfanyl-2-(2-pyridyl)pyrazolo[3,4-d]pyrimidin-3-one (P45)

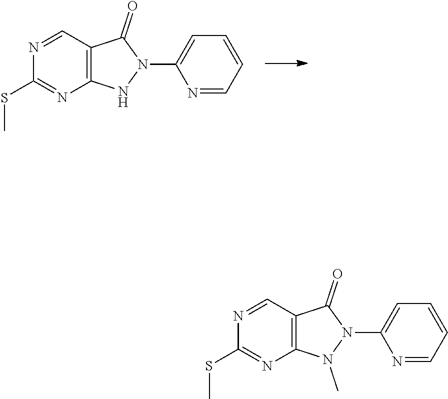 [SMILES: CSc1ncc2c(=O)n(-c3ccccn3)[nH]c2n1 ; CSc1ncc2c(=O)n(-c3ccccn3)n(C)c2n1]

Iodomethane (0.285 mL, 2.56 mmol) was added to a mixture of 6-methylsulfanyl-2-(2-pyridyl)-1H-pyrazolo[3,4-d]pyrimidin-3-one (P44, 0.634 g, 2.45 mmol) and potassium carbonate (0.676 g, 4.9 mmol) in MeCN (15 mL), resulting suspension was stirred under reflux for 6 h. The reaction mixture was cooled to ambient temperature and diluted with water (25 mL) before being extracted with EtOAc (2×30 mL). The combined organic extracts were washed with brine, dried over Na₂SO₄, and evaporated to dryness before the crude material was purified by chromatography on silica gel with hexane-EtOAc (1:1) to yield 1-methyl-6-methylsulfanyl-2-(2-pyridyl)pyrazolo[3,4-d]pyrimidin-3-one (P45, 0.27 g, 40%). ¹H NMR (400 MHz, DMSO-d₆) δ 8.95 (s, 1H), 8.57 (ddd, J=4.9, 1.9, 0.9 Hz, 1H), 8.03 (ddd, J=8.2, 7.4, 1.9 Hz, 1H), 7.86 (dt, J=8.2, 0.9 Hz, 1H), 7.38 (ddd, J=7.3, 4.9, 1.0 Hz, 1H), 3.57 (s, 3H), 2.61 (s, 3H).

### Preparation 46. 6-Chloro-1-methyl-2-pyridin-2-yl-1,2-dihydro-3H-pyrazolo[3,4-d]pyrimidin-3-one (P46)

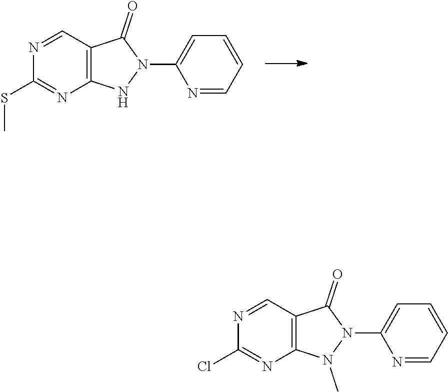 [SMILES: CSc1ncc2c(=O)n(-c3ccccn3)[nH]c2n1 ; Cn1c2nc(Cl)ncc2c(=O)n1-c1ccccn1]

To a solution of 1-methyl-6-methylsulfanyl-2-(2-pyridyl)pyrazolo[3,4-d]pyrimidin-3-one (P45, 0.26 g, 0.95 mmol) in 30 mL DCM-MeCN, 2:1, neat sulfuryl chloride was added (0.64 g, 4.75 mmol) dropwise at 5° C. The mixture was stirred at ambient temperature for 2 h and poured into aq. NaHCO₃. The mixture was extracted with DCM, the combined organic extracts were dried with Na₂SO₄ and evaporated giving 6-chloro-1-methyl-2-pyridin-2-yl-1,2-dihydro-3H-pyrazolo[3,4-d]pyrimidin-3-one (P46) that was used for the next step without additional purification. ¹H NMR (400 MHz, DMSO-d₆) δ 9.11 (s, 1H), 8.60 (d, J=3.7 Hz, 1H), 8.06 (t, J=7.8 Hz, 1H), 7.85 (d, J=8.2 Hz, 1H), 7.57-7.38 (m, 1H), 3.59 (s, 3H).

### Preparation 47. 1-[6-(1-Hydroxy-1-methyl-ethyl)-2-pyridyl]-2-methyl-6-methylsulfanyl-pyrazolo[3,4-d]pyrimidin-3-one (P47)

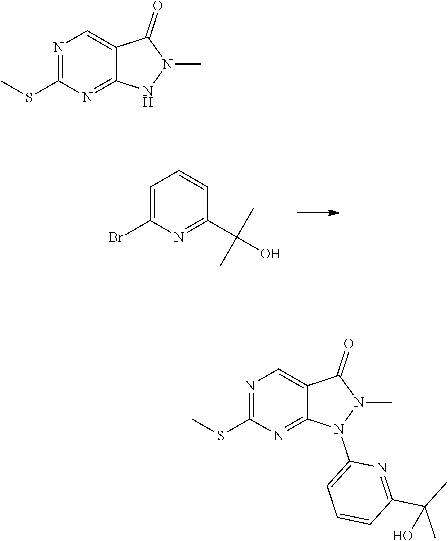

N,N′-Dimethylethylenediamine (0.898 g, 10.19 mmol) was added to a solution of the 2-methyl-6-methylsulfanyl-1H-pyrazolo[3,4-d]pyrimidin-3-one (P1, 1.0 g, 5.09 mmol), 2-(6-bromo-2-pyridyl) propan-2-ol (P11, 1.43 g, 6.61 mmol), CuI (0.971 g, 5.09 mmol) and K₂CO₃ (0.986 g, 7.12 mmol) in 1,4-dioxane (10 mL) at 80° C. The resultant suspension was heated at 95° C. for 18 h, over which time a color change of orange to dark green occurred. The reaction mixture was cooled to ambient temperature and diluted with NH₄OH (50 mL) before being extracted with EtOAc (2×50 mL). The combined organic extracts were washed with brine, dried over Na₂SO₄, and evaporated to dryness before the crude material was purified via chromatography on silica with hexane-EtOAc (2:1) to yield 1-[6-(1-hydroxy-1-methyl-ethyl)-2-pyridyl]-2-methyl-6-methylsulfanyl-pyrazolo[3,4-d]pyrimidin-3-one (P47, 0.796 g, 47%) as a white solid. ¹H NMR (400 MHz, DMSO-d₆) δ 8.99 (s, 1H), 8.05 (t, J=7.9 Hz, 1H), 7.78 (d, J=8.1 Hz, 1H), 7.66 (d, J=7.8 Hz, 1H), 5.32 (s, 1H), 3.49 (s, 3H), 2.56 (s, 3H), 1.45 (s, 6H).

### Preparation 48. 4-(4-Fluoro-3-{[4-(4-nitrophenyl) piperazin-1-yl]carbonyl}benzyl)phthalazin-1(2H)-one (P48)

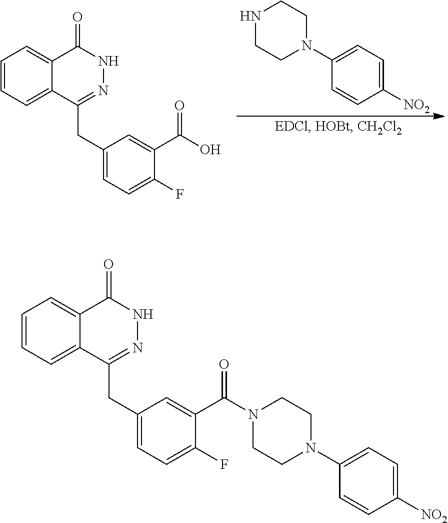 [SMILES: O=C(O)c1cc(Cc2n[nH]c(=O)c3ccccc23)ccc1F ; O=[N+]([O-])c1ccc(N2CCNCC2)cc1 ; O=C(c1cc(Cc2n[nH]c(=O)c3ccccc23)ccc1F)N1CCN(c2ccc([N+](=O)[O-])cc2)CC1]

To a solution of 2-fluoro-5-[(4-oxo-3,4-dihydrophthalazin-1-yl)methyl]benzoic acid (2.25 g, 7.5 mmol) in 50 mL DCM, 1-(4-nitrophenyl) piperazine (1.56 g, 7.5 mmol) was added, following by addition of EDCI (2.89 g, 15.0 mmol), HOBt (2.03 g, 15.0 mmol), and Et₃N (3.0 mL), and the mixture was stirred for 15 h at ambient temperature. The formed yellow pellet was filtered off, washed with DCM and dried, giving 4-(4-fluoro-3-{[4-(4-nitrophenyl) piperazin-1-yl]carbonyl}benzyl)phthalazin-1(2H)-one (P48, 2.62 g, 71%) that was used for the next step without additional purification. ¹H NMR (400 MHz, DMSO-d₆) δ 12.59 (s, 1H), 8.27 (d, J=7.5 Hz, 1H), 8.08 (d, J=9.3 Hz, 2H), 7.97 (d, J=7.9 Hz, 1H), 7.90 (t, J=7.5 Hz, 1H), 7.84 (t, J=7.4 Hz, 1H), 7.46 (s, 1H), 7.39 (d, J=4.9 Hz, 1H), 7.25 (t, J=9.0 Hz, 1H), 7.01 (d, J=9.4 Hz, 2H), 4.34 (s, 2H), 3.76 (s, 2H), 3.58 (s, 2H), 3.38 (d, J=13.5 Hz, 2H), 2.50 (s, 2H).

### Preparation 49. 4-(3-{[4-(4-Aminophenyl) piperazin-1-yl]carbonyl}-4-fluorobenzyl)phthalazin-1(2H)-one (P49)

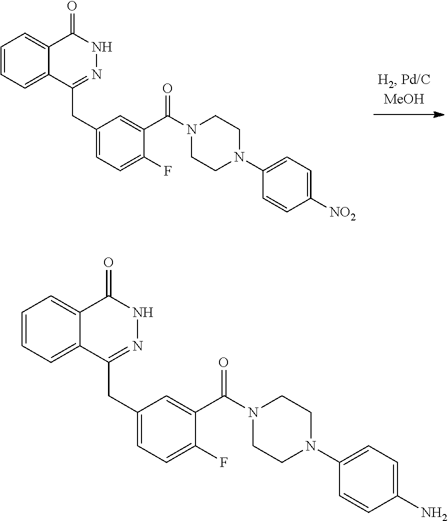 [SMILES: O=C(c1cc(Cc2n[nH]c(=O)c3ccccc23)ccc1F)N1CCN(c2ccc([N+](=O)[O-])cc2)CC1 ; Nc1ccc(N2CCN(C(=O)c3cc(Cc4n[nH]c(=O)c5ccccc45)ccc3F)CC2)cc1]

To a solution of 4-(4-fluoro-3-{[4-(4-nitrophenyl) piperazin-1-yl]carbonyl}benzyl)phthalazin-1(2H)-one (P48, 2.62 g, 5.4 mmol) in 30 mL DMF, 10% Pd/C was added (0.26 g), and the mixture was hydrogenated at 40° C. and 3 MPa for 48 h. The catalyst was filtered off through Celite, and the filtrate was evaporated to dryness giving 4-(3-{[4-(4-aminophenyl) piperazin-1-yl]carbonyl}-4-fluorobenzyl)phthalazin-1(2H)-one (P49, 2.37 g, 96%) that was used for the next step without additional purification. ¹H NMR (400 MHz, DMSO-d₆) δ 12.58 (s, 1H), 8.26 (d, J=7.7 Hz, 1H), 7.97 (d, J=8.6 Hz, 2H), 7.92-7.86 (m, 1H), 7.82(t, J=7.5 Hz, 1H), 7.43 (dd, J=7.2, 4.2 Hz, 1H), 7.38-7.33 (m, 1H), 7.23 (t, J=9.0 Hz, 1H), 7.17-7.01 (m, 1H), 6.69 (d, J=8.7 Hz, 2H), 6.51 (d, J=8.7 Hz, 2H), 4.76 (s, 2H), 4.33 (s, 2H), 3.73 (s, 2H), 2.93 (s, 2H), 2.78 (s, 2H).

### Preparation 50. tert-Butyl 4-{2-fluoro-5-[(4-oxo-3,4-dihydrophthalazin-1-yl)methyl]benzoyl}piperazine-1-carboxylate (P50)

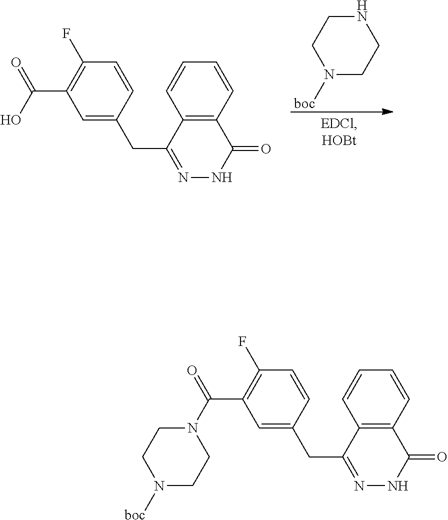

To a solution of 2-fluoro-5-[(4-oxo-3,4-dihydrophthalazin-1-yl)methyl]benzoic acid (0.25 g, 0.83 mmol) in 20 mL DCM, tert-butyl piperazine-1-carboxylate (0.23 g, 1.24 mmol) was added, following by addition of EDCI (0.32 g, 1.66 mmol), HOBt (0.23 g, 1.66 mmol), and Et₃N (0.5 mL), and the mixture was stirred for 15 h at ambient temperature. The mixture was quenched with aq. NaHCO₃, extracted with DCM, the combined organic extracts were dried with Na₂SO₄ and evaporated. The residue was purified on silica gel eluting with 10-50% EtOAc in hexane, giving tert-butyl 4-{2-fluoro-5-[(4-oxo-3,4-dihydrophthalazin-1-yl)methyl]benzoyl}piperazine-1-carboxylate (P50, 0.38 g, 97%). ¹H NMR (400 MHz, DMSO-d₆) δ 12.59 (s, 1H), 8.26 (d, J=7.3 Hz, 1H), 7.96 (d, J=8.0 Hz, 1H), 7.89 (t, J=7.1 Hz, 1H), 7.83 (t, J=7.3 Hz, 1H), 7.44 (d, J=5.7 Hz, 1H), 7.34 (d, J=4.4 Hz, 1H), 7.23 (t, J=9.0 Hz, 1H), 4.32(s, 2H), 3.58 (s, 2H), 3.38 (s, 2H), 3.23 (s, 2H), 3.14 (s, 2H), 1.40 (s, 9H).

### Preparation 51. 4-[4-Fluoro-3-(piperazin-1-ylcarbonyl)benzyl]phthalazin-1(2H)-one (P51)

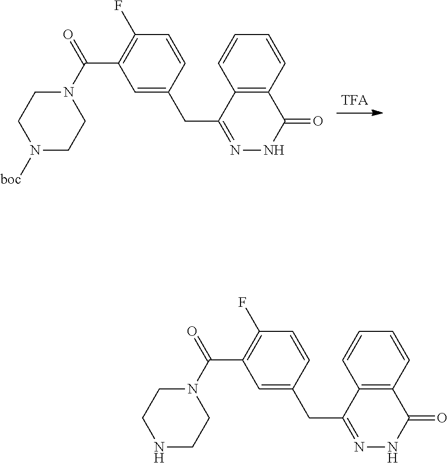

To a solution of tert-butyl 4-{2-fluoro-5-[(4-oxo-3,4-dihydrophthalazin-1-yl)methyl]benzoyl}piperazine-1-carboxylate (P50, 0.38 g, 0.80 mmol) in 10 mL DCM, 5 mL TFA was added, and the mixture was kept for 5 h at ambient temperature. The solvents were evaporated, and the residue was diluted with H₂O and basified with aq. NaOH. The mixture was extracted with DCM, the combined organic extracts were dried with Na₂SO₄ and evaporated giving 4-[4-fluoro-3-(piperazin-1-ylcarbonyl)benzyl]phthalazin-1(2H)-one (P51, 0.26 g, 89%) that was used for the next step without additional purification. ¹H NMR (400 MHz, DMSO-d₆) δ 12.59 (s, 1H), 8.26 (d, J=7.1 Hz, 1H), 7.96 (d, J=7.8 Hz, 1H), 7.88 (t, J=7.0 Hz, 1H), 7.82(t, J=7.5 Hz, 1H), 7.45-7.36 (m, 1H), 7.31 (d, J=4.4 Hz, 1H), 7.20 (t, J=9.0 Hz, 1H), 4.32(s, 2H), 4.18 (s, 1H), 3.52(s, 2H), 3.05 (s, 2H), 2.69 (t, J=4.8 Hz, 2H), 2.53 (d, J=12.2 Hz, 2H).

### Preparation 52. tert-Butyl [2-({2-fluoro-5-[(4-oxo-3,4-dihydrophthalazin-1-yl)methyl]benzoyl}amino)ethyl]carbamate (P52)

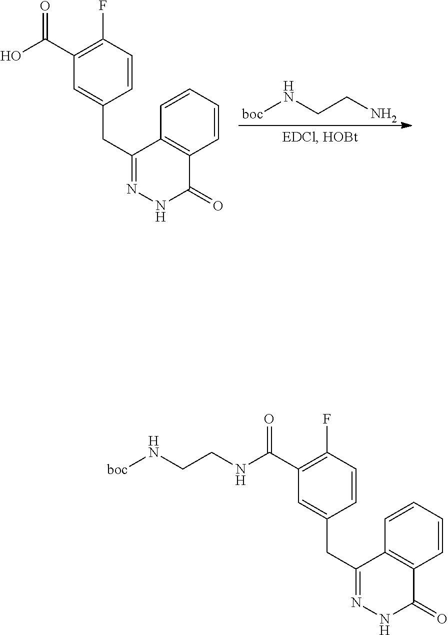

To a solution of 2-fluoro-5-[(4-oxo-3,4-dihydrophthalazin-1-yl)methyl]benzoic acid (0.25 g, 0.83 mmol) in 20 mL CH₂Cl₂, tert-butyl(2-aminoethyl)carbamate (0.21 g, 1.24 mmol) was added, following by addition of EDCI (0.32 g, 1.66 mmol), HOBt (0.23 g, 1.66 mmol), and Et₃N (0.5 mL), and the mixture was stirred for 15 h at ambient temperature. The mixture was quenched with aq. NaHCO₃, extracted with CH₂Cl₂, the combined organic extracts were dried with Na₂SO₄ and evaporated. The residue was purified on silica gel eluting with 10-50% EtOAc in hexane, giving tert-butyl [2-({2-fluoro-5-[(4-oxo-3,4-dihydrophthalazin-1-yl)methyl]benzoyl}amino)ethyl]carbamate (P52, 0.16 g, 43%). ¹H NMR (400 MHz, DMSO-d₆) δ 12.59 (s, 1H), 8.26 (d, J=7.6 Hz, 1H), 8.21 (s, 1H), 7.96 (d, J=7.8 Hz, 1H), 7.89 (t, J=6.9 Hz, 1H), 7.82(t, J=7.5 Hz, 1H), 7.60 (d, J=4.9 Hz, 1H), 7.45 (s, 1H), 7.26-7.11 (m, 1H), 6.85 (s, 1H), 4.32(s, 2H), 3.25 (dd, J=12.0, 5.9 Hz, 2H), 3.07 (d, J=6.0 Hz, 2H), 1.34 (s, 9H).

### Preparation 53. N-(2-Aminoethyl)-2-fluoro-5-[(4-oxo-3,4-dihydrophthalazin-1-yl)methyl]benzamide (P53)

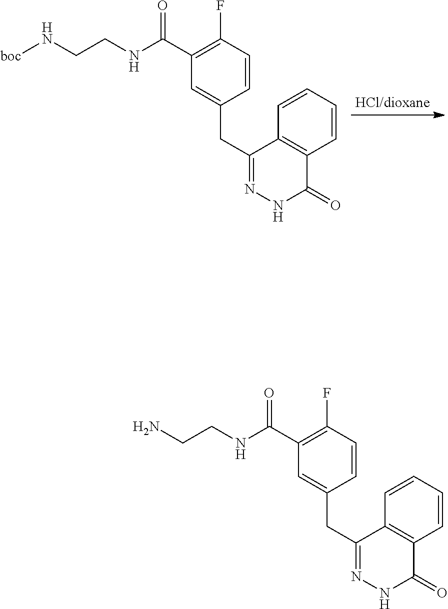

To a solution of tert-butyl [2-({2-fluoro-5-[(4-oxo-3,4-dihydrophthalazin-1-yl)methyl]benzoyl}amino)ethyl]carbamate (P52, 0.16 g, 0.36 mmol) in 5 mL dioxane, 5 mL 3M HCl in dioxane was added and the mixture was kept for 15 h at ambient temperature. The solvents were evaporated, and the residue was triturated with Et₂O giving N-(2-aminoethyl)-2-fluoro-5-[(4-oxo-3,4-dihydrophthalazin-1-yl)methyl]benzamide (P53, 0.18 g, 99%) as hydrochloride that was used for the next step without additional purification. ¹H NMR (400 MHz, DMSO-d₆) δ 12.60 (s, 1H), 8.44 (s, 1H), 8.26 (d, J=7.8 Hz, 1H), 8.05 (s, 2H), 7.97 (d, J=7.9 Hz, 1H), 7.88 (dd, J=10.9, 4.3 Hz, 1H), 7.82(t, J=7.0 Hz, 1H), 7.68 (d, J=6.9 Hz, 1H), 7.55-7.40 (m, 1H), 7.23 (t, J=9.5 Hz, 1H), 5.42(s, 2H), 4.33 (s, 2H), 2.94 (dd, J=11.7, 5.9 Hz, 2H).

### Preparation 54. tert-Butyl (1-{2-fluoro-5-[(4-oxo-3,4-dihydrophthalazin-1-yl)methyl]benzoyl}piperidin-4-yl)carbamate (P54)

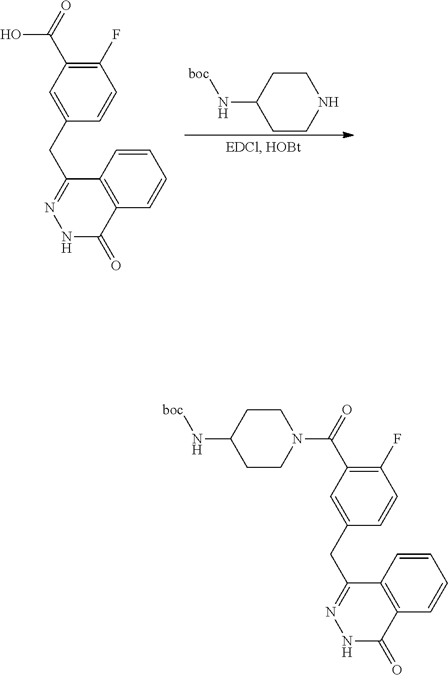

To a solution of 2-fluoro-5-[(4-oxo-3,4-dihydrophthalazin-1-yl)methyl]benzoic acid (0.25 g, 0.83 mmol) in 20 mL DCM, tert-butyl piperidin-4-ylcarbamate (0.25 g, 1.24 mmol) was added, following by addition of EDCI (0.32 g, 1.66 mmol), HOBt (0.23 g, 1.66 mmol), and Et₃N (0.5 mL), and the mixture was stirred for 15 h at ambient temperature. The mixture was quenched with aq. NaHCO₃, extracted with DCM, the combined organic extracts were dried with Na₂SO₄ and evaporated. The residue was purified on silica gel eluting with 10-50% EtOAc in hexane, giving tert-butyl (1-{2-fluoro-5-[(4-oxo-3,4-dihydrophthalazin-1-yl)methyl]benzoyl}piperidin-4-yl)carbamate (P54, 0.31 g, 77%). ¹H NMR (400 MHz, DMSO-d₆) § 12.58 (s, 1H), 8.26 (d, J=7.9 Hz, 1H), 7.97 (d, J=7.9 Hz, 1H), 7.89 (t, J=7.6 Hz, 1H), 7.82(t, J=7.3 Hz, 1H), 7.40 (s, 1H), 7.30 (d, J=4.7 Hz, 1H), 7.21 (t, J=9.0 Hz, 1H), 6.88 (s, 1H), 4.32(s, 2H), 3.45 (d, J=33.5 Hz, 1H), 3.28 (s, 1H), 3.02(s, 1H), 2.91 (t, J=11.4 Hz, 1H), 1.79 (d, J=10.8 Hz, 1H), 1.63 (s, 1H), 1.36 (d, J=10.6 Hz, 6H).

### Preparation 55. 4-{3-[(4-Aminopiperidin-1-yl)carbonyl]-4-fluorobenzyl}phthalazin-1(2H)-one (P55)

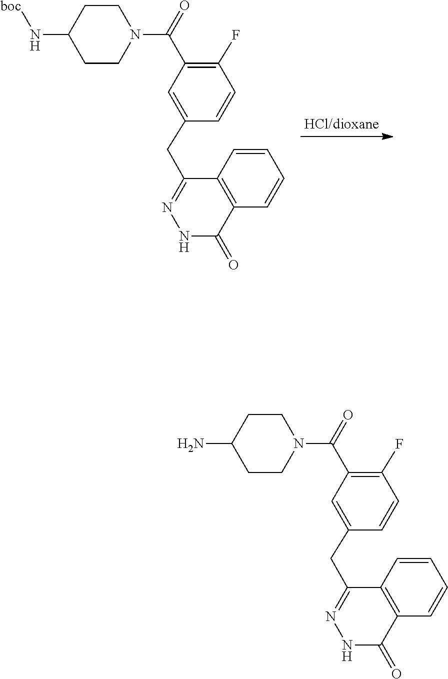

To a solution of tert-butyl (1-{2-fluoro-5-[(4-oxo-3,4-dihydrophthalazin-1-yl)methyl]benzoyl}piperidin-4-yl)carbamate (P54, 0.31 g, 0.64 mmol) in 5 mL dioxane, 5 mL 3M HCl in dioxane was added and the mixture was kept for 15 h at ambient temperature. The solvents were evaporated, and the residue was triturated with Et₂O giving 4-{3-[(4-aminopiperidin-1-yl)carbonyl]-4-fluorobenzyl}phthalazin-1(2H)-one (P55, 0.26 g, 99%) as hydrochloride that was used for the next step without additional purification. ¹H NMR (400 MHz, DMSO-d₆) δ 12.59 (s, 1H), 8.26 (d, J=7.9 Hz, 1H), 8.17 (m, 2H), 7.97 (d, J=7.7 Hz, 1H), 7.89 (t, J=7.0 Hz, 1H), 7.83 (t, J=7.4 Hz, 1H), 7.51-7.37 (m, 1H), 7.31 (d, J=4.8 Hz, 1H), 7.23 (t, J=9.0 Hz, 1H), 5.38 (s, 3H), 4.47 (d, J=13.2 Hz, 1H), 3.38 (d, J=12.4 Hz, 1H), 3.28 (s, 1H), 3.08 (s, 1H), 2.86 (t, J=11.6 Hz, 1H), 2.01 (d, J=11.2 Hz, 1H), 1.84 (s, 1H), 1.56-1.28 (m, 2H).

### Preparation 56. Methyl 2-fluoro-5-[(4-oxo-3,4-dihydrophthalazin-1-yl)methyl]benzoate (P56)

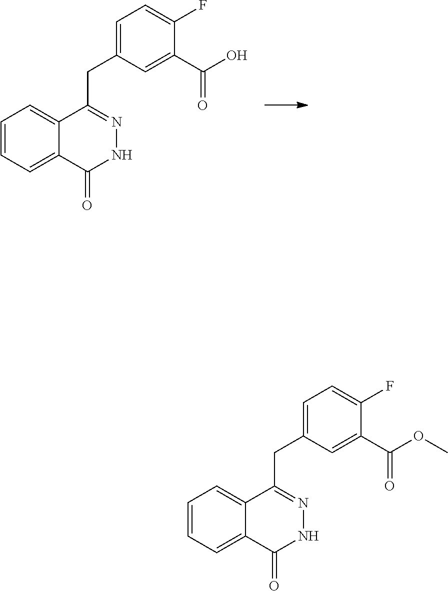 [SMILES: O=C(O)c1cc(Cc2n[nH]c(=O)c3ccccc23)ccc1F ; COC(=O)c1cc(Cc2n[nH]c(=O)c3ccccc23)ccc1F]

To a solution of 2-fluoro-5-[(4-oxo-3,4-dihydrophthalazin-1-yl)methyl]benzoic acid (1.0 g, 3.4 mmol) in 20 mL DMF, methyl iodide was added (1.43 g, 10.2 mmol) following by addition of K₂CO₃ (0.70 g, 5.1 mmol) and the mixture was stirred for 0.5 h at 50° C. The mixture was quenched with 5% HCl, extracted with EtOAc, the combined organic extracts were dried with Na₂SO₄ and evaporated. The residue was suspended in Et₂O, the formed pellets was filtered off, washed with Et₂O and dried giving methyl 2-fluoro-5-[(4-oxo-3,4-dihydrophthalazin-1-yl)methyl]benzoate (P56, 0.81 g, 77%). ¹H NMR (400 MHz, DMSO-d₆) δ 12.57 (s, 1H), 8.26 (d, J=6.8 Hz, 1H), 7.98 (d, J=7.9 Hz, 1H), 7.93-7.87 (m, 1H), 7.83 (t, J=7.5 Hz, 2H), 7.67-7.51 (m, 1H), 7.28 (dd, J=10.9, 8.6 Hz, 1H), 4.36 (s, 2H), 3.82(s, 3H).

### Preparation 57. 4-[4-Fluoro-3-(hydroxymethyl)benzyl]phthalazin-1(2H)-one (P57)

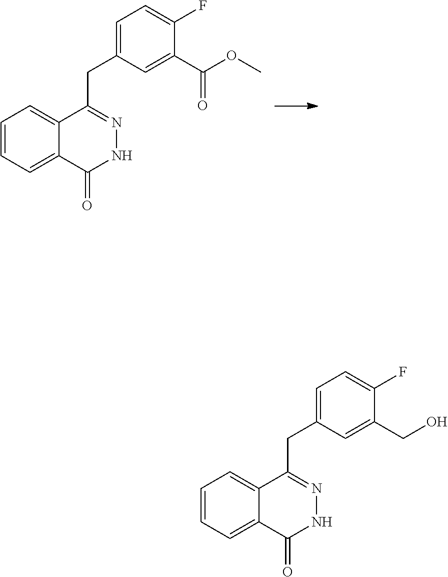 [SMILES: COC(=O)c1cc(Cc2n[nH]c(=O)c3ccccc23)ccc1F ; O=c1[nH]nc(Cc2ccc(F)c(CO)c2)c2ccccc12]

To a solution of methyl 2-fluoro-5-[(4-oxo-3,4-dihydrophthalazin-1-yl)methyl]benzoate (P56, 0.81 g, 2.6 mmol) in 50 mL THF, lithium borohydride was added (0.45 g, 20.8 mmol) and the mixture was stirred at ambient temperature for 15 h. The mixture was quenched with 5% HCl, extracted with EtOAc, the combined organic extracts were dried with Na₂SO₄ and evaporated giving 4-[4-fluoro-3-(hydroxymethyl)benzyl]phthalazin-1(2H)-one (P57, 0.67 g, 91%) that was used for the next step without additional purification. ¹H NMR (400 MHz, DMSO-d₆) δ 12.57 (s, 1H), 8.37-8.17 (m, 1H), 7.93 (d, J=7.8 Hz, 1H), 7.90-7.74 (m, 2H), 7.39 (d, J=7.1 Hz, 1H), 7.31-7.19 (m, 1H), 7.05 (dd, J=10.0, 8.5 Hz, 1H), 6.49 (s, 1H), 5.19 (t, J=5.5 Hz, 1H), 4.48 (d, J=4.4 Hz, 2H), 4.28 (s, 2H).

### Preparation 58. 2-Fluoro-5-[(4-oxo-3,4-dihydrophthalazin-1-yl)methyl]benzaldehyde (P58)

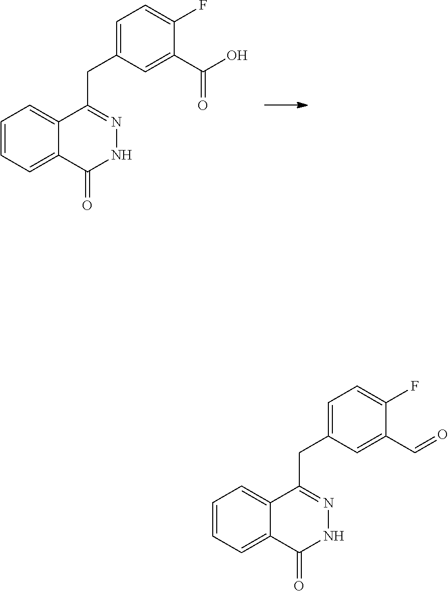 [SMILES: O=C(O)c1cc(Cc2n[nH]c(=O)c3ccccc23)ccc1F ; O=Cc1cc(Cc2n[nH]c(=O)c3ccccc23)ccc1F]

To a solution of 4-[4-fluoro-3-(hydroxymethyl)benzyl]phthalazin-1(2H)-one (P57, 0.47 g, 1.66 mmol) in 150 mL CHCl₃, activated MnO₂ was added (1.44 g, 16.6 mmol) and the mixture was stirred at 50-60° C. for 48 h. The catalyst was filtered off through Celite pad, and the filtrate was washed with aq. NaHCO₃, dried over Na₂SO₄, and evaporated giving 2-fluoro-5-[(4-oxo-3,4-dihydrophthalazin-1-yl)methyl]benzaldehyde (P58, 0.42 g, 90%). ¹H NMR (400 MHz, DMSO-d₆) δ 12.57 (s, 1H), 10.18 (s, 1H), 8.26 (d, J=7.8 Hz, 1H), 7.98 (d, J=8.1 Hz, 1H), 7.89 (t, J=7.5 Hz, 1H), 7.83 (t, J=7.5 Hz, 1H), 7.80-7.75 (m, 1H), 7.74-7.65 (m, 1H), 7.34 (dd, J=10.5, 8.6 Hz, 1H), 4.38 (s, 2H).

### Preparation 59. tert-Butyl 4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazine-1-carboxylate (P59)

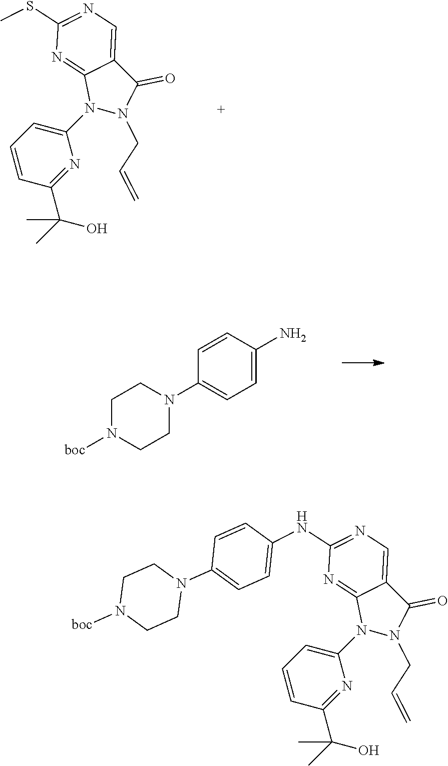

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.35 g, 1.0 mmol) in 10 mL of PhMe, 77% m-CPBA (0.31 g, 1.40 mmol) was added. The reaction mixture was stirred for 1 h at ambient temperature. After completion of the reaction as indicated by TLC, tert-butyl 4-(4-aminophenyl) piperazine-1-carboxylate (0.27 g, 1.0 mmol) and DIPEA (1.0 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified on silica gel eluting with 30-100% EtOAc in hexane to provide tert-butyl 4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazine-1-carboxylate (P59, 0.36 g, 62%). ¹H NMR (400 MHz, DMSO-d₆) δ 8.82(s, 1H), 8.03 (dd, J=20.0, 12.4 Hz, 1H), 7.75 (d, J=8.0 Hz, 1H), 7.60 (t, J=7.5 Hz, 3H), 6.94 (d, J=8.9 Hz, 2H), 5.66 (dq, J=10.9, 5.9 Hz, 1H), 5.30 (s, 1H), 4.99 (d, J=10.2 Hz, 1H), 4.83 (d, J=17.1 Hz, 1H), 4.68 (d, J=5.5 Hz, 2H), 3.46 (d, J=4.9 Hz, 4H), 3.06 (d, J=4.7 Hz, 4H), 1.46 (s, 6H), 1.42(s, 9H).

### Preparation 60. 2-Allyl-1-[6-(1-hydroxy-1-methylethyl)pyridin-2-yl]-6-[(4-piperazin-1-ylphenyl)amino]-1,2-dihydro-3H-pyrazolo[3,4-d]pyrimidin-3-one (P60)

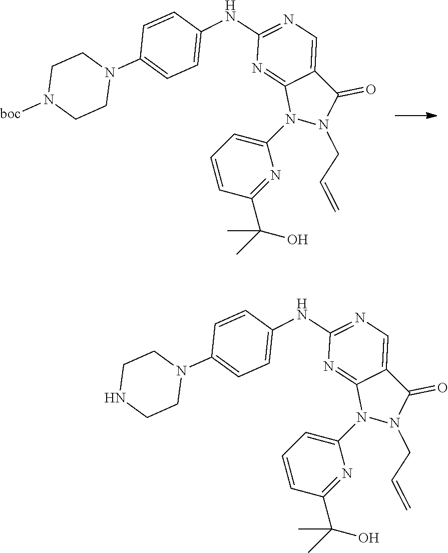

To a solution of tert-butyl 4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazine-1-carboxylate (P59, 0.36 g, 0.61 mmol) in 20 mL DCM, TFA was added (10 mL) and the mixture was kept at ambient temperature for 15 h. The solvents were evaporated, and the residue was basified with aq. NaOH, extracted with DCM, the combined organic extracts were dried with Na₂SO₄ and evaporated giving 2-allyl-1-[6-(1-hydroxy-1-methylethyl)pyridin-2-yl]-6-[(4-piperazin-1-ylphenyl)amino]-1,2-dihydro-3H-pyrazolo[3,4-d]pyrimidin-3-one (P60, 0.29 g, 97%) that was used for the next step without additional purification. ¹H NMR (400 MHz, DMSO-d₆) δ 8.84 (s, 1H), 8.72(s, 1H), 8.02 (t, J=7.7 Hz, 1H), 7.75 (d, J=8.0 Hz, 1H), 7.61 (d, J=7.6 Hz, 3H), 7.03 (d, J=8.7 Hz, 1H), 6.99 (d, J=9.2 Hz, 1H), 5.66 (dd, J=16.7, 10.1 Hz, 1H), 4.99 (d, J=10.3 Hz, 4H), 4.84 (d, J=6.8 Hz, 5H), 3.50 (s, 1H), 3.28 (d, J=13.4 Hz, 4H), 3.12(s, 1H), 1.46 (s, 6H).

### Preparation 61. tert-Butyl 4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazine-1-carboxylate (P61)

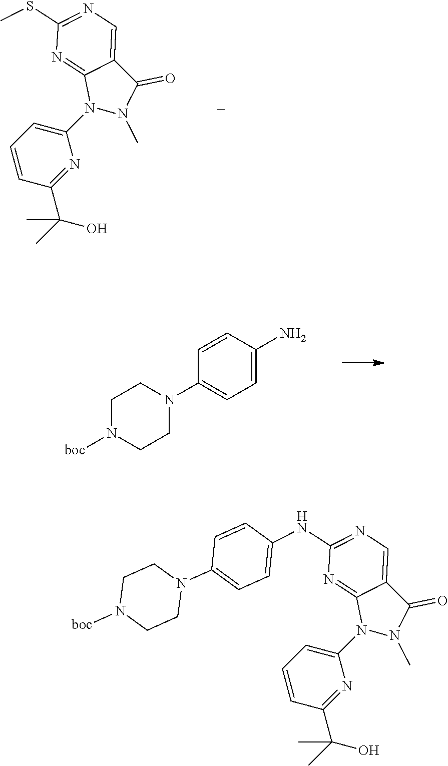

To a solution of 1-[6-(1-hydroxy-1-methylethyl)pyridin-2-yl]-2-methyl-6-(methylthio)-1,2-dihydro-3H-pyrazolo[3,4-d]pyrimidin-3-one (P47, 0.24 g, 0.72 mmol) in 10 mL of PhMe, 77% m-CPBA (0.23 g, 1.00 mmol) was added. The reaction mixture was stirred for 1 h at ambient temperature. After completion of the reaction as indicated by TLC, tert-butyl 4-(4-aminophenyl) piperazine-1-carboxylate (0.20 g, 0.72 mmol) and DIPEA (1.0 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified on silica gel eluting with 30-100% EtOAc in hexane to provide tert-butyl 4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazine-1-carboxylate (P61, 0.17 g, 41%). 1H NMR (400 MHz, DMSO-d₆) δ 8.80 (s, 1H), 8.07 (s, 1H), 7.80 (s, 1H), 7.62(d, J=7.7 Hz, 3H), 6.95 (d, J=9.1 Hz, 2H), 5.29 (s, 1H), 3.45 (d, J=16.9 Hz, 4H), 3.41 (s, 3H), 3.06 (s, 4H), 1.45 (s, 6H), 1.43 (s, 9H).

### Preparation 62. 1-[6-(1-Hydroxy-1-methylethyl)pyridin-2-yl]-2-methyl-6-[(4-piperazin-1-ylphenyl)amino]-1,2-dihydro-3H-pyrazolo[3,4-d]pyrimidin-3-one (P62)

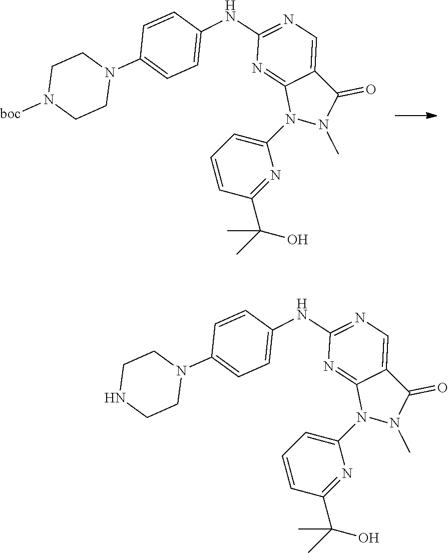

To a solution of tert-butyl 4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazine-1-carboxylate (P61, 0.17 g, 0.29 mmol) in 20 mL DCM, TFA was added (10 mL) and the mixture was kept at ambient temperature for 15 h. The solvents were evaporated, and the residue was basified with aq. NaOH, extracted with DCM, the combined organic extracts were dried with Na₂SO₄ and evaporated giving 1-[6-(1-hydroxy-1-methylethyl)pyridin-2-yl]-2-methyl-6-[(4-piperazin-1-ylphenyl)amino]-1,2-dihydro-3H-pyrazolo[3,4-d]pyrimidin-3-one (P62, 0.14 g, 98%) that was used for the next step without additional purification. LCMS (m/z): 461.5 [MH]⁺, 443.6 [M-H₂O]⁺

### Preparation 63. 2-Methyl-6-methylsulfanyl-1-(2-pyridyl)pyrazolo[3,4-d]pyrimidin-3-one (P63)

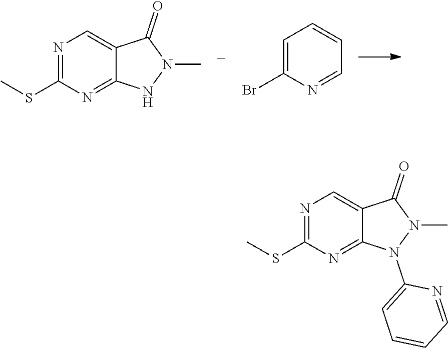

N,N′-Dimethylethylenediamine (0.898 g, 10.19 mmol) was added to a solution of the 2-methyl-6-methylsulfanyl-1H-pyrazolo[3,4-d]pyrimidin-3-one (1.0 g, 5.09 mmol), 2-bromopyridine (1.04 g, 6.61 mmol), copper iodide (0.971 g, 5.09 mmol) and K₂CO₃ (0.99 g, 7.12 mmol) in 1,4-dioxane (10 mL) at 80° C. The resultant suspension was heated at 95° C. for 18 h, over which time a color change of orange to dark green occurred. The reaction mixture was cooled to ambient temperature and diluted with NH₄OH (50 mL) before being extracted with EtOAc (2×50 mL). The combined organic extracts were washed with brine, dried over Na₂SO₄, and evaporated to dryness before the crude material was purified via chromatography on silica with hexane-EtOAc (1:1) to yield 2-methyl-6-methylsulfanyl-1-(2-pyridyl)pyrazolo[3,4-d]pyrimidin-3-one (P63, 0.99 g, 70%) as a white solid. ¹H NMR (400 MHz, DMSO-d₆) δ 9.00 (s, 1H), 8.56 (ddd, J=4.9, 1.9, 0.9 Hz, 1H), 8.10 (ddd, J=8.2, 7.4, 1.9 Hz, 1H), 7.94 (dt, J=8.2, 0.9 Hz, 1H), 7.44 (ddd, J=7.4, 4.8, 1.0 Hz, 1H), 3.44 (s, 3H), 2.55 (s, 3H).

### Preparation 64. 6-Chloro-2-(2-methoxyethyl)-1-pyridin-2-yl-1,2-dihydro-3H-pyrazolo[3,4-d]pyrimidin-3-one (P64)

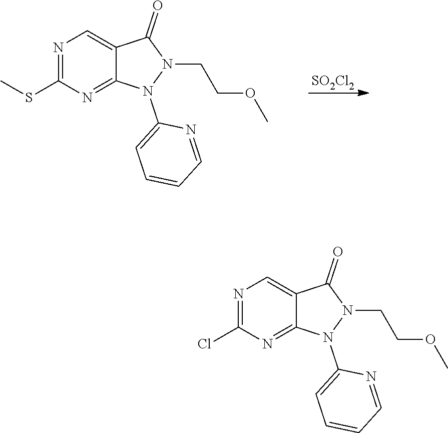

To a solution of 2-(2-methoxyethyl)-6-methylsulfanyl-1-(2-pyridyl)pyrazolo[3,4-d]pyrimidin-3-one (P16, 0.19 g, 0.58 mmol) in CH₂Cl₂-MeCN, 3:1 (10 mL), SO₂Cl₂(0.39 g, 2.9 mmol) was added portionwise at ambient temperature. The mixture was stirred at ambient temperature for 1 h and carefully poured into aq. NaHCO₃. The mixture was extracted with CH₂Cl₂, the combined organic extracts were dried with Na₂SO₄ and evaporated giving 6-chloro-2-(2-methoxyethyl)-1-pyridin-2-yl-1,2-dihydro-3H-pyrazolo[3,4-d]pyrimidin-3-one (P64, 0.18 g, 99%) as a white solid that was used for the next steps without additional purification. ¹H NMR (400 MHz, DMSO-d₆) δ 9.18 (s, 1H), 8.56-8.49 (m, 1H), 8.14-8.06 (m, 1H), 8.07-7.99 (m, 1H), 7.47-7.39 (m, 1H), 4.25 (t, J=5.3 Hz, 2H), 3.35 (t, J=5.2 Hz, 2H), 3.08 (s, 3H).

### Preparation 65. 5-Fluoro-1-[4-fluoro-3-(hydroxymethyl)benzyl]quinazoline-2,4 (1H,3H)-dione (P65)

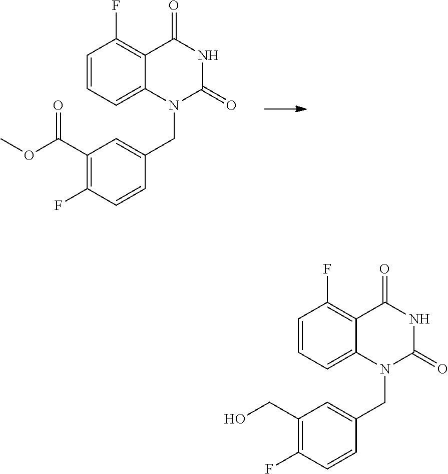 [SMILES: COC(=O)c1cc(Cn2c(=O)[nH]c(=O)c3c(F)cccc32)ccc1F ; O=c1[nH]c(=O)n(Cc2ccc(F)c(CO)c2)c2cccc(F)c12]

To a solution of methyl 2-fluoro-5-[(5-fluoro-2,4-dioxo-3,4-dihydroquinazolin-1(2/I)-yl)methyl]benzoate (0.78 g, 2.2 mmol) in 50 mL THF, lithium borohydride was added (1.69 g, 44.0 mmol) and the mixture was stirred at ambient temperature for 15 h. The mixture was quenched with 5% HCl, extracted with EtOAc, the combined organic extracts were dried with Na₂SO₄ and evaporated giving 5-fluoro-1-[4-fluoro-3-(hydroxymethyl)benzyl]quinazoline-2,4 (1H,3H)-dione (P65, 0.25 g, 35%) that was used for the next step without additional purification. ¹H NMR (400 MHz, DMSO-d₆) δ 11.67 (s, 1H), 7.62(dd, J=14.2, 8.4 Hz, 1H), 7.39 (d, J=6.7 Hz, 1H), 7.24 (s, 1H), 7.18-6.92(m, 3H), 6.51 (s, 1H), 5.44-5.17 (m, 2H), 4.49 (d, J=5.1 Hz, 2H).

### Preparation 66. 2-Fluoro-5-[(5-fluoro-2,4-dioxo-3,4-dihydroquinazolin-1(2H)-yl)methyl]benzaldehyde (P66)

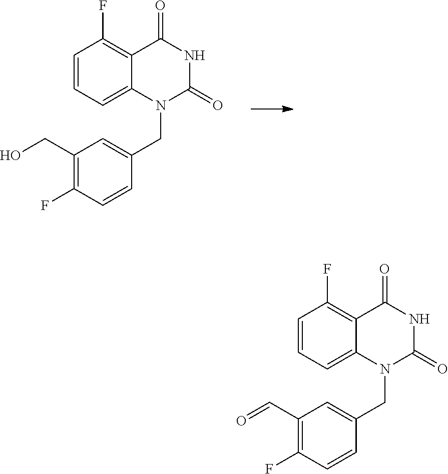 [SMILES: O=c1[nH]c(=O)n(Cc2ccc(F)c(CO)c2)c2cccc(F)c12 ; O=Cc1cc(Cn2c(=O)[nH]c(=O)c3c(F)cccc32)ccc1F]

To a solution of 5-fluoro-1-[4-fluoro-3-(hydroxymethyl)benzyl]quinazoline-2,4 (1H,3H)-dione (P65, 0.25 g, 0.79 mmol) in 100 mL CHCl₃, activated MnO₂ was added (0.68 g, 7.9 mmol) and the mixture was stirred at 50-60° C. for 48 h. The catalyst was filtered off through Celite, and the filtrate was washed with aq. NaHCO₃, dried over Na₂SO₄, and evaporated giving 2-fluoro-5-[(5-fluoro-2,4-dioxo-3,4-dihydroquinazolin-1(2H)-yl)methyl]benzaldehyde (P66, 0.19 g, 78%). 1H NMR (400 MHz, DMSO-d₆) δ 11.71 (s, 1H), 10.18 (s, 1H), 7.79 (d, J=6.4 Hz, 1H), 7.67 (d, J=17.4 Hz, 1H), 7.62(dd, J=14.4, 8.5 Hz, 1H), 7.37 (t, J=9.6 Hz, 1H), 7.12-6.95 (m, 2H), 5.34 (s, 2H).

### Synthesis of the Representative Examples of the Compound

### Example 1. 1-[[3-[4-[4-[[2-Allyl-1-[6-(1-hydroxy-1-methyl-ethyl)-2-pyridyl]-3-oxo-pyrazolo[3,4-d]pyrimidin-6-yl]amino]phenyl]piperazine-1-carbonyl]-4-fluoro-phenyl]methyl]-5-fluoro-quinazoline-2,4-dione (16)

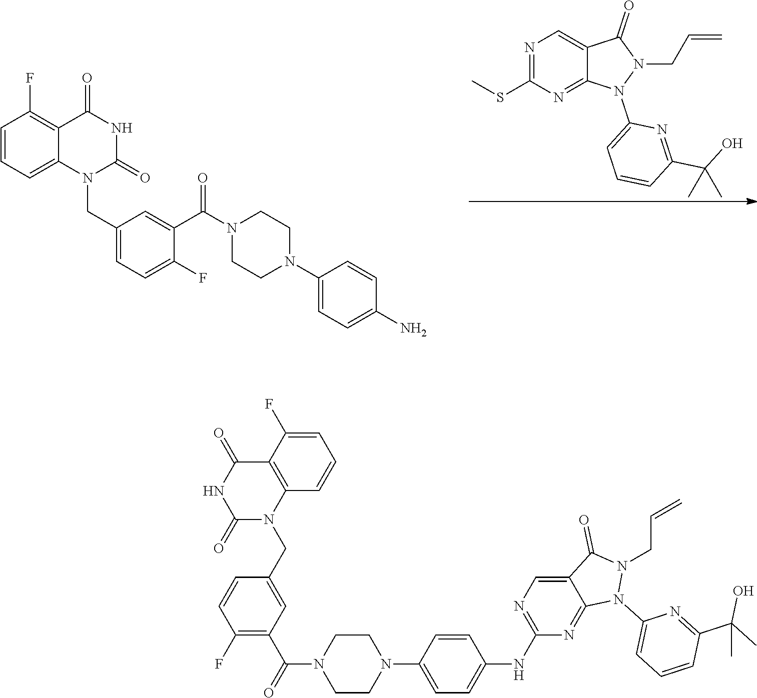 [SMILES: Nc1ccc(N2CCN(C(=O)c3cc(Cn4c(=O)[nH]c(=O)c5c(F)cccc54)ccc3F)CC2)cc1 ; C=CCn1c(=O)c2cnc(SC)nc2n1-c1cccc(C(C)(C)O)n1 ; C=CCn1c(=O)c2cnc(Nc3ccc(N4CCN(C(=O)c5cc(Cn6c(=O)[nH]c(=O)c7c(F)cccc76)ccc5F)CC4)cc3)nc2n1-c1cccc(C(C)(C)O)n1]

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.087 g, 0.24 mmol) in 2 mL of PhMe, 77% m-CPBA (0.093 g, 0.52 mmol) was added. The reaction mixture was stirred for 1 h at ambient temperature. After completion of the reaction as indicated by TLC, 1-(3-{[4-(4-aminophenyl) piperidin-1-yl]carbonyl}-4-fluorobenzyl)-5-fluoroquinazoline-2,4 (1H,3H)-dione (P8, 0.12 g, 0.24 mmol) and DIPEA (0.5 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 1-[3-({4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}carbonyl)-4-fluorobenzyl]-5-fluoroquinazoline-2,4 (1H,3H)-dione (16, 0.02 g, 10%). ¹H NMR (400 MHz, DMSO-d₆) § 11.66 (s, 1H), 10.15 (s, 1H), 8.83 (s, 1H), 8.05 (s, 1H), 7.75 (d, J=8.2 Hz, 1H), 7.62(t, J=10.6 Hz, 3H), 7.45 (s, 1H), 7.41 (s, 1H), 7.29 (t, J=8.6 Hz, 1H), 7.10-6.99 (m, 2H), 6.93 (d, J=8.5 Hz, 2H), 5.66 (d, J=6.4 Hz, 1H), 5.32(s, 3H), 4.99 (d, J=9.8 Hz, 1H), 4.83 (d, J=17.1 Hz, 2H), 4.69 (s, 2H), 3.77 (s, 2H), 3.33 (s, 2H), 3.17 (s, 2H), 2.97 (s, 2H), 1.46 (s, 6H).

### Example 2. 2-Fluoro-5-[(5-fluoro-2,4-dioxo-3,4-dihydroquinazolin-1(2H)-yl)methyl]-N-{4-[(2-methyl-3-oxo-1-pyridin-2-yl-2,3-dihydro-1H-pyrazolo[3,4-d]pyrimidin-6-yl)amino]phenyl}benzamide (33)

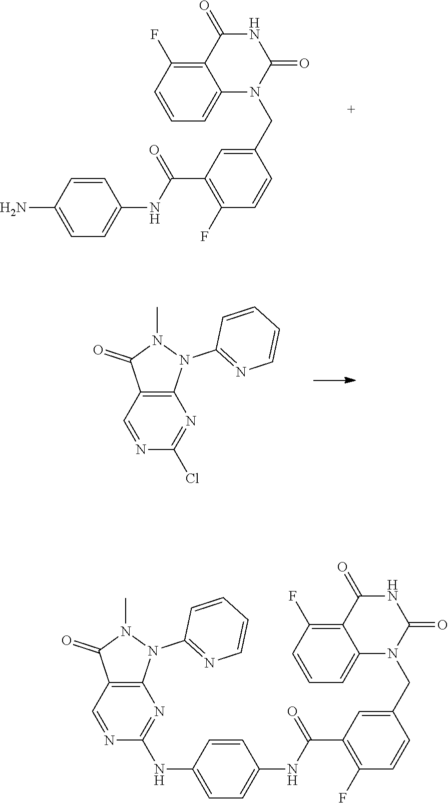 [SMILES: Nc1ccc(NC(=O)c2cc(Cn3c(=O)[nH]c(=O)c4c(F)cccc43)ccc2F)cc1 ; Cn1c(=O)c2cnc(Cl)nc2n1-c1ccccn1 ; Cn1c(=O)c2cnc(Nc3ccc(NC(=O)c4cc(Cn5c(=O)[nH]c(=O)c6c(F)cccc65)ccc4F)cc3)nc2n1-c1ccccn1]

To a solution of N-(4-Aminophenyl)-2-fluoro-5-[(5-fluoro-2,4-dioxo-3,4-dihydroquinazolin-1(2H)-yl)methyl]benzamide (P10, 0.10 g, 0.23 mmol) in 5 mL DMF, and 6-chloro-2-methyl-1-pyridin-2-yl-1,2-dihydro-3H-pyrazolo[3,4-d]pyrimidin-3-one (P24, 0.062 g, 0.23 mmol), p-TsOH was added (0.041 g, 0.23 mmol) and the mixture was stirred at 80° C. for 15 h. The reaction mixture was quenched with aq NaHCO₃, the formed pellet was filtered off, washed with H₂O and EtOAc and dried giving 2-fluoro-5-[(5-fluoro-2,4-dioxo-3,4-dihydroquinazolin-1(2H)-yl)methyl]-N-{4-[(2-methyl-3-oxo-1-pyridin-2-yl-2,3-dihydro-1H-pyrazolo[3,4-d]pyrimidin-6-yl)amino]phenyl}benzamide (33, 0.061 g, 30%). ¹H NMR (400 MHz, DMSO-d₆) δ 11.69 (s, 1H), 10.34 (s, 1H), 10.25 (s, 1H), 8.87 (s, 1H), 8.55 (s, 1H), 8.05 (d, J=7.2 Hz, 1H), 7.93 (d, J=8.3 Hz, 1H), 7.71 (d, J=9.1 Hz, 2H), 7.64 (d, J=7.3 Hz, 4H), 7.49 (s, 1H), 7.46-7.38 (m, 1H), 7.30 (t, J=9.3 Hz, 1H), 7.10 (d, J=8.2 Hz, 1H), 7.06-6.98 (m, 1H), 5.34 (s, 2H), 3.37 (s, 3H).

### Example 3. 5-Fluoro-1-{4-fluoro-3-[(4-{4-[(2-methyl-3-oxo-1-pyridin-2-yl-2,3-dihydro-1H-pyrazolo[3,4-d]pyrimidin-6-yl)amino]phenyl}piperazin-1-yl)carbonyl]benzyl}quinazoline-2,4 (1H,3H)-dione (18)

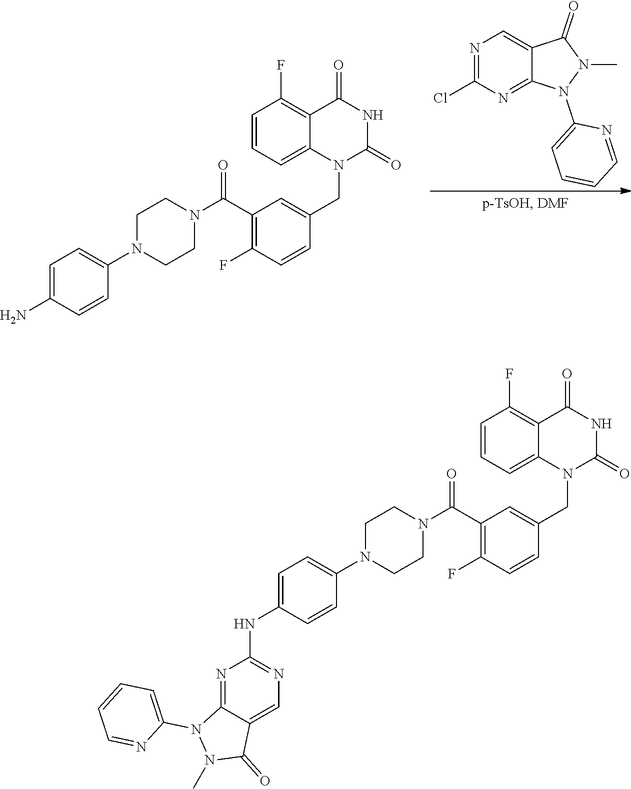 [SMILES: Nc1ccc(N2CCN(C(=O)c3cc(Cn4c(=O)[nH]c(=O)c5c(F)cccc54)ccc3F)CC2)cc1 ; Cn1c(=O)c2cnc(Cl)nc2n1-c1ccccn1 ; Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1]

To a solution of 1-(3-{[4-(4-aminophenyl) piperazin-1-yl]carbonyl}-4-fluorobenzyl)-5-fluoroquinazoline-2,4 (1H,3H)-dione (P8, 0.065 g, 0.13 mmol) and 6-chloro-2-methyl-1-pyridin-2-yl-1,2-dihydro-3H-pyrazolo[3,4-d]pyrimidin-3-one (P24, 0.035 g, 0.13 mmol) in 10 mL DMF, p-TsOH was added (0.023 g, 0.13 mmol) and the mixture was stirred at 80° C. for 15 h. The mixture was quenched with aq. NaHCO₃, extracted with CHCl₃, the combined organic extracts were dried with Na₂SO₄ and evaporated. The residue was washed with MeOH, MeCN and dried giving 5-fluoro-1-{4-fluoro-3-[(4-{4-[(2-methyl-3-oxo-1-pyridin-2-yl-2,3-dihydro-1H-pyrazolo[3,4-d]pyrimidin-6-yl)amino]phenyl}piperazin-1-yl)carbonyl]benzyl}quinazoline-2,4 (1H,3H)-dione (18, 0.036 g, 38%). ¹H NMR (400 MHz, DMSO-d₆) δ 11.68 (s, 1H), 10.13 (s, 1H), 8.82(s, 1H), 8.54 (d, J=4.2 Hz, 1H), 8.11 (s, 1H), 7.92(d, J=8.1 Hz, 1H), 7.73-7.54 (m, 3H), 7.46 (s, 1H), 7.39 (t, J=6.0 Hz, 2H), 7.29 (t, J=8.9 Hz, 1H), 7.11-6.98 (m, 2H), 6.94 (d, J=8.8 Hz, 2H), 5.32(s, 2H), 3.77 (s, 2H), 3.37 (s, 3H), 3.31 (s, 2H), 3.17 (s, 2H), 2.98 (s, 2H).

### Example 4. 5-Fluoro-1-{4-fluoro-3-[(4-{4-[(1-methyl-3-oxo-2-pyridin-2-yl-2,3-dihydro-1H-pyrazolo[3,4-d]pyrimidin-6-yl)amino]phenyl}piperazin-1-yl)carbonyl]benzyl}quinazoline-2,4 (1H,3H)-dione (17)

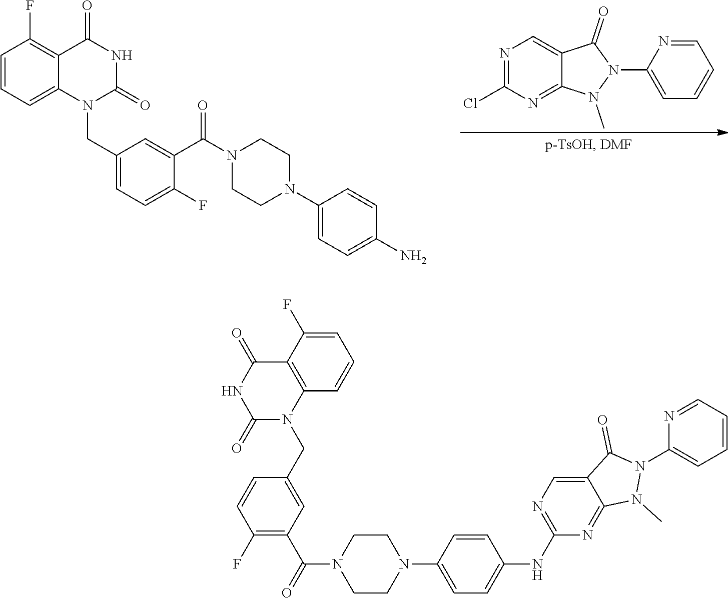 [SMILES: Nc1ccc(N2CCN(C(=O)c3cc(Cn4c(=O)[nH]c(=O)c5c(F)cccc54)ccc3F)CC2)cc1 ; Cn1c2nc(Cl)ncc2c(=O)n1-c1ccccn1 ; Cn1c2nc(Nc3ccc(N4CCN(C(=O)c5cc(Cn6c(=O)[nH]c(=O)c7c(F)cccc76)ccc5F)CC4)cc3)ncc2c(=O)n1-c1ccccn1]

To a solution of 1-(3-{[4-(4-aminophenyl) piperazin-1-yl]carbonyl}-4-fluorobenzyl)-5-fluoroquinazoline-2,4 (1H,3H)-dione (P8, 0.056 g, 0.11 mmol) and 6-chloro-1-methyl-2-pyridin-2-yl-1,2-dihydro-3H-pyrazolo[3,4-d]pyrimidin-3-one (P46, 0.030 g, 0.11 mmol) in 10 mL DMF, p-TsOH was added (0.020 g, 0.11 mmol) and the mixture was stirred at 80° C. for 15 h. The mixture was quenched with aq. NaHCO₃, extracted with CHCl₃, the combined organic extracts were dried with Na₂SO₄ and evaporated. The residue was washed with MeOH, MeCN and dried giving 5-fluoro-1-{4-fluoro-3-[(4-{4-[(1-methyl-3-oxo-2-pyridin-2-yl-2,3-dihydro-1H-pyrazolo[3,4-d]pyrimidin-6-yl)amino]phenyl}piperazin-1-yl)carbonyl]benzyl}quinazoline-2,4 (1H,3H)-dione (17, 0.034 g, 42%). ¹H NMR (400 MHz, DMSO-d₆) δ 10.16 (s, 1H), 8.80 (s, 1H), 8.53 (d, J=4.2 Hz, 1H), 7.97 (d, J=7.4 Hz, 1H), 7.84 (d, J=8.1 Hz, 1H), 7.68 (s, 2H), 7.40 (s, 2H), 7.36-7.21 (m, 4H), 6.95 (d, J=8.7 Hz, 2H), 6.87 (d, J=8.6 Hz, 1H), 6.81 (s, 1H), 5.26 (s, 2H), 3.77 (s, 2H), 3.49 (s, 3H), 3.34 (s, 2H), 3.16 (s, 2H), 2.97 (s, 2H).

### Example 5. 1-{3-[(4-{4-[(2-Allyl-3-oxo-1-pyridin-2-yl-2,3-dihydro-1H-pyrazolo[3,4-d]pyrimidin-6-yl)amino]phenyl}piperazin-1-yl)carbonyl]-4-fluorobenzyl}-5-fluoroquinazoline-2,4 (1H,3H)-dione (28)

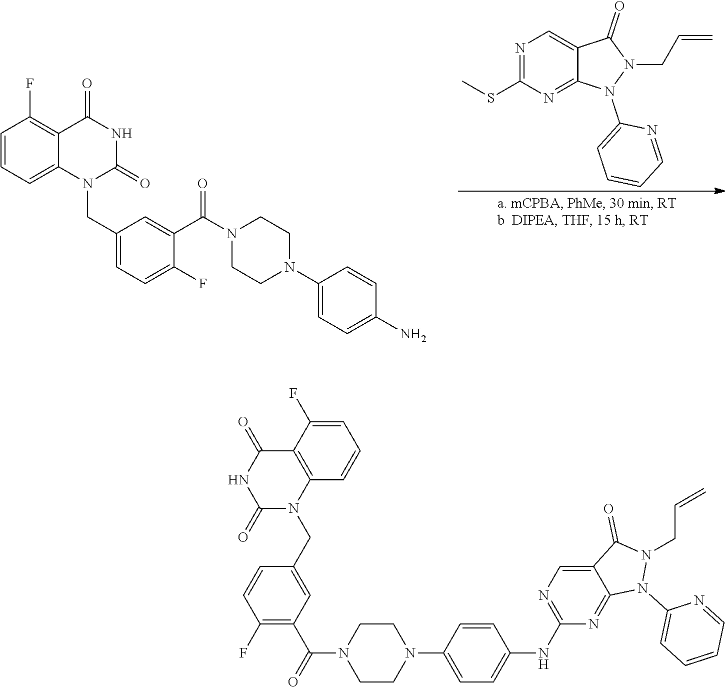 [SMILES: Nc1ccc(N2CCN(C(=O)c3cc(Cn4c(=O)[nH]c(=O)c5c(F)cccc54)ccc3F)CC2)cc1 ; C=CCn1c(=O)c2cnc(SC)nc2n1-c1ccccn1 ; C=CCn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1]

To a solution of 2-allyl-6-(methylthio)-1-pyridin-2-yl-1,2-dihydro-3H-pyrazolo[3,4-d]pyrimidin-3-one (P14, 0.073 g, 0.24 mmol) in 2 mL of PhMe, 77% m-CPBA (0.093 g, 0.52 mmol) was added. The reaction mixture was stirred for 1 h at ambient temperature. After completion of the reaction as indicated by TLC, 1-(3-{[4-(4-aminophenyl) piperidin-1-yl]carbonyl}-4-fluorobenzyl)-5-fluoroquinazoline-2,4 (1H,3H)-dione (P8, 0.12 g, 0.24 mmol) and DIPEA (0.5 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 1-{3-[(4-{4-[(2-allyl-3-oxo-1-pyridin-2-yl-2,3-dihydro-1H-pyrazolo[3,4-d]pyrimidin-6-yl)amino]phenyl}piperazin-1-yl)carbonyl]-4-fluorobenzyl}-5-fluoroquinazoline-2,4 (1H,3H)-dione (28, 0.02 g, 11%). ¹H NMR (400 MHz, DMSO-d₆) δ 11.66 (s, 1H), 10.54-9.57 (m, 1H), 8.84 (s, 1H), 8.54 (s, 1H), 8.08 (s, 1H), 7.88 (s, 1H), 7.61 (br. s, 2H), 7.39 (br. s, 4H), 7.33-7.20 (m, 1H), 7.03 (br. s, 2H), 6.93 (br. s, 2H), 5.68 (s, 1H), 5.32(s, 2H), 5.03 (s, 1H), 4.88 (s, 1H), 4.60 (s, 2H), 3.78 (s, 2H), 3.25-3.08 (m, 4H), 2.98 (s, 2H).

### Example 6. 5-Fluoro-1-{4-fluoro-3-[(4-{4-[(2-methyl-3-oxo-1-pyrimidin-2-yl-2,3-dihydro-1H-pyrazolo[3,4-d]pyrimidin-6-yl)amino]phenyl}piperazin-1-yl)carbonyl]benzyl}quinazoline-2,4 (1H,3H)-dione (29)

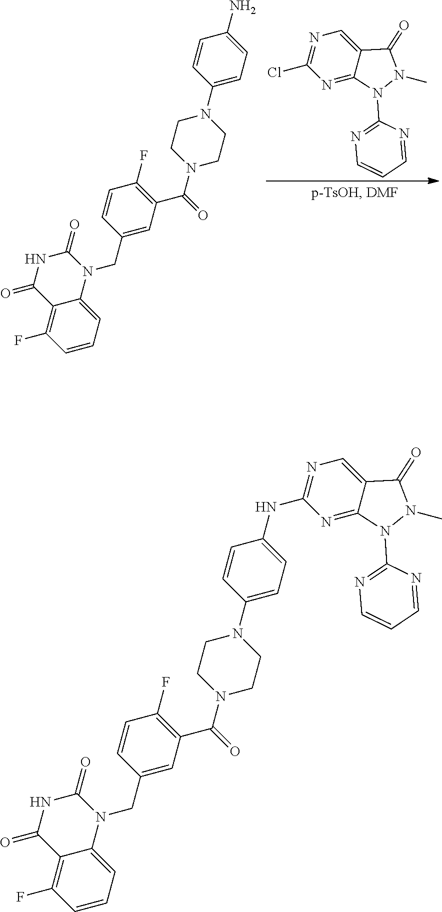

To a solution of 1-(3-{[4-(4-aminophenyl) piperazin-1-yl]carbonyl}-4-fluorobenzyl)-5-fluoroquinazoline-2,4 (1H,3H)-dione (P3, 0.080 g, 0.16 mmol) and 6-chloro-2-methyl-1-pyrimidin-2-yl-1,2-dihydro-3H-pyrazolo[3,4-d]pyrimidin-3-one (P10, 0.043 g, 0.16 mmol) in 10 mL DMF, p-TsOH was added (0.028 g, 0.16 mmol) and the mixture was stirred at 80° C. for 15 h. The mixture was quenched with aq. NaHCO₃, extracted with CHCl₃, the combined organic extracts were dried with Na₂SO₄ and evaporated. The residue was washed with MeOH, MeCN and dried. The solid was further purified on silica gel eluting with 1-10% MeOH—CHCl₃ 5-fluoro-1-{4-fluoro-3-[(4-{4-[(2-methyl-3-oxo-1-pyrimidin-2-yl-2,3-dihydro-1H-pyrazolo[3,4-d]pyrimidin-6-yl)amino]phenyl}piperazin-1-yl)carbonyl]benzyl}quinazoline-2,4 (1H,3H)-dione (29, 0.034 g, 29%). ¹H NMR (400 MHz, DMSO-d₆) δ 11.67 (s, 1H), 10.22(s, 1H), 9.01 (s, 1H), 8.83 (s, 1H), 7.93 (m, 2H), 7.64 (s, 1H), 7.57-7.35 (m, 3H), 7.29 (s, 2H), 7.01 (d, J=29.5 Hz, 4H), 5.32(s, 2H), 3.78 (s, 2H), 3.30 (s, 2H), 3.41 (s, 3H), 3.18 (s, 2H), 2.98 (s, 2H).

### Example 7. 2-[4-({4-[4-({2-Allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)phenyl]-8-fluoro-1,3,4,5-tetrahydro-6H-azepino[5,4,3-cd]indol-6-one (35)

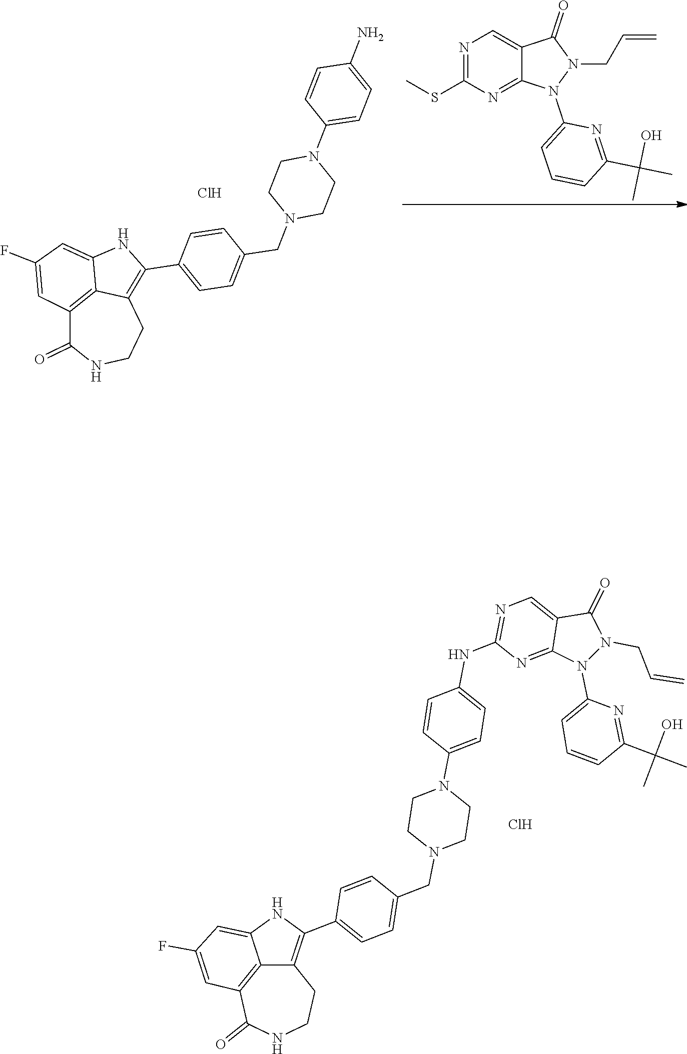

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.15 g, 0.43 mmol) in 2 mL of PhMe, 77% m-CPBA (0.12 g, 0.55 mmol) was added. The reaction mixture was stirred for 1 h at ambient temperature. After completion of the reaction as indicated by TLC, 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.20 g, 0.40 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 2-[4-({4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)phenyl]-8-fluoro-1,3,4,5-tetrahydro-6H-azepino[5,4,3-cd]indol-6-one (35, 0.028 g, 9%). ¹H NMR (400 MHz, DMSO-d₆) δ 11.84 (s, 1H), 11.19 (s, 1H), 10.19 (s, 1H), 8.84 (s, 1H), 8.28 (s, 1H), 8.02(t, J=7.7 Hz, 1H), 7.80 (d, J=8.3 Hz, 2H), 7.74 (d, J=8.2 Hz, 3H), 7.61 (d, J=7.4 Hz, 3H), 7.44 (dd, J=11.0, 2.4 Hz, 1H), 7.36 (dd, J=9.1, 2.4 Hz, 1H), 6.98 (d, J=9.0 Hz, 2H), 5.77-5.50 (m, 1H), 4.99 (d, J=10.2 Hz, 1H), 4.82(d, J=16.0 Hz, 1H), 4.67 (d, J=5.3 Hz, 2H), 4.45 (s, 2H), 3.44 (dd, J=12.7, 5.7 Hz, 5H), 3.18 (d, J=8.7 Hz, 4H), 3.08 (s, 4H), 1.46 (s, 6H).

### Example 8. 2-{4-[(4-{2-Allyl-1-[6-(1-hydroxy-1-methylethyl)pyridin-2-yl]-3-oxo-2,3-dihydro-1H-pyrazolo[3,4-d]pyrimidin-6-yl}piperazin-1-yl)methyl]phenyl}-8-fluoro-1,3,4,5-tetrahydro-6H-azepino[5,4,3-cd]indol-6-one (44)

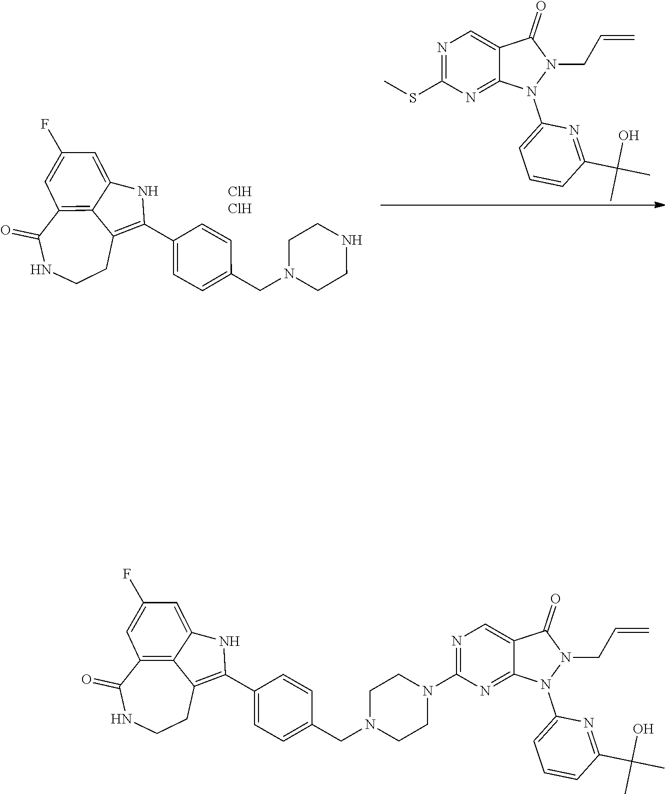

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.029 g, 0.08 mmol) in 2 mL of PhMe, 77% m-CPBA (0.027 g, 0.12 mmol) was added. The reaction mixture was stirred for 1 h at ambient temperature. After completion of the reaction as indicated by TLC, 8-fluoro-2-[4-(piperazin-1-ylmethyl)phenyl]-1,3,4,5-tetrahydro-6H-azepino[5,4,3-cd]indol-6-one (P41, 0.037 g, 0.08 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 2-{4-[(4-{2-allyl-1-[6-(1-hydroxy-1-methylethyl)pyridin-2-yl]-3-oxo-2,3-dihydro-1H-pyrazolo[3,4-d]pyrimidin-6-yl}piperazin-1-yl)methyl]phenyl}-8-fluoro-1,3,4,5-tetrahydro-6H-azepino[5,4,3-cd]indol-6-one (44, 0.018 g, 32%). ¹H NMR (400 MHz, DMSO-d₆) δ 11.66 (s, 1H), 8.78 (s, 1H), 8.25 (s, 1H), 7.94 (t, J=7.9 Hz, 1H), 7.71 (d, J=8.1 Hz, 1H), 7.61 (d, J=8.2 Hz, 2H), 7.56 (d, J=7.9 Hz, 1H), 7.49 (d, J=8.0 Hz, 2H), 7.42(dd, J=11.0, 2.4 Hz, 1H), 7.32(dd, J=9.2, 2.3 Hz, 1H), 5.63 (dd, J=17.1, 10.4 Hz, 1H), 5.29 (s, 1H), 4.98 (d, J=10.3 Hz, 1H), 4.80 (d, J=17.1 Hz, 1H), 4.66 (d, J=5.8 Hz, 2H), 3.89 (s, 4H), 3.59 (s, 2H), 3.39 (s, 2H), 3.05 (s, 2H), 2.50 (s, 4H), 1.45 (s, 6H).

### Example 9. 2-(4-{[4-({2-Allyl-1-[6-(1-hydroxy-1-methylethyl)pyridin-2-yl]-3-oxo-2,3-dihydro-1H-pyrazolo[3,4-d]pyrimidin-6-yl}amino) piperidin-1-yl]methyl}phenyl)-8-fluoro-1,3,4,5-tetrahydro-6H-azepino[5,4,3-cd]indol-6-one (47)

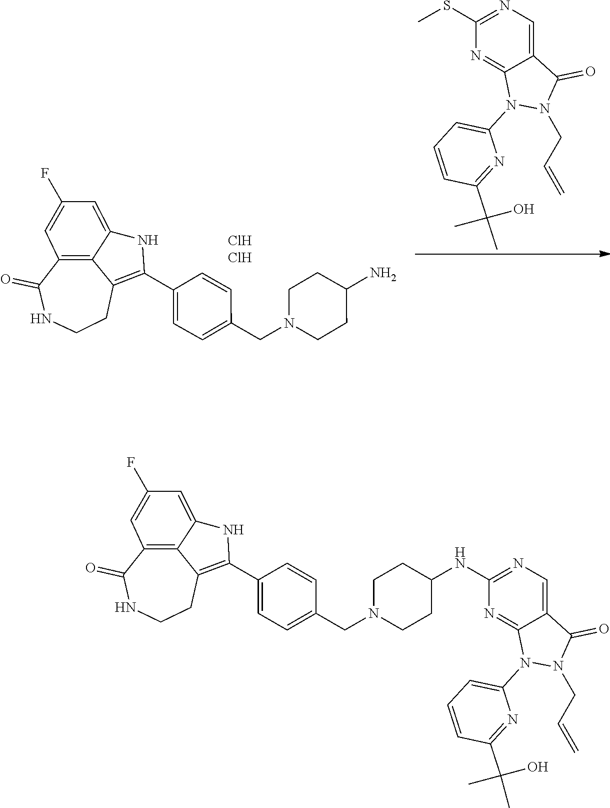

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.04 g, 0.11 mmol) in 2 mL of PhMe, 77% m-CPBA (0.029 g, 0.22 mmol) was added. The reaction mixture was stirred for 1 h at ambient temperature. After completion of the reaction as indicated by TLC, 2-{4-[(4-aminopiperidin-1-yl)methyl]phenyl}-8-fluoro-1,3,4,5-tetrahydro-6H-azepino[5,4,3-cd]indol-6-one (P43, 0.052 g, 0.11 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 2-(4-{[4-({2-allyl-1-[6-(1-hydroxy-1-methylethyl)pyridin-2-yl]-3-oxo-2,3-dihydro-1H-pyrazolo[3,4-d]pyrimidin-6-yl}amino) piperidin-1-yl]methyl}phenyl)-8-fluoro-1,3,4,5-tetrahydro-6H-azepino[5,4,3-cd]indol-6-one (47, 0.012 g, 15%). ¹H NMR (400 MHz, DMSO-d₆) δ 11.66 (s, 1H), 8.35-8.10 (m, 2H), 7.94 (s, 1H), 7.73 (d, J=8.4 Hz, 2H), 7.58 (dd, J=16.0, 8.2 Hz, 3H), 7.44 (dd, J=16.0, 9.2 Hz, 3H), 7.32(d, J=8.9 Hz, 1H), 3.71 (s, 1H), 3.53 (d, J=8.6 Hz, 3H), 3.05 (s, 3H), 2.84 (s, 2H), 2.07 (d, J=11.5 Hz, 3H), 2.07 (d, J=11.5 Hz, 3H), 1.90 (s, 2H), 1.65-1.48 (m, 4H), 1.45 (s, 6H).

### Example 10. 8-Fluoro-2-{4-[(4-{4-[(2-methyl-3-oxo-1-pyridin-2-yl-2,3-dihydro-1H-pyrazolo[3,4-d]pyrimidin-6-yl)amino]phenyl}piperazin-1-yl)methyl]phenyl}-1,3,4,5-tetrahydro-6H-azepino[5,4,3-cd]indol-6-one (36)

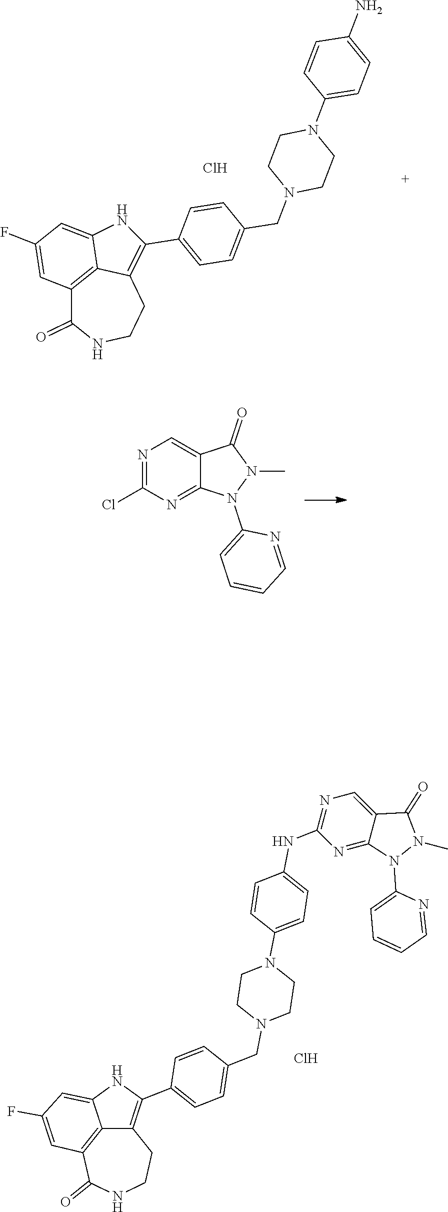 [SMILES: Nc1ccc(N2CCN(Cc3ccc(-c4[nH]c5cc(F)cc6c5c4CCNC6=O)cc3)CC2)cc1 ; Cl ; Cn1c(=O)c2cnc(Cl)nc2n1-c1ccccn1 ; Cn1c(=O)c2cnc(Nc3ccc(N4CCN(Cc5ccc(-c6[nH]c7cc(F)cc8c7c6CCNC8=O)cc5)CC4)cc3)nc2n1-c1ccccn1 ; Cl]

To a solution of N-(4-aminophenyl)-2-fluoro-5-[(5-fluoro-2,4-dioxo-3,4-dihydroquinazolin-1(2H)-yl)methyl]benzamide (P39, 0.15 g, 0.29 mmol) in 5 mL DMF, and 6-chloro-2-methyl-1-pyridin-2-yl-1,2-dihydro-3H-pyrazolo[3,4-d]pyrimidin-3-one (P24, 0.077 g, 0.29 mmol), p-TsOH was added (0.056 g, 0.29 mmol) and the mixture was stirred at 80° C. for 15 h. The reaction mixture was quenched with aq. NaHCO₃, the formed pellets were filtered off, washed with H₂O and EtOAc and dried. The residue was purified on a silica gel column eluting with 1→10% MeOH in CHCl₃ giving 8-fluoro-2-{4-[(4-{4-[(2-methyl-3-oxo-1-pyridin-2-yl-2,3-dihydro-1H-pyrazolo[3,4-d]pyrimidin-6-yl)amino]phenyl}piperazin-1-yl)methyl]phenyl}-1,3,4,5-tetrahydro-6H-azepino[5,4,3-cd]indol-6-one (36, 0.051 g, 24%). ¹H NMR (400 MHz, DMSO-d₆) δ 11.83 (s, 1H), 11.20 (s, 1H), 10.12(s, 1H), 8.81 (s, 2H), 8.53 (s, 2H), 8.26 (s, 1H), 8.07 (s, 1H), 7.89 (d, J=7.1 Hz, 1H), 7.79 (s, 2H), 7.74 (s, 2H), 7.61 (s, 2H), 7.39 (m, 4H), 6.97 (d, J=7.1 Hz, 2H), 4.44 (s, 2H), 3.50 (br. s, 4H), 3.78 (s, 2H), 3.18 (s, 4H), 3.07 (s, 2H).

### Example 11. 8-Fluoro-2-(4-{[4-(4-{[2-(2-methoxyethyl)-3-oxo-1-pyridin-2-yl-2,3-dihydro-1H-pyrazolo[3,4-d]pyrimidin-6-yl]amino}phenyl) piperazin-1-yl]methyl}phenyl)-1,3,4,5-tetrahydro-6H-azepino[5,4,3-cd]indol-6-one (38)

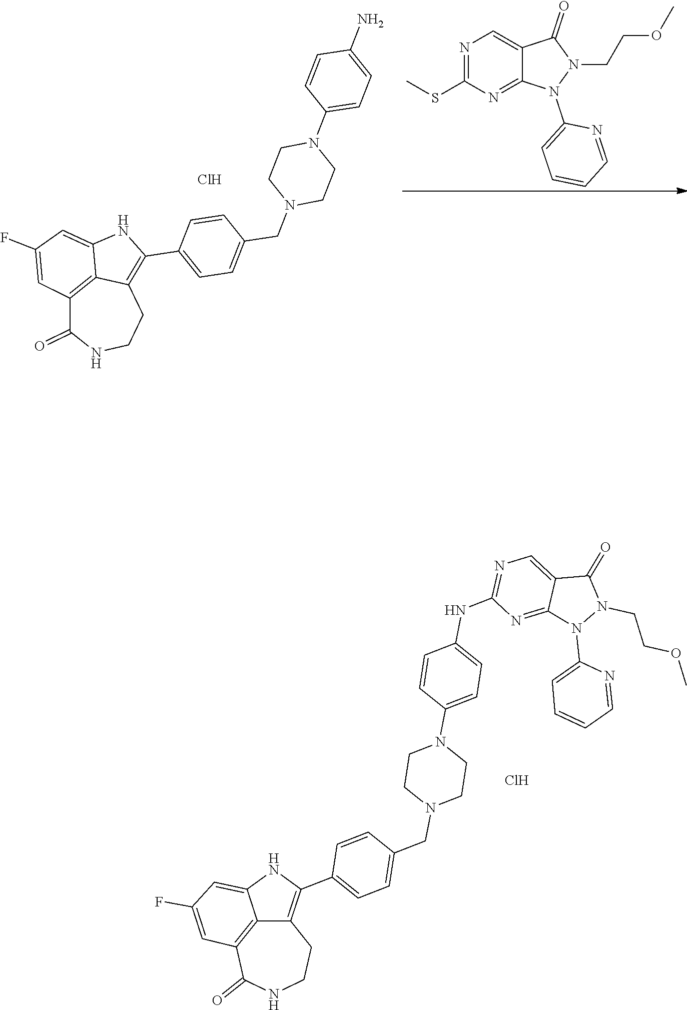

To a solution of 2-(2-methoxyethyl)-6-(methylthio)-1-pyridin-2-yl-1,2-dihydro-3H-pyrazolo[3,4-d]pyrimidin-3-one (P16, 0.094 g, 0.29 mmol) in 2 mL of PhMe, 77% m-CPBA (0.13 g, 0.58 mmol) was added. The reaction mixture was stirred for 1 h at ambient temperature. After completion of the reaction as indicated by TLC, 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.15 g, 0.29 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 8-fluoro-2-(4-{[4-(4-{[2-(2-methoxyethyl)-3-oxo-1-pyridin-2-yl-2,3-dihydro-1H-pyrazolo[3,4-d]pyrimidin-6-yl]amino}phenyl) piperazin-1-yl]methyl}phenyl)-1,3,4,5-tetrahydro-6H-azepino[5,4,3-cd]indol-6-one (38, 0.008 g, 4%).

### Example 12. 2-[4-[[4-[4-[[2-Allyl-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4,6,8 (13)-tetraen-9-one (39)

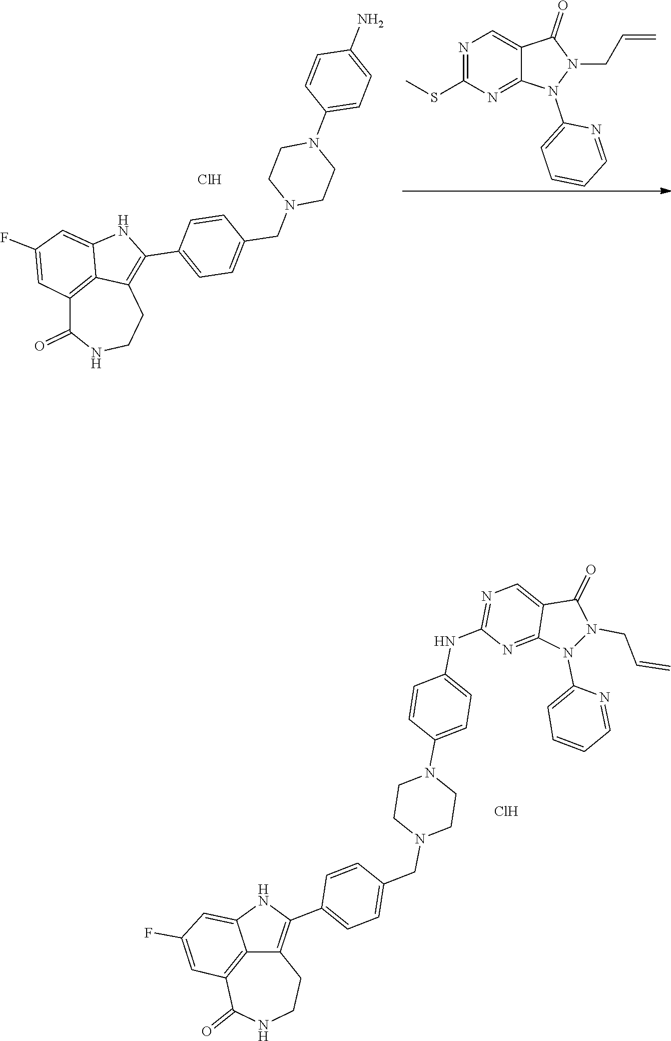

To a solution of 2-allyl-6-methylsulfanyl-1-(2-pyridyl)pyrazolo[3,4-d]pyrimidin-3-one (P14, 0.05 g, 0.16 mmol) in 1 mL of DMA, 77% m-CPBA (0.078 g, 0.33 mmol) was added. The reaction mixture was stirred for 1 h at ambient temperature. After completion of the reaction as indicated by TLC, 2-[4-[[4-(4-aminophenyl) piperazin-1-yl]methyl]phenyl]-6-fluoro-3,10-diazatricyclo[6.4.1.04,13]trideca-1,4,6,8 (13)-tetraen-9-one (P39, 0.078 g, 0.16 mmol) and DIPEA (0.108 g, 0.8 mmol) were added to mixture. The reaction mixture was stirred for 15 h at ambient temperature, and then concentrated. The residue was washed with NaHCO₃, water and dried. The obtained crude residue was purified by RP-HPLC eluting with a gradient MeCN-H₂O+0.1% TFA to provide 2-[4-[[4-[4-[[2-allyl-3-oxo-1-(2-pyridyl)pyrazolo[3,4-d]pyrimidin-6-yl]amino]phenyl]piperazin-1-yl]methyl]phenyl]-6-fluoro-3,10-diazatricyclo[6.4.1.04,13]trideca-1,4,6,8 (13)-tetraen-9-one (39, 0.016 g, 13%). ¹H NMR (400 MHz, DMSO-d₆) δ 11.86 (s, 1H), 11.36 (s, 1H), 10.18 (s, 1H), 8.84 (s, 1H), 8.57-8.51 (m, 1H), 8.27 (d, J=6.1 Hz, 1H), 8.07 (t, J=7.7 Hz, 1H), 7.87 (d, J=8.3 Hz, 1H), 7.82(d, J=8.1 Hz, 2H), 7.74 (d, J=8.1 Hz, 2H), 7.62(d, J=8.5 Hz, 2H), 7.48-7.33 (m, 3H), 6.98 (d, J=8.9 Hz, 2H), 5.74-5.60 (m, 1H), 5.05-4.97 (m, 1H), 4.87 (dd, J=17.1, 1.5 Hz, 1H), 4.62-4.57 (m, 2H), 4.47-4.43 (m, 2H), 3.81-3.75 (m, 2H), 3.44-3.39 (m, 4H), 3.27-3.14 (m, 4H), 3.11-3.06 (m, 2H).

### Example 13. 8-Fluoro-2-[4-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}methyl)phenyl]-1,3,4,5-tetrahydro-6H-azepino[5,4,3-cd]indol-6-one (40)

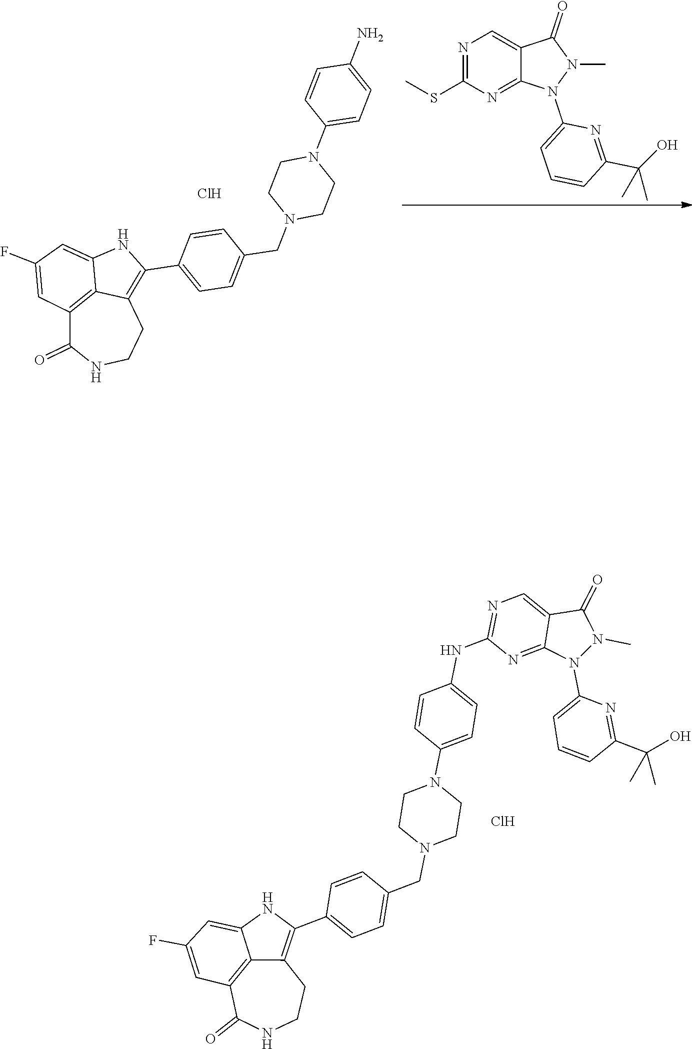

To a solution of 1-[6-(1-hydroxy-1-methylethyl)pyridin-2-yl]-2-methyl-6-(methylthio)-1,2-dihydro-3H-pyrazolo[3,4-d]pyrimidin-3-one (P47, 0.098 g, 0.29 mmol) in 2 mL of PhMe, 77% m-CPBA (0.13 g, 0.58 mmol) was added. The reaction mixture was stirred for 1 h at ambient temperature. After completion of the reaction as indicated by TLC, 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.15 g, 0.29 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 8-fluoro-2-[4-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}methyl)phenyl]-1,3,4,5-tetrahydro-6H-azepino[5,4,3-cd]indol-6-one (40, 0.006 g, 3%).

### Example 14. 4-[3-({4-[4-({2-Allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}carbonyl)-4-fluorobenzyl]phthalazin-1(2H)-one (1)

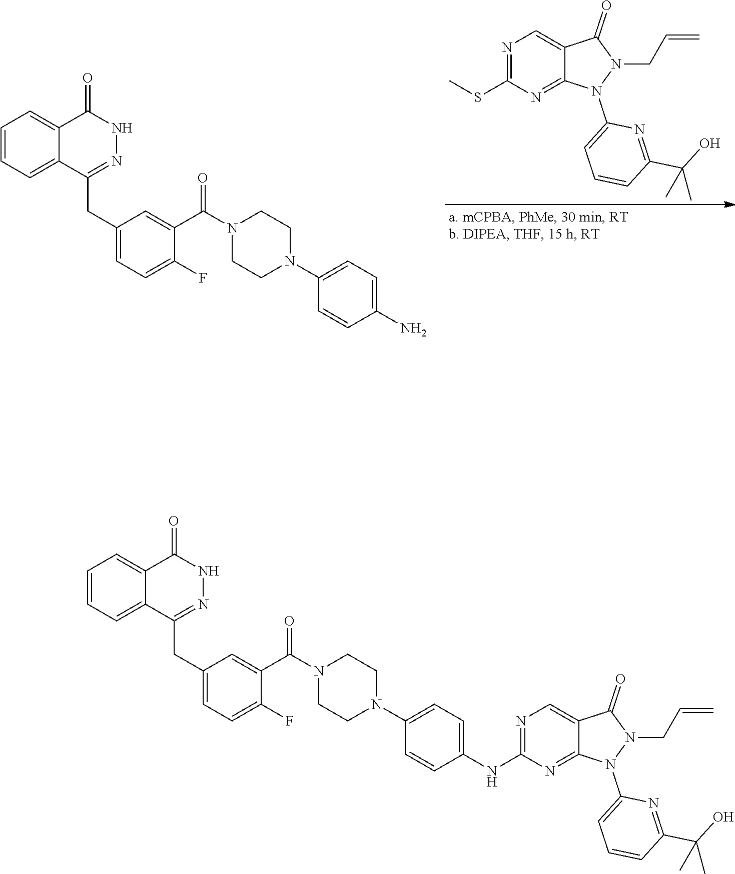 [SMILES: Nc1ccc(N2CCN(C(=O)c3cc(Cc4n[nH]c(=O)c5ccccc45)ccc3F)CC2)cc1 ; C=CCn1c(=O)c2cnc(SC)nc2n1-c1cccc(C(C)(C)O)n1 ; C=CCn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)n1]

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.10 g, 0.28 mmol) in 2 mL of THF, 77% m-CPBA (0.072 g, 0.42 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.13 g, 0.28 mmol) and DIPEA (0.5 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-[3-({4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}carbonyl)-4-fluorobenzyl]phthalazin-1(2H)-one (1, 0.035 g, 16%). ¹H NMR (400 MHz, DMSO-d₆) δ 12.59 (s, 1H), 10.16 (s, 1H), 8.83 (s, 1H), 8.27 (d, J=7.8 Hz, 1H), 8.04 (d, J=7.4 Hz, 1H), 7.98 (d, J=7.5 Hz, 1H), 7.90 (dd, J=11.6, 3.7 Hz, 1H), 7.83 (t, J=7.5 Hz, 1H), 7.75 (d, J=8.0 Hz, 1H), 7.61 (d, J=7.7 Hz, 3H), 7.46 (d, J=5.7 Hz, 1H), 7.42-7.36 (m, 1H), 7.25 (t, J=9.0 Hz, 1H), 6.95 (d, J=9.0 Hz, 2H), 5.66 (dq, J=10.2, 6.0 Hz, 1H), 4.99 (d, J=10.2 Hz, 1H), 4.82(d, J=15.8 Hz, 1H), 4.68 (d, J=5.6 Hz, 2H), 4.35 (s, 2H), 3.78 (s, 2H), 3.32(s, 2H), 3.17 (s, 2H), 3.01 (s, 2H), 1.46 (s, 6H).

### Example 15. 4-{3-[(4-{2-Allyl-1-[6-(1-hydroxy-1-methylethyl)pyridin-2-yl]-3-oxo-2,3-dihydro-1H-pyrazolo[3,4-d]pyrimidin-6-yl}piperazin-1-yl)carbonyl]-4-fluorobenzyl}phthalazin-1(2H)-one (2)

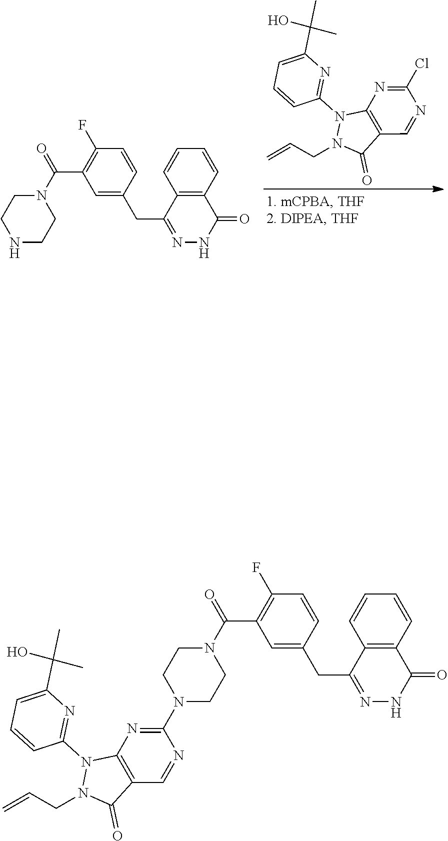 [SMILES: O=C(c1cc(Cc2n[nH]c(=O)c3ccccc23)ccc1F)N1CCNCC1 ; C=CCn1c(=O)c2cnc(Cl)nc2n1-c1cccc(C(C)(C)O)n1 ; C=CCn1c(=O)c2cnc(N3CCN(C(=O)c4cc(Cc5n[nH]c(=O)c6ccccc56)ccc4F)CC3)nc2n1-c1cccc(C(C)(C)O)n1]

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.049 g, 0.13 mmol) in 2 mL of THF, 77% m-CPBA (0.044 g, 0.16 mmol) was added. The reaction mixture was stirred for 1 h at ambient temperature. After completion of the reaction as indicated by TLC, 4-[4-fluoro-3-(piperazin-1-ylcarbonyl)benzyl]phthalazin-1(2H)-one (P51, 0.05 g, 0.13 mmol) and DIPEA (0.15 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-{3-[(4-{2-allyl-1-[6-(1-hydroxy-1-methylethyl)pyridin-2-yl]-3-oxo-2,3-dihydro-1H-pyrazolo[3,4-d]pyrimidin-6-yl}piperazin-1-yl)carbonyl]-4-fluorobenzyl}phthalazin-1(2H)-one (2, 0.067 g, 73%). ¹H NMR (400 MHz, DMSO-d₆) δ 12.59 (s, 1H), 8.82(s, 1H), 8.26 (d, J=7.4 Hz, 1H), 7.98 (d, J=8.1 Hz, 2H), 7.89 (t, J=7.2 Hz, 1H), 7.83 (d, J=7.5 Hz, 1H), 7.72(d, J=7.8 Hz, 1H), 7.57 (d, J=7.5 Hz, 1H), 7.45 (s, 1H), 7.39 (d, J=5.3 Hz, 1H), 7.25 (t, J=9.0 Hz, 2H), 5.63 (dq, J=11.1, 6.1 Hz, 1H), 4.98 (d, J=10.1 Hz, 1H), 4.79 (d, J=17.1 Hz, 1H), 4.68 (d, J=5.8 Hz, 2H), 4.34 (s, 6H), 4.25-3.59 (m, 4H), 1.45 (s, 6H).

### Example 16. N-[2-({2-Allyl-1-[6-(1-hydroxy-1-methylethyl)pyridin-2-yl]-3-oxo-2,3-dihydro-1H-pyrazolo[3,4-d]pyrimidin-6-yl}amino)ethyl]-2-fluoro-5-[(4-oxo-3,4-dihydrophthalazin-1-yl)methyl]benzamide (6)

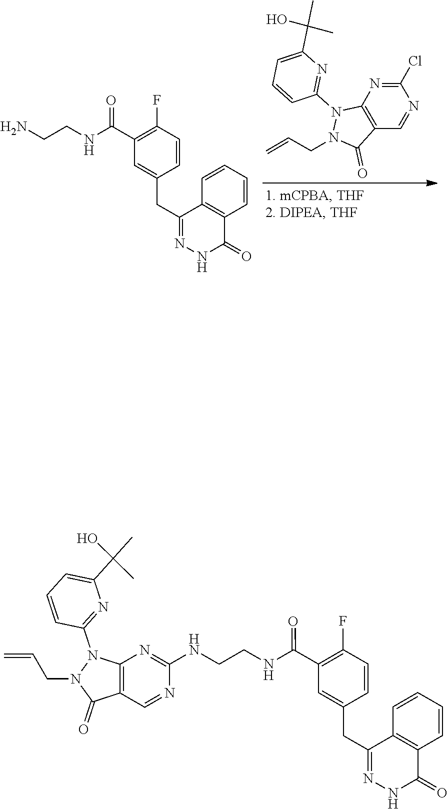

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.043 g, 0.12 mmol) in 2 mL of THF, 77% m-CPBA (0.040 g, 0.18 mmol) was added. The reaction mixture was stirred for 1 h at ambient temperature. After completion of the reaction as indicated by TLC, N-(2-aminoethyl)-2-fluoro-5-[(4-oxo-3,4-dihydrophthalazin-1-yl)methyl]benzamide (P53, 0.05 g, 0.12 mmol) and DIPEA (0.15 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide N-[2-({2-allyl-1-[6-(1-hydroxy-1-methylethyl)pyridin-2-yl]-3-oxo-2,3-dihydro-1H-pyrazolo[3,4-d]pyrimidin-6-yl}amino)ethyl]-2-fluoro-5-[(4-oxo-3,4-dihydrophthalazin-1-yl)methyl]benzamide (6, 0.012 g, 16%). ¹H NMR (400 MHz, DMSO-d₆) δ 12.58 (s, 1H), 8.69 (s, 1H), 8.33 (s, 1H), 8.26 (d, J=6.7 Hz, 1H), 8.19 (s, 1H), 7.98-7.76 (m, 5H), 7.71 (d, J=6.9 Hz, 1H), 7.66-7.48 (m, 2H), 7.44 (s, 1H), 7.18 (t, J=9.5 Hz, 1H), 5.59 (dd, J=16.7, 10.1 Hz, 1H), 4.96 (t, J=10.3 Hz, 1H), 4.79 (dd, J=16.9, 8.6 Hz, 1H), 4.68 (d, J=5.6 Hz, 2H), 3.48 (t, J=23.8 Hz, 6H), 1.44 (d, J=4.9 Hz, 6H).

### Example 17. 4-(4-Fluoro-3-{[4-(2-methyl-3-oxo-1-pyridin-2-yl-2,3-dihydro-1H-pyrazolo[3,4-d]pyrimidin-6-yl) piperazin-1-yl]carbonyl}benzyl)phthalazin-1(2H)-one (5)

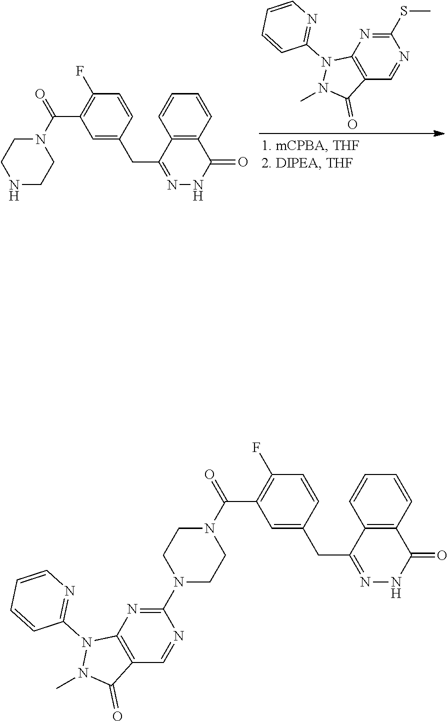 [SMILES: O=C(c1cc(Cc2n[nH]c(=O)c3ccccc23)ccc1F)N1CCNCC1 ; CSc1ncc2c(=O)n(C)n(-c3ccccn3)c2n1 ; Cn1c(=O)c2cnc(N3CCN(C(=O)c4cc(Cc5n[nH]c(=O)c6ccccc56)ccc4F)CC3)nc2n1-c1ccccn1]

To a solution of 2-methyl-6-(methylthio)-1-pyridin-2-yl-1,2-dihydro-3H-pyrazolo[3,4-d]pyrimidin-3-one (P63, 0.043 g, 0.12 mmol) in 2 mL of THF, 77% m-CPBA (0.040 g, 0.18 mmol) was added. The reaction mixture was stirred for 1 h at ambient temperature. After completion of the reaction as indicated by TLC, 4-[4-fluoro-3-(piperazin-1-ylcarbonyl)benzyl]phthalazin-1(2H)-one (P51, 0.05 g, 0.12 mmol) and DIPEA (0.15 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-(4-fluoro-3-{[4-(2-methyl-3-oxo-1-pyridin-2-yl-2,3-dihydro-1H-pyrazolo[3,4-d]pyrimidin-6-yl) piperazin-1-yl]carbonyl}benzyl)phthalazin-1(2H)-one (5, 0.077 g, 75%). ¹H NMR (400 MHz, DMSO-d₆) δ 12.59 (s, 1H), 8.80 (s, 1H), 8.49 (s, 1H), 8.26 (d, J=7.8 Hz, 1H), 8.01 (s, 1H), 7.98 (d, J=7.7 Hz, 1H), 7.95-7.86 (m, 2H), 7.82(t, J=7.4 Hz, 1H), 7.45 (s, 1H), 7.41-7.34 (m, 2H), 7.25 (t, J=9.0 Hz, 1H), 4.34 (s, 2H), 3.94 (s, 2H), 3.75 (d, J=27.1 Hz, 6H), 3.36 (s, 3H).

### Example 18. 4-(3-{[4-({2-Allyl-1-[6-(1-hydroxy-1-methylethyl)pyridin-2-yl]-3-oxo-2,3-dihydro-1H-pyrazolo[3,4-d]pyrimidin-6-yl}amino) piperidin-1-yl]carbonyl}-4-fluorobenzyl)phthalazin-1(2H)-one (4)

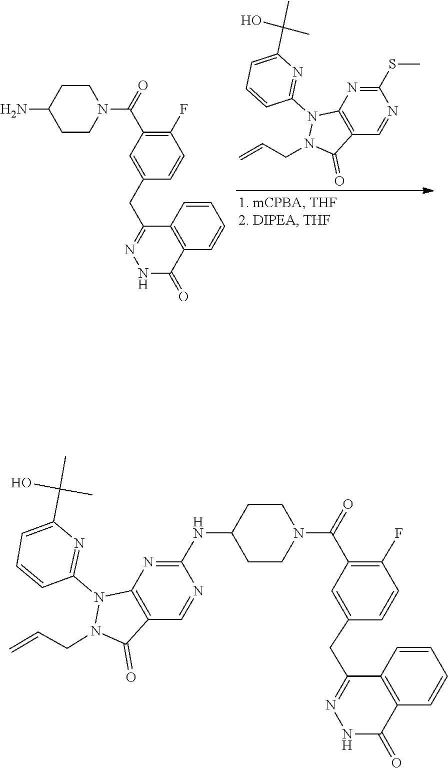 [SMILES: NC1CCN(C(=O)c2cc(Cc3n[nH]c(=O)c4ccccc34)ccc2F)CC1 ; C=CCn1c(=O)c2cnc(SC)nc2n1-c1cccc(C(C)(C)O)n1 ; C=CCn1c(=O)c2cnc(NC3CCN(C(=O)c4cc(Cc5n[nH]c(=O)c6ccccc56)ccc4F)CC3)nc2n1-c1cccc(C(C)(C)O)n1]

To a solution of 2-allyl-1-[6-(1-hydroxy-1-methylethyl)pyridin-2-yl]-6-(methylthio)-1,2-dihydro-3H-pyrazolo[3,4-d]pyrimidin-3-one (P13, 0.043 g, 0.12 mmol) in 2 mL of THF, 77% m-CPBA (0.040 g, 0.18 mmol) was added. The reaction mixture was stirred for 1 h at ambient temperature. After completion of the reaction as indicated by TLC, 4-{3-[(4-aminopiperidin-1-yl)carbonyl]-4-fluorobenzyl}phthalazin-1(2H)-one (P55, 0.05 g, 0.12 mmol) and DIPEA (0.15 mL) in THF were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-(3-{[4-({2-allyl-1-[6-(1-hydroxy-1-methylethyl)pyridin-2-yl]-3-oxo-2,3-dihydro-1H-pyrazolo[3,4-d]pyrimidin-6-yl}amino) piperidin-1-yl]carbonyl}-4-fluorobenzyl)phthalazin-1(2H)-one (4, 0.049 g, 59%). ¹H NMR (400 MHz, DMSO-d₆) δ 12.58 (s, 1H), 8.71 (s, 1H), 8.25 (d, J=7.3 Hz, 2H), 7.97 (d, J=7.8 Hz, 2H), 7.89 (s, 1H), 7.81 (d, J=6.3 Hz, 1H), 7.74 (dd, J=15.1, 8.0 Hz, 1H), 7.62-7.51 (m, 1H), 7.41 (s, 1H), 7.32(s, 1H), 7.22(t, J=8.9 Hz, 1H), 5.73-5.44 (m, 1H), 4.98 (d, J=10.0 Hz, 1H), 4.81 (d, J=16.7 Hz, 1H), 4.68 (d, J=5.2 Hz, 1H), 4.40 (s, 2H), 4.33 (s, 2H), 3.89-4.10 (m, 8H), 1.45 (d, J=3.8 Hz, 6H).

### Example 19. 4-{4-Fluoro-3-[(4-{4-[(2-methyl-3-oxo-1-pyridin-2-yl-2,3-dihydro-1H-pyrazolo[3,4-d]pyrimidin-6-yl)amino]phenyl}piperazin-1-yl)carbonyl]benzyl}phthalazin-1(2H)-one (3)

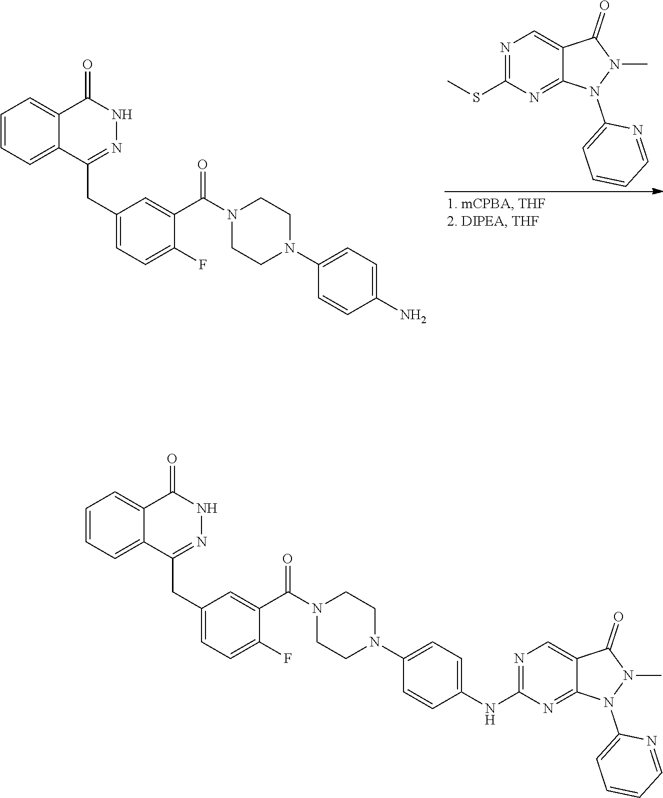 [SMILES: Nc1ccc(N2CCN(C(=O)c3cc(Cc4n[nH]c(=O)c5ccccc45)ccc3F)CC2)cc1 ; CSc1ncc2c(=O)n(C)n(-c3ccccn3)c2n1 ; Cn1c(=O)c2cnc(Nc3ccc(N4CCN(C(=O)c5cc(Cc6n[nH]c(=O)c7ccccc67)ccc5F)CC4)cc3)nc2n1-c1ccccn1]

To a solution of 2-methyl-6-(methylthio)-1-pyridin-2-yl-1,2-dihydro-3H-pyrazolo[3,4-d]pyrimidin-3-one (P63, 0.05 g, 0.18 mmol) in 2 mL of THF, 77% m-CPBA (0.067 g, 0.27 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.084 g, 0.18 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-{4-fluoro-3-[(4-{4-[(2-methyl-3-oxo-1-pyridin-2-yl-2,3-dihydro-1H-pyrazolo[3,4-d]pyrimidin-6-yl)amino]phenyl}piperazin-1-yl)carbonyl]benzyl}phthalazin-1(2H)-one (3, 0.023 g, 18%). ¹H NMR (400 MHz, DMSO-d₆) δ 12.58 (s, 1H), 10.12(s, 1H), 8.82(s, 1H), 8.55 (s, 1H), 8.27 (d, J=7.0 Hz, 1H), 8.10 (s, 1H), 8.04-7.75 (m, 3H), 7.61 (s, 2H), 7.42(d, J=21.6 Hz, 3H), 7.26 (d, J=8.6 Hz, 1H), 6.95 (d, J=8.3 Hz, 2H), 4.35 (s, 3H), 3.78 (s, 2H), 3.30 (s, 2H), 3.17 (s, 2H), 3.01 (s, 2H).

### Example 20. 4-{4-Fluoro-3-[(4-{4-[(1-methyl-3-oxo-2-pyridin-2-yl-2,3-dihydro-1H-pyrazolo[3,4-d]pyrimidin-6-yl)amino]phenyl}piperazin-1-yl)carbonyl]benzyl}phthalazin-1(2H)-one (12)

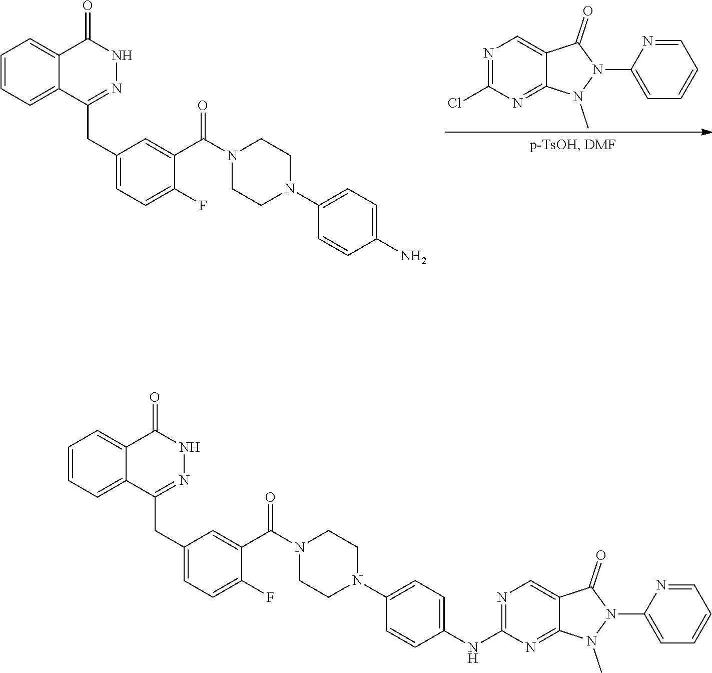 [SMILES: Nc1ccc(N2CCN(C(=O)c3cc(Cc4n[nH]c(=O)c5ccccc45)ccc3F)CC2)cc1 ; Cn1c2nc(Cl)ncc2c(=O)n1-c1ccccn1 ; Cn1c2nc(Nc3ccc(N4CCN(C(=O)c5cc(Cc6n[nH]c(=O)c7ccccc67)ccc5F)CC4)cc3)ncc2c(=O)n1-c1ccccn1]

To a solution of 4-(3-{[4-(4-aminophenyl) piperazin-1-yl]carbonyl}-4-fluorobenzyl)phthalazin-1(2H)-one (P49, 0.053 g, 0.12 mmol) in 5 mL DMF, and 6-chloro-1-methyl-2-pyridin-2-yl-1,2-dihydro-3H-pyrazolo[3,4-d]pyrimidin-3-one (P46, 0.030 g, 0.12 mmol), p-TsOH was added (0.02 g, 0.12 mmol) and the mixture was stirred at 80° C. for 15 h. The reaction mixture was quenched with aq. NaHCO₃, the formed pellet was filtered off, washed with H₂O and EtOAc and dried. The obtained crude residue was purified by RP-HPLC eluting with a gradient MeCN-H₂O+0.1% TFA to provide 4-{4-fluoro-3-[(4-{4-[(1-methyl-3-oxo-2-pyridin-2-yl-2,3-dihydro-1H-pyrazolo[3,4-d]pyrimidin-6-yl)amino]phenyl}piperazin-1-yl)carbonyl]benzyl}phthalazin-1(2H)-one (12, 0.020 g, 25%). ¹H NMR (400 MHz, DMSO-d₆) δ 12.58 (s, 1H), 10.15 (s, 1H), 8.80 (s, 1H), 8.53 (d, J=4.6 Hz, 1H), 8.27 (d, J=7.9 Hz, 1H), 7.98 (d, J=5.9 Hz, 2H), 7.90 (t, J=7.3 Hz, 1H), 7.84 (d, J=5.9 Hz, 2H), 7.68 (s, 2H), 7.45 (s, 1H), 7.38 (d, J=5.5 Hz, 1H), 7.36-7.29 (m, 1H), 7.25 (t, J=8.9 Hz, 1H), 6.96 (d, J=8.5 Hz, 2H), 4.34 (s, 2H), 3.78 (s, 2H), 3.49 (s, 3H), 3.30 (s, 2H), 3.17 (s, 2H), 3.01 (s, 2H).

### Example 21. 4-(4-Fluoro-3-{[4-(4-{[2-(2-methoxyethyl)-3-oxo-1-pyridin-2-yl-2,3-dihydro-1H-pyrazolo[3,4-d]pyrimidin-6-yl]amino}phenyl) piperazin-1-yl]carbonyl}benzyl)phthalazin-1(2H)-one (9)

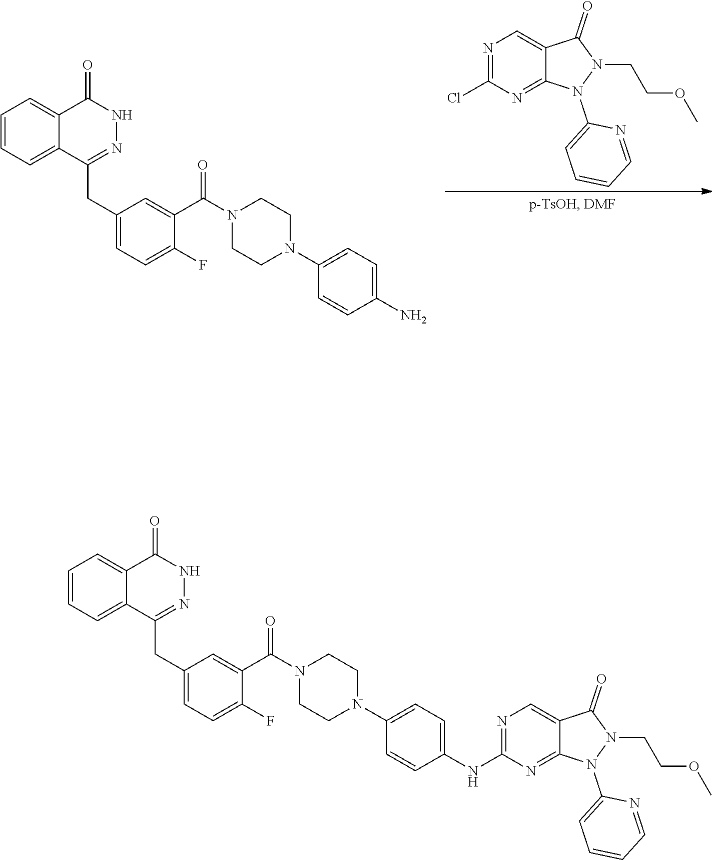 [SMILES: Nc1ccc(N2CCN(C(=O)c3cc(Cc4n[nH]c(=O)c5ccccc45)ccc3F)CC2)cc1 ; COCCn1c(=O)c2cnc(Cl)nc2n1-c1ccccn1 ; COCCn1c(=O)c2cnc(Nc3ccc(N4CCN(C(=O)c5cc(Cc6n[nH]c(=O)c7ccccc67)ccc5F)CC4)cc3)nc2n1-c1ccccn1]

To a solution of 4-(3-{[4-(4-aminophenyl) piperazin-1-yl]carbonyl}-4-fluorobenzyl)phthalazin-1(2H)-one (P49, 0.26 g, 0.57 mmol) in 15 mL DMF, and 6-chloro-2-(2-methoxyethyl)-1-pyridin-2-yl-1,2-dihydro-3H-pyrazolo[3,4-d]pyrimidin-3-one (P64, 0.17 g, 0.57 mmol), p-TsOH was added (0.098 g, 0.57 mmol) and the mixture was stirred at 80° C. for 15 h. The reaction mixture was quenched with aq. NaHCO₃, the formed pellet was filtered off, washed with H₂O and EtOAc and dried. The obtained crude residue was purified by RP-HPLC eluting with a gradient MeCN-H₂O+0.1% TFA to provide 4-(4-fluoro-3-{[4-(4-{[2-(2-methoxyethyl)-3-oxo-1-pyridin-2-yl-2,3-dihydro-1H-pyrazolo[3,4-d]pyrimidin-6-yl]amino}phenyl) piperazin-1-yl]carbonyl}benzyl)phthalazin-1(2H)-one (9, 0.12 g, 29%). ¹H NMR (400 MHz, DMSO-d₆) δ 12.59 (s, 1H), 10.14 (s, 1H), 8.81 (s, 1H), 8.52(d, J=3.9 Hz, 1H), 8.27 (d, J=7.0 Hz, 1H), 8.10 (s, 1H), 8.03-7.80 (m, 4H), 7.59 (s, 2H), 7.45 (dd, J=7.2, 4.4 Hz, 1H), 7.42-7.34 (m, 2H), 7.25 (t, J=9.0 Hz, 1H), 6.95 (d, J=9.0 Hz, 2H), 4.35 (s, 2H), 4.12(d, J=25.2 Hz, 2H), 3.78 (s, 2H), 3.40-3.33 (m, 4H), 3.17 (s, 2H), 3.08 (s, 3H), 3.01 (s, 2H).

### Example 22. 4-(4-Fluoro-3-{[4-(4-{[2-(2-hydroxyethyl)-3-oxo-1-pyridin-2-yl-2,3-dihydro-1H-pyrazolo[3,4-d]pyrimidin-6-yl]amino}phenyl) piperazin-1-yl]carbonyl}benzyl)phthalazin-1(2H)-one (15)

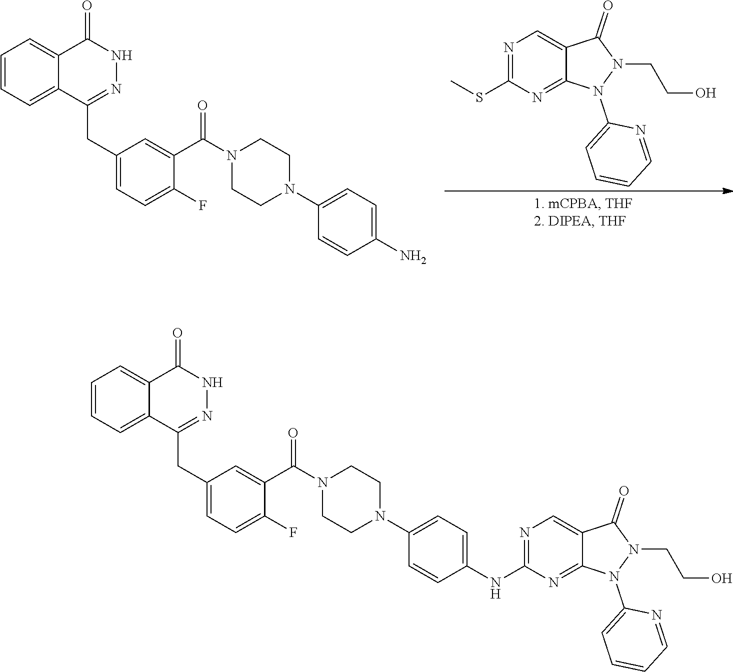 [SMILES: Nc1ccc(N2CCN(C(=O)c3cc(Cc4n[nH]c(=O)c5ccccc45)ccc3F)CC2)cc1 ; CSc1ncc2c(=O)n(CCO)n(-c3ccccn3)c2n1 ; O=C(c1cc(Cc2n[nH]c(=O)c3ccccc23)ccc1F)N1CCN(c2ccc(Nc3ncc4c(=O)n(CCO)n(-c5ccccn5)c4n3)cc2)CC1]

To a solution of 2-(2-hydroxyethyl)-6-(methylthio)-1-pyridin-2-yl-1,2-dihydro-3H-pyrazolo[3,4-d]pyrimidin-3-one (P19, 0.086 g, 0.28 mmol) in 3 mL of THF, 77% m-CPBA (0.14 g, 0.61 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.13 g, 0.28 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-(4-fluoro-3-{[4-(4-{[2-(2-hydroxyethyl)-3-oxo-1-pyridin-2-yl-2,3-dihydro-1H-pyrazolo[3,4-d]pyrimidin-6-yl]amino}phenyl) piperazin-1-yl]carbonyl}benzyl)phthalazin-1(2H)-one (15, 0.018 g, 9%). ¹H NMR (400 MHz, DMSO-d₆) δ 12.60 (s, 1H), 10.13 (s, 1H), 8.81 (s, 1H), 8.52(d, J=3.9 Hz, 1H), 8.27 (d, J=7.7 Hz, 1H), 8.11 (s, 1H), 8.03-7.79 (m, 4H), 7.61 (s, 1H), 7.45 (s, 1H), 7.39 (d, J=6.4 Hz, 2H), 7.25 (t, J=8.9 Hz, 1H), 6.96 (d, J=8.4 Hz, 2H), 4.35 (s, 2H), 4.04 (s, 3H), 3.78 (s, 2H), 3.38 (t, J=5.7 Hz, 2H), 3.33 (s, 2H), 3.18 (s, 2H), 3.01 (s, 2H).

### Example 23. 4-{3-[(4-{4-[(2-Allyl-3-oxo-1-pyridin-2-yl-2,3-dihydro-1H-pyrazolo[3,4-d]pyrimidin-6-yl)amino]phenyl}piperazin-1-yl)carbonyl]-4-fluorobenzyl}phthalazin-1(2H)-one (10)

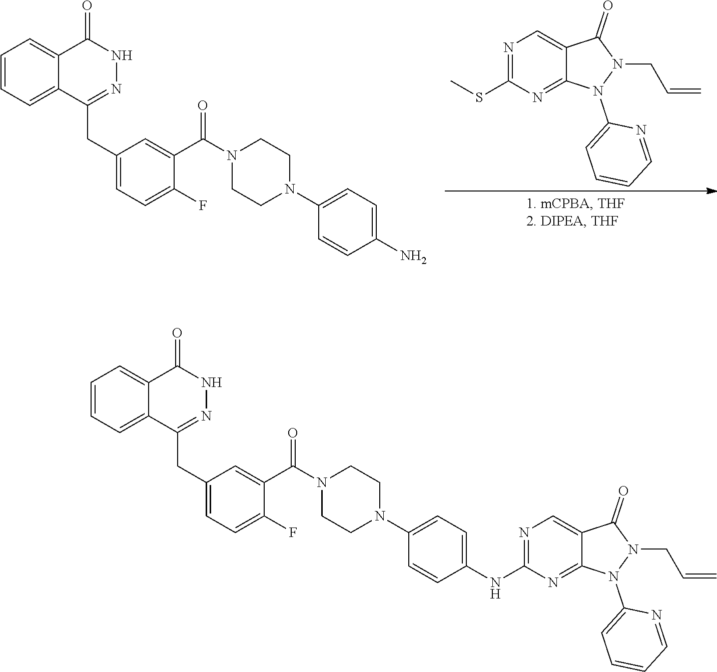 [SMILES: Nc1ccc(N2CCN(C(=O)c3cc(Cc4n[nH]c(=O)c5ccccc45)ccc3F)CC2)cc1 ; C=CCn1c(=O)c2cnc(SC)nc2n1-c1ccccn1 ; C=CCn1c(=O)c2cnc(Nc3ccc(N4CCN(C(=O)c5cc(Cc6n[nH]c(=O)c7ccccc67)ccc5F)CC4)cc3)nc2n1-c1ccccn1]

To a solution of 2-allyl-6-(methylthio)-1-pyridin-2-yl-1,2-dihydro-3H-pyrazolo[3,4-d]pyrimidin-3-one (P14, 0.066 g, 0.22 mmol) in 3 mL of THF, 77% m-CPBA (0.11 g, 0.48 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.10 g, 0.22 mmol) and DIPEA (0.4 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-{3-[(4-{4-[(2-allyl-3-oxo-1-pyridin-2-yl-2,3-dihydro-1H-pyrazolo[3,4-d]pyrimidin-6-yl)amino]phenyl}piperazin-1-yl)carbonyl]-4-fluorobenzyl}phthalazin-1(2H)-one (10, 0.034 g, 22%). ¹H NMR (400 MHz, DMSO-d₆) δ 12.57 (s, 1H), 10.14 (s, 1H), 8.83 (s, 1H), 8.54 (d, J=4.5 Hz, 1H), 8.27 (d, J=7.8 Hz, 1H), 8.08 (s, 1H), 7.98 (d, J=7.8 Hz, 1H), 7.94-7.77 (m, 2H), 7.60 (d, J=8.0 Hz, 2H), 7.45 (s, 1H), 7.41-7.34 (m, 2H), 7.24 (dd, J=18.2, 9.2 Hz, 1H), 6.94 (d, J=9.0 Hz, 2H), 5.82-5.52(m, 1H), 5.02(d, J=10.2 Hz, 1H), 4.88 (d, J=16.6 Hz, 1H), 4.59 (s, 2H), 4.35 (s, 2H), 3.78 (s, 3H), 3.32(s, 2H), 3.17 (s, 2H), 3.01 (s, 2H).

### Example 24. 4-{4-Fluoro-3-[(4-{4-[(2-methyl-3-oxo-1-pyrimidin-2-yl-2,3-dihydro-1H-pyrazolo[3,4-d]pyrimidin-6-yl)amino]phenyl}piperazin-1-yl)carbonyl]benzyl}phthalazin-1(2H)-one (11)

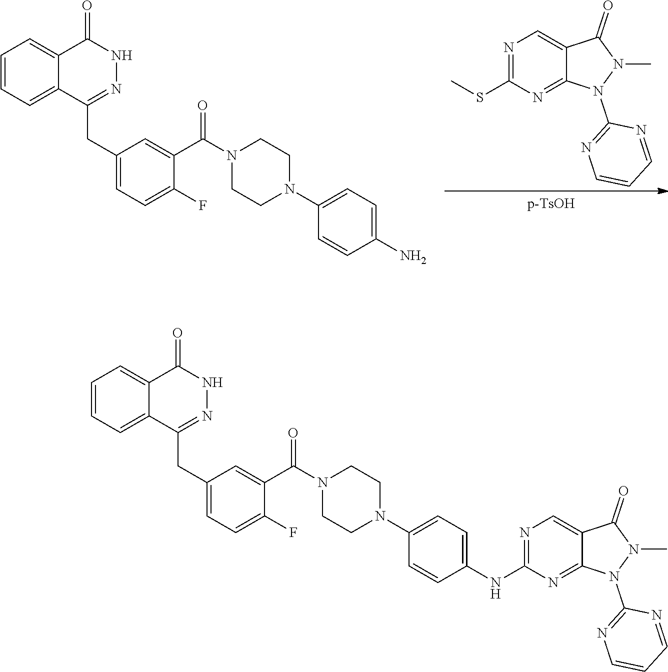 [SMILES: Nc1ccc(N2CCN(C(=O)c3cc(Cc4n[nH]c(=O)c5ccccc45)ccc3F)CC2)cc1 ; CSc1ncc2c(=O)n(C)n(-c3ncccn3)c2n1 ; Cn1c(=O)c2cnc(Nc3ccc(N4CCN(C(=O)c5cc(Cc6n[nH]c(=O)c7ccccc67)ccc5F)CC4)cc3)nc2n1-c1ncccn1]

To a solution of 4-(3-{[4-(4-aminophenyl) piperazin-1-yl]carbonyl}-4-fluorobenzyl)phthalazin-1(2H)-one (P49, 0.08 g, 0.18 mmol) in 5 mL DMF, and 2-methyl-6-(methylthio)-1-pyrimidin-2-yl-1,2-dihydro-3H-pyrazolo[3,4-d]pyrimidin-3-one (P2, 0.046 g, 0.18 mmol), p-TsOH was added (0.03 g, 0.18 mmol) and the mixture was stirred at 80° C. for 15 h. The reaction mixture was quenched with aq. NaHCO₃, the formed pellet was filtered off, washed with H₂O and EtOAc and dried. The obtained crude residue was purified by RP-HPLC eluting with a gradient MeCN—H₂O +0.1% TFA to provide 4-{4-fluoro-3-[(4-{4-[(2-methyl-3-oxo-1-pyrimidin-2-yl-2,3-dihydro-1H-pyrazolo[3,4-d]pyrimidin-6-yl)amino]phenyl}piperazin-1-yl)carbonyl]benzyl}phthalazin-1(2H)-one (11, 0.023 g, 19%). ¹H NMR (400 MHz, DMSO-d₆) δ 12.59 (s, 1H), 10.23 (s, 1H), 9.01 (d, J=4.3 Hz, 2H), 8.84 (s, 1H), 8.27 (d, J=7.8 Hz, 1H), 7.98 (d, J=7.9 Hz, 2H), 7.90 (t, J=7.5 Hz, 1H), 7.84 (t, J=7.5 Hz, 1H), 7.51 (t, J=4.8 Hz, 1H), 7.45 (s, 1H), 7.39 (d, J=6.4 Hz, 1H), 7.25 (t, J=9.0 Hz, 1H), 6.98 (d, J=8.5 Hz, 2H), 4.35 (s, 2H), 3.78 (s, 3H), 3.41 (s, 3H), 3.33 (s, 2H), 3.18 (s, 2H), 3.02(s, 2H).

### Example 25. 4-[3-({4-[4-({2-Allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)-4-fluorobenzyl]phthalazin-1(2H)-one (14)

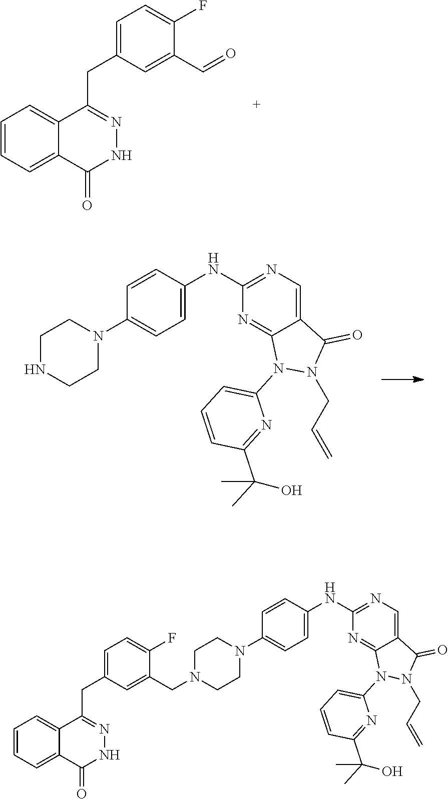 [SMILES: O=Cc1cc(Cc2n[nH]c(=O)c3ccccc23)ccc1F ; C=CCn1c(=O)c2cnc(Nc3ccc(N4CCNCC4)cc3)nc2n1-c1cccc(C(C)(C)O)n1 ; C=CCn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1]

To a mixture of 2-fluoro-5-[(4-oxo-3,4-dihydrophthalazin-1-yl)methyl]benzaldehyde (P58, 0.08 g, 0.28 mmol) and 2-allyl-1-[6-(1-hydroxy-1-methylethyl)pyridin-2-yl]-6-[(4-piperazin-1-ylphenyl)amino]-1,2-dihydro-3H-pyrazolo[3,4-d]pyrimidin-3-one (P60, 0.14 g, 0.28 mmol) in 20 mL MeOH, acetic acid was added (17 μl, 0.28 mmol) and the mixture was stirred for 1 h at ambient temperature. Sodium cyanoborohydride was added (0.11 g, 1.68 mmol) and the mixture was stirred at ambient temperature for 15 h. The mixture was quenched with aq. NaHCO₃, extracted with CHCl₃, the combined organic extracts were dried with Na₂SO₄ and evaporated. The obtained crude residue was purified by RP-HPLC eluting with a gradient MeCN-H₂O+0.1% TFA to provide 4-[3-({4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)-4-fluorobenzyl]phthalazin-1(2H)-one (14, 0.034 g, 16%). ¹H NMR (400 MHz, DMSO-d₆) δ 12.61 (s, 1H), 10.99 (s, 1H), 10.16 (s, 1H), 8.83 (s, 1H), 8.26 (d, J=7.2 Hz, 1H), 8.08-7.92(m, 2H), 7.93-7.78 (m, 2H), 7.73 (d, J=16.3 Hz, 2H), 7.62(s, 2H), 7.50 (s, 1H), 7.29 (t, J=25.4 Hz, 2H), 6.97 (s, 2H), 5.66 (s, 1H), 4.99 (d, J=10.1 Hz, 1H), 4.82(d, J=17.1 Hz, 1H), 4.68 (s, 2H), 4.39 (s, 2H), 4.33 (s, 2H), 3.78-3.67 (m, 4H), 3.40 (s, 2H), 3.13 (s, 4H), 1.46 (s, 6H).

### Example 26. 4-[4-Fluoro-3-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}methyl)benzyl]phthalazin-1(2H)-one (8)

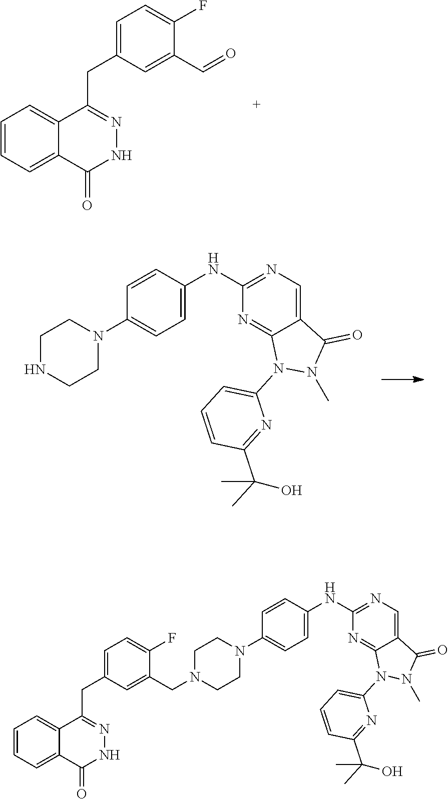 [SMILES: O=Cc1cc(Cc2n[nH]c(=O)c3ccccc23)ccc1F ; Cn1c(=O)c2cnc(Nc3ccc(N4CCNCC4)cc3)nc2n1-c1cccc(C(C)(C)O)n1 ; Cn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1]

To a mixture of 2-fluoro-5-[(4-oxo-3,4-dihydrophthalazin-1-yl)methyl]benzaldehyde (P58, 0.075 g, 0.27 mmol) and 1-[6-(1-hydroxy-1-methylethyl)pyridin-2-yl]-2-methyl-6-[(4-piperazin-1-ylphenyl)amino]-1,2-dihydro-3H-pyrazolo[3,4-d]pyrimidin-3-one (P62, 0.12 g, 0.27 mmol) in 20 mL MeOH, acetic acid was added (16 μl, 0.27 mmol) and the mixture was stirred for 1 h at ambient temperature. Sodium cyanoborohydride was added (0.11 g, 1.68 mmol) and the mixture was stirred at ambient temperature for 15 h. The mixture was quenched with aq. NaHCO₃, extracted with CHCl₃, the combined organic extracts were dried with Na₂SO₄ and evaporated. The obtained crude residue was purified by RP-HPLC eluting with a gradient MeCN-H₂O+0.1% TFA to provide 4-[4-fluoro-3-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}methyl)benzyl]phthalazin-1(2H)-one (8, 0.024 g, 12%). ¹H NMR (400 MHz, DMSO-d₆) δ 12.61 (s, 1H), 11.00 (s, 1H), 10.13 (s, 1H), 8.82(s, 1H), 8.27 (d, J=7.5 Hz, 1H), 8.05 (t, J=7.7 Hz, 1H), 7.97 (d, J=8.1 Hz, 1H), 7.93-7.74 (m, 3H), 7.71 (d, J=6.3 Hz, 1H), 7.62(d, J=7.5 Hz, 3H), 7.50 (s, 1H), 7.29 (t, J=9.0 Hz, 1H), 6.97 (d, J=8.7 Hz, 2H), 4.39 (s, 3H), 4.33 (s, 2H), 3.74 (d, J=11.1 Hz, 2H), 3.41 (s, 5H), 3.12(d, J=12.3 Hz, 4H), 1.45 (s, 6H).

### Example 27. 4-[4-Fluoro-3-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}carbonyl)benzyl]phthalazin-1(2H)-one (7)

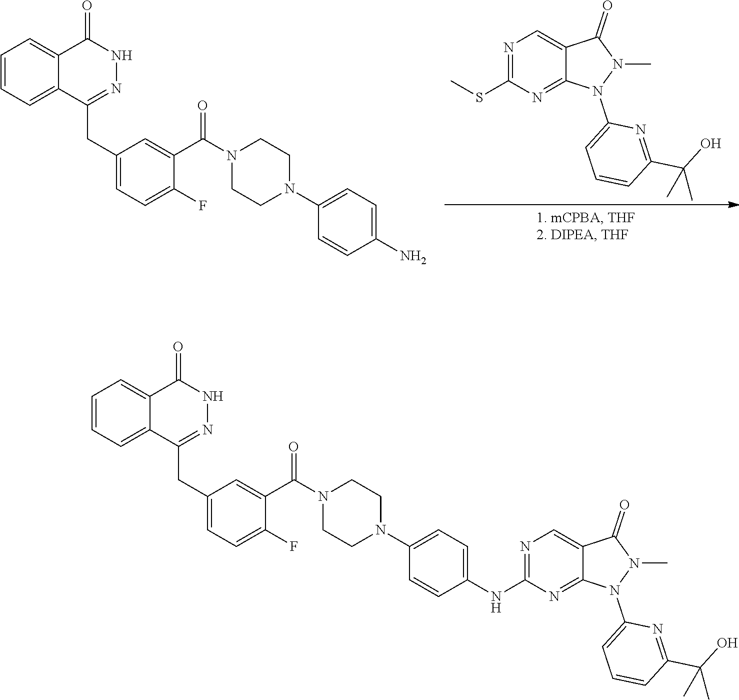 [SMILES: Nc1ccc(N2CCN(C(=O)c3cc(Cc4n[nH]c(=O)c5ccccc45)ccc3F)CC2)cc1 ; CSc1ncc2c(=O)n(C)n(-c3cccc(C(C)(C)O)n3)c2n1 ; Cn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)n1]

To a solution of 1-[6-(1-hydroxy-1-methylethyl)pyridin-2-yl]-2-methyl-6-(methylthio)-1,2-dihydro-3H-pyrazolo[3,4-d]pyrimidin-3-one (P47, 0.15 g, 0.43 mmol) in 10 mL of THF, 77% m-CPBA (0.22 g, 0.95 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.20 g, 0.43 mmol) and DIPEA (0.8 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-[4-fluoro-3-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}carbonyl)benzyl]phthalazin-1(2H)-one (7, 0.031 g, 10%). 1H NMR (400 MHz, DMSO-d₆) δ 12.60 (s, 1H), 10.15 (s, 1H), 8.81 (s, 1H), 8.81 (s, 1H), 8.27 (d, J=7.8 Hz, 1H), 8.07 (s, 1H), 7.98 (d, J=7.7 Hz, 1H), 7.90 (t, J=7.0 Hz, 1H), 7.83 (t, J=8.0 Hz, 2H), 7.61 (d, J=7.6 Hz, 3H), 7.46 (d, J=5.4 Hz, 1H), 7.39 (d, J=6.5 Hz, 1H), 7.25 (t, J=9.0 Hz, 1H), 6.97 (d, J=9.1 Hz, 2H), 4.35 (s, 2H), 4.21 (s, 3H), 3.79 (s, 2H), 3.19 (s, 2H), 3.07 (s, 2H), 3.02(s, 2H), 1.43 (s, 6H).

### Example 28. 4-(4-Fluoro-3-{[4-(4-{[1-[6-(1-hydroxy-1-methylethyl)pyridin-2-yl]-2-(2-methoxyethyl)-3-oxo-2,3-dihydro-1H-pyrazolo[3,4-d]pyrimidin-6-yl]amino}phenyl) piperazin-1-yl]carbonyl}benzyl)phthalazin-1(2H)-one (13)

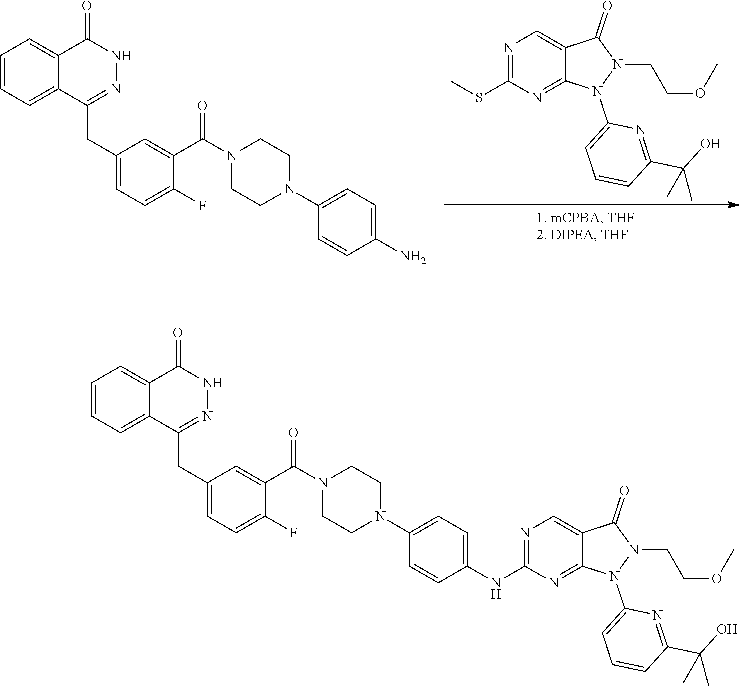

To a solution of 1-[6-(1-hydroxy-1-methylethyl)pyridin-2-yl]-2-(2-methoxyethyl)-6-(methylthio)-1,2-dihydro-3H-pyrazolo[3,4-d]pyrimidin-3-one (P17, 0.16 g, 0.43 mmol) in 10 mL of THF, 77% m-CPBA (0.22 g, 0.95 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.20 g, 0.43 mmol) and DIPEA (0.8 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-(4-fluoro-3-{[4-(4-{[1-[6-(1-hydroxy-1-methylethyl)pyridin-2-yl]-2-(2-methoxyethyl)-3-oxo-2,3-dihydro-1H-pyrazolo[3,4-d]pyrimidin-6-yl]amino}phenyl) piperazin-1-yl]carbonyl}benzyl)phthalazin-1(2H)-one (13, 0.025 g, 7%). ¹H NMR (400 MHz, DMSO-d₆) § 12.60 (s, 1H), 10.15 (s, 1H), 8.81 (s, 1H), 8.81 (s, 1H), 8.27 (d, J=7.8 Hz, 1H), 8.07 (s, 1H), 7.98 (d, J=7.7 Hz, 1H), 7.90 (t, J=7.0 Hz, 1H), 7.83 (t, J=8.0 Hz, 2H), 7.61 (d, J=7.6 Hz, 3H), 7.46 (d, J=5.4 Hz, 1H), 7.39 (d, J=6.5 Hz, 1H), 7.25 (t, J=9.0 Hz, 1H), 6.97 (d, J=9.1 Hz, 2H), 4.35 (s, 2H), 4.21 (s, 3H), 3.79 (s, 2H), 3.34 (t, J=5.3 Hz, 4H), 3.19 (s, 2H), 3.07 (s, 2H), 3.02(s, 2H), 1.43 (s, 6H).

### Example 29. 4-[4-Fluoro-3-({4-[4-({2-(2-hydroxyethyl)-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}carbonyl)benzyl]phthalazin-1(2H)-one (98)

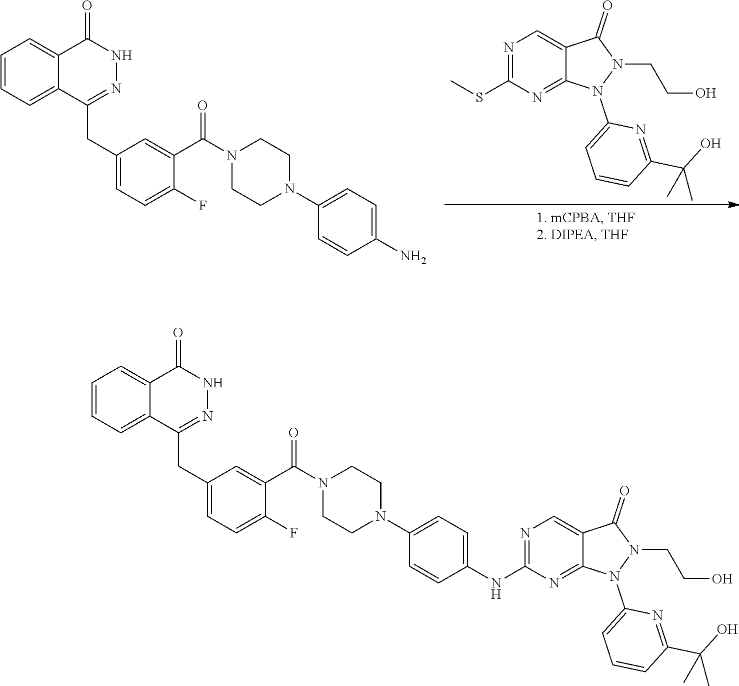 [SMILES: Nc1ccc(N2CCN(C(=O)c3cc(Cc4n[nH]c(=O)c5ccccc45)ccc3F)CC2)cc1 ; CSc1ncc2c(=O)n(CCO)n(-c3cccc(C(C)(C)O)n3)c2n1 ; CC(C)(O)c1cccc(-n2c3nc(Nc4ccc(N5CCN(C(=O)c6cc(Cc7n[nH]c(=O)c8ccccc78)ccc6F)CC5)cc4)ncc3c(=O)n2CCO)n1]

To a solution of 2-(2-hydroxyethyl)-1-[6-(1-hydroxy-1-methylethyl)pyridin-2-yl]-6-(methylthio)-1,2-dihydro-3H-pyrazolo[3,4-d]pyrimidin-3-one (P20, 0.15 g, 0.43 mmol) in 10 mL of THF, 77% m-CPBA (0.22 g, 0.95 mmol) was added. The reaction mixture was stirred for 1 h at ambient temperature. After completion of the reaction as indicated by TLC, 4-(3-{[4-(4-aminophenyl) piperazin-1-yl]carbonyl}-4-fluorobenzyl)phthalazin-1(2H)-one (P49, 0.20 g, 0.43 mmol) and DIPEA (0.8 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 4-[4-fluoro-3-({4-[4-({2-(2-hydroxyethyl)-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}carbonyl)benzyl]phthalazin-1(2H)-one (98, 0.018 g, 5%). ¹H NMR (400 MHz, DMSO-d₆) § 12.60 (s, 1H), 8.80 (s, 1H), 8.27 (d, J=7.6 Hz, 1H), 8.07 (s, 1H), 7.98 (d, J=8.2 Hz, 1H), 7.90 (t, J=7.2 Hz, 1H), 7.83 (dd, J=16.3, 8.3 Hz, 2H), 7.61 (d, J=7.5 Hz, 3H), 7.45 (s, 1H), 7.39 (d, J=4.4 Hz, 1H), 7.25 (t, J=9.1 Hz, 2H), 6.95 (d, J=8.4 Hz, 2H), 5.30 (br. s, 1H), 4.35 (s, 2H), 4.10 (s, 2H), 3.78 (s, 2H), 3.30 (m, 4H), 3.17 (s, 2H), 3.01 (s, 2H), 1.43 (s, 6H).

### Example 30. 6-Fluoro-2-[4-[[4-[4-[[1-methyl-3-oxo-2-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one (37)

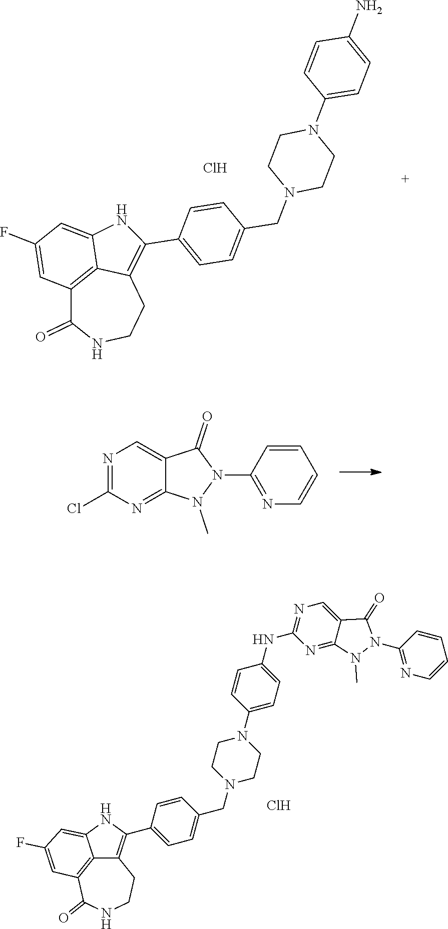 [SMILES: Nc1ccc(N2CCN(Cc3ccc(-c4[nH]c5cc(F)cc6c5c4CCNC6=O)cc3)CC2)cc1 ; Cl ; Cn1c2nc(Cl)ncc2c(=O)n1-c1ccccn1 ; Cn1c2nc(Nc3ccc(N4CCN(Cc5ccc(-c6[nH]c7cc(F)cc8c7c6CCNC8=O)cc5)CC4)cc3)ncc2c(=O)n1-c1ccccn1 ; Cl]

To a solution of 2-(4-{[4-(4-Aminophenyl) piperazin-1-yl]methyl}phenyl)-8-fluoro-1,3,4,5-tetrahydro-6H-azepino[5,4,3-cd]indol-6-one (P39, 0.15 g, 0.29 mmol) in 5 mL DMF, and 6-chloro-1-methyl-2-pyridin-2-yl-1,2-dihydro-3H-pyrazolo[3,4-d]pyrimidin-3-one (P46, 0.077 g, 0.29 mmol), p-TsOH was added (0.056 g, 0.29 mmol) and the mixture was stirred at 80° C. for 15 h. The reaction mixture was quenched with aq. NaHCO₃, the formed pellet was filtered off, washed with H₂O and EtOAc and dried. The residue was purified on a silica gel column eluting with 1→10% MeOH in CHCl₃ 6-fluoro-2-[4-[[4-[4-[[1-methyl-3-oxo-2-(2-pyridyl)pyrazolo[3,4-d]pyrimidin-6-yl]amino]phenyl]piperazin-1-yl]methyl]phenyl]-3,10-diazatricyclo[6.4.1.04,13]trideca-1,4,6,8 (13)-tetraen-9-one (37, 0.075 g, 34%). ¹H NMR (400 MHz, DMSO-d₆) δ 11.86 (s, 1H), 11.29 (s, 1H), 10.21 (s, 1H), 8.81 (s, 1H), 8.53 (s, 1H), 8.29 (s, 1H), 7.98 (t, J=6.8 Hz, 1H), 7.88-7.79 (m, 3H), 7.74 (d, J=8.0 Hz, 4H), 7.44 (d, J=10.9 Hz, 1H), 7.40-7.28 (m, 1H), 7.01 (d, J=8.9 Hz, 1H), 4.45 (s, 1H), 3.80 (s, 2H), 3.49 (br. s, 4H), 3.41 (s, 3H), 3.30 (m, 4H), 3.21 (s, 2H), 3.08 (s, 2H).

### Example 31. 8-Fluoro-2-(4-{[4-(4-{[1-[6-(1-hydroxy-1-methylethyl)pyridin-2-yl]-2-(2-methoxyethyl)-3-oxo-2,3-dihydro-1H-pyrazolo[3,4-d]pyrimidin-6-yl]amino}phenyl) piperazin-1-yl]methyl}phenyl)-1,3,4,5-tetrahydro-6H-azepino[5,4,3-cd]indol-6-one (41)

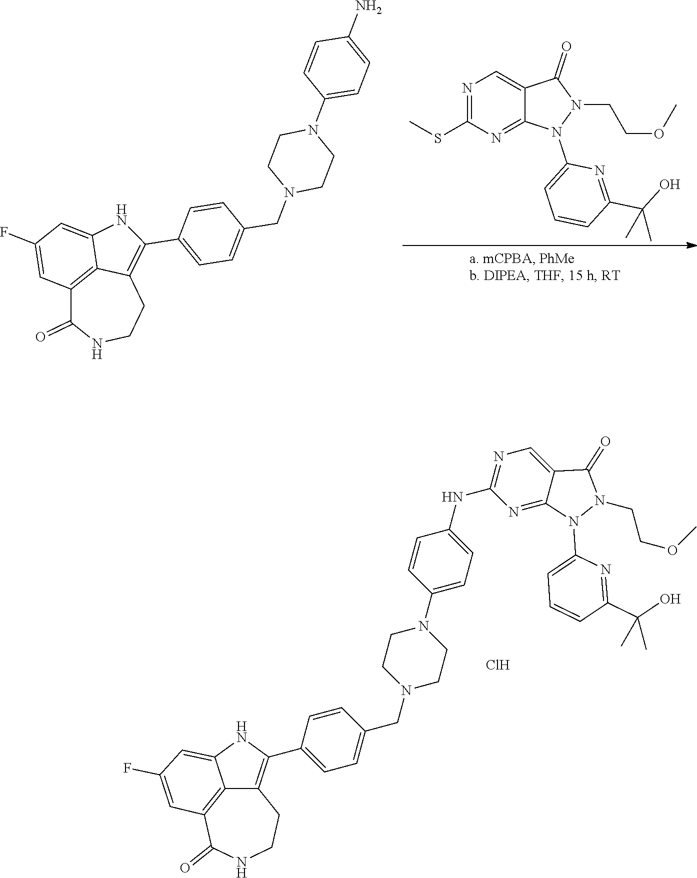

To a solution of 1-[6-(1-hydroxy-1-methylethyl)pyridin-2-yl]-2-(2-methoxyethyl)-6-(methylthio)-1,2-dihydro-3H-pyrazolo[3,4-d]pyrimidin-3-one (P17, 0.17 g, 0.42 mmol) in 2 mL of PhMe, 77% m-CPBA (0.16 g, 0.70 mmol) was added. The reaction mixture was stirred for 1 h at ambient temperature. After completion of the reaction as indicated by TLC, 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.17 g, 0.35 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 8-fluoro-2-(4-{[4-(4-{[1-[6-(1-hydroxy-1-methylethyl)pyridin-2-yl]-2-(2-methoxyethyl)-3-oxo-2,3-dihydro-1H-pyrazolo[3,4-d]pyrimidin-6-yl]amino}phenyl) piperazin-1-yl]methyl}phenyl)-1,3,4,5-tetrahydro-6H-azepino[5,4,3-cd]indol-6-one (41, 0.024 g, 9%). ¹H NMR (400 MHz, DMSO-d₆) δ 11.86 (s, 1H), 11.29 (s, 1H), 10.18 (s, 1H), 8.82(s, 1H), 8.29 (s, 1H), 8.04 (d, J=7.9 Hz, 1H), 7.81 (d, J=7.7 Hz, 3H), 7.75 (d, J=8.1 Hz, 2H), 7.61 (d, J=7.4 Hz, 3H), 7.44 (d, J=10.9 Hz, 1H), 7.37 (d, J=8.9 Hz, 1H), 6.99 (d, J=8.6 Hz, 2H), 4.46 (s, 2H), 4.17 (d, J=29.4 Hz, 6H), 3.79 (d, J=8.8 Hz, 2H), 3.42(s, 4H), 3.35 (d, J=4.8 Hz, 2H), 3.19 (d, J=9.0 Hz, 4H), 3.08 (s, 2H), 1.68 (s, 6H).

### Example 32. 8-Fluoro-2-[4-({4-[4-({2-(2-hydroxyethyl)-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)phenyl]-1,3,4,5-tetrahydro-6H-azepino[5,4,3-cd]indol-6-one (42)

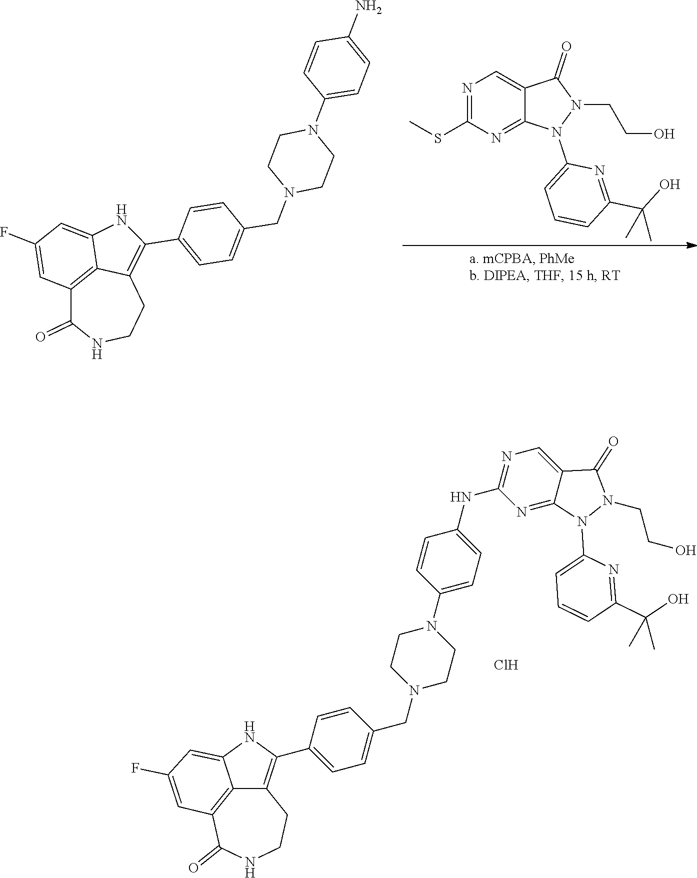 [SMILES: Nc1ccc(N2CCN(Cc3ccc(-c4[nH]c5cc(F)cc6c5c4CCNC6=O)cc3)CC2)cc1 ; CSc1ncc2c(=O)n(CCO)n(-c3cccc(C(C)(C)O)n3)c2n1 ; CC(C)(O)c1cccc(-n2c3nc(Nc4ccc(N5CCN(Cc6ccc(-c7[nH]c8cc(F)cc9c8c7CCNC9=O)cc6)CC5)cc4)ncc3c(=O)n2CCO)n1 ; Cl]

To a solution of 2-(2-hydroxyethyl)-1-[6-(1-hydroxy-1-methylethyl)pyridin-2-yl]-6-(methylthio)-1,2-dihydro-3H-pyrazolo[3,4-d]pyrimidin-3-one (P20, 0.17 g, 0.42 mmol) in 2 mL of PhMe, 77% m-CPBA (0.16 g, 0.70 mmol) was added. The reaction mixture was stirred for 1 h at ambient temperature. After completion of the reaction as indicated by TLC, 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.17 g, 0.35 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 8-fluoro-2-[4-({4-[4-({2-(2-hydroxyethyl)-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)phenyl]-1,3,4,5-tetrahydro-6H-azepino[5,4,3-cd]indol-6-one (42, 0.061 g, 22%). ¹H NMR (400 MHz, DMSO-d₆) δ 11.86 (s, 1H), 11.27 (s, 1H), 10.13 (s, 1H), 8.81 (s, 1H), 8.29 (s, 1H), 8.05 (t, J=7.8 Hz, 1H), 7.81 (d, J=8.0 Hz, 4H), 7.75 (d, J=8.1 Hz, 3H), 7.61 (d, J=7.8 Hz, 2H), 7.44 (dd, J=11.0, 2.3 Hz, 1H), 7.37 (dd, J=9.0, 2.3 Hz, 1H), 6.99 (d, J=8.9 Hz, 2H), 4.46 (s, 3H), 4.10 (s, 2H), 3.82-3.75 (m, 2H), 3.38 (dd, J=15.8, 10.3 Hz, 8H), 3.19 (d, J=9.7 Hz, 2H), 3.09 (s, 2H), 1.43 (s, 6H).

### Example 33. 8-Fluoro-2-(4-{[4-(4-{[2-(2-hydroxyethyl)-3-oxo-1-pyridin-2-yl-2,3-dihydro-1H-pyrazolo[3,4-d]pyrimidin-6-yl]amino}phenyl) piperazin-1-yl]methyl}phenyl)-1,3,4,5-tetrahydro-6H-azepino[5,4,3-cd]indol-6-one (43)

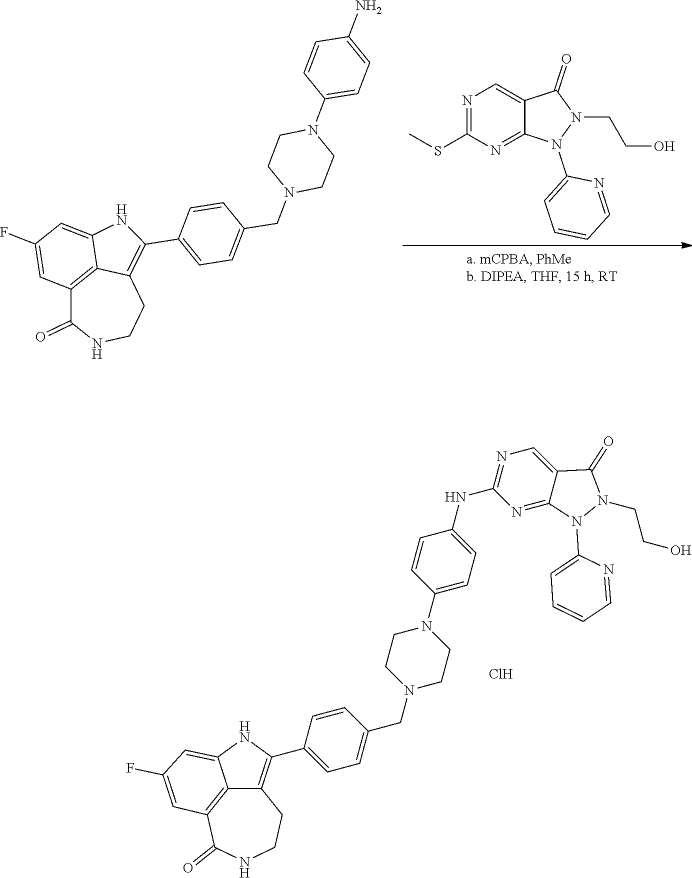 [SMILES: Nc1ccc(N2CCN(Cc3ccc(-c4[nH]c5cc(F)cc6c5c4CCNC6=O)cc3)CC2)cc1 ; CSc1ncc2c(=O)n(CCO)n(-c3ccccn3)c2n1 ; O=C1NCCc2c(-c3ccc(CN4CCN(c5ccc(Nc6ncc7c(=O)n(CCO)n(-c8ccccn8)c7n6)cc5)CC4)cc3)[nH]c3cc(F)cc1c23 ; Cl]

To a solution of 2-(2-hydroxyethyl)-6-(methylthio)-1-pyridin-2-yl-1,2-dihydro-3H-pyrazolo[3,4-d]pyrimidin-3-one (P19, 0.17 g, 0.39 mmol) in 2 mL of PhMe, 77% m-CPBA (0.13 g, 0.58 mmol) was added. The reaction mixture was stirred for 1 h at ambient temperature. After completion of the reaction as indicated by TLC, 2-(4-{[4-(4-aminophenyl) piperazin-1-yl]methyl}phenyl)-8-fluoro-1,3,4,5-tetrahydro-6H-azepino[5,4,3-cd]indol-6-one (P39, 0.18 g, 0.38 mmol) and DIPEA (0.5 mL) were added to mixture. The reaction mixture was stirred for 15 h at ambient temperature, and then concentrated. The residue was partitioned between dichloromethane and water. The organic layer was separated, washed with NaHCO₃, water and brine, dried over sodium sulfate and concentrated. The obtained crude residue was purified by RP-HPLC eluting with a gradient MeCN-H₂O+0.1% TFA to provide 8-fluoro-2-(4-{[4-(4-{[2-(2-hydroxyethyl)-3-oxo-1-pyridin-2-yl-2,3-dihydro-1H-pyrazolo[3,4-d]pyrimidin-6-yl]amino}phenyl) piperazin-1-yl]methyl}phenyl)-1,3,4,5-tetrahydro-6H-azepino[5,4,3-cd]indol-6-one (43, 0.065 g, 23%). 1H NMR (400 MHz, DMSO-d₆) δ 11.86 (s, 1H), 11.29 (s, 1H), 10.14 (s, 1H), 8.81 (s, 1H), 8.52(d, J=4.3 Hz, 1H), 8.29 (s, 1H), 8.08 (d, J=7.4 Hz, 1H), 7.93 (d, J=8.3 Hz, 1H), 7.81 (d, J=8.0 Hz, 2H), 7.74 (d, J=8.2 Hz, 2H), 7.62(s, 2H), 7.44 (dd, J=10.9, 2.2 Hz, 1H), 7.38 (t, J=8.3 Hz, 2H), 6.99 (d, J=8.7 Hz, 2H), 4.45 (s, 2H), 4.03 (s, 2H), 3.52-3.31 (m, 8H), 3.19 (d, J=8.7 Hz, 5H), 3.08 (s, 2H).

### Example 34. 5-Fluoro-1-(4-fluoro-3-{[4-(4-{[2-(2-methoxyethyl)-3-oxo-1-pyridin-2-yl-2,3-dihydro-1H-pyrazolo[3,4-d]pyrimidin-6-yl]amino}phenyl) piperazin-1-yl]carbonyl}benzyl) quinazoline-2,4 (1H,3H)-dione (23)

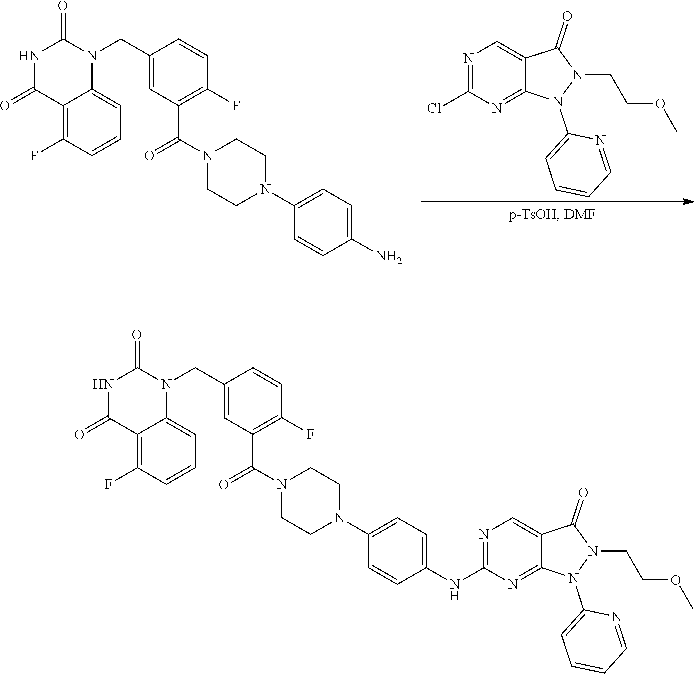 [SMILES: Nc1ccc(N2CCN(C(=O)c3cc(Cn4c(=O)[nH]c(=O)c5c(F)cccc54)ccc3F)CC2)cc1 ; COCCn1c(=O)c2cnc(Cl)nc2n1-c1ccccn1 ; COCCn1c(=O)c2cnc(Nc3ccc(N4CCN(C(=O)c5cc(Cn6c(=O)[nH]c(=O)c7c(F)cccc76)ccc5F)CC4)cc3)nc2n1-c1ccccn1]

To a solution of 1-(3-{[4-(4-aminophenyl) piperazin-1-yl]carbonyl}-4-fluorobenzyl)-5-fluoroquinazoline-2,4 (1H,3H)-dione (P3, 0.080 g, 0.16 mmol) and 6-chloro-2-(2-methoxyethyl)-1-pyridin-2-yl-1,2-dihydro-3H-pyrazolo[3,4-d]pyrimidin-3-one (P64, 0.045 g, 0.16 mmol) in 10 mL DMF, p-TsOH was added (0.028 g, 0.16 mmol) and the mixture was stirred at 80° C. for 15 h. The mixture was quenched with aq. NaHCO₃, extracted with CHCl₃, the combined organic extracts were dried with Na₂SO₄ and evaporated. The residue was washed with MeOH, MeCN and dried. The solid was further purified on silica gel eluting with 1-10% MeOH—CHCl₃5-fluoro-1-(4-fluoro-3-{[4-(4-{[2-(2-methoxyethyl)-3-oxo-1-pyridin-2-yl-2,3-dihydro-1H-pyrazolo[3,4-d]pyrimidin-6-yl]amino}phenyl) piperazin-1-yl]carbonyl}benzyl) quinazoline-2,4 (1H,3H)-dione (23, 0.052 g, 45%). ¹H NMR (400 MHz, DMSO-d₆) § 11.68 (s, 1H), 10.47-9.75 (m, 1H), 8.81 (s, 1H), 8.52 (s, 1H), 8.11 (s, 1H), 7.95 (s, 2H), 7.61 (s, 3H), 7.45 (s, 1H), 7.40 (s, 4H), 7.29 (s, 1H), 7.05 (d, J=7.9 Hz, 1H), 6.94 (d, J=8.5 Hz, 2H), 5.32(s, 2H), 4.15 (s, 2H), 3.78 (s, 2H), 3.30 (s, 2H), 3.17 (s, 2H), 3.08 (s, 3H), 2.98 (s, 2H).

### Example 35. 1-[3-({4-[4-({2-Allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)-4-fluorobenzyl]-5-fluoroquinazoline-2,4 (1H,3H)-dione (34)

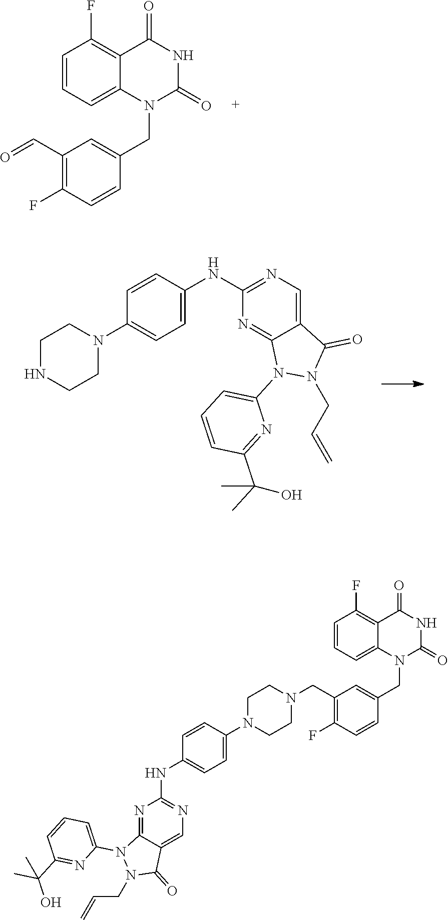 [SMILES: O=Cc1cc(Cn2c(=O)[nH]c(=O)c3c(F)cccc32)ccc1F ; C=CCn1c(=O)c2cnc(Nc3ccc(N4CCNCC4)cc3)nc2n1-c1cccc(C(C)(C)O)n1 ; C=CCn1c(=O)c2cnc(Nc3ccc(N4CCN(Cc5cc(Cn6c(=O)[nH]c(=O)c7c(F)cccc76)ccc5F)CC4)cc3)nc2n1-c1cccc(C(C)(C)O)n1]

To a mixture of 2-fluoro-5-[(5-fluoro-2,4-dioxo-3,4-dihydroquinazolin-1(2H)-yl)methyl]benzaldehyde (P66, 0.095 g, 0.30 mmol) and 2-allyl-1-[6-(1-hydroxy-1-methylethyl)pyridin-2-yl]-6-[(4-piperazin-1-ylphenyl)amino]-1,2-dihydro-3H-pyrazolo[3,4-d]pyrimidin-3-one (P60, 0.15 g, 0.30 mmol) in 20 mL MeOH, acetic acid was added (18 μl, 0.30 mmol) and the mixture was stirred for 1 h at ambient temperature. Sodium cyanoborohydride was added (0.11 g, 1.68 mmol) and the mixture was stirred at ambient temperature for 15 h. The mixture was quenched with aq. NaHCO₃, extracted with CHCl₃, the combined organic extracts were dried with Na₂SO₄ and evaporated. The obtained crude residue was purified by RP-HPLC eluting with a gradient MeCN-H₂O+0.1% TFA to provide 1-[3-({4-[4-({2-allyl-1-[6-(1-hydroxy-1-methylethyl)pyridin-2-yl]-3-oxo-2,3-dihydro-1H-pyrazolo[3,4-d]pyrimidin-6-yl}amino)phenyl]piperazin-1-yl}methyl)-4-fluorobenzyl]-5-fluoroquinazoline-2,4 (1H,3H)-dione (34, 0.011 g, 5%). ¹H NMR (400 MHz, DMSO-d₆) δ 11.72(s, 1H), 10.98 (s, 1H), 10.20 (s, 1H), 8.84 (s, 1H), 8.03 (t, J=7.8 Hz, 1H), 7.75 (d, J=8.1 Hz, 1H), 7.67 (d, J=5.1 Hz, 1H), 7.65-7.57 (m, 3H), 7.50 (s, 1H), 7.33 (t, J=9.1 Hz, 1H), 7.12-7.02(m, 2H), 6.96 (d, J=8.8 Hz, 2H), 5.89-5.57 (m, 1H), 5.32(s, 2H), 4.99 (d, J=10.2 Hz, 1H), 4.82(d, J=17.1 Hz, 1H), 4.68 (d, J=5.6 Hz, 2H), 4.40 (s, 2H), 3.70 (s, 2H), 3.39 (d, J=9.2 Hz, 4H), 3.24-2.98 (m, 4H), 1.46 (s, 6H).

### Example 36. 5-Fluoro-1-[4-fluoro-3-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}carbonyl)benzyl]quinazoline-2,4 (1H,3H)-dione (30)

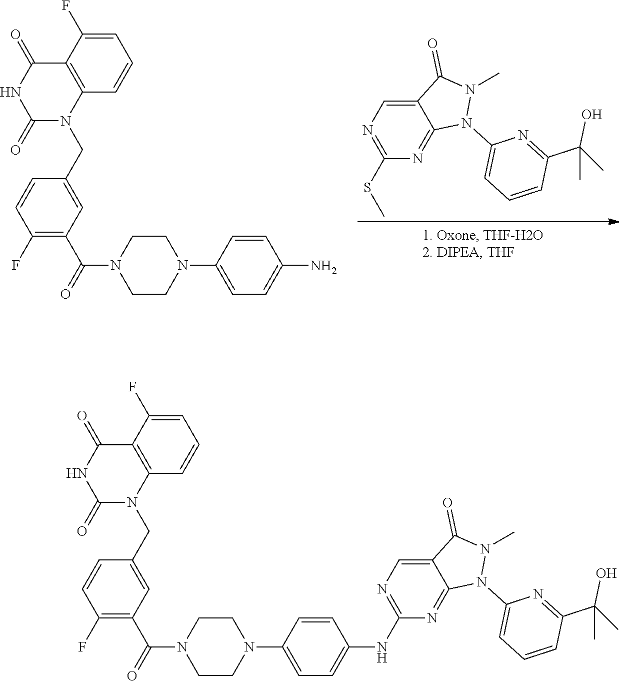 [SMILES: Nc1ccc(N2CCN(C(=O)c3cc(Cn4c(=O)[nH]c(=O)c5c(F)cccc54)ccc3F)CC2)cc1 ; CSc1ncc2c(=O)n(C)n(-c3cccc(C(C)(C)O)n3)c2n1 ; Cn1c(=O)c2cnc(Nc3ccc(N4CCN(C(=O)c5cc(Cn6c(=O)[nH]c(=O)c7c(F)cccc76)ccc5F)CC4)cc3)nc2n1-c1cccc(C(C)(C)O)n1]

To a mixture of 1-[6-(1-hydroxy-1-methylethyl)pyridin-2-yl]-2-methyl-6-(methylthio)-1,2-dihydro-3H-pyrazolo[3,4-d]pyrimidin-3-one (P47, 0.10 g, 0.30 mmol) in THF-H₂O, 2:1 (30 mL), Oxone was added (0.17 g, 0.60 mmol) and the mixture was stirred at ambient temperature for 2 h. The mixture was diluted with H₂O, extracted with CH₂Cl₂, the combined organic extracts were dried with Na₂SO₄ and evaporated. The obtained 1-[6-(1-hydroxy-1-methylethyl)pyridin-2-yl]-2-methyl-6-(methylsulfinyl)-1,2-dihydro-3H-pyrazolo[3,4-d]pyrimidin-3-one (0.082 g) was dissolved in THF, and 1-(3-{[4-(4-aminophenyl) piperazin-1-yl]carbonyl}-4-fluorobenzyl)-5-fluoroquinazoline-2,4 (1H,3H)-dione was added (P8, 0.12 g, 0.24 mmol) followed by addition of DIPEA (0.31 g, 2.3 mmol) and the mixture was stirred art ambient temperature for 15 h. The mixture was quenched with aq. NaHCO₃, extracted with CHCl₃, the combined organic extracts were dried with Na₂SO₄ and evaporated. The obtained crude residue was purified by RP-HPLC eluting with a gradient MeCN-H₂O+0.1% TFA to provide 5-fluoro-1-[4-fluoro-3-({4-[4-({1-[6-(1-hydroxy-1-methylethyl)pyridin-2-yl]-2-methyl-3-oxo-2,3-dihydro-1H-pyrazolo[3,4-d]pyrimidin-6-yl}amino)phenyl]piperazin-1-yl}carbonyl)benzyl]quinazoline-2,4 (1H,3H)-dione (30, 0.034 g, 19%). ¹H NMR (400 MHz, DMSO-d₆) δ 11.69 (s, 1H), 10.13 (s, 1H), 8.82(s, 1H), 8.08 (s, 1H), 7.80 (s, 1H), 7.62(s, 4H), 7.42(s, 2H), 7.30 (s, 1H), 7.04 (s, 2H), 6.94 (s, 2H), 5.32 (s, 2H), 3.78 (s, 2H), 3.30 (s, 4H), 3.18 (s, 4H), 2.98 (s, 2H), 1.45 (s, 6H).

Other examples presented in the Table 1 were obtained using procedures described above with slight modifications or without them using appropriate building blocks described in preparative part.

### Biological Assays

Example A. Primary Assay used to determine potency of Wee1 enzymatic activity inhibition.

Compound activity was determined using recombinant Wee1 protein (BPS, Cat #40412) and PolyE4Y1 Substrate (Poly(Glu,Tyr) sodium salt, Sigma, Cat #P0275-25 MG) in an in vitro enzymatic reaction. The enzymatic assay used to determine activity was a Luminescence assay using a Microplate Reader (BMG, ClarioStar Plus). The enzymatic reaction was carried out in assay buffer (40 mM TRIS-HCl pH 7.4-7.6, 20 mM MgCl₂, 0.05 mM DTT, 0.1 mg/ml BSA, 5 mM MnCl₂). The compounds were dispensed on a 384 well Diamond Well Plate (Axigen, Cat #P-384-120 SQ-C—S) using the Biomek FX liquid handling system at 100× solutions of compounds in DMSO. 2× Wee1-PolyE4Y1 mix (final concentration 0.85 ng/μl of Wee1 and 0.2 μg/μl of PolyE4Y1) was prepared in 1× Assay buffer and 5.5 μl of mixture per well was added into 384w white Reaction plate with NBS (Corning, Cat #4513). 5.5 μl of PolyE4Y1 substrate w/o Wee1 in 1× buffer was used for negative control. Plates were centrifuged for 1 min at 100 g. Next step the Compounds were added to Reaction plate using Biomek station via following steps: 1 ul of 100× compounds (in DMSO) were mixed thoroughly with 49 μl of 2×10 μM ATP in Assay Buffer, then 5.5 μl of this mixture was added to Reaction plate with 5.5 μl of Wee1-PolyE4Y1 mix. Plates were centrifuged for 1 min at 100 g and incubated for 1 h at room temperature. Next 3 μL of ADP-Glo reagent (Promega, ADP-Glo™ Kinase Assay, Cat #V9102) per well was added. Plates were incubated for 30 minutes at room temperature. Then 6 μL of Kinase detection reagent (Promega, ADP-Glo™ Kinase Assay, Cat #V9102) per well was added and the Luminescence was measured using Microplate Reader. The % inhibition was then used to calculate the IC₅₀ values. The IC₅₀ values are shown in Table A, wherein “A” corresponds to IC₅₀<0.01 μM, “B” corresponds to 0.01 UM≤IC₅₀<0.1 μM, “C”0.1 UM≤IC₅₀<1 μM, and “D” corresponds to 1 nM≤IC₅₀.

TABLE A

| Compound No. | Wee1 EC₅₀ (μM) |
|---|---|
| 1 | A |
| 2 | D |
| 3 | C |
| 4 | D |
| 5 | D |
| 6 | D |
| 39 | A |
| 7 | A |
| 16 | A |
| 96 | A |
| 28 | A |
| 34 | A |
| 8 | A |
| 10 | B |
| 83 | B |
| 18 | B |
| 95 | B |
| 35 | B |
| 11 | B |
| 33 | C |
| 29 | C |
| 12 | D |
| 17 | D |
| 47 | D |
| 44 | D |
| 36 | B |
| 40 | B |
| 38 | C |
| 13 | B |
| 14 | B |
| 9 | B |
| 15 | C |
| 97 | C |
| 86 | C |
| 91 | D |

Example B. Tracer Displacement Fluorescence Polarization Assays. Displacement of AlexaFlour488 marked tracer from the NAD+-binding site of recombinant PARP1(AA662-1014) protein (OriGene, Cat #: AR09368PU-L) was measured in vitro using Fluorescence polarization assay. The reaction was carried out in assay buffer (50 mM TRIS-HCl pH 7.4-7.8, 150 mM NaCl, 10 mM MgCl₂, 0.001% Triton-X). 2×PARP1(final concentration 25 nM) was diluted in 1× Assay buffer and 10 μl of the solution per well was added into 384-well Black Non-Treated Reaction plate (Corning, Cat #4511). Plates were centrifuged for 1 min at 200 g. The compounds were prepared in a 384-well Diamond Well Plate (Axygen, Cat #P-384-120 SQ-C—S) as 400× solutions in DMSO and were transferred into Reaction plate using the Biomek FX liquid handling system with intermediate dilution down to 10× working solution in assay buffer in a separate 384-well Diamond Well Plate (Axygen, Cat #P-384-120 SQ-C—S) (2 μl per well). Plates were centrifuged for 1 min at 200 g and incubated for 15 min at room temperature in the dark. Then 8 μL per well of 2.5× Tracer AlexaFluor488 (final concentration 1 nM) diluted in 1× Assay buffer was added into Reaction plate. 1× Assay buffer was used to make blank samples for instrument calculations. Plates were incubated for 30 minutes at room temperature in the dark. Fluorescence polarization was measured using Microplate Reader (BMG, ClarioStar Plus). The % tracer displacement was used to calculate the IC₅₀ values. The IC₅₀ values are shown in Table A, wherein “A” corresponds to IC₅₀<0.01 μM, “B” corresponds to 0.01 UM≤IC₅₀<0.1 μM, “C” 0.1 μM≤IC₅₀<1 μM, and “D” corresponds to 1 nM≤IC₅₀.

TABLE B

| Compound No. | PARP1 EC₅₀ (μM) |
|---|---|
| 1 | A |
| 2 | B |
| 3 | A |
| 4 | C |
| 5 | B |
| 6 | C |
| 39 | B |
| 7 | A |
| 16 | A |
| 96 | B |
| 28 | A |
| 34 | C |
| 8 | C |
| 10 | A |
| 83 | B |
| 18 | A |
| 95 | B |
| 35 | B |
| 11 | A |
| 33 | D |
| 29 | A |
| 12 | A |
| 17 | A |
| 47 | A |
| 44 | A |
| 36 | A |
| 40 | B |
| 38 | B |
| 13 | A |
| 14 | C |
| 9 | A |
| 15 | A |
| 97 | B |
| 86 | B |
| 91 | B |

### Example C. Trapping Assay Used to Determine Potency of PARP1 enzymatic activity Inhibition

Compound activity was determined using recombinant PARP1 protein (Antibodies-online, Cat #: ABIN1741729), actDNA (ChemRar, prepared from dsDNA with 1:2 For/Rev ratio) and NAD+ (Sigma-Aldrich, Cat #N1511) in an in vitro enzymatic reaction. The enzymatic assay used to determine activity was a Trapping assay. The enzymatic reaction was carried out in assay buffer (50 mM TRIS-HCl pH 7.4-7.8, 100 mM NaCl, 4 mM MgCl₂, 100 ng/μl BSA). 10 μl per well of 2×PARP1(final concentration 10 nM) diluted in 1× Assay buffer was added into 384-well Black Reaction plate with Non-Binding Surface (Corning, Cat #3676). Plates were centrifuged for 1 min at 200 g. The compounds were prepared in a 384-well Diamond Well Plate (Axygen, Cat #P-384-120 SQ-C—S) as 400× solutions in DMSO and were transferred into Reaction plate using the Biomek FX liquid handling system with intermediate dilution down to 10× working solution in assay buffer in a separate 384-well Diamond Well Plate (Axygen, Cat #P-384-120 SQ-C—S) (2 μl per well). Plates were centrifuged for 1 min at 200 g and incubated for 15 min at room temperature in the dark. Next 4 μL per well of 5× actDNA (final concentration 0.25 nM) diluted in 1× Assay buffer was added into Reaction plate. Plates were centrifuged for 1 min at 200 g and were incubated for 30 minutes at room temperature in the dark. Then 4 μL per well of 5×NAD+ (final concentration 0.5 nM) diluted in 1× Assay buffer was added into Reaction plate. 1× Assay buffer was used to make blank samples for instrument calculations. Plates were centrifuged for 1 min at 200 g and were incubated for 1 h at room temperature in the dark. Fluorescence polarization was measured using Microplate Reader (BMG, ClarioStar Plus). The % inhibition was then used to calculate the IC₅₀ values.

### Example D. Preparation of PARP1 dsDNA substrate (https://doi.org/10.1101/2020.10.18.333070)

Single stranded oligonucleotides were hybridized by combining in 1:2 For/Rev ratio of the following sequences:

For:
5′-AlexaFluor488-ACCCTGCTGTGGGCdUGGAGAACAAGGTGAT-3′
Rev:
5′-ATCACCTTGTTCTCCAGCCCACAGCAGGGT-3′

This mixture was then heated to 95° C. for 5 min and slowly cooled to room temperature. Hybridized oligonucleotide (“dsDNA”) was then incubated with APE1(NEB, Cat #: M0282S) and UDG (NEB, Cat #: M0280S) in NEBuffer 4 (NEB, Cat #: B7004S) at 37° C. for 1 h to create a single strand break (“actDNA”) recognized by the PARP1 enzyme.

### Example E. Cellular Growth Inhibition Assay

HEK293 (Institute of Cytology Russian Academy of Science), MDA-MB-231 (ATCC, CRM-HTB-26), HCC1395 (ATCC, CRL-2324), HCC1937 (ATCC, CRL-2336), UWB1.289+BRCA1 (ATCC, CRL-2946) were seeded at a density of 500 cells per well in a 384-well clear bottom plate (Greiner Cat #781090) in 45 μl total volume of DMEM (PanEco, Cat #C420) or RPMI (PanEco, Cat #C330) or RPMI/MEBM (Lonza, Cat #CC-3151+CC-4136) with 10% FBS (HyClone, Cat #SV30160.03). Cells were allowed to adhere overnight at 37° C., 5% CO₂. 500× compounds solutions in DMSO (Sigma Cat #D2650) were prepared into Cmpnds plate (Diamond Well Plate, Axigen, Cat #P-384-120 SQ-C—S) and DMSO only control was included. 1 ul of 500× compounds (Cmpnds plate) was added to 49 μl of culture medium into Dilution plate (Diamond Well Plate, Axigen, Cat #P-384-120 SQ-C—S), mixed and then 5 μl of 10× compounds solutions were transferred to cells followed by centrifugation at 100 g for 1 min. Final DMSO concentration was 0.2%. After 3 days of incubation, 10 μl of 1× compounds were added to cells. After 7 days of incubation, 12 μl of CellTiter-Glo (Promega, CAT #G7572) were added to the cells, plate was centrifuged at 100 g for 1 min and luminescence signal was measured using Microplate Reader (CLARIOStar). The IC₅₀ values are shown in Table E.

TABLE E

| | MDA-MB-231 EC₅₀ (μM) | | |
|---|---|---|---|
| Compound No. | 5 days | 7 days | 10 days |
| 1 | 0.69 | 0.66 | 0.11 |
| 16 | 0.96 | 0.8 | 0.18 |
| 35 | 0.98 | 0.57 | 0.08 |
| 3 | 3.75 | — | — |
| 33 | >10 | — | — |
| 18 | >10 | — | — |
| 96 | 2.77 | — | — |
| 10 | 4.89 | 2.59 | 1.49 |
| 11 | 8.76 | 6.28 | 3.6 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (I): 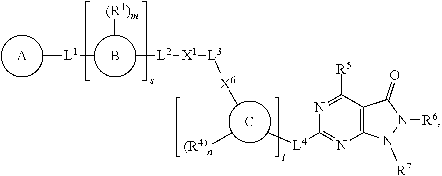
or a pharmaceutically acceptable salt, stereoisomer, solvate, or tautomer thereof, wherein
Ring A is 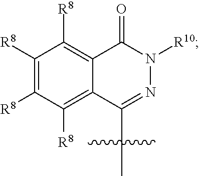
Ring B and Ring C are each independently selected from C₃-C₁₀ cycloalkyl, aryl, 3- to 10-membered heterocyclyl, and heteroaryl;
L¹, L², and L⁴ are independently selected from bond, C₁-C₆ alkylenyl, C₂-C₆ alkenylenyl, C₂-C₆ alkynylenyl, C(O)—, C(O)NR^{L}—, —C(O)O, —NR^{L}—, —NR^{L}C(O)—, —NR^{L}C(O)NR^{L}—, —NR^{L}C(O)O—, —O—, —OC(O)—, OC(O)NR^{L}—, —OC(O)O—, and —S(O)ₒ—, wherein the alkylenyl, alkenylenyl, and alkynylenyl is optionally substituted with one or more substituents independently selected from halogen, —OH, —CN, —NO₂, C₁-C₆ alkyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
L³ is selected from C₁-C₆ alkylenyl, C₂-C₆ alkenylenyl, and C₂-C₆ alkynylenyl, wherein the alkylenyl, alkenylenyl, and alkynylenyl is optionally substituted with one or more halogen, —OH, —CN, —NO₂, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
provided that when s is 0 then L¹ and L² form a single bond;
each R^{L} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
X¹ is selected from NR², CR², and C(R²)₂;
X⁶ is selected from NR³, CR³, and C(R³)₂;
R¹ and R⁴ are each independently selected from halogen, OH, CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
R² and R³ are each independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² and R³, together with the atoms to which they are attached, may come together to form 4- to 10-membered heterocyclyl, aryl, or heteroaryl, optionally substituted with one or more substituents independently selected from halogen, OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² may come together with any part of L² to form 3- to 10-membered heterocyclyl or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R² may come together with any part of L³ to form 3- to 10-membered heterocyclyl or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
or R³ may come together with any part of L³ to form 3- to 10-membered heterocyclyl, aryl, or heteroaryl, optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
R⁵ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
each R⁶, R⁷, and R¹⁰ is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more R¹¹;
R⁸ is selected from hydrogen, halogen, —OH, —CN, —NO₂, —C(O)R^{x}, —C(O)N(R^{x})₂, —C(O)OR^{x}, —N(R^{x})₂, —OR^{x}, —S(O)ₒR^{x}, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
each R^{X} is independently selected from hydrogen, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl, wherein the alkyl, alkenyl, alkynyl, alkoxy, cycloalkyl, aryl, heterocyclyl, or heteroaryl is optionally substituted with one or more substituents independently selected from halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl;
m is an integer selected from 0, 1, 2, 3, 4, 5, and 6;
n is an integer selected from 0, 1, 2, 3, 4, 5, and 6;
o is an integer selected from 0, 1, and 2;
s is an integer selected from 0 and 1;
t is an integer selected from 0 and 1;
wherein
aryl is cyclic, aromatic hydrocarbon groups that have 1 to 3 aromatic rings where the aromatic rings may be joined at a single point, or fused;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is monovalent monocyclic or polycyclic aromatic radical of 5 to 24 ring atoms, containing one or more ring heteroatoms selected from N, O, S, P, Se, or B, the remaining ring atoms being C.

2. The compound of claim 1, wherein the compound is of Formula (I-E): 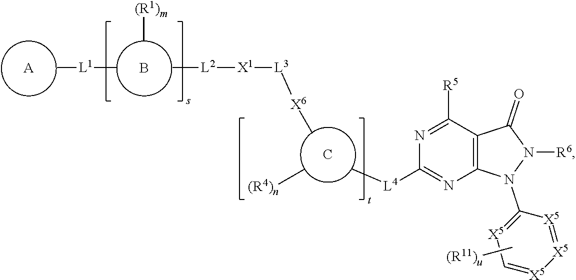
or a pharmaceutically acceptable salt, stereoisomer, solvate, or tautomer thereof, wherein each X⁵ is independently selected from CR¹¹ and N; each R¹¹ is independently selected from hydrogen, halogen, —OH, —CN, —NO₂, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, aryl, 3- to 10-membered heterocyclyl, and heteroaryl; and u is an integer selected from 0, and 1.

3. The compound of claim 1, wherein Ring A is

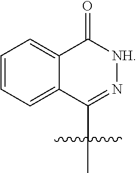 [SMILES: *c1n[nH]c(=O)c2ccccc12]

4. The compound of claim 1, wherein Ring B is aryl.

5. The compound of claim 1, wherein Ring B is selected from

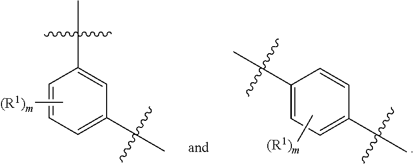

6. The compound of claim 1, wherein Ring B is selected from

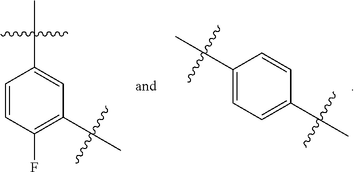 [SMILES: *c1ccc(F)c(*)c1 ; *c1ccc(*)cc1]

7. The compound of claim 1, wherein s is 0.

8. The compound of claim 1, wherein s is 1.

9. The compound of claim 1, wherein Ring C is aryl.

10. The compound of claim 1, wherein Ring C is

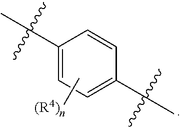

11. The compound of claim 1, wherein Ring C is

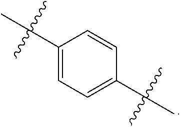 [SMILES: *c1ccc(*)cc1]

12. The compound of claim 1, wherein t is 0.

13. The compound of claim 1, wherein t is 1.

14. The compound of claim 1, wherein L¹ is bond.

15. The compound of claim 1, wherein L¹ is C₁-C₆ alkylenyl.

16. The compound of claim 1, wherein L² is bond.

17. The compound of claim 1, wherein L² is C₁-C₆ alkylenyl.

18. The compound of claim 1, wherein L² is —C(O)—.

19. The compound of claim 1, wherein L³ is C₁-C₆ alkylenyl.

20. The compound of claim 1, wherein L⁴ is bond.

21. The compound of claim 1, wherein L⁴ is —NR^{L}—.

22. The compound of claim 1, wherein L⁴ is —NH—.

23. A compound selected from: 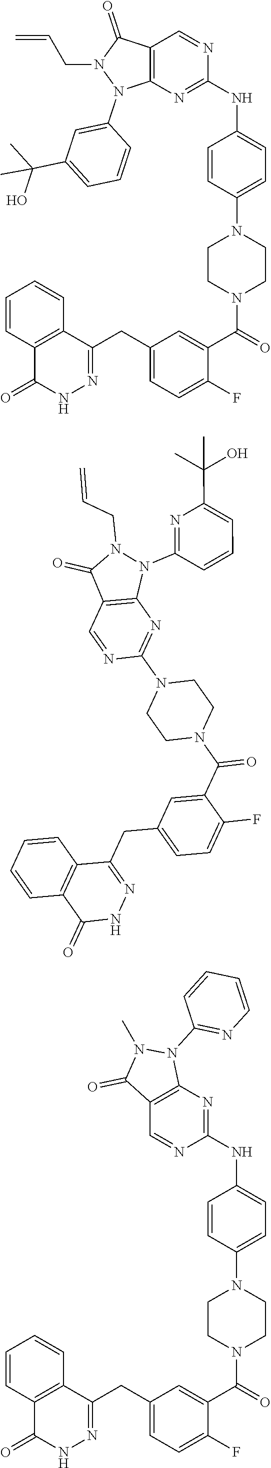 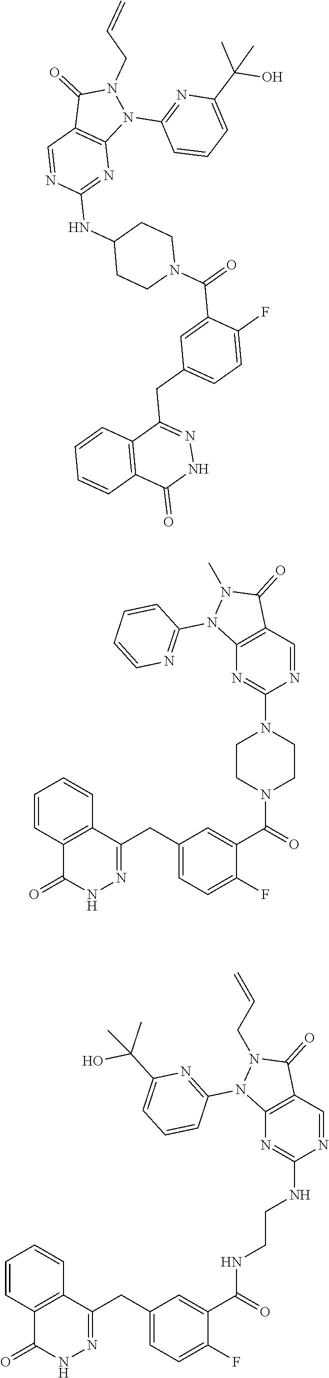 [SMILES: C=CCn1c(=O)c2cnc(NC3CCN(C(=O)c4cc(Cc5n[nH]c(=O)c6ccccc56)ccc4F)CC3)nc2n1-c1cccc(C(C)(C)O)n1 ; Cn1c(=O)c2cnc(N3CCN(C(=O)c4cc(Cc5n[nH]c(=O)c6ccccc56)ccc4F)CC3)nc2n1-c1ccccn1 ; C=CCn1c(=O)c2cnc(NCCNC(=O)c3cc(Cc4n[nH]c(=O)c5ccccc45)ccc3F)nc2n1-c1cccc(C(C)(C)O)n1] 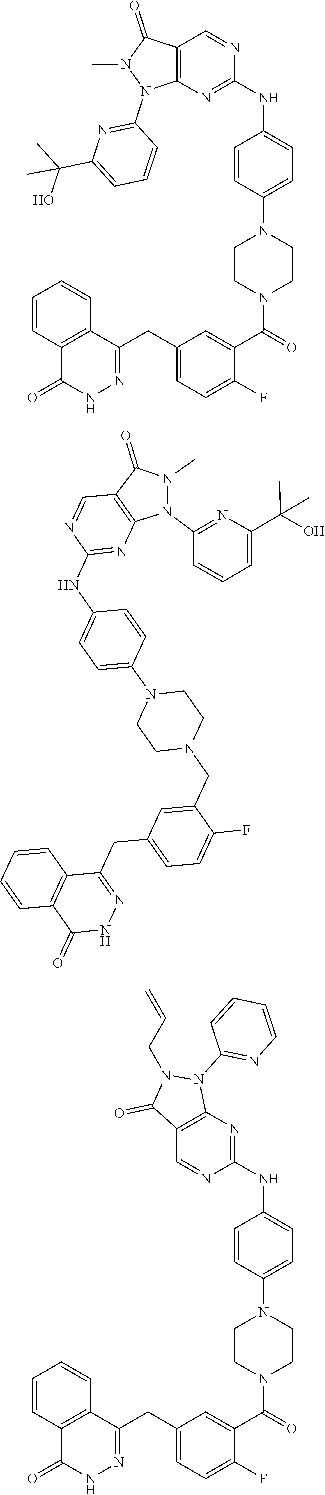 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)n1 ; Cn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1 ; C=CCn1c(=O)c2cnc(Nc3ccc(N4CCN(C(=O)c5cc(Cc6n[nH]c(=O)c7ccccc67)ccc5F)CC4)cc3)nc2n1-c1ccccn1] 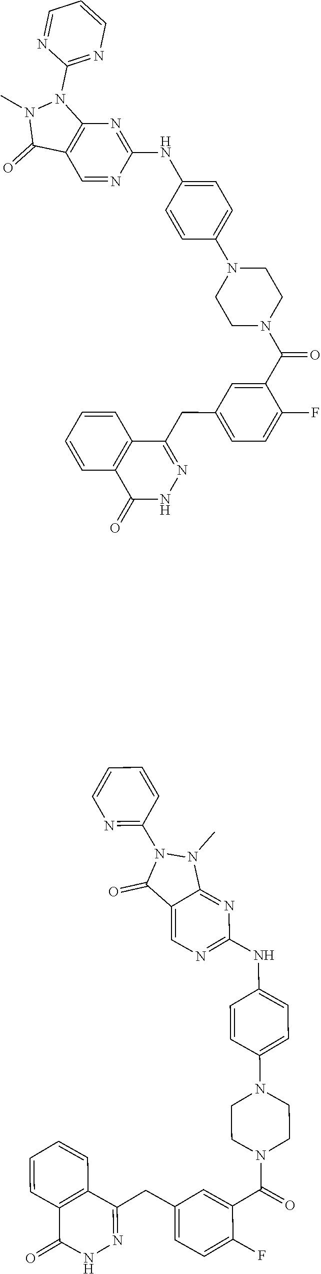 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1ncccn1 ; Cn1c2nc(Nc3ccc(N4CCN(C(=O)c5cc(Cc6n[nH]c(=O)c7ccccc67)ccc5F)CC4)cc3)ncc2c(=O)n1-c1ccccn1] 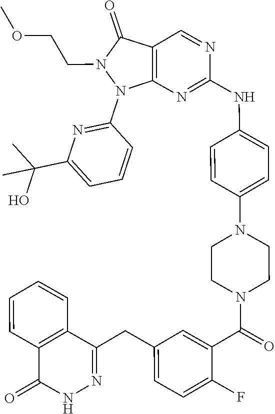 [SMILES: COCCn1c(=O)c2cnc(Nc3ccc(N4CCN(C(=O)c5cc(Cc6n[nH]c(=O)c7ccccc67)ccc5F)CC4)cc3)nc2n1-c1cccc(C(C)(C)O)n1] 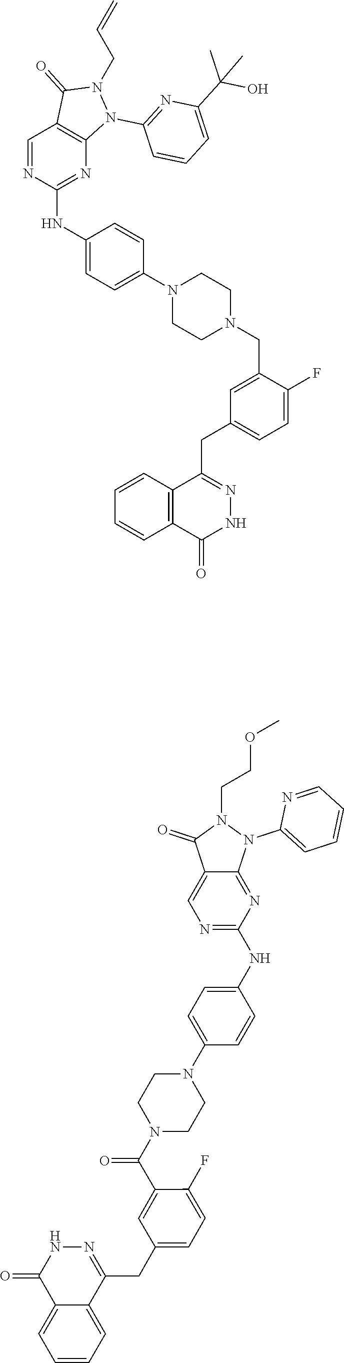 [SMILES: C=CCn1c(=O)c2cnc(Nc3ccc(N4CCN(Cc5cc(Cc6n[nH]c(=O)c7ccccc67)ccc5F)CC4)cc3)nc2n1-c1cccc(C(C)(C)O)n1 ; COCCn1c(=O)c2cnc(Nc3ccc(N4CCN(C(=O)c5cc(Cc6n[nH]c(=O)c7ccccc67)ccc5F)CC4)cc3)nc2n1-c1ccccn1] 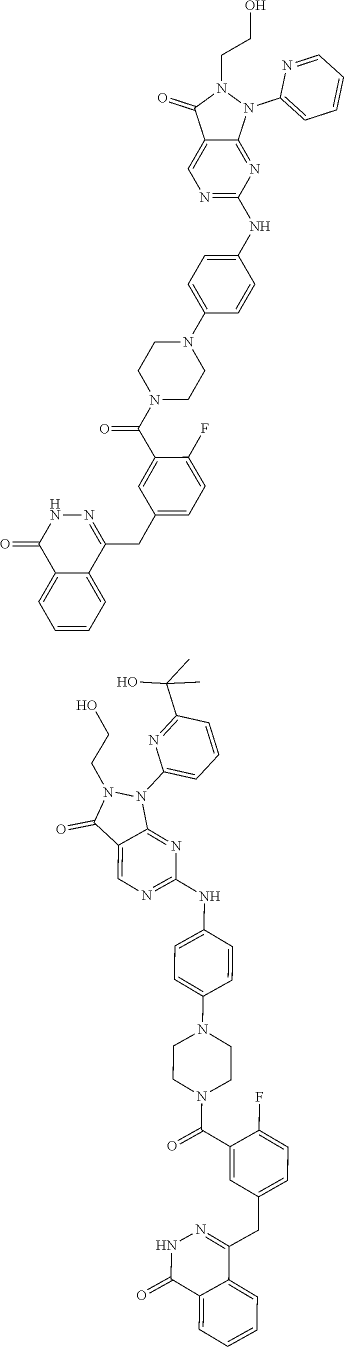 [SMILES: O=C(c1cc(Cc2n[nH]c(=O)c3ccccc23)ccc1F)N1CCN(c2ccc(Nc3ncc4c(=O)n(CCO)n(-c5ccccn5)c4n3)cc2)CC1 ; CC(C)(O)c1cccc(-n2c3nc(Nc4ccc(N5CCN(C(=O)c6cc(Cc7n[nH]c(=O)c8ccccc78)ccc6F)CC5)cc4)ncc3c(=O)n2CCO)n1] 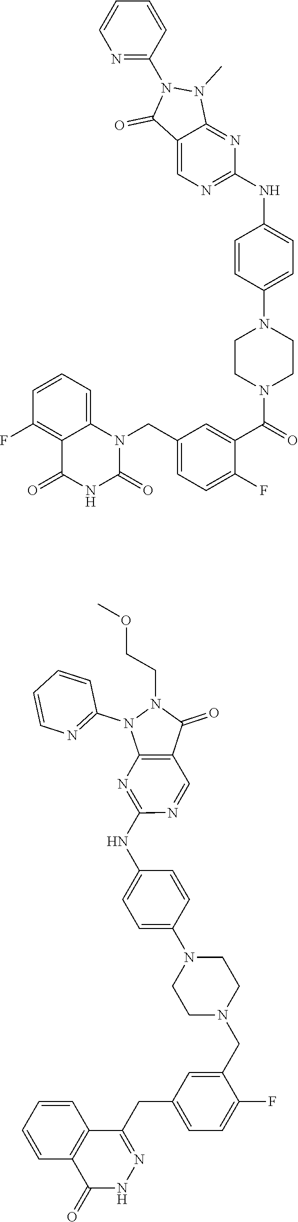 [SMILES: Cn1c2nc(Nc3ccc(N4CCN(C(=O)c5cc(Cn6c(=O)[nH]c(=O)c7c(F)cccc76)ccc5F)CC4)cc3)ncc2c(=O)n1-c1ccccn1 ; COCCn1c(=O)c2cnc(Nc3ccc(N4CCN(Cc5cc(Cc6n[nH]c(=O)c7ccccc67)ccc5F)CC4)cc3)nc2n1-c1ccccn1] 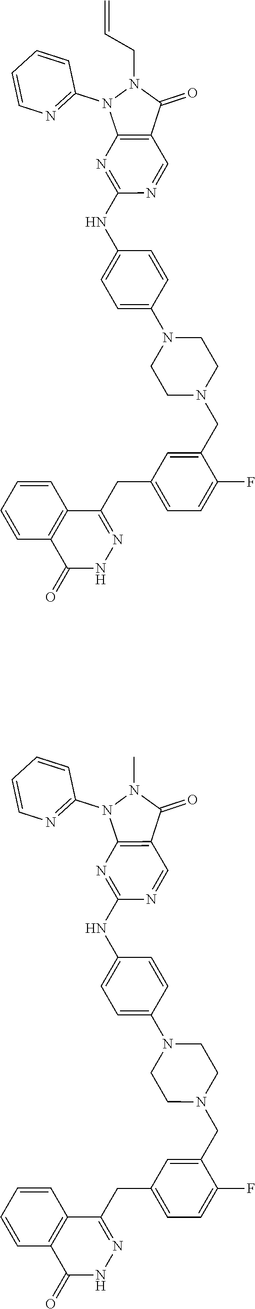 [SMILES: C=CCn1c(=O)c2cnc(Nc3ccc(N4CCN(Cc5cc(Cc6n[nH]c(=O)c7ccccc67)ccc5F)CC4)cc3)nc2n1-c1ccccn1 ; Cn1c(=O)c2cnc(Nc3ccc(N4CCN(Cc5cc(Cc6n[nH]c(=O)c7ccccc67)ccc5F)CC4)cc3)nc2n1-c1ccccn1] 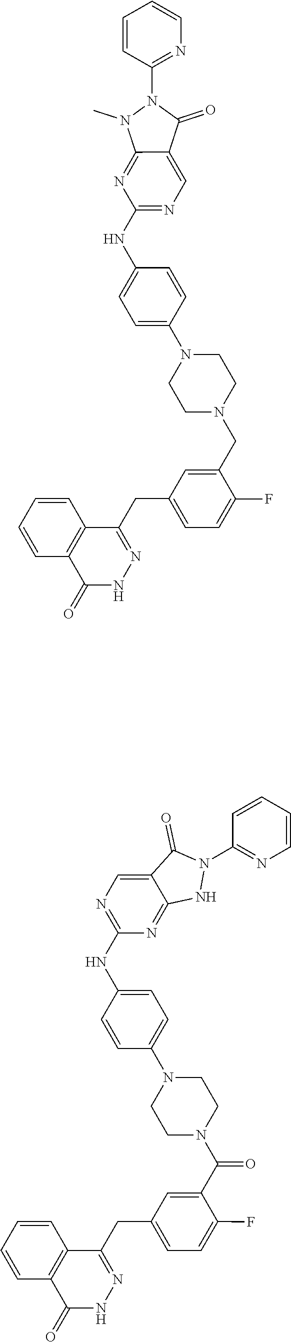 [SMILES: Cn1c2nc(Nc3ccc(N4CCN(Cc5cc(Cc6n[nH]c(=O)c7ccccc67)ccc5F)CC4)cc3)ncc2c(=O)n1-c1ccccn1 ; O=C(c1cc(Cc2n[nH]c(=O)c3ccccc23)ccc1F)N1CCN(c2ccc(Nc3ncc4c(=O)n(-c5ccccn5)[nH]c4n3)cc2)CC1] 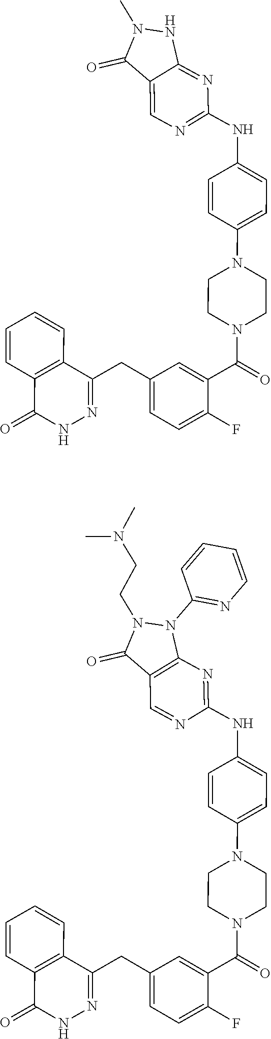 [SMILES: Cn1[nH]c2nc(Nc3ccc(N4CCN(C(=O)c5cc(Cc6n[nH]c(=O)c7ccccc67)ccc5F)CC4)cc3)ncc2c1=O ; CN(C)CCn1c(=O)c2cnc(Nc3ccc(N4CCN(C(=O)c5cc(Cc6n[nH]c(=O)c7ccccc67)ccc5F)CC4)cc3)nc2n1-c1ccccn1] 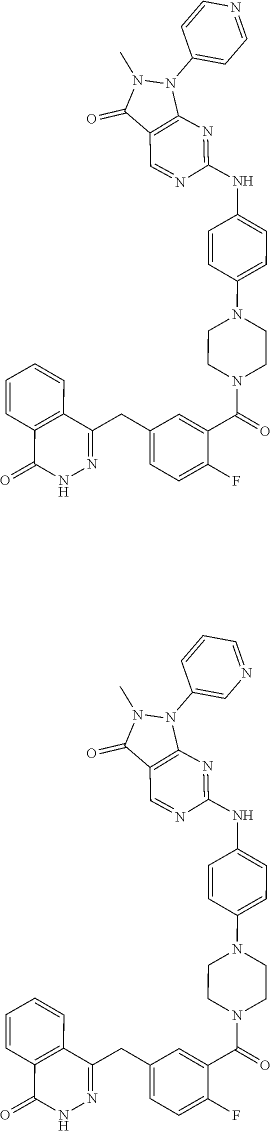 [SMILES: Cn1c(=O)c2cnc(Nc3ccc(N4CCN(C(=O)c5cc(Cc6n[nH]c(=O)c7ccccc67)ccc5F)CC4)cc3)nc2n1-c1ccncc1 ; Cn1c(=O)c2cnc(Nc3ccc(N4CCN(C(=O)c5cc(Cc6n[nH]c(=O)c7ccccc67)ccc5F)CC4)cc3)nc2n1-c1cccnc1] 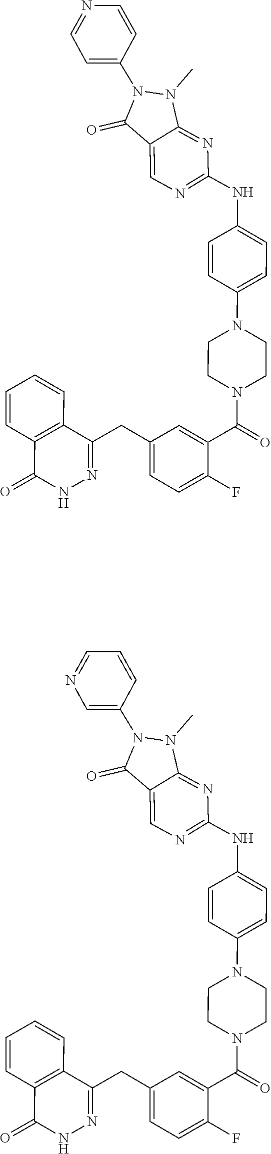 [SMILES: Cn1c2nc(Nc3ccc(N4CCN(C(=O)c5cc(Cc6n[nH]c(=O)c7ccccc67)ccc5F)CC4)cc3)ncc2c(=O)n1-c1ccncc1 ; Cn1c2nc(Nc3ccc(N4CCN(C(=O)c5cc(Cc6n[nH]c(=O)c7ccccc67)ccc5F)CC4)cc3)ncc2c(=O)n1-c1cccnc1] 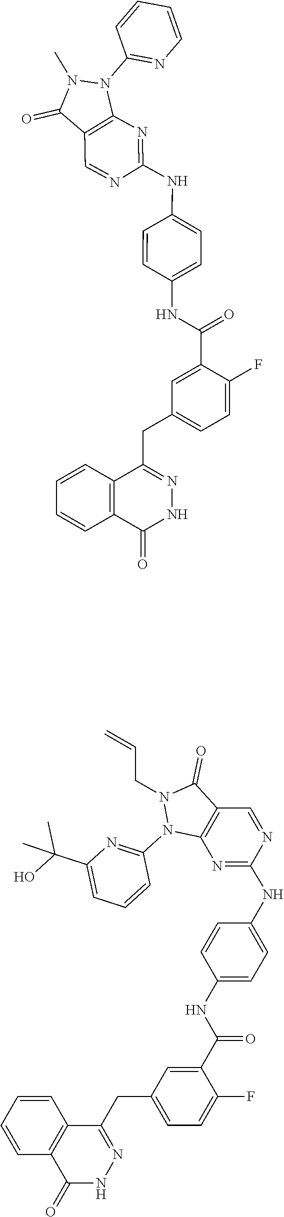 [SMILES: Cn1c(=O)c2cnc(Nc3ccc(NC(=O)c4cc(Cc5n[nH]c(=O)c6ccccc56)ccc4F)cc3)nc2n1-c1ccccn1 ; C=CCn1c(=O)c2cnc(Nc3ccc(NC(=O)c4cc(Cc5n[nH]c(=O)c6ccccc56)ccc4F)cc3)nc2n1-c1cccc(C(C)(C)O)n1] 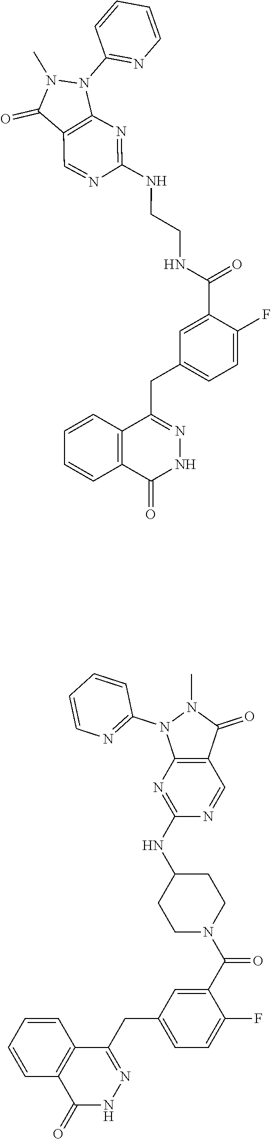 [SMILES: Cn1c(=O)c2cnc(NCCNC(=O)c3cc(Cc4n[nH]c(=O)c5ccccc45)ccc3F)nc2n1-c1ccccn1 ; Cn1c(=O)c2cnc(NC3CCN(C(=O)c4cc(Cc5n[nH]c(=O)c6ccccc56)ccc4F)CC3)nc2n1-c1ccccn1] 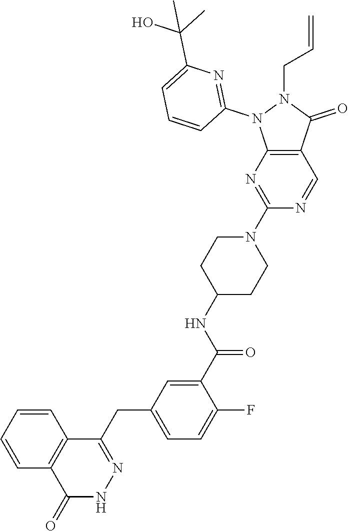 [SMILES: C=CCn1c(=O)c2cnc(N3CCC(NC(=O)c4cc(Cc5n[nH]c(=O)c6ccccc56)ccc4F)CC3)nc2n1-c1cccc(C(C)(C)O)n1]
or a pharmaceutically acceptable salt, stereoisomer, solvate, or tautomer thereof.

24. A pharmaceutical composition comprising a compound of Formula I or a pharmaceutically acceptable salt, stereoisomer, solvate, or tautomer thereof, and a pharmaceutically acceptable carrier.

25. The pharmaceutical composition of claim 24 further comprising one or more additional pharmaceutically active agents.

26. A method of inhibiting a poly ADP-ribose polymerase (PARP) enzyme and Wee1 in a cell, comprising contacting the cell with a compound of Formula I or a pharmaceutical composition of claim 24.

27. A method for the treatment or prevention of a disease or disorder associated with the inhibition of a poly ADP-ribose polymerase (PARP) enzyme and Wee1, comprising administering to a subject in need thereof a compound of Formula I or a pharmaceutical composition of claim 24.

28. A method of inhibiting a poly ADP-ribose polymerase (PARP) enzyme in a cell, comprising contacting the cell with a compound of Formula I or a pharmaceutical composition of claim 24.

29. A method of the treatment or prevention of a disease or disorder associated with the inhibition of a poly ADP-ribose polymerase (PARP) enzyme, comprising administering to a subject in need thereof a compound of Formula I or a pharmaceutical composition of claim 24.

30. The method of any one of claims 26-29, wherein the PARP enzyme is selected from PARP1, PARP 2, PARP3, PARP4, PARP-5A, PARP-5B, PARP6, PARP7, PARP8, PARP9, PARP10, PARP11, PARP12, PARTP14, PARP15, and PARP16.

31. The method of claim 30, wherein the PARP enzyme is PARP1.

32. The method of claim 30, wherein the PARP enzyme is PARP2.

33. A method of inhibiting Wee1 in a cell, comprising contacting the cell with a compound of Formula I or a pharmaceutical composition of 24.

34. A method of the treatment or prevention of a disease or disorder associated with the inhibition of Wee1, comprising administering to a subject in need thereof a compound of Formula I or a pharmaceutical composition of claim 24.

35. The method of any one of claims 27, 29, and 34, wherein the disease or disorder is cancer.

36. The method of claim 35, wherein the cancer is selected from bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, marginal zone lymphoma (MZL), follicular lymphoma (FL), diffuse large B-cell lymphoma (DLBCL), fallopian tube cancer, peritoneal cancer, and chronic lymphocytic leukemia/small lymphocytic lymphoma (CLL/SLL).
